(19) United States
(12) Patent Application Publication
(10) Pub. No.: US 2024/0182876 A1
(43) Pub. Date: Jun. 6, 2024

(54) Engineered Terminal Deoxynucleotidyl Transferase Variants

(71) Applicant: Codexis, Inc., Redwood City, CA (US)

(72) Inventors: David Entwistle, San Carlos, CA (US); Stephanie Forget, Redwood City, CA (US); Michelle Li, Millbrae, CA (US); Niusha Mahmoodi, San Francisco, CA (US); Ryan Reeves, Redwood City, CA (US); Ljubica Vojcic, San Jose, CA (US); Jonathan Vroom, South San Francisco, CA (US); David Watts, San Mateo, CA (US); Leland Ken Wong, San Bruno, CA (US)

(73) Assignee: Codexis, Inc., Redwood City, CA (US)

(21) Appl. No.: 18/485,855

(22) Filed: Oct. 12, 2023

Related U.S. Application Data
(60) Provisional application No. 63/379,439, filed on Oct. 13, 2022.
(60) Provisional application No. 63/499,770, filed on May 3, 2023.

(51) Int. Cl. C12N 9/12; C12N 15/63

(52) U.S. Cl. CPC C12N 9/1264; C12N 15/63; C12Y 207/07031

(57) ABSTRACT

The present invention provides engineered terminal deoxynucleotidyl transferase (TdT) polypeptides useful in template-independent polynucleotide synthesis, as well as compositions, methods of utilizing these engineered polypeptides, and polynucleotides encoding the engineered terminal deoxynucleotidyl transferases.

### CROSS REFERENCE TO RELATED APPLICATIONS

This application claims the priority benefit of U.S. Provisional Patent Application No. 63/499,770, filed May 3, 2023, and U.S. Provisional Patent Application 63/379,439, filed on Oct. 13, 2022, the entirety of each of which is incorporated herein by reference.

### TECHNICAL FIELD

The present invention provides engineered terminal deoxynucleotidyl transferase (TdT) polypeptides useful in template-independent polynucleotide synthesis, as well as compositions and methods of utilizing these engineered polypeptides.

### REFERENCE TO SEQUENCE LISTING, TABLE OR COMPUTER PROGRAM

The official copy of the Sequence Listing is submitted concurrently with the specification as an XML file, with a file name of “CX10-238WO3_ST26.xml”, a creation date of Oct. 11, 2023, and a size of 13,670,249 bytes. The Sequence Listing filed is part of the specification and is incorporated in its entirety by reference herein.

### BACKGROUND

Synthetic biology is becoming established in a diverse range of high value, high growth markets. From food and agriculture to therapeutics, diagnostics, and vaccines; tools such as gene editing, DNA sequencing and gene synthesis are being used to build value-added products with advanced functionality (e.g., cell bioreactors, etc.) and desired end products (e.g., drugs, chemicals, etc.). The barrier to widespread implementation of these technologies is the ability to efficiently synthesize RNA, DNA, and other polynucleotides.

In particular, silencing RNA (siRNA) therapeutics are a promising class of drugs that have the potential to treat numerous difficult to treat conditions in a highly targeted manner by binding to known mRNA targets (Hu et al. (2020). Sig Transduct Target Ther 5, 101; Zhang et al. (2021). Bioch. Pharmac., 189, 114432.) As these therapies become more common and are targeted at larger patient populations, the ability to produce large amounts of the oligonucleotide active pharmaceutical ingredient (API) becomes critical.

To date, short RNA oligonucleotides have been synthesized almost exclusively by iterative addition of nucleotides in the form of activated phosphoramidites, plus additional processing steps, to a growing immobilized nucleotide chain (Brown, T. Nucleic Acids Book. See at: www.atdbio.com/nucleic-acids-book (accessed 2022-10-10).)

Phosphoramidite chemistry has been developed extensively over the years to synthesize small amounts of DNA and for more complex therapeutic RNA syntheses but suffers from several cost and sustainability issues that are potentially limiting as API demand grows to triple-quadruple digit kilograms per year (Andrews et al. (2021). J. Org. Chem. 86, 49-61). Additionally, RNA synthesis using phosphoramidite synthesis chemistry is limited to producing short oligonucleotides of approximately 200 basepairs (Beaucage & Caruthers. (1981). Tetrahedron Lett. 22 (20): 1859.)

The phosphoramidite iterative methodology is multi-step and based on phosphorous (III) coupling chemistry that requires (i) coupling (ii) capping (iii) oxidation to P(V) forming phosphodiester or phoshorothioate diester (iv) deblocking of 5′O group. After chain synthesis is complete, the final oligo is cleaved from the support where deblocking of phosphate cyanoethyl group and nucleobases can also occur (Brown, T. Nucleic Acids Book. See at: www.atdbio.com/nucleic-acids-book (accessed 2022-10-10).) Washes with organic solvents at each step are also required. The phosphate cyanoethyl blocking group and nucleobase protecting groups can be removed in parallel to oligonucleotide cleavage from the solid support to generate the oligonucleotide product, or the cyanoethyl group can be removed under milder condition before chain cleavage, if required.

Many aspects of the environmental impact of the current phosphoramidite methodology and potential advances have been reviewed (Andrews et al. (2021). J. Org. Chem. 86, 49-61). Even at an aspirational high oligonucleotide loading of 20% mass final oligonucleotide to mass solid support, at least five-fold mass of support is required over the final product mass.

In addition to the high cost of mass support required to immobilize the oligonucleotide, the use of organic solvents and 5′-O-blocking groups entail additional waste and process inefficiencies. Organic solvents such as acetonitrile are required for solubilization of the phosphoramidite coupling partners, or dichloromethane or toluene for deprotection steps. These solvents need to be anhydrous to reduce undesired hydrolysis of the phosphoramidite partners and can come from non-sustainable sources, adding cost, sustainability questions, and potential supply issues to the process.

The phosphoramidite coupling partners themselves carry a required blocking group at the 5′O-position, the nucleobase nitrogen atom (in A, C and G), and the nascent phosphate. The most common 5′O-blocking group, dimethoxytrityl, has a molecular mass of ˜303 Da that approaches that of the heaviest native ribonucleotide fragment Gp with a mass of ˜345 Da. This protecting group requires energy, resources, and effort to produce and append, and then requires disposal when separated from the desired materials.

In conclusion, a paradigm shift in oligonucleotide synthesis is necessary to enable siRNA therapeutics by lowering environmental impact, improving economic efficiency, and increasing scalability. New methods of oligonucleotide synthesis are, therefore, of great interest to the pharmaceutical industry.

Template-Independent Enzymatic Synthesis

Enzymatic synthesis may facilitate production of high volumes of complex or long polynucleotides (>200 base pairs) while minimizing toxic waste. A variety of prokaryotic and eukaryotic DNA and RNA polymerases are known to naturally synthesize polynucleotides of thousands of base pairs or more. Most of these polymerases function during DNA replication associated with cell division or transcription of RNA from DNA associated with gene or protein expression. Both of these processes involve template-dependent polynucleotide synthesis, wherein the polymerase uses an existing template polynucleotide strand to synthesize a complementary polynucleotide strand.

The potential of template-independent enzymatic polynucleotide synthesis to produce defined sequences has long been recognized. One early report suggested using NTPs with blocked 3′ groups to allow stepwise addition of specific nucleotide residues (Bollum. (1962). JBC, 237, 1945-1949).

However, few polymerases are known to catalyze template-independent polynucleotide synthesis. These include polymerase lambda, polymerase mu, and terminal deoxynucleotidyl transferase (TdT), all members of the X family of DNA polymerases, many of which participate in DNA repair processes (Dominguez et al. (2000). EMBO, 19(7), 1731-1742.) Of these, TdT is known to generate diversity in antigen receptors by indiscriminately adding nucleosides to the 3′ end of a single-stranded polynucleotide in a template-independent process (Bentolila et al. (1995). EMBO, 14(17), 4221-4229.)

Others have published a method of polynucleotide synthesis using a nucleoside 5′-triphosphate with a 3′-OH position protected with a removable blocking moiety and, specifically, a template-independent polynucleotide polymerase, including a terminal deoxynucleotidyl transferase (U.S. Pat. No. 5,763,594). The blocking group, also known to those skilled in the art as an inhibitor or reversible terminating group, may include a variety of groups that prevent the TdT from adding additional NTPs to the nascent polynucleotide chain. This may include charged molecules, large molecules and moieties, or other blocking groups known to those skilled in the art. Appropriate removable blocking groups may include carbonitriles, phosphates, carbonates, carbamates, esters, ethers, borates, nitrates, sugars, phosphoramidates, phenylsulfenates, and sulfates. Other 3′ blocking groups are also known in the art, including 3′-O-amines and methylamines (U.S. Pat. No. 7,544,794) and 3′-O-azides (U.S. Pat. No. 10,407,721).

Although initially promising, use of 3′-blocked NTPs in template-independent synthesis catalyzed by TdT has proven difficult in practice, as TdT struggles to accept 3′-O-blocked NTPs as substrates. Further, wild-type TdTs have low tolerance for oligo acceptor substrates containing one or more modified nucleotides (e.g. 2′ modifications).

Additionally, synthesis of RNA strands present unique challenges due to the additional, reactive 2′-OH on the ribose. While protection of the 2′ position facilitates RNA synthesis, this approach reduces efficiency because of steric hindrance by the 2′ protecting groups and requires maintenance and removal of the protecting group (CB Reese. (2005). Org Biomol Chem 3, 3851-3868.)

Recently several reports have described template-independent synthesis methods that use modified NTPs with blocking groups attached to the purine or pyrimidine base, leaving the 3′-OH unmodified and available for additional rounds of synthesis. These base blocking groups may include a cleavable linker that allows removal of the blocking group after each NTP addition step. The cleavable linker may also be attached to a detectable label (U.S. Pat. No. 7,057,026, among others). A variety of cleavable linkers are known to those skilled in the art. These include linkers attached via reducible disulfide bonds, photocleavable, electrophilic or nucleophilic, pH sensitive, temperature sensitive, and linkers cleaved by enzymes. One drawback to using cleavable linkers is that, typically, some atoms of the linker moiety remain attached to the NTP following cleavage, leaving a “scar” that may interfere with synthesis of a complementary strand after initial template-independent synthesis of the primary polynucleotide strand.

Recently, modified NTPs with bases attached to blocking groups with cleavable linkers that are “scarless” and leave the nascent DNA ready for the next round of synthesis have been developed. In one example, the blocking group and cleavable linker are attached to the base via a disulfide bond. Upon addition of a reducing agent, the blocking group is removed and the remaining atoms of the linker self-cyclize to leave the nascent DNA free of any linker atoms (U.S. Pat. Nos. 8,808,989, 9,695,470, U.S. Pat. 10,041,110). Methods of using NTPs attached to cleavable blocking groups to synthesize polynucleotides are known, including using a microfluidic device or ink jet printing technology (U.S. Pat. No. 9,279,149). An exonuclease may also be used in a method to synthesize polynucleotides to shorten or completely degrade polynucleotide strands that have not successfully added an NTP after the polynucleotide extension step and prior to removing the blocking group (U.S. Pat. No. 9,771,613).

However, NTP bases with bulky blocking groups attached via cleavable linkers are not optimal for efficient synthesis of complex or long oligonucleotides. The large labels may negatively impact enzyme kinetics, and linker scars may lead to an unacceptable rate of misincorporation when synthesizing the oligonucleotide strand. Additionally, larger linkers and necessary deblocking steps may increase the cost, time, and inefficiency of the process as a whole, rendering these methods economically infeasible.

Recently, several groups have explored modifying the structure or amino acid sequence of TdT or other polymerases to allow template-independent synthesis using 3′-O-blocked groups. Efcavitch et al. describes incorporation of 3′ modified dNTPs by TdT in template-independent synthesis using a murine or bacterial TdT with substituted amino acid residues (U.S. Pat. No. 10,059,929). Other reports describe engineered bovine and gar (Lepisosteus oculatus) TdTs that displayed improved activity over wild-type TdT (U.S. Pat. No. 10,745,727, PCT/GB2020/050247). Similarly, a variety of mutations have been described to improve the activity of Pol X family enzymes (WO 2017216472 A2). Finally, an N-terminal truncation of the BRCT domain (or alternatively mutation of the BRCT domain) of TdT has also been described as enhancing activity in the addition of reversibly blocked NTPs to the 3′-OH of a nucleic acid (US20210164008A1).

However, no feasible methods of template-independent enzymatic synthesis of complex or long polynucleotides are currently known or commercially available, despite the recognized value of this technology and intensive research efforts devoted to resolving challenges in this field. Improved engineered TdT enzymes are necessary to enable template-independent enzymatic synthesis of complex or long polynucleotides or oligonucleotides of defined sequence using nucleoside triphosphates with 3′-O-removable blocking groups, with 2′ modifications, and/or with other modifications.

### SUMMARY

The present invention provides engineered terminal deoxynucleotidyl transferase (TdT) polypeptides useful in template-independent polynucleotide synthesis, as well as compositions and methods of utilizing these engineered polypeptides. The TdTs of the present invention are variants of a predicted splice variant of the wild-type gene from Monodelphis domestica (SEQ ID NO: 2). These engineered TdTs are capable of adding nucleoside triphosphates with a 3′-O-removable blocking group and other natural or modified NTPs to the 3′-OH end of a growing oligonucleotide or polynucleotide chain in a template-independent manner. After removal of the blocking group, additional rounds of NTP addition can be used to synthesize a polynucleotide with a defined sequence of bases without using a complementary template strand as a guide for NTP incorporation (template-independent synthesis).

In some embodiments, the present invention provides an engineered TdT polypeptide comprising an amino acid sequence having at least 80%, 85%, 90%, 91%, 92%, 93%, 94%, 95%, 96%, 97%, 98%, 99% sequence identity to a reference sequence of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246 comprising at least one substitution or one substitution set at one or more positions, wherein the positions are numbered with reference to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246 and wherein the engineered TdT polypeptide has improved thermostability, increased activity at elevated temperatures, increased soluble expression or isolated protein yield, decreased by-product formation, increased specific activity on NTP-3′-O-RBG and other natural or modified NTP substrates, and/or increased activity on various oligo acceptor substrates as compared to a wild-type TdT or other TdTs or template-independent polymerases known to those of skill in the art. These engineered TdT polypeptides with one or more amino acid substitutions or substitution sets are described, below, in the detailed description of the invention.

In some additional embodiments, the engineered polypeptide comprises an amino acid sequence with at least 60% sequence identity to any even-numbered sequence set forth in SEQ ID NO: 4-1960, 2004-3920, 4048-5466, and 5476.

In an additional embodiment, the engineered polypeptide of the present invention further comprises an N-terminal truncation of 1-156 amino acids of the polypeptide sequence relative to any even-numbered sequence set forth in SEQ ID NO: 4-1960, 2004-3920, 4048-5466, and 5476.

In an additional embodiment, the engineered polypeptide of the present invention is fused with a second polypeptide; optionally, wherein the second polypeptide has inorganic pyrophosphatase (IPP) activity (e.g., an IPP with an amino acid sequence selected from SEQ ID NO: 3942 and 3944). In one embodiment, the engineered polypeptide of the present invention fused with a second polypeptide with IPP activity comprises a sequence selected from SEQ ID NO: 5468, 5470, 5472, and 5474.

The present invention also provides an engineered polynucleotide encoding at least one engineered polypeptide described in the above paragraphs. In some embodiments, the engineered polynucleotide comprises the odd-numbered sequences set forth in SEQ ID NOs: 3-1959, 2003-3919, 4047-5465, and 5475.

The present invention further provides vectors comprising at least one engineered polynucleotide described above. In some embodiments, the vectors further comprise at least one control sequence.

The present invention also provides host cells comprising the vectors provided herein. In some embodiments, the host cell produces at least one engineered polypeptide provided herein.

The present invention further provides methods of producing an engineered TdT polypeptide, comprising the steps of culturing the host cell provided herein under conditions such that the engineered polynucleotide is expressed and the engineered polypeptide is produced. In some embodiments, the methods further comprise the step of recovering the engineered polypeptide.

The present invention further provides a method of template-independent synthesis, comprising a TdT or template-independent polymerase with activity on various oligo acceptor substrates and NTP-3′-O-RBG and other natural or modified NTP substrates, wherein the method may comprise an immobilized TdT or an immobilized oligo acceptor substrate or neither an immobilized TdT nor an immobilized oligo acceptor substrate.

### DESCRIPTION OF THE INVENTION

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses. All patents, patent applications, articles and publications mentioned herein, both supra and infra, are hereby expressly incorporated herein by reference.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the invention as a whole.

It is to be understood that both the foregoing general description and the following detailed description are exemplary and explanatory only and are not restrictive of the present invention. The section headings used herein are for organizational purposes only and not to be construed as limiting the subject matter described. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used in this specification and the appended claims, the singular forms “a”, “an” and “the” include plural referents unless the context clearly indicates otherwise. Thus, for example, reference to “a polypeptide” includes more than one polypeptide. Similarly, “comprise,” “comprises,” “comprising” “include,” “includes,” and “including” are interchangeable and not intended to be limiting.

It is to be understood that where descriptions of various embodiments use the term “comprising,” those skilled in the art would understand that in some specific instances, an embodiment can be alternatively described using language “consisting essentially of” or “consisting of.” It is to be further understood that where descriptions of various embodiments use the term “optional” or “optionally” the subsequently described event or circumstance may or may not occur, and that the description includes instances where the event or circumstance occurs and instances in which it does not. It is to be understood that both the foregoing general description, and the following detailed description are exemplary and explanatory only and are not restrictive of this disclosure. The section headings used herein are for organizational purposes only and not to be construed as limiting the subject matter described.

Abbreviations

The abbreviations used for the genetically encoded amino acids are conventional and are as follows:

| Amino Acid | Three-Letter | One-Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | HIS | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D” or clear from the context in which the abbreviation is used, the amino acid may be in either the L- or D-configuration about α-carbon (Ca). For example, whereas “Ala” designates alanine without specifying the configuration about the α-carbon, “D-Ala” and “L-Ala” designate D-alanine and L-alanine, respectively.

When the one-letter abbreviations are used, upper case letters designate amino acids in the L-configuration about the α-carbon and lower-case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

The abbreviations used for the genetically encoding nucleosides are conventional and are as follows: adenosine (A); guanosine (G); cytidine (C); thymidine (T); and uridine (U). These abbreviations are also used interchangeably for nucleosides and nucleotides (nucleosides with one or more phosphate groups). Unless specifically delineated, the abbreviated nucleosides or nucleotides may be either ribonucleosides (or ribonucleotides) or 2′-deoxyribonucleosides (or 2′-deoxyribonucleotides). The nucleosides or nucleotides may also be modified at the 3′ position. The nucleosides or nucleotides may be specified as being either ribonucleosides (or ribonucleotides) or 2′-deoxyribonucleosides (or 2′-deoxyribonucleotides) on an individual basis or on an aggregate basis. When nucleic acid sequences are presented as a string of one-letter abbreviations, the sequences are presented in the 5′ to 3′ direction in accordance with common convention, and the phosphates are not indicated.

### Definitions

In reference to the present invention, the technical and scientific terms used in the descriptions herein will have the meanings commonly understood by one of ordinary skill in the art, unless specifically defined otherwise. Accordingly, the following terms are intended to have the following meanings.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids, as well as polymers comprising D- and L-amino acids, and mixtures of D- and L-amino acids.

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes.

As used herein, “polynucleotide,” “oligonucleotide,” and “nucleic acid” are used interchangeably herein and refer to two or more nucleosides or nucleotides that are covalently linked together. The polynucleotide may be wholly comprised of ribonucleotides (i.e., RNA), wholly comprised of 2′ deoxyribonucleotides (i.e., DNA), wholly comprised of other synthetic nucleotides or comprised of mixtures of synthetic, ribo- and/or 2′ deoxyribonucleotides. The polynucleotides may also include modified nucleotides with substitutions, including 2′ substitutions (e.g., 2′-fluoro, 2′-O-methyl, 2′-O-methoxyethyl, locked or constrained ethyl modifications, and others known to those skilled in the art). Nucleosides will be linked together via standard phosphodiester linkages or via one or more non-standard linkages, including but not limited to phosphorothioate linkages. The polynucleotide may be single-stranded or double-stranded or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino-acid sequences. Nucleobases that are modified or synthetic may comprise any known or hypothetical or future discovered modification or structure that would be recognized by one of skill in the art as a modified or synthetic nucleobase. Similarly, the terms “polynucleotide,” “oligonucleotide,” and “nucleic acid” are intended to comprise any modified or synthetic structure that is now known or discovered in the future that would be recognized by one of skill in the art as being or having the function of a “polynucleotide,” “oligonucleotide,” or “nucleic acid.” An example of a modified or synthetic structure having the function of a “polynucleotide,” “oligonucleotide,” or “nucleic acid” is PNA or peptide nucleic acid.

As used herein, “oligo acceptor substrate” and “acceptor substrate” and “growing oligo acceptor substrate strand” and “growing oligonucleotide chain” and “growing polynucleotide strand” are used interchangeably herein and refer to any oligo or nucleotide chain or similar moiety with an exposed 3′-OH or equivalent thereof that may be recognized by a wild-type TdT or polymerase or an engineered TdT or template-independent polymerase of the current disclosure as a substrate for nucleoside addition or synthesis. In some embodiments, the acceptor substrate may be single stranded. In yet other embodiments, the acceptor substrate may be double stranded or partially doubled stranded. In some embodiments, the acceptor substrate may comprise a nucleotide chain consisting of 1-10 nucleotides, 5-20 nucleotides, 15-50 nucleotides, 30-100 nucleotides, or greater than 100 nucleotides. In some embodiments, the acceptor substrate may comprise a chemical moiety that is not a nucleotide chain but contains a free —OH capable of being recognized as a substrate by a wild-type or engineered TdT, referred to herein as a “3′-OH equivalent”. Exemplary oligo acceptor substrates are provided in the Examples.

As used herein, “nucleoside triphosphate-3′-O-removable blocking group” and “nucleotide triphosphate-3′-O-removable blocking group” and “reversible terminator” and “NTP-3′-O-RBG” are used interchangeably herein and refer to a ribonucleoside triphosphate or a deoxyribonucleoside triphosphate or a synthetic or nucleoside triphosphate composed of an alternate or modified sugar with a removable blocking group attached at the 3′ position of the sugar moiety. An NTP-3′-O-RBG may also include other modifications as described herein, including but not limited to modifications at the 2′ position, modifications to the nucleobase, and modifications to the phosphates. A nucleotide may also have a 3′-O-RBG, as is expected after reaction of an NTP-3′-O-RBG with an engineered TdT of the present disclosure and an oligo acceptor substrate.

As used herein, “oligo acceptor product” and “growing oligonucleotide chain” and “oligo acceptor extension product” are used interchangeably herein and refer to the product of a NTP-3′-O-RBG or other natural or modified NTP substrate and an oligo acceptor substrate, wherein a TdT or related polymerase has catalyzed the extension or addition of a nucleotide-3′-O-RBG or other natural or modified nucleotide substrate to an oligo acceptor substrate via reaction with one or more NTP-3′-O-RBGs or other natural or modified NTP substrates.

As used herein, “removable blocking group” and “blocking group” and “terminator group” and “reversible terminating group” and “inhibitor group” and related variations of these terms are used interchangeably herein and refer to a chemical group that would hinder addition of a second NTP-3′-O-RBG or other natural or modified NTP substrate to the 3′ end of the growing oligo acceptor substrate strand prior to removal of the removable blocking from the first round of addition. In some embodiments, the NTP-3′-O-RBG or other natural or modified NTP substrate may comprise a removable blocking group selected from the group consisting of NTP-3′-O—NH₂, or NTP-3′-O—PO₃. In some embodiments, the NTP-3′-O-RBG or other natural or modified NTP substrate may have a natural purine or pyrimidine base, such as adenine, guanine, cytosine, thymine, or uridine. In some embodiments, NTP-3′-O-RBG or other natural or modified NTP substrates may have an unnatural base analog such as inosine, xanthine, hypoxanthine or another base analog, as is known in the art. In some embodiments the blocking group may comprise or may additionally comprise a modification at the 2′ position.

As used herein, “template-independent synthesis” refers to synthesis of an oligonucleotide or a polynucleotide without the use of template strand as a guide for synthesis of a complementary oligo or polynucleotide strand. Thus, template-independent synthesis refers to an iterative process, whereby, successive nucleotides are added to a growing oligo or nucleotide chain or acceptor substrate. Template-independent synthesis may be in a sequence defined manner or may be random, as is the case with the wild-type TdT in creating antigen receptor diversity. Processes for template-independent synthesis are further described herein.

“Coding sequence” refers to that portion of a nucleic acid (e.g., a gene) that encodes an amino acid sequence of a protein.

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

As used herein, “recombinant,” “engineered,” and “non-naturally occurring” when used with reference to a cell, nucleic acid, or polypeptide, refer to a material, or a material corresponding to the natural or native form of the material, that has been modified in a manner that would not otherwise exist in nature. In some embodiments, the cell, nucleic acid or polypeptide is identical to a naturally occurring cell, nucleic acid or polypeptide, but is produced or derived from synthetic materials and/or by manipulation using recombinant techniques. Non-limiting examples include, among others, recombinant cells expressing genes that are not found within the native (non-recombinant) form of the cell or expressed native genes that are otherwise expressed at a different level.

“Percentage of sequence identity” and “percentage homology” are used interchangeably herein to refer to comparisons among polynucleotides or polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 2:482 [1981]), by the homology alignment algorithm of Needleman and Wunsch (Needleman and Wunsch, J. Mol. Biol., 48:443 [1970]), by the search for similarity method of Pearson and Lipman (Pearson and Lipman, Proc. Natl. Acad. Sci. USA 85:2444 [1988]), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See, Altschul et al., J. Mol. Biol., 215: 403-410 [1990]; and Altschul et al., Nucl. Acids Res., 3389-3402 [1977], respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSum62 scoring matrix (See, Henikoff and Henikoff, Proc. Natl. Acad. Sci. USA 89:10915 [1989]). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, a “reference sequence based on SEQ ID NO:4 having at the residue corresponding to X14 a valine” or X14V refers to a reference sequence in which the corresponding residue at X14 in SEQ ID NO:4, which is a tyrosine, has been changed to valine.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

As used herein, “substantial identity” refers to a polynucleotide or polypeptide sequence that has at least 80 percent sequence identity, at least 85 percent identity, at least between 89 to 95 percent sequence identity, or more usually, at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In some specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, at least 95 percent sequence identity or more (e.g., 99 percent sequence identity). In some embodiments, residue positions that are not identical in sequences being compared differ by conservative amino acid substitutions.

“Corresponding to,” “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered TdT, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Amino acid difference” or “residue difference” refers to a change in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X25 as compared to SEQ ID NO: 2” refers to a change of the amino acid residue at the polypeptide position corresponding to position 25 of SEQ ID NO:2. Thus, if the reference polypeptide of SEQ ID NO: 2 has a valine at position 25, then a “residue difference at position X25 as compared to SEQ ID NO:2” an amino acid substitution of any residue other than valine at the position of the polypeptide corresponding to position 25 of SEQ ID NO: 2. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some embodiments, more than one amino acid can appear in a specified residue position (i.e., the alternative amino acids can be listed in the form XnY/Z, where Y and Z represent alternate amino acid residues). In some instances (e.g., in Tables 5.1, 6.2, 7.2, 8.2, 9.2, 10.2, 11.2, 12.2, 13.2, 14.2, 15.2, 16.2, 17.2, 18.2, 19.2, 20.2, 21.2, 22.2, 23.2, 24.2, 25.2, 26.2, 26.3, 26.4, 27.2, 27.3, 27.4, 27.5, 28.1, 28.2, 28.3, 29.2, 30.2, 31.2, 32.2, 33.2, 34.2, 35.2, 36.2, 37.2, 38.2, 39.2, 40.2, 41.2, 42.2, 43.2, 44.2, 45.2 46.2, 47.2, 48.2, 49.2, 50.2, 51.2, 52.2, 53.2, 54.2, 55.2, 56.2, 56.3, 56.4, 61.2, 63.2, 64.2, 65.2, 66.2, 67.2, 68.2, 69.2, 70.2, 71.2, 72.2, 73.2, 74.2, 75.2, 76.2, 77.2, 78.2, 79.2, and 80.1) the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. Furthermore, in some instances, a polypeptide of the present invention can include one or more amino acid residue differences relative to a reference sequence, which is indicated by a list of the specified positions where changes are made relative to the reference sequence. In some additional embodiments, the present invention provides engineered polypeptide sequences comprising both conservative and non-conservative amino acid substitutions.

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain is substituted with another aliphatic amino acid (e.g., alanine, valine, leucine, and isoleucine); an amino acid with an hydroxyl side chain is substituted with another amino acid with a hydroxyl side chain (e.g., serine and threonine); an amino acid having an aromatic side chain is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. Exemplary conservative substitutions are provided in Table 1 below.

TABLE 1
Conservative Amino Acid Substitution Examples

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | None |

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered TdT enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. In some embodiments, the improved engineered TdT enzymes comprise insertions of one or more amino acids to the naturally occurring polypeptide as well as insertions of one or more amino acids to other improved TdT polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Fragment” as used herein refers to a polypeptide that has an amino-terminal and/or carboxy-terminal deletion, but where the remaining amino acid sequence is identical to the corresponding positions in the sequence. Fragments can be at least 14 amino acids long, at least 20 amino acids long, at least 50 amino acids long or longer, and up to 70%, 80%, 90%, 95%, 98%, and 99% of the full-length TdT polypeptide, for example the polypeptide of SEQ ID NO: 2 or an TdT provided in the even-numbered sequences of SEQ ID NO: 4-1960, 2004-3920, 4048-5466, and 5476.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The engineered TdT enzymes may be present within a cell, present in the cellular medium, or prepared in various forms, such as lysates or isolated preparations. As such, in some embodiments, the engineered TdT enzyme can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure TdT composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated engineered TdT polypeptide is a substantially pure polypeptide composition.

As used herein, “improved enzyme property” refers to at least one improved property of an enzyme. In some embodiments, the present invention provides engineered TdT polypeptides that exhibit an improvement in any enzyme property as compared to a reference TdT polypeptide and/or a wild-type TdT polypeptide, and/or another engineered TdT polypeptide. For the engineered TdT polypeptides described herein, the comparison is generally made to the wild-type enzyme from which the TdT is derived, although in some embodiments, the reference enzyme can be another improved engineered TdT. Thus, the level of “improvement” can be determined and compared between various TdT polypeptides, including wild-type, as well as engineered TdTs. Improved properties include, but are not limited, to such properties as enzymatic activity (which can be expressed in terms of percent conversion of the substrate), thermostability, solvent stability, pH activity profile, cofactor requirements, refractoriness to inhibitors (e.g., substrate or product inhibition), activity at elevated temperatures, increased soluble expression, decreased by-product formation, increased specific activity on NTP-3′-O-RBG substrates, increased incorporation efficiency in extension of oligo acceptor substrates, and/or increased activity on various oligo acceptor substrates (including enantioselectivity).

“Increased enzymatic activity” refers to an improved property of the TdT polypeptides, which can be represented by an increase in specific activity (e.g., product produced/time/weight protein) or an increase in percent conversion of the substrate to the product (e.g., percent conversion of starting amount of substrate to product in a specified time period using a specified amount of TdT) as compared to the reference TdT enzyme. Exemplary methods to determine enzyme activity are provided in the Examples. Any property relating to enzyme activity may be affected, including the classical enzyme properties of Kₘ, Vₘₐₓ or k_{car}, changes of which can lead to increased enzymatic activity. Improvements in enzyme activity can be from about 1.2 times the enzymatic activity of the corresponding wild-type enzyme, to as much as 2 times, 5 times, 10 times, 20 times, 25 times, 50 times or more enzymatic activity than the naturally occurring or another engineered TdT from which the TdT polypeptides were derived. TdT activity can be measured by any one of standard assays, such as by monitoring changes in properties of substrates, cofactors, or products. In some embodiments, the amount of products generated can be measured by Liquid Chromatography-Mass Spectrometry (LC-MS), HPLC, or other methods, as known in the art. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

“Conversion” refers to the enzymatic conversion of the substrate(s) to the corresponding product(s). “Percent conversion” refers to the percent of the substrate that is converted to the product within a period of time under specified conditions. Thus, the “enzymatic activity” or “activity” of a TdT polypeptide can be expressed as “percent conversion” of the substrate to the product.

“Thermostable” refers to a polypeptide that maintains similar activity (more than 60% to 80% for example) after exposure to elevated temperatures (e.g., 40-80° C.) for a period of time (e.g., 0.5-24 hrs) compared to the wild-type enzyme exposed to the same elevated temperature.

“Solvent stable” refers to a polypeptide that maintains similar activity (more than e.g., 60% to 80%) after exposure to varying concentrations (e.g., 5-99%) of solvent (ethanol, isopropyl alcohol, dimethylsulfoxide (DMSO), tetrahydrofuran, 2-methyltetrahydrofuran, acetone, toluene, butyl acetate, methyl tert-butyl ether, etc.) for a period of time (e.g., 0.5-24 hrs) compared to the wild-type enzyme exposed to the same concentration of the same solvent.

“Thermo- and solvent stable” refers to a polypeptide that is both thermostable and solvent stable.

The term “stringent hybridization conditions” is used herein to refer to conditions under which nucleic acid hybrids are stable. As known to those of skill in the art, the stability of hybrids is reflected in the melting temperature (Tₘ) of the hybrids. In general, the stability of a hybrid is a function of ion strength, temperature, G/C content, and the presence of chaotropic agents. The Tₘ values for polynucleotides can be calculated using known methods for predicting melting temperatures (See e.g., Baldino et al., Meth. Enzymol., 168:761-777 [1989]; Bolton et al., Proc. Natl. Acad. Sci. USA 48:1390 [1962]; Bresslauer et al., Proc. Natl. Acad. Sci. USA 83:8893-8897 [1986]; Freier et al., Proc. Natl. Acad. Sci. USA 83:9373-9377 [1986]; Kierzek et al., Biochem., 25:7840-7846 [1986]; Rychlik et al., 1990, Nucl. Acids Res., 18:6409-6412 [1990] (erratum, Nucl. Acids Res., 19:698 [1991]); Sambrook et al., supra); Suggs et al., 1981, in Developmental Biology Using Purified Genes, Brown et al. [eds.], pp. 683-693, Academic Press, Cambridge, MA [1981]; and Wetmur, Crit. Rev. Biochem. Mol. Biol., 26:227-259 [1991]). In some embodiments, the polynucleotide encodes the polypeptide disclosed herein and hybridizes under defined conditions, such as moderately stringent or highly stringent conditions, to the complement of a sequence encoding an engineered TdT enzyme of the present invention.

“Hybridization stringency” relates to hybridization conditions, such as washing conditions, in the hybridization of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature T, as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Heterologous” polynucleotide refers to any polynucleotide that is introduced into a host cell by laboratory techniques and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the TdT enzymes may be codon optimized for optimal production from the host organism selected for expression.

As used herein, “preferred, optimal, high codon usage bias codons” refers interchangeably to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (See e.g., GCG CodonPreference, Genetics Computer Group Wisconsin Package; CodonW, Peden, University of Nottingham; McInerney, Bioinform., 14:372-73 [1998]; Stenico et al., Nucl. Acids Res., 222437-46 [1994]; Wright, Gene 87:23-29 [1990]). Codon usage tables are available for many different organisms (See e.g., Wada et al., Nucl. Acids Res., 20:2111-2118 [1992]; Nakamura et al., Nucl. Acids Res., 28:292 [2000]; Duret, et al., supra; Henaut and Danchin, in Escherichia coli and Salmonella, Neidhardt, et al. (eds.), ASM Press, Washington D.C., p. 2047-2066 [1996]). The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (See e.g., Mount, Bioinformatics: Sequence and Genome Analysis, Chapter 8, Cold Spring Harbor Laboratory Press, Cold Spring Harbor, N.Y. [2001]; Uberbacher, Meth. Enzymol., 266:259-281 [1996]; and Tiwari et al., Comput. Appl. Biosci., 13:263-270 [1997]).

“Control sequence” is defined herein to include all components, which are necessary or advantageous for the expression of a polynucleotide and/or polypeptide of the present invention. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” refer to those conditions in the biocatalytic reaction solution (e.g., ranges of enzyme loading, substrate loading, cofactor loading, temperature, pH, buffers, co-solvents, etc.) under which a TdT polypeptide of the present invention is capable of converting one or more substrate compounds to a product compound (e.g., addition of a nucleotide-3′-O-RBG or other natural or modified nucleotide substrate to an oligo acceptor substrate via reaction with NTP-3′-O-RBG or other natural or modified NTP substrate). Exemplary “suitable reaction conditions” are provided in the present invention and illustrated by the Examples.

“Composition” refers to a mixture or combination of one or more substances, wherein each substance or component of the composition retains its individual properties. As used herein, a biocatalytic composition refers to a combination of one or more substances useful for biocatalysis.

“Loading”, such as in “compound loading” or “enzyme loading” or “cofactor loading” refers to the concentration or amount of a component in a reaction mixture at the start of the reaction.

“Substrate” in the context of a biocatalyst mediated process refers to the compound or molecule acted on by the biocatalyst. For example, a TdT biocatalyst used in the synthesis processes disclosed herein acts on an NTP-3′-O-RBG substrate or other natural or modified NTP substrate and an oligo acceptor substrate.

“Product” in the context of a biocatalyst mediated process refers to the compound or molecule resulting from the action of the biocatalyst. For example, an exemplary product for a TdT biocatalyst used in a process disclosed herein is an oligo acceptor extension product, as depicted in Schemes 1 and 2.

“Alkyl” refers to saturated hydrocarbon groups of from 1 to 18 carbon atoms inclusively, either straight chained or branched, more preferably from 1 to 8 carbon atoms inclusively, and most preferably 1 to 6 carbon atoms inclusively. An alkyl with a specified number of carbon atoms is denoted in parenthesis (e.g., (C₁-C₆)alkyl refers to an alkyl of 1 to 6 carbon atoms).

“Alkenyl” refers to hydrocarbon groups of from 2 to 12 carbon atoms inclusively, either straight or branched containing at least one double bond but optionally containing more than one double bond.

“Alkynyl” refers to hydrocarbon groups of from 2 to 12 carbon atoms inclusively, either straight or branched containing at least one triple bond but optionally containing more than one triple bond, and additionally optionally containing one or more double bonded moieties.

“Heteroalkyl, “heteroalkenyl,” and heteroalkynyl,” refer respectively, to alkyl, alkenyl and alkynyl as defined herein in which one or more of the carbon atoms are each independently replaced with the same or different heteroatoms or heteroatomic groups. Heteroatoms and/or heteroatomic groups which can replace the carbon atoms include, but are not limited to —O—, —S—, —S—O—, —NR^{γ}—, —PH—, —S(O)—, —S(O)2-, —S(O) NR^{γ}—, —S(O)₂NR^{γ}, and the like, including combinations thereof, where each R^{γ} is independently selected from hydrogen, alkyl, cycloalkyl, heterocycloalkyl, aryl, and heteroaryl.

“Amino” refers to the group —NH₂. Substituted amino refers to the group —NHR^{η}, NR^{η}R^{η}, and NR^{η}R^{η}R^{η}, where each R^{η} is independently selected from substituted or unsubstituted alkyl, cycloalkyl, cycloheteroalkyl, alkoxy, aryl, heteroaryl, heteroarylalkyl, acyl, alkoxycarbonyl, sulfanyl, sulfinyl, sulfonyl, and the like. Typical amino groups include, but are limited to, dimethylamino, diethylamino, trimethylammonium, triethylammonium, methylysulfonylamino, furanyl-oxy-sulfamino, and the like.

“Aminoalkyl” refers to an alkyl group in which one or more of the hydrogen atoms are replaced with one or more amino groups, including substituted amino groups.

“Aminocarbonyl” refers to —C(O)NH₂. Substituted aminocarbonyl refers to —C(O)NR^{η}R^{η}, where the amino group NR^{η}R^{η} is as defined herein.

“Oxy” refers to a divalent group —O—, which may have various substituents to form different oxy groups, including ethers and esters.

“Alkoxy” or “alkyloxy” are used interchangeably herein to refer to the group —OR, wherein R is an alkyl group, including optionally substituted alkyl groups.

“Carboxy” refers to —COOH.

“Carbonyl” refers to —C(O)—, which may have a variety of substituents to form different carbonyl groups including acids, acid halides, aldehydes, amides, esters, and ketones.

“Carboxyalkyl” refers to an alkyl in which one or more of the hydrogen atoms are replaced with one or more carboxy groups.

“Aminocarbonylalkyl” refers to an alkyl substituted with an aminocarbonyl group, as defined herein.

“Halogen” or “halo” refers to fluoro, chloro, bromo and iodo.

“Haloalkyl” refers to an alkyl group in which one or more of the hydrogen atoms are replaced with a halogen. Thus, the term “haloalkyl” is meant to include monohaloalkyls, dihaloalkyls, trihaloalkyls, etc. up to perhaloalkyls. For example, the expression “(C₁-C₂) haloalkyl” includes 1-fluoromethyl, difluoromethyl, trifluoromethyl, 1-fluoroethyl, 1,1-difluoroethyl, 1,2-difluoroethyl, 1,1,1 trifluoroethyl, perfluoroethyl, etc.

“Hydroxy” refers to —OH.

“Hydroxyalkyl” refers to an alkyl group in which in which one or more of the hydrogen atoms are replaced with one or more hydroxy groups.

“Thiol” or “sulfanyl” refers to —SH. Substituted thiol or sulfanyl refers to —S—R^{η}, where R^{η} is an alkyl, aryl or other suitable substituent.

“Sulfonyl” refers to —SO₂-R^{η}. Substituted sulfonyl refers to —SO₂—R^{η}, where R^{η} is an alkyl, aryl or other suitable substituent.

“Alkylsulfonyl” refers to —SO₂—R^{ζ}, where R^{ζ} is an alkyl, which can be optionally substituted. Typical alkylsulfonyl groups include, but are not limited to, methylsulfonyl, ethylsulfonyl, n-propylsulfonyl, and the like.

“Phosphate” as used herein refers to a functional group comprised of an orthophosphate ion (phosphorous atom covalently linked to four oxygen atoms). The orthophosphate ion is commonly found with one or more hydrogen atoms or organic groups.

“Phosphorylated” as used herein refers to the addition or presence of one of more phosphoryl groups (phosphorous atom covalently linked to the three oxygen atoms).

“Optionally substituted” as used herein with respect to the foregoing chemical groups means that positions of the chemical group occupied by hydrogen can be substituted with another atom (unless otherwise specified) exemplified by, but not limited to carbon, oxygen, nitrogen, or sulfur, or a chemical group, exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; where preferred heteroatoms are oxygen, nitrogen, and sulfur. Additionally, where open valences exist on these substitute chemical groups they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further contemplated that the above substitutions can be made provided that replacing the hydrogen with the substituent does not introduce unacceptable instability to the molecules of the present invention and is otherwise chemically reasonable. One of ordinary skill in the art would understand that with respect to any chemical group described as optionally substituted, only sterically practical and/or synthetically feasible chemical groups are meant to be included. “Optionally substituted” as used herein refers to all subsequent modifiers in a term or series of chemical groups. For example, in the term “optionally substituted arylalkyl,” the “alkyl” portion and the “aryl” portion of the molecule may or may not be substituted, and for the series “optionally substituted alkyl, cycloalkyl, aryl and heteroaryl,” the alkyl, cycloalkyl, aryl, and heteroaryl groups, independently of the others, may or may not be substituted.

“Reaction” as used herein refers to a process in which one or more substances or compounds or substrates is converted into one or more different substances, compounds, or processes.

Template-Independent Synthesis by Engineered TdTs

New methods of efficiently synthesizing high purity strands of DNA, RNA, and other polynucleotides are necessary to overcome the limitations of existing phosphoramidite chemical synthesis methods in order to enable a range of emerging and existing synthetic biology applications.

The present invention provides novel terminal deoxynucleotidyl transferases that have improved activity in the template-independent synthesis of polynucleotides using 5′-nucleoside triphosphates (“NTPs”) modified with a 3′-O-removable blocking group (NTP-3′-O-RBG) or other natural or modified NTP substrates. The TdTs of the present disclosure have improved thermostability, activity at elevated temperatures, increased soluble expression or isolated protein yield, decreased by-product formation, increased affinity for NTP-3′-O-RBG and other natural or modified NTP substrates, increased affinity for oligo acceptor substrates, increased activity or specific activity on NTP-3′-O-RBG and other natural or modified NTP substrates, and/or increased activity or specific activity on various oligo acceptor substrates as compared to a wild-type TdT or other TdTs or template-independent polymerases known to those of skill in the art. The engineered polypeptides of the present disclosure are variants of SEQ ID NO: 2, a predicted splice variant encoded by the genome of species Monodelphis domestica. These engineered TdTs are capable of template-independent synthesis of oligonucleotides and polynucleotides.

Template-independent synthesis of a defined polynucleotide sequence using an engineered TdT is a multistep process. In one embodiment, an oligo acceptor substrate with a 3′-OH allows addition of a defined modified NTP substrate (in this example, an NTP-3′-O-RBG) by an engineered TdT, as depicted in Scheme 1, below.

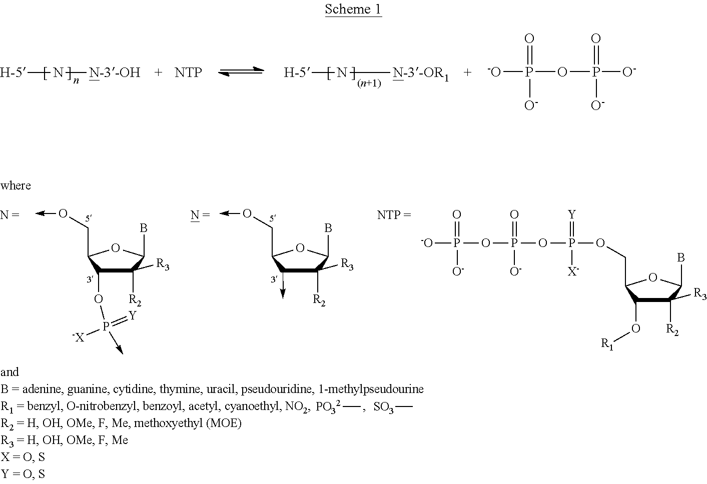

After reaction of the NTP-3′-O-RBG with the 3′-OH of oligo acceptor substrate or the growing polynucleotide chain, the TdT is blocked from further reaction by the 3′-O-RBG. The RBG is then removed, exposing the 3′-OH and allowing another round of addition. After each round of addition, the blocking group of the nucleotide-3′-O-RBG or natural or modified nucleotide from the previous round is removed and a new NTP-3′-O-RBG or natural or modified NTP substrate is added to sequentially and efficiently create a defined polynucleotide sequence by addition at the 3′-OH end of the polynucleotide or oligo acceptor substrate without a complimentary strand or templating primer sequence. After synthesis of the defined polynucleotide is complete, the oligonucleotide chain may be cleaved or released from the oligo acceptor substrate.

A variety of oligo acceptor substrates and NTP-3′-O-RBG or natural or modified NTP substrates may be used in this process, as may be envisioned by one of skill in the art. An example of one reaction is detailed in Scheme 2, below. Scheme 2 depicts the TdT catalyzed reaction of 5′-6-FAM-[N]₁₅AT*mC and 3′-phos-mATP as described in Example 27, while other examples of suitable oligo acceptor substrate and NTP-3′-O-RBG or natural or modified NTP pairs are described in other Examples. These examples are non-limiting.

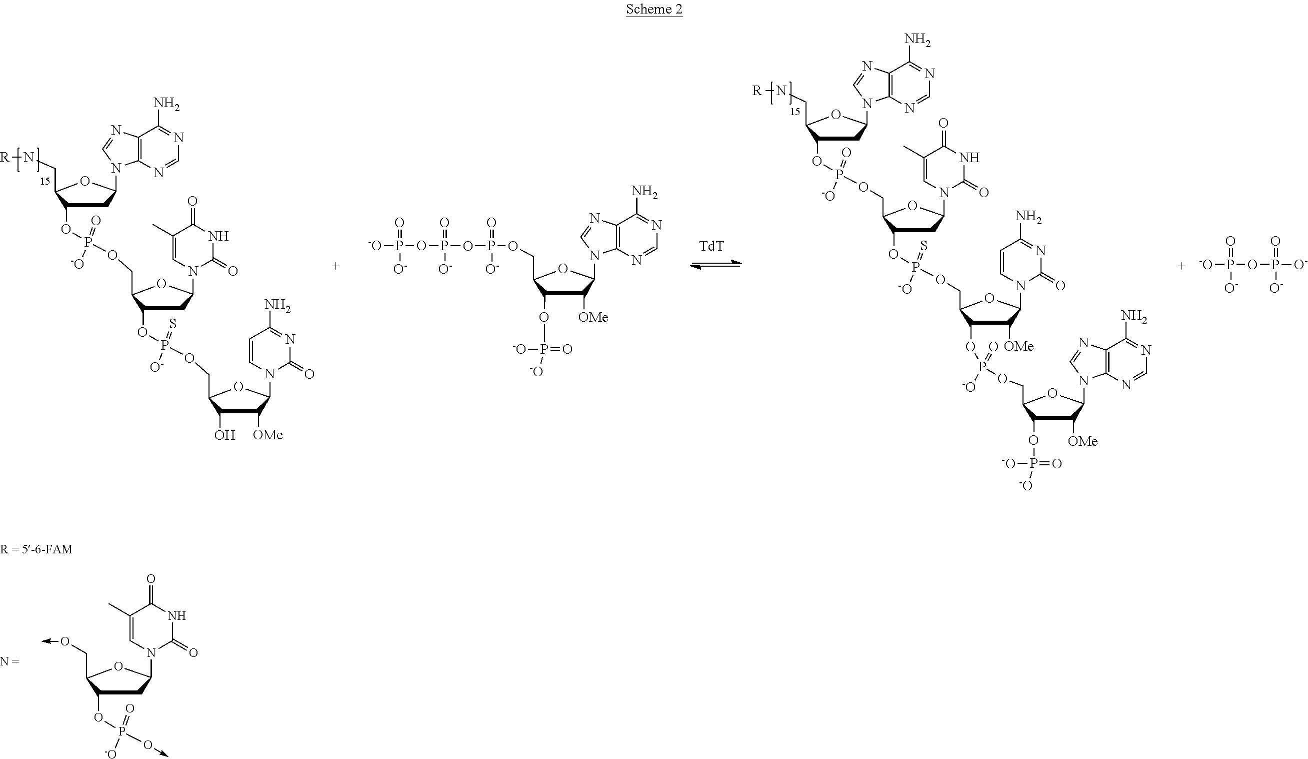

Occasionally, undesired synthesis products are created by the TdT during the addition step. This includes incorporation of NTPs that have lost their blocking group, addition of more than one NTP, or the excision or pyrophosphorolysis of the TdT on the growing polynucleotide chain.

In some embodiments, one or more additional quality control steps are used, such as adding an exonuclease prior to removing the blocking group and initiating a new round of synthesis. In some embodiments, a phosphatase, such as a pyrophosphatase, is used to breakdown inorganic phosphate and push the reversible TdT reaction toward synthesis.

As described further herein, the engineered TdT polypeptides of the current disclosure exhibit one of more improved properties in the template-independent polynucleotide synthesis process depicted in Schemes 1 and 2.

In some embodiments, the present invention provides an engineered TdT polypeptide comprising an amino acid sequence having at least 60% sequence identity to an amino acid reference sequence of SEQ ID NO: 2 and further comprising one or more amino acid residue differences as compared to the reference amino acid sequence, wherein the engineered TdT polypeptide has improved thermostability, increased activity at elevated temperatures, increased soluble expression or isolated protein yield, decreased by-product formation, increased specific activity on NTP-3′-O-RBG or natural or modified NTP substrates, and/or increased activity on various oligo acceptor substrates as compared to a wild-type TdT or other TdTs or template-independent polymerases known to those of skill in the art.

In particular, the engineered TdTs polypeptides of the present disclosure have been engineered for efficient synthesis of polynucleotides having a defined sequence using NTP-3′-O-RBG or natural or modified NTP substrates in the process described above.

A variety of suitable reaction conditions are known to those skilled in the art, as detailed below and in the Examples.

Engineered Terminal Deoxynucleotidyl Transferase (TdT) Polypeptides

The present invention provides engineered terminal deoxynucleotidyl transferase (TDT) polypeptides useful in template-independent polynucleotide synthesis using an NTP-3′-O-RBG or natural or modified NTP substrate, as well as compositions and methods of utilizing these engineered polypeptides in template-independent oligonucleotide synthesis.

The present invention provides TdT polypeptides, polynucleotides encoding the polypeptides, methods of preparing the polypeptides, and methods for using the polypeptides. Where the description relates to polypeptides, it is to be understood that it can describe the polynucleotides encoding the polypeptides.

Suitable reaction conditions under which the above-described improved properties of the engineered polypeptides carry out the desired reaction can be determined with respect to concentrations or amounts of polypeptide, substrate, co-substrate, buffer, solvent, pH, conditions including temperature and reaction time, and/or conditions with the polypeptide immobilized on a solid support, as further described below and in the Examples.

In some embodiments, exemplary engineered TdTs comprise an amino acid sequence that has one or more residue differences as compared to SEQ ID NO: 2 at the residue positions indicated in Tables 5.1, 6.2, 7.2, 8.2, 9.2, 10.2, 11.2, 12.2, 13.2, 14.2, 15.2, 16.2, 17.2, 18.2, 19.2, 20.2, 21.2, 22.2, 23.2, 24.2, 25.2, 26.2, 26.3, 26.4, 27.2, 27.3, 27.4, 27.5, 28.1, 28.2, 28.3, 29.2, 30.2, 31.2, 32.2, 33.2, 34.2, 35.2, 36.2, 37.2, 38.2, 39.2, 40.2, 41.2, 42.2, 43.2, 44.2, 45.2 46.2, 47.2, 48.2, 49.2, 50.2, 51.2, 52.2, 53.2, 54.2, 55.2, 56.2, 56.3, 56.4, 61.2, 63.2, 64.2, 65.2, 66.2, 67.2, 68.2, 69.2, 70.2, 71.2, 72.2, 73.2, 74.2, 75.2, 76.2, 77.2, 78.2, 79.2, and 80.1.

The structure and function information for the exemplary engineered polypeptides of the present invention are based on the conversion of an oligo acceptor substrate and a NTP-3′-O-RBG or a dideoxy NTP (e.g. a 2′,3′-dideoxy NTP), the results of which are shown below in Tables 5.1, 6.2, 7.2, 8.2, 9.2, 10.2, 11.2, 12.2, 13.2, 14.2, 15.2, 16.2, 17.2, 18.2, 19.2, 20.2, 21.2, 22.2, 23.2, 24.2, 25.2, 26.2, 26.3, 26.4, 27.2, 27.3, 27.4, 27.5, 28.1, 28.2, 28.3, 29.2, 30.2, 31.2, 32.2, 33.2, 34.2, 35.2, 36.2, 37.2, 38.2, 39.2, 40.2, 41.2, 42.2, 43.2, 44.2, 45.2 46.2, 47.2, 48.2, 49.2, 50.2, 51.2, 52.2, 53.2, 54.2, 55.2, 56.2, 56.3, 56.4, 61.2, 63.2, 64.2, 65.2, 66.2, 67.2, 68.2, 69.2, 70.2, 71.2, 72.2, 73.2, 74.2, 75.2, 76.2, 77.2, 78.2, 79.2, and 80.1, as further described in the Examples. The odd numbered sequence identifiers (i.e., SEQ ID NOs) in these Tables refer to the nucleotide sequence encoding the amino acid sequence provided by the even numbered SEQ ID NOs in these Tables. Exemplary sequences are provided in the electronic sequence listing file accompanying this invention, which is hereby incorporated by reference herein. The amino acid residue differences are based on comparison to the reference sequence of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, as indicated.

Terminal deoxynucleotidyl transferase, a member of the Pol X family, has been identified in many species. Members of the diverse Pol X family are known to share certain residues, which are conserved across family members. TdT also has a high level of conservation across species for residues thought to be involved in binding divalent metal ions, ternary complex formation, and binding dNTP and DNA ligands (Dominguez et al. (2000). EMBO, 19(7), 1731-1742.) Additionally, TdTs are known to have splice variants which are N-terminal truncations, lacking a BRCT domain. Other template-independent polymerases (including, but not limited to polyA polymerases, polyU polymerases and terminal urildylytransferases) are also known in the art and may be used to practice the invention. Similarly, other polymerases are known to be capable of template-independent synthesis (including but not limited to reverse transcriptases) and may be used to practice the invention.

The wild-type TdT from Monodelphis domestica (SEQ ID NO: 2) was selected for evolution. The TdT polypeptides of the present disclosure are engineered variants of SEQ ID NO: 2 with a N-terminal 6-histidine tag.

The polypeptides of the present disclosure have residue differences that result in improved properties necessary to develop an efficient TdT enzyme, capable of template-independent synthesis of polynucleotides having a defined sequence. Various residue differences, at both conserved and non-conserved positions, have been discovered to be related to improvements in various enzymes properties, including improved thermostability, increased activity at elevated temperatures, increased soluble expression or isolated protein yield, decreased by-product formation, increased specific activity on NTP-3′-O-RBG or natural or modified NTP substrates, increased incorporation efficiency in extension of oligo acceptor substrates, and/or increased activity on various oligo acceptor substrates as compared to a wild-type TdT or other TdTs or template-independent polymerases known to those of skill in the art. In some embodiments, the engineered TdT polypeptides exhibit increased incorporation efficiency in extension of an oligo acceptor substrate by addition of an NTP or NQP of greater than 60%, 65%, 70%, 75%, 80%, 85%, 90%, 91%, 92%, 93%, 94%, 95%, 96%, 97%, 98%, or 99%. Exemplary incorporation efficiency of engineered TdTs are provided in the Examples, e.g., Example 92.

The activity of each engineered TdT relative to the reference polypeptide of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246 was determined as conversion of the substrates described in the Examples herein. In some embodiments, a shake flask purified enzyme (SFP) is used to assess the properties of the engineered TdTs, the results of which are provided in the Examples.

In some embodiments, the specific enzyme properties are associated with the residues differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246 at the residue positions indicated herein. In some embodiments, residue differences affecting polypeptide expression can be used to increase expression of the engineered TdTs.

In light of the guidance provided herein, it is further contemplated that any of the exemplary engineered polypeptides comprising the sequences of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246 find use as the starting amino acid sequence for synthesizing other TdT polypeptides, for example by subsequent rounds of evolution that incorporate new combinations of various amino acid differences from other polypeptides in Tables 5.1, 6.2, 7.2, 8.2, 9.2, 10.2, 11.2, 12.2, 13.2, 14.2, 15.2, 16.2, 17.2, 18.2, 19.2, 20.2, 21.2, 22.2, 23.2, 24.2, 25.2, 26.2, 26.3, 26.4, 27.2, 27.3, 27.4, 27.5, 28.1, 28.2, 28.3, 29.2, 30.2, 31.2, 32.2, 33.2, 34.2, 35.2, 36.2, 37.2, 38.2, 39.2, 40.2, 41.2, 42.2, 43.2, 44.2, 45.2 46.2, 47.2, 48.2, 49.2, 50.2, 51.2, 52.2, 53.2, 54.2, 55.2, 56.2, 56.3, 56.4, 61.2, 63.2, 64.2, 65.2, 66.2, 67.2, 68.2, 69.2, 70.2, 71.2, 72.2, 73.2, 74.2, 75.2, 76.2, 77.2, 78.2, 79.2, and 80.1, and other residue positions described herein. Further improvements may be generated by including amino acid differences at residue positions that had been maintained as unchanged throughout earlier rounds of evolution.

In some embodiments, the engineered TdT polypeptide has increased soluble protein expression and comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2 and one or more residue differences as compared to SEQ ID NO: 2, selected from

80/106/121/185/190/205/289/290/293/313/315/336/342/359/391/470/474/499/522/523,
80/106/121/185/190/205/289/290/293/313/342/470/474/499/523,
80/106/121/185/190/244/289/290/293/307/342/359/470/474/499,
80/121/131/185/190/205/244/289/290/293/313/315/336/342/359/391/414/470/474/499/522/523,
80/121/131/185/190/205/244/289/290/293/313/336/342/359/391/414/470/474/499/522/523,
80/121/131/185/190/289/290/293/313/342/470/474/499/522/523,
80/121/174/179/185/190/236/244/288/289/290/293/313/315/317/336/342/359/363/391/394/408/426/462/470/474/499/522/523,
80/121/174/185/186/190/236/244/273/284/288/289/290/293/313/315/317/336/342/352/359/391/394/395/419/428/431/462/470/474/499/522/523,
80/121/174/185/190/193/196/244/273/284/288/289/290/293/297/313/315/317/324/336/342/352/359/376/3
80/391/394/401/415/419/428/431/435/441/462/470/474/499/522/523,
80/121/174/185/190/193/244/273/284/288/289/290/293/297/313/315/317/336/342/352/359/391/394/415/419/428/431/462/470/474/499/522/523,
80/121/174/185/190/196/244/266/273/284/288/289/290/293/313/315/317/324/336/342/352/359/391/394/397/401/419/428/431/462/470/474/499/522/523,
80/121/174/185/190/236/244/273/282/284/288/289/290/293/313/315/317/336/342/352/359/391/394/395/419/428/431/462/470/474/499/522/523,
80/121/174/185/190/244/273/284/288/289/290/293/313/315/317/336/342/352/359/391/394/419/428/431/462/470/474/499/522/523,
80/121/174/185/190/244/273/284/288/289/290/293/313/315/317/336/342/359/391/394/428/431/462/470/474/499/522/523,
80/121/174/185/190/244/284/288/289/290/293/313/315/317/336/342/352/359/391/394/419/428/431/462/470/474/499/522/523,
80/121/174/185/190/244/284/288/289/290/293/313/315/317/336/342/352/359/391/394/428/431/462/470/474/499/522/523,
80/121/174/185/190/244/284/288/289/290/293/313/315/317/336/342/359/391/394/428/431/462/470/474/4
99/522/523, 80/121/185/190/196/244/289/290/293/313/315/317/336/342/359/391/470/474/499/522/523,
80/121/185/190/201/289/290/293/313/342/470/474/499/522,
80/121/185/190/244/273/289/290/293/313/315/317/336/342/352/359/391/419/435/470/474/499/522/523,
80/121/185/190/244/289/290/293/300/313/315/317/336/342/359/391/470/474/499/522/523,
80/121/185/190/244/289/290/293/313/315/317/336/342/359/380/391/401/419/470/474/499/522/523,
80/121/185/190/244/289/290/293/313/315/317/336/342/359/391/392/470/474/499/522/523,
80/121/185/190/244/289/290/293/313/315/317/336/342/359/391/395/470/474/499/522/523,
80/121/185/190/244/289/290/293/313/315/317/336/342/359/391/470/474/499/522/523,
80/121/185/190/244/289/290/293/313/336/342/359/391/414/470/474/499/522/523,
80/121/185/190/289/290/293/313/315/336/342/359/391/414/470/474/499/522/523,
80/121/185/190/289/290/293/313/336/342/359/391/470/474/499/522/523,
80/121/185/190/289/290/293/313/342/499, 80/121/185/315, 80/121/190/289/290, 80/185/236/289/293,
121/185/190/213/289/290/293, and 185/289/290/293. In some embodiments, the engineered TdT polypeptide has increased soluble protein expression and comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2 and one or more residue differences as compared to SEQ ID NO: 2, selected from
80D/106D/121S/185L/190E/205R/289D/290V/293S/313A/315G/336D/342E/359L/391G/470T/474M/49 9L/522L/523E, 80D/106D/121S/185L/190E/205R/289D/290V/293S/313A/342E/470T/474M/499L/523E, 80D/106D/121S/185L/190E/244V/289D/290V/293S/307K/342E/359L/470T/474M/499L, 80D/121S/131E/185L/190E/205R/244V/289D/290V/293S/313A/315G/336D/342E/359L/391G/414H/470 T/474M/499L/522L/523E,

### 80D/121S/131E/185L/190E/205R/244V/289D/290V/293S/313A/336D/342E/359L/391G/414H/470T/474M/499L/522L/523E,

### 80D/121S/131E/185L/190E/289D/290V/293S/313A/342E/470T/474M/499L/522L/523E,

### 80D/121S/174L/179K/185L/190E/236P/244V/288E/289D/290V/293S/313A/315G/317T/336D/342E/359 L/363I/391G/394R/408P/426P/462F/470T/474M/499L/522L/523E,

80D/121S/174L/185L/186G/190E/236P/244V/273V/284L/288E/289D/290V/293S/313A/315G/317T/336 D/342E/352P/359L/391G/394R/395R/419A/428V/431S/462F/470T/474M/499L/522L/523E, 80D/121S/174L/185L/190E/193G/196Y/244V/273V/284L/288E/289D/290V/293S/297R/313A/315G/317 T/324I/336D/342E/352P/359L/376H/380D/391G/394R/401G/415S/419A/428V/431S/435T/441M/462F/470T/474M/499L/522L/523E,

### 80D/121S/174L/185L/190E/193G/244V/273V/284L/288E/289D/290V/293S/297R/313A/315G/317T/336 D/342E/352P/359L/391G/394R/415S/419A/428V/431S/462F/470T/474M/499L/522L/523E,

### 80D/121S/174L/185L/190E/196R/244V/266K/273V/284L/288E/289D/290V/293S/313A/315G/317T/324 I/336D/342E/352P/359L/391G/394R/397R/401G/419A/428V/431S/462F/470T/474M/499L/522L/523E,

### 80D/121S/174L/185L/190E/236P/244V/273V/282R/284L/288E/289D/290V/293S/313A/315G/317T/336 D/342E/352P/359L/391G/394R/395R/419A/428V/431S/462F/470T/474M/499L/522L/523E,

### 80D/121S/174L/185L/190E/244V/273V/284L/288E/289D/290V/293S/313A/315G/317T/336D/342E/352P/359L/391G/394R/419A/428V/431S/462F/470T/474M/499L/522L/523E,

### 80D/121S/174L/185L/190E/244V/273V/284L/288E/289D/290V/293S/313A/315G/317T/336D/342E/359 L/391G/394R/428V/431S/462F/470T/474M/499L/522L/523E,

80D/121S/174L/185L/190E/244V/284L/288E/289D/290V/293S/313A/315G/317T/336D/342E/352P/359 L/391G/394R/419A/428V/431S/462F/470T/474M/499L/522L/523E, 80D/121S/174L/185L/190E/244V/284L/288E/289D/290V/293S/313A/315G/317T/336D/342E/352P/359 L/391G/394R/428V/431S/462F/470T/474M/499L/522L/523E,

### 80D/121S/174L/185L/190E/244V/284L/288E/289D/290V/293S/313A/315G/317T/336D/342E/359L/391 G/394R/428V/431S/462F/470T/474M/499L/522L/523E,

### 80D/121S/185L/190E/196R/244V/289D/290V/293S/313A/315G/317T/336D/342E/359L/391G/470T/474M/499L/522L/523E, 80D/121S/185L/190E/201R/289D/290V/293S/313A/342E/470T/474M/499L/522L,

### 80D/121S/185L/190E/244V/273V/289D/290V/293S/313A/315G/317T/336D/342E/352P/359L/391G/419 A/435T/470T/474M/499L/522L/523E,

### 80D/121S/185L/190E/244V/289D/290V/293S/300R/313A/315G/317T/336D/342E/359L/391G/470T/474M/499L/522L/523E,

### 80D/121S/185L/190E/244V/289D/290V/293S/313A/315G/317T/336D/342E/359L/380D/391G/401G/419 A/470T/474M/499L/522L/523E,

### 80D/121S/185L/190E/244V/289D/290V/293S/313A/315G/317T/336D/342E/359L/391G/392R/470T/474M/499L/522L/523E,

### 80D/121S/185L/190E/244V/289D/290V/293S/313A/315G/317T/336D/342E/359L/391G/395W/470T/47 4M/499L/522L/523E,

### 80D/121S/185L/190E/244V/289D/290V/293S/313A/315G/317T/336D/342E/359L/391G/470T/474M/499 L/522L/523E,

### 80D/121S/185L/190E/244V/289D/290V/293S/313A/336D/342E/359L/391G/414H/470T/474M/499L/522 L/523E,

### 80D/121S/185L/190E/289D/290V/293S/313A/315G/336D/342E/359L/391G/414H/470T/474M/499L/522 L/523E,

### 80D/121S/185L/190E/289D/290V/293S/313A/336D/342E/359L/391G/470T/474M/499L/522L/523E,

### 80D/121S/185L/190E/289D/290V/293S/313A/342E/499L, 80D/121S/185L/315G,

80D/121S/190E/289D/290V, 80D/185L/236P/289D/293S, 121S/185L/190E/213S/289D/290V/293S, and 185L/289D/290V/293S. In some embodiments, the engineered TdT polypeptide has increased soluble protein expression and comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2 and one or more residue differences as compared to SEQ ID NO: 2, selected from

### N80D/K106D/C121S/F185L/K190E/M205R/K289D/I290V/D293S/I313A/C315G/L336D/T342E/F359L/C391G/S470T/Q474M/K499L/I522L/Q523E,

### N80D/K106D/C121S/F185L/K190E/M205R/K289D/I290V/D293S/I313A/T342E/S470T/Q474M/K499L/Q523E,

### N80D/K106D/C121S/F185L/K190E/D244V/K289D/I290V/D293S/C307K/T342E/F359L/S470T/Q474M/K499L,

### N80D/C121S/K131E/F185L/K190E/M205R/D244V/K289D/I290V/D293S/I313A/C315G/L336D/T342E/F359L/C391G/F414H/S470T/Q474M/K499L/I522L/Q523E,

### N80D/C121S/K131E/F185L/K190E/M205R/D244V/K289D/I290V/D293S/I313A/L336D/T342E/F359L/C391G/F414H/S470T/Q474M/K499L/I522L/Q523E,

### N80D/C121S/K131E/F185L/K190E/K289D/I290V/D293S/I313A/T342E/S470T/Q474M/K499L/I522L/Q523E,

### N80D/C121S/I174L/Q179K/F185L/K190E/G236P/D244V/N288E/K289D/I290V/D293S/I313A/C315G/S317T/L336D/T342E/F359L/S363I/C391G/I394R/I408P/H426P/Y462F/S470T/Q474M/K499L/I522L/Q 523E,

### N80D/C121S/I174L/F185L/E186G/K190E/G236P/D244V/L273V/F284L/N288E/K289D/I290V/D293S/I 313A/C315G/S317T/L336D/T342E/E352P/F359L/C391G/I394R/E395R/L419A/E428V/R431S/Y462F/S 470T/Q474M/K499L/I522L/Q523E,

### N80D/C121S/I174L/F185L/K190E/E193G/E196Y/D244V/L273V/F284L/N288E/K289D/I290V/D293S/K297R/I313A/C315G/S317T/V324I/L336D/T342E/E352P/F359L/Q376H/N380D/C391G/I394R/L401G/Q415S/L419A/E428V/R431S/M435T/E441M/Y462F/S470T/Q474M/K499L/I522L/Q523E,

### N80D/C121S/I174L/F185L/K190E/E193G/D244V/L273V/F284L/N288E/K289D/I290V/D293S/K297R/I 313A/C315G/S317T/L336D/T342E/E352P/F359L/C391G/I394R/Q415S/L419A/E428V/R431S/Y462F/S 470T/Q474M/K499L/I522L/Q523E,

### N80D/C121S/I174L/F185L/K190E/E196R/D244V/T266K/L273V/F284L/N288E/K289D/I290V/D293S/I 313A/C315G/S317T/V324I/L336D/T342E/E352P/F359L/C391G/I394R/T397R/L401G/L419A/E428V/R 431S/Y462F/S470T/Q474M/K499L/I522L/Q523E,

### N80D/C121S/I174L/F185L/K190E/G236P/D244V/L273V/M282R/F284L/N288E/K289D/I290V/D293S/I313A/C315G/S317T/L336D/T342E/E352P/F359L/C391G/I394R/E395R/L419A/E428V/R431S/Y462F/S470T/Q474M/K499L/I522L/Q523E,

### N80D/C121S/I174L/F185L/K190E/D244V/L273V/F284L/N288E/K289D/I290V/D293S/I313A/C315G/S 317T/L336D/T342E/E352P/F359L/C391G/I394R/L419A/E428V/R431S/Y462F/S470T/Q474M/K499L/I 522L/Q523E,

### N80D/C121S/I174L/F185L/K190E/D244V/L273V/F284L/N288E/K289D/I290V/D293S/I313A/C315G/S 317T/L336D/T342E/F359L/C391G/I394R/E428V/R431S/Y462F/S470T/Q474M/K499L/I522L/Q523E,

### N80D/C121S/I174L/F185L/K190E/D244V/F284L/N288E/K289D/I290V/D293S/I313A/C315G/S317T/L 336D/T342E/E352P/F359L/C391G/I394R/L419A/E428V/R431S/Y462F/S470T/Q474M/K499L/I522L/Q 523E,

### N80D/C121S/I174L/F185L/K190E/D244V/F284L/N288E/K289D/I290V/D293S/I313A/C315G/S317T/L 336D/T342E/E352P/F359L/C391G/I394R/E428V/R431S/Y462F/S470T/Q474M/K499L/I522L/Q523E,

### N80D/C121S/I174L/F185L/K190E/D244V/F284L/N288E/K289D/I290V/D293S/I313A/C315G/S317T/L 336D/T342E/F359L/C391G/I394R/E428V/R431S/Y462F/S470T/Q474M/K499L/I522L/Q523E,

### N80D/C121S/F185L/K190E/E196R/D244V/K289D/I290V/D293S/I313A/C315G/S317T/L336D/T342E/F359L/C391G/S470T/Q474M/K499L/I522L/Q523E,

### N80D/C121S/F185L/K190E/C201R/K289D/I290V/D293S/I313A/T342E/S470T/Q474M/K499L/I522L,

### N80D/C121S/F185L/K190E/D244V/L273V/K289D/I290V/D293S/I313A/C315G/S317T/L336D/T342E/E352P/F359L/C391G/L419A/M435T/S470T/Q474M/K499L/I522L/Q523E,

### N80D/C121S/F185L/K190E/D244V/K289D/I290V/D293S/K300R/I313A/C315G/S317T/L336D/T342E/F359L/C391G/S470T/Q474M/K499L/I522L/Q523E,

### N80D/C121S/F185L/K190E/D244V/K289D/I290V/D293S/I313A/C315G/S317T/L336D/T342E/F359L/N380D/C391G/L401G/L419A/S470T/Q474M/K499L/I522L/Q523E,

### N80D/C121S/F185L/K190E/D244V/K289D/I290V/D293S/I313A/C315G/S317T/L336D/T342E/F359L/C391G/D392R/S470T/Q474M/K499L/I522L/Q523E,

### N80D/C121S/F185L/K190E/D244V/K289D/I290V/D293S/I313A/C315G/S317T/L336D/T342E/F359L/C391G/E395W/S470T/Q474M/K499L/I522L/Q523E,

### N80D/C121S/F185L/K190E/D244V/K289D/I290V/D293S/I313A/C315G/S317T/L336D/T342E/F359L/C391G/S470T/Q474M/K499L/I522L/Q523E,

### N80D/C121S/F185L/K190E/D244V/K289D/I290V/D293S/I313A/L336D/T342E/F359L/C391G/F414H/S470T/Q474M/K499L/I522L/Q523E,

### N80D/C121S/F185L/K190E/K289D/I290V/D293S/I313A/C315G/L336D/T342E/F359L/C391G/F414H/S470T/Q474M/K499L/I522L/Q523E,

### N80D/C121S/F185L/K190E/K289D/I290V/D293S/I313A/L336D/T342E/F359L/C391G/S470T/Q474M/K499L/I522L/Q523E, N80D/C121S/F185L/K190E/K289D/I290V/D293S/I313A/T342E/K499L,

### N80D/C121S/F185L/C315G, N80D/C121S/K190E/K289D/I290V, N80D/F185L/G236P/K289D/D293S, C121S/F185L/K190E/C213S/K289D/I290V/D293S, and F185L/K289D/I290V/D293S.

In some embodiments, the engineered TdT polypeptide has increased thermal stability and comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 36 and one or more residue differences as compared to SEQ ID NO: 36, selected from 174/244/273/284/288/315/317/336/352/359/391/394/419/428/431/462/470/474/522/523, 174/244/284/288/315/317/336/359/391/394/428/431/462/470/474/522/523, 244/315/317/336/359/391/470/474/522/523, and 336/359/391/470/474/522/523. In some embodiments, the engineered TdT polypeptide has increased thermal stability and comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 36 and one or more residue differences as compared to SEQ ID NO: 36, selected from

### 174L/244V/273V/284L/288E/315G/317T/336D/352P/359L/391G/394R/419A/428V/431S/462F/470T/474M/522L/523E,

174L/244V/284L/288E/315G/317T/336D/359L/391G/394R/428V/431S/462F/470T/474M/522L/523E, 244V/315G/317T/336D/359L/391G/470T/474M/522L/523E, and 336D/359L/391G/470T/474M/522L/523E. In some embodiments, the engineered TdT polypeptide has
increased thermal stability and comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 36 and one or more residue differences as compared to SEQ ID NO: 36, selected from

### I174L/D244V/L273V/F284L/N288E/C315G/S317T/L336D/E352P/F359L/C391G/I394R/L419A/E428V/R431S/Y462F/S470T/Q474M/I522L/Q523E,

### I174L/D244V/F284L/N288E/C315G/S317T/L336D/F359L/C391G/I394R/E428V/R431S/Y462F/S470T/Q474M/I522L/Q523E, D244V/C315G/S317T/L336D/F359L/C391G/S470T/Q474M/I522L/Q523E, and L336D/F359L/C391G/S470T/Q474M/I522L/Q523E.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 8 and one or more residue differences as compared to SEQ ID NO: 8, selected from 129/196, 173, 183, 186, 193, 195, 196, 263, 266, 268, 281, 282, 297, 300, 303, 316, 318, 320, 324, 343, 360, 392, 395, 397, 411, 415, 417, 421, 454, 456, 477, 481, and 492. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 8 and one or more residue differences as compared to SEQ ID NO: 8, selected from 129G/196G, 173L, 183R, 186A, 186G, 186L, 186T, 193C, 193G, 193N, 193V, 195R, 195W, 196A, 196G, 196R, 196W, 196Y, 263R, 266K, 268C, 281A, 282Q, 282R, 297F, 297Q, 297R, 297T, 300P, 300R, 303A, 303E, 303M, 316C, 316I, 316T, 318E, 318S, 318T, 318V, 320N, 324I, 343V, 360C, 360V, 392A, 392C, 392R, 395A, 395L, 395R, 395S, 395T, 395W, 395Y, 397R, 411A, 411G, 411R, 415A, 415S, 417G, 417V, 421I, 421M, 454V, 456K, 456R, 477T, 481E, 481V, and 492T. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 8 and one or more residue differences as compared to SEQ ID NO: 8, selected from D129G/E196G, T173L, D183R, E186A, E186G, E186L, E186T, E193C, E193G, E193N, E193V, K195R, K195W, E196A, E196G, E196R, E196W, E196Y, K263R, T266K, V268C, R281A, M282Q, M282R, K297F, K297Q, K297R, K297T, K300P, K300R, K303A, K303E, K303M, V316C, V316I, V316T, K318E, K318S, K318T, K318V, E320N, V324I, 1343V, L360C, L360V, D392A, D392C, D392R, E395A, E395L, E395R, E395S, E395T, E395W, E395Y, T397R, L411A, L411G, L411R, Q415A, Q415S, C417G, C417V, L421I, L421M, L454V, V456K, V456R, R477T, R481E, R481V, and D492T.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 16 and one or more residue differences as compared to SEQ ID NO: 16, selected from 186, 186/236/318, 186/236/395, 186/282/318, 193/196, 193/196/266/324/376/380, 193/196/297, 193/196/297/324/376/380/401/415/435/441, 193/196/324, 193/196/324/397/401/441, 193/196/376/380, 193/297/324/376/435, 193/297/324/380, 193/297/415, 193/435, 196, 196/266, 196/266/324/397/401, 196/297/324/435, 236/282, 236/282/395, 236/318/481, 266/297/380/397/401, 282, 282/318, 282/481, 297/380/401/441, 297/435, 318/395, 376/401/441, 415, and 435/441. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 16 and one or more residue differences as compared to SEQ ID NO: 16, selected from 186G, 186G/236P/318S, 186G/236P/395R, 186G/282R/318S, 193G/196R/297R, 193G/196Y, 193G/196Y/266K/324I/376H/380D, 193G/196Y/297R/324I/376H/380D/401G/415S/435T/441M, 193G/196Y/324I/397R/401G/441M, 193G/196Y/376H/380D, 193G/297R/324I/376H/435T, 193G/297R/415S, 193G/435T, 193N/196Y/324I, 193N/297R/324I/380D, 196R, 196R/266K, 196R/266K/324I/397R/401G, 196Y/297R/324I/435T, 236P/282R, 236P/282R/395R, 236P/318S/481E, 266K/297R/380D/397R/401G, 282R, 282R/318S, 282R/481E, 297R/380D/401G/441M, 297R/435T, 318S/395R, 376H/401G/441M, 415S, and 435T/441M. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 16 and one or more residue differences as compared to SEQ ID NO: 16, selected from E186G, E186G/G236P/K318S, E186G/G236P/E395R,

### E186G/M282R/K318S, E193G/E196R/K297R, E193G/E196Y,

### E193G/E196Y/T266K/V324I/Q376H/N380D,

### E193G/E196Y/K297R/V324I/Q376H/N380D/L401G/Q415S/M435T/E441M,

### E193G/E196Y/V324I/T397R/L401G/E441M, E193G/E196Y/Q376H/N380D,

### E193G/K297R/V324I/Q376H/M435T, E193G/K297R/Q415S, E193G/M435T, E193N/E196Y/V324I,

### E193N/K297R/V324I/N380D, E196R, E196R/T266K, E196R/T266K/V324I/T397R/L401G,

### E196Y/K297R/V324I/M435T, G236P/M282R, G236P/M282R/E395R, G236P/K318S/R481E,

### T266K/K297R/N380D/T397R/L401G, M282R, M282R/K318S, M282R/R481E,

### K297R/N380D/L401G/E441M, K297R/M435T, K318S/E395R, Q376H/L401G/E441M, Q415S, and

### M435T/E441M.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 24 and one or more residue differences as compared to SEQ ID NO: 24, selected from 12, 13, 14, 17, 18, 20, 21, 22, 23, 24, 26, 27, 29, 30, 31, 33, 34, 35, 37, 41, 53, 57, 58, 61, 92, 94, 97, 101, 102, 103, 104, 105, 106, 107, 108, 124, 126, 133, 135, 137, 138, 139, 140, 141, 142, 144, 145, 147, 149, 150, 152, 153, 154, 155, 156, 156/294, 159, 160, 161, 162, and 163. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 24 and one or more residue differences as compared to SEQ ID NO: 24, selected from 12S, 13G, 13K, 13R, 13S, 14G, 14Q, 17A, 17G, 18D, 18R, 20H, 20T, 21E, 22G, 22L, 23E, 23P, 24G, 26G, 27D, 29P, 29R, 30E, 30G, 30V, 31S, 33G, 33K, 33P, 34D, 34K, 34R, 34S, 35E, 35G, 37A, 37F, 37G, 37S, 37T, 37V, 41V, 53E, 57H, 58A, 58S, 61H, 61L, 92M, 92R, 92S, 92V, 92Y, 94E, 94R, 97D, 101E, 102L, 103M, 104G, 104I, 104P, 105N, 106G, 106H, 106S, 107R, 107W, 108D, 108K, 124E, 124I, 126V, 133G, 135R, 137A, 138Q, 139A, 140E, 140G, 141E, 141M, 141R, 142M, 142S, 144C, 145E, 147L, 149R, 150E, 150G, 152G, 152R, 153E, 153G, 153K, 153M, 153P, 153Q, 153V, 154G, 155E, 155T, 156D, 156E/294T, 156M, 156Q, 159D, 160G, 161D, 161E, 161G, 161R, 161S, 162E, 163L, and 163V. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 24 and one or more residue differences as compared to SEQ ID NO: 24, selected from M12S, H13G, H13K, H13R, H13S, R14G, R14Q, T17A, T17G, I18D, I18R, S20H, S20T, D21E, F22G, F22L, G23E, G23P, K24G, R26G, Q27D, K29P, K29R, M30E, M30G, M30V, D31S, H33G, H33K, H33P, I34D, I34K, I34R, I34S, S35E, S35G, M37A, M37F, M37G, M37S, M37T, M37V, I41V, K53E, A57H, T58A, T58S, T61H, T61L, G92M, G92R, G92S, G92V, G92Y, D94E, D94R, A97D, T101E, H102L, K103M, M104G, M104I, M104P, E105N, K106G, K106H, K106S, T107R, T107W, T108D, T108K, V124E, V124I, K126V, K133G, Q135R, M137A, E138Q, S139A, R140E, R140G, V141E, V141M, V141R, D142M, D142S, A144C, N145E, D147L, T149R, A150E, A150G, T152G, T152R, L153E, L153G, L153K, L153M, L153P, L153Q, L153V, N154G, I155E, I155T, L156D, L156E/K294T, L156M, L156Q, T159D, T160G, K161D, K161E, K161G, K161R, K161S, T162E, I163L, and I163V.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 24 and one or more residue differences as compared to SEQ ID NO: 24, selected from 7/135, 12, 13, 14, 15, 16, 17/131, 18, 20, 23, 24, 25, 26, 27, 28, 29, 31, 32, 33, 34, 35, 44, 45, 46, 57, 65, 77, 85, 89, 93, 94, 97, 101, 102, 103, 105, 106, 108, 109, 110, 119, 123, 124, 126, 130, 131, 132, 133, 134, 135, 137, 138, 139, 149, 150, 153, and 156. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 24 and one or more residue differences as compared to SEQ ID NO: 24, selected from 7Y/135C, 12F, 13E, 14G, 14N, 14Y, 15L, 16V, 17A/131R, 18Y, 20P, 23C, 23E, 23T, 24A, 24M, 24P, 25N, 26G, 27D, 27F, 28R, 29G, 29I, 31V, 32E, 33A, 33C, 34S, 35H, 35W, 44S, 45R, 46M, 57T, 65S, 77C, 77S, 85V, 89A, 93V, 94N, 97T, 101E, 101G, 101V, 102L, 103M, 105N, 105W, 106V, 108G, 108M, 109M, 109N, 109T, 110M, 110V, 119F, 119Q, 123M, 123Q, 124E, 124G, 124I, 124M, 124S, 126C, 130A, 130M, 130Q, 130S, 131G, 131W, 132S, 133G, 133M, 133Q, 134M, 134W, 135E, 135H, 135K, 137A, 137E, 138A, 139G, 149R, 150E, 153E, 153G, 153P, 153Q, and 156D. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 24 and one or more residue differences as compared to SEQ ID NO: 24, selected from H7Y/Q135C, M12F, H13E, R14G, R14N, R14Y, I15L, R16V, T17A/K131R, I18Y, S20P, G23C, G23E, G23T, K24A, K24M, K24P, K25N, R26G, Q27D, Q27F, K28R, K29G, K29I, D31V, N32E, H33A, H33C, I34S, S35H, S35W, H44S, E45R, F46M, A57T, D65S, E77C, E77S, I85V, N89A, S93V, D94N, A97T, T101E, T101G, T101V, H102L, K103M, E105N, E105W, K106V, T108G, T108M, Q109M, Q109N, Q109T, F110M, F110V, I119F, I119Q, K123M, K123Q, V124E, V124G, V124I, V124M, V124S, K126C, T130A, T130M, T130Q, T130S, K131G, K131W, G132S, K133G, K133M, K133Q, Y134M, Y134W, Q135E, Q135H, Q135K, M137A, M137E, E138A, S139G, T149R, A150E, L153E, L153G, L153P, L153Q, and L156D.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 268 and one or more residue differences as compared to SEQ ID NO: 268, selected from 14/53/300, 14/53/419, 106/300/415/419/456, 140, and 300/395/419. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 268 and one or more residue differences as compared to SEQ ID NO: 268, selected from 14G/53K/300P, 14G/53K/419L, 106V/300P/415A/419L/456R, 140G, and 300P/395Y/419L. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 268 and one or more residue differences as compared to SEQ ID NO: 268, selected from R14G/E53K/K300P, R14G/E53K/A419L, K106V/K300P/Q415A/A419L/V456R, R140G, and K300P/E395Y/A419L.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 648 and one or more residue differences as compared to SEQ ID NO: 648, selected from 12/14/34, 12/14/34/37/94/140/141/145, 12/14/34/37/106/140/142/150/152/153, 12/14/34/37/141/142, 12/14/34/37/142/145, 12/14/34/37/142/161, 12/14/34/37/150, 12/14/34/37/150/153, 12/14/34/140/142, 12/14/34/140/150, 12/14/34/142/150/153, 12/14/92/94, 12/14/94/150/152, 12/14/106/107/141/142, 12/14/106/108/140/141/145/150, 12/14/106/108/152, 12/14/141/142, 12/14/150/152/153, 12/14/153, 12/34/92/140, 12/34/150/152, 12/37/94/141/150/152/153, 12/37/140/141/150/162, 12/161, 14, 14/31/34/37/140/141/145/161/162, 14/34/37, 14/34/37/145, 14/34/37/152, 14/34/94/106/108/141, 14/34/150/153, 14/106, 14/140, 14/141/161, 14/142, 14/142/161/162, 14/153, 14/161, 20/21/24/33/58/104/106/124/155/156, 20/21/33/58/101/104/106/124/155, 20/21/58/104/106/155/156, 20/33/104/106/124/156, 20/58/101/104/106/156, 20/58/101/106, 20/101/106/156, 21/33/58/101/106, 21/33/58/106/155/156, 21/33/101/104/106, 21/33/106, 21/58/101/104/106/155, 21/58/106/155/156, 21/101/104/106/156, 21/104/106, 21/104/106/124, 21/104/106/156, 30/33/58/104/106/155/156, 30/33/101/106/156, 30/33/104/106/155/156, 30/101/104/106/155/156, 30/104/106/155, 30/104/106/155/156, 30/106/155, 33/58/104/106, 33/101/104/106/155, 34/37, 34/37/92, 34/37/140/141/142/145, 34/37/141/142, 34/37/150/153, 34/92/94/141/142, 34/141/142/145, 34/150/152/153, 37/92/142, 37/141/142, 37/153, 58/101/104/106/156, 58/101/106/155, 58/104/106/155/156, 92/94/106/142/145, 101/104/106, 101/104/106/155/156, 101/104/106/156, 101/106, 101/106/124/155, 101/106/155/156, 104/106, 104/106/124, 104/106/155, 104/106/155/156, 104/106/156, 106/107/108/142/220, 106/108/140/141/152/153, 106/108/140/142/150/153, 106/156, 140/141/142, 140/145, 140/145/150/152, 141, 141/152, and 161. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 648 and one or more residue differences as compared to SEQ ID NO: 648, selected from 12S/14G/34D, 12S/14G/34D/37A/106K/140G/142M/150E/152R/153E, 12S/14G/34D/37A/141E/142S, 12S/14G/34D/37A/150E, 12S/14G/34D/37F/94E/140G/141E/145E, 12S/14G/34D/37F/142S/145E, 12S/14G/34D/37F/150E/153E, 12S/14G/34D/140G/142S, 12S/14G/34D/140G/150E, 12S/14G/34S/37A/142M/161E, 12S/14G/34S/142S/150E/153E, 12S/14G/92R/94E, 12S/14G/94E/150E/152R, 12S/14G/106K/107R/141E/142M, 12S/14G/106K/108K/140G/141E/145E/150E, 12S/14G/106K/108K/152R, 12S/14G/141E/142S, 12S/14G/150E/152R/153E, 12S/14G/153E, 12S/34D/92R/140G, 12S/34D/150E/152R, 12S/37A/94E/141E/150E/152R/153E, 12S/37F/140G/141E/150E/162E, 12S/161E, 14G, 14G/31G/34D/37A/140G/141E/145E/161E/162E, 14G/34D/94E/106K/108K/141E, 14G/34D/150E/153E, 14G/34S/37A, 14G/34S/37A/152R, 14G/34S/37F/145E, 14G/34S/150E/153E, 14G/106K, 14G/140G, 14G/141E/161E, 14G/142M, 14G/142S/161E/162E, 14G/153E, 14G/161E, 20T/21E/24G/33K/58A/104P/106S/124E/155E/156E, 20T/21E/33K/58A/101E/104P/106K/124E/155E, 20T/21E/58A/104I/106S/155E/156E, 20T/33K/104P/106K/124E/156E, 20T/58A/101E/106K, 20T/58S/101E/104P/106S/156E, 20T/101E/106K/156E, 21E/33K/58A/101E/106K, 21E/33K/58S/106S/155E/156E, 21E/33K/101E/104P/106S, 21E/33K/106S, 21E/58A/106K/155E/156E, 21E/58S/101E/104I/106S/155E, 21E/101E/104P/106S/156E, 21E/104I/106S/124E, 21E/104P/106S, 21E/104P/106S/156E, 30G/33K/58S/104P/106S/155E/156E, 30G/33K/101E/106S/156E, 30G/33K/104I/106S/155E/156E, 30G/101E/104P/106S/155E/156E, 30G/104P/106S/155E, 30G/104P/106S/155E/156E, 30G/106K/155E, 33K/58A/104I/106S, 33K/101E/104P/106K/155E, 34D/37A, 34D/37A/92R, 34D/37F/141E/142S, 34D/92R/94E/141E/142M, 34D/141E/142S/145E, 34D/150E/152R/153E, 34S/37A/150E/153E, 34S/37F/140G/141E/142M/145E, 37A/141E/142S, 37F/92R/142M, 37F/153E, 58A/101E/106K/155E, 58S/101E/104P/106S/156E, 58S/104I/106K/155E/156E, 92R/94E/106K/142S/145E, 101E/104I/106K, 101E/104I/106K/156E, 101E/104I/106S/156E, 101E/104P/106K/155E/156E, 101E/106K, 101E/106K/155E/156E, 101E/106S/124E/155E, 104I/106K, 104I/106K/124E, 104I/106S, 104I/106S/155E/156E, 104P/106K, 104P/106K/155E, 104P/106K/156E, 104P/106S, 104P/106S/155E, 106K/107R/108K/142S/220R, 106K/108K/140G/141E/152R/153E, 106K/108K/140G/142S/150E/153E, 106K/156E, 140G/141E/142M, 140G/145E, 140G/145E/150E/152R, 141E, 141E/152R, and 161E. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 648 and one or more residue differences as compared to SEQ ID NO: 648, selected from M12S/R14G/I34D, M12S/R14G/I34D/M37A/V106K/R140G/D142M/A150E/T152R/L153E, M12S/R14G/I34D/M37A/V141E/D142S, M12S/R14G/I34D/M37A/A150E, M12S/R14G/I34D/M37F/D94E/R140G/V141E/N145E, M12S/R14G/I34D/M37F/D142S/N145E, M12S/R14G/I34D/M37F/A150E/L153E, M12S/R14G/I34D/R140G/D142S, M12S/R14G/I34D/R140G/A150E, M12S/R14G/I34S/M37A/D142M/K161E, M12S/R14G/I34S/D142S/A150E/L153E, M12S/R14G/G92R/D94E, M12S/R14G/D94E/A150E/T152R, M12S/R14G/V106K/T107R/V141E/D142M, M12S/R14G/V106K/T108K/R140G/V141E/N145E/A150E, M12S/R14G/V106K/T108K/T152R, M12S/R14G/V141E/D142S, M12S/R14G/A150E/T152R/L153E, M12S/R14G/L153E, M12S/I34D/G92R/R140G, M12S/I34D/A150E/T152R, M12S/M37A/D94E/V141E/A150E/T152R/L153E, M12S/M37F/R140G/V141E/A150E/T162E, M12S/K161E, R14G, R14G/D31G/I34D/M37A/R140G/V141E/N145E/K161E/T162E, R14G/I34D/D94E/V106K/T108K/V141E, R14G/I34D/A150E/L153E, R14G/I34S/M37A, R14G/I34S/M37A/T152R, R14G/I34S/M37F/N145E, R14G/I34S/A150E/L153E, R14G/V106K, R14G/R140G, R14G/V141E/K161E, R14G/D142M, R14G/D142S/K161E/T162E, R14G/L153E, R14G/K161E, S20T/D21E/K24G/H33K/T58A/M104P/V106S/V124E/I155E/L156E, S20T/D21E/H33K/T58A/T101E/M104P/V106K/V124E/I155E, S20T/D21E/T58A/M104I/V106S/I155E/L156E, S20T/H33K/M104P/V106K/V124E/L156E, S20T/T58A/T101E/V106K, S20T/T58S/T101E/M104P/V106S/L156E, S20T/T101E/V106K/L156E, D21E/H33K/T58A/T101E/V106K, D21E/H33K/T58S/V106S/I155E/L156E, D21E/H33K/T101E/M104P/V106S, D21E/H33K/V106S, D21E/T58A/V106K/I155E/L156E, D21E/T58S/T101E/M104I/V106S/I155E, D21E/T101E/M104P/V106S/L156E, D21E/M104I/V106S/V124E, D21E/M104P/V106S, D21E/M104P/V106S/L156E, M30G/H33K/T58S/M104P/V106S/I155E/L156E, M30G/H33K/T101E/V106S/L156E, M30G/H33K/M104I/V106S/I155E/L156E, M30G/T101E/M104P/V106S/I155E/L156E, M30G/M104P/V106S/I155E, M30G/M104P/V106S/I155E/L156E, M30G/V106K/I155E, H33K/T58A/M104I/V106S, H33K/T101E/M104P/V106K/I155E, I34D/M37A, I34D/M37A/G92R, I34D/M37F/V141E/D142S, I34D/G92R/D94E/V141E/D142M, I34D/V141E/D142S/N145E, I34D/A150E/T152R/L153E, I34S/M37A/A150E/L153E, I34S/M37F/R140G/V141E/D142M/N145E, M37A/V141E/D142S, M37F/G92R/D142M, M37F/L153E, T58A/T101E/V106K/I155E, T58S/T101E/M104P/V106S/L156E, T58S/M104I/V106K/I155E/L156E, G92R/D94E/V106K/D142S/N145E, T101E/M104I/V106K, T101E/M104I/V106K/L156E, T101E/M104I/V106S/L156E, T101E/M104P/V106K/I155E/L156E, T101E/V106K, T101E/V106K/I155E/L156E, T101E/V106S/V124E/I155E, M104I/V106K, M104I/V106K/V124E, M104I/V106S, M104I/V106S/I155E/L156E, M104P/V106K, M104P/V106K/I155E, M104P/V106K/L156E, M104P/V106S, M104P/V106S/I155E, V106K/T107R/T108K/D142S/S220R, V106K/T108K/R140G/V141E/T152R/L153E, V106K/T108K/R140G/D142S/A150E/L153E, V106K/L156E, R140G/V141E/D142M, R140G/N145E, R140G/N145E/A150E/T152R, V141E, V141E/T152R, and K161E.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 660 and one or more residue differences as compared to SEQ ID NO: 660, selected from 16/29/30, 16/29/30/33/153, 16/29/30/101/104, 16/30/104, 16/33, 29/30, 58, 92/94/108/141/155/392, 92/101/137/155/476,94/101/156/476, 101, 101/104, 101/137/155, 101/141/155/156, and 108. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 660 and one or more residue differences as compared to SEQ ID NO: 660, selected from 16V/29I/30E, 16V/29I/30E/33K/153P, 16V/29I/30E/101E/104V, 16V/30L/104V, 16V/33K, 29I/30E, 58A, 92R/94E/108K/141E/155E/392R, 92R/101E/137A/155E/476R, 94E/101E/156E/476R, 101E, 101E/104V, 101E/137E/155E, 101E/141E/155E/156E, and 108K. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 660 and one or more residue differences as compared to SEQ ID NO: 660, selected from R16V/K29I/M30E, R16V/K29I/M30E/H33K/E153P, R16V/K29I/M30E/T101E/M104V, R16V/M30L/M104V, R16V/H33K, K29I/M30E, T58A, G92R/D94E/T108K/V141E/I155E/D392R, G92R/T101E/M137A/I155E/E476R, D94E/T101E/L156E/E476R, T101E, T101E/M104V, T101E/M137E/I155E, T101E/V141E/I155E/L156E, and T108K.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 660 and one or more residue differences as compared to SEQ ID NO: 660, selected from 195, 197, 204/342, 205, 236/297, 258, 261, 262, 264, 268, 269, 276, 278, 280, 281, 282, 290, 291, 297, 300, 303, 306, 308, 309, 310, 312, 315, 316, 342, 344, 353, 360, 385, 391, 410, 413, 419, 421, 448, 454, 456, 473, 476, 515, and 525. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 660 and one or more residue differences as compared to SEQ ID NO: 660, selected from 195E, 197M, 204L/342W, 205E, 205L, 236E/297L, 258A, 258C, 258G, 258L, 258M, 258S, 258W, 261G, 261R, 261V, 262I, 264A, 264E, 264R, 264S, 268L, 269W, 276S, 278C, 278E, 278I, 278R, 278T, 278V, 280F, 281A, 281C, 281G, 281L, 281S, 281T, 281V, 282C, 282G, 282H, 282W, 290A, 290L, 291S, 297C, 297D, 297S, 297V, 300R, 303A, 303N, 303Q, 303S, 303V, 306F, 308F, 308W, 309F, 310A, 310G, 310H, 310R, 310S, 312V, 315A, 315S, 316A, 316L, 342R, 342V, 344V, 353A, 353K, 353M, 353R, 353S, 360I, 385R, 391L, 391R, 410E, 413C, 413F, 413V, 419G, 419H, 421F, 448R, 454M, 456S, 473V, 476V, 515V, 525F, and 525H. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 660 and one or more residue differences as compared to SEQ ID NO: 660, selected from K195E, N197M, F204L/E342W, M205E, M205L, G236E/K297L, R258A, R258C, R258G, R258L, R258M, R258S, R258W, S261G, S261R, S261V, F262I, L264A, L264E, L264R, L264S, V268L, F269W, A276S, K278C, K278E, K278I, K278R, K278T, K278V, Y280F, R281A, R281C, R281G, R281L, R281S, R281T, R281V, M282C, M282G, M282H, M282W, V290A, V290L, R291S, K297C, K297D, K297S, K297V, P300R, K303A, K303N, K303Q, K303S, K303V, L306F, Y308F, Y308W, Y309F, E310A, E310G, E310H, E310R, E310S, L312V, G315A, G315S, V316A, V316L, E342R, E342V, T344V, F353A, F353K, F353M, F353R, F353S, L360I, Q385R, G391L, G391R, A410E, H413C, H413F, H413V, L419G, L419H, L421F, K448R, L454M, R456S, R473V, E476V, A515V, S525F, and S525H.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 882 and one or more residue differences as compared to SEQ ID NO: 882, selected from 175, 196, 199, 203, 208, 275, 313, 314, 317, 321, 322, 325, 329/462, 379, 394, 397, 403/462, 406, 408, 457, 461, 462, 469, 477, 481, 484, and 495. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 882 and one or more residue differences as compared to SEQ ID NO: 882, selected from 175L, 196G, 196Y, 199G, 199M, 199Q, 199R, 199S, 199V, 203A, 203G, 203L, 203R, 203S, 208V, 275V, 313I, 314G, 314K, 314L, 314R, 314V, 314Y, 317G, 321C, 321K, 321S, 322A, 325F, 325T, 325V, 325W, 329R/462E, 379C, 394E, 394T, 397D, 397T, 403F/462H, 406G, 406V, 408A, 408T, 457S, 457V, 461G, 461V, 462I, 462R, 462W, 469Q, 477T, 481D, 481M, 481T, 484M, 484R, and 495S. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 882 and one or more residue differences as compared to SEQ ID NO: 882, selected from N175L, R196G, R196Y, D199G, D199M, D199Q, D199R, D199S, D199V, T203A, T203G, T203L, T203R, T203S, I208V, T275V, A313I, D314G, D314K, D314L, D314R, D314V, D314Y, T317G, A321C, A321K, A321S, D322A, S325F, S325T, S325V, S325W, Q329R/F462E, T379C, R394E, R394T, R397D, R397T, L403F/F462H, R406G, R406V, I408A, I408T, C457S, C457V, R461G, R461V, F462I, F462R, F462W, W469Q, R477T, R481D, R481M, R481T, T484M, T484R, and A495S.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 882 and one or more residue differences as compared to SEQ ID NO: 882, selected from 175, 179, 196, 199, 201, 203, 272, 273, 275, 307, 313, 314, 319, 321, 322, 324, 325, 350, 376, 394, 404, 406, 408, 461, 462, 477, 481, 484, 491, 492, 495, and 523. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 882 and one or more residue differences as compared to SEQ ID NO: 882, selected from 175D, 175I, 175V, 179R, 196G, 199A, 199E, 199G, 199H, 199I, 199Q, 199R, 199V, 201A, 201W, 203M, 203R, 272T, 273M, 273W, 275D, 307M, 307V, 313M, 313Q, 313R, 313S, 314G, 314I, 319G, 319R, 321K, 322K, 322Q, 324V, 325A, 325V, 350S, 376V, 394L, 394M, 394S, 394T, 404F, 404W, 406V, 408G, 408L, 408R, 461A, 461G, 461Q, 461S, 462L, 462Q, 477Q, 481W, 484M, 484R, 491I, 492S, 492T, 495G, 495S, and 523H. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 882 and one or more residue differences as compared to SEQ ID NO: 882, selected from N175D, N175I, N175V, Q179R, R196G, D199A, D199E, D199G, D199H, D199I, D199Q, D199R, D199V, C201A, C201W, T203M, T203R, G272T, V273M, V273W, T275D, C307M, C307V, A313M, A313Q, A313R, A313S, D314G, D314I, A319G, A319R, A321K, D322K, D322Q, I324V, S325A, S325V, G350S, Q376V, R394L, R394M, R394S, R394T, P404F, P404W, R406V, I408G, I408L, I408R, R461A, R461G, R461Q, R461S, F462L, F462Q, R477Q, R481W, T484M, T484R, L491I, D492S, D492T, A495G, A495S, and E523H.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1336 and one or more residue differences as compared to SEQ ID NO: 1336, selected from 15/199/203/394, 15/199/394, 28/344/353/395, 195/199/203/278/297/394, 195/199/203/278/314/353/394, 195/203/278/394/395, 195/203/297/314/394, 195/203/297/394/419, 195/203/394, 195/278/297/394, 195/278/297/394/395, 195/314/344, 195/394/395, 199/203/297/394/395, 203/278/297/394, 203/297/314/394/395, 203/310/314/394/395/419, 203/344/394, 203/353, 203/394, 203/394/395, 297/394, 314/394/395, 344/394/395, 353, and 394. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1336 and one or more residue differences as compared to SEQ ID NO: 1336, selected from 15F/199R/203A/394E, 15F/199R/394E, 28N/344V/353K/395W, 195E/199R/203A/278E/297D/394E, 195E/199R/203A/278E/314R/353K/394E, 195E/203A/278E/394E/395W, 195E/203A/297D/314R/394E, 195E/203A/297D/394E/419M, 195E/203A/394E, 195E/278E/297D/394E, 195E/278E/297D/394E/395W, 195E/314R/344V, 195E/394E/395W, 199R/203A/297D/394E/395W, 203A/278E/297D/394E, 203A/297D/314R/394E/395W, 203A/310R/314R/394E/395W/419M, 203A/344V/394E, 203A/353K, 203A/394E, 203A/394E/395W, 297D/394E, 314R/394E/395W, 344V/394E/395W, 353K, and 394E. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1336 and one or more residue differences as compared to SEQ ID NO: 1336, selected from I15F/D199R/T203A/R394E, I15F/D199R/R394E, K28N/T344V/F353K/E395W, K195E/D199R/T203A/K278E/K297D/R394E, K195E/D199R/T203A/K278E/D314R/F353K/R394E, K195E/T203A/K278E/R394E/E395W, K195E/T203A/K297D/D314R/R394E, K195E/T203A/K297D/R394E/L419M, K195E/T203A/R394E, K195E/K278E/K297D/R394E, K195E/K278E/K297D/R394E/E395W, K195E/D314R/T344V, K195E/R394E/E395W, D199R/T203A/K297D/R394E/E395W, T203A/K278E/K297D/R394E, T203A/K297D/D314R/R394E/E395W, T203A/E310R/D314R/R394E/E395W/L419M, T203A/T344V/R394E, T203A/F353K, T203A/R394E, T203A/R394E/E395W, K297D/R394E, D314R/R394E/E395W, T344V/R394E/E395W, F353K, and R394E.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1336 and one or more residue differences as compared to SEQ ID NO: 1336, selected from 61, 152/503, 160, 162, 165, 177, 200, 200/425, 213, 217, 219, 223, 236, 246, 248, 292, 292/411, 295, 326, 329, 330, 333, 334, 338, 340, 340/438, 363, 369, 370, 372, 373, 383, 400/401/402, 425, 427, 435, 435/503, 437, 440, 441, 442, 443, 444, 446, 459, 460, 488, 490, 501, 502, 503, 504, and 506. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1336 and one or more residue differences as compared to SEQ ID NO: 1336, selected from 61A, 152H/503S, 160M, 160N, 160S, 160V, 162A, 165K, 177L, 200C, 200C/425K, 200K, 200V, 213S, 217Q, 219L, 219R, 223Q, 236I, 236P, 236V, 246K, 248C, 248S, 292L/411P, 292T, 295S, 326C, 326M, 326N, 326S, 326T, 329K, 329R, 330E, 333A, 333D, 333G, 333H, 333R, 334E, 334R, 334S, 338T, 340A, 340G, 340M, 340M/438V, 340R, 340S, 363C, 369G, 369M, 369N, 370G, 372G, 373H, 373N, 383R, 400A/401E/402F, 425D, 425R, 425T, 427E, 427Q, 435A, 435C, 435E, 435G, 435K, 435Q, 435S, 435S/503M, 435T, 437Q, 437S, 440E, 440V, 441N, 442G, 443T, 444R, 446E, 446P, 459Q, 460G, 488S, 490E, 490H, 490R, 490V, 490W, 501A, 502G, 502R, 503E, 503Q, 503R, 503V, 504N, 504R, 504W, and 506E. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1336 and one or more residue differences as compared to SEQ ID NO: 1336, selected from T61A, R152H/1I503S, T160M, T160N, T160S, T160V, T162A, Q165K, H177L, T200C, T200C/H425K, T200K, T200V, C213S, E217Q, V219L, V219R, D223Q, G236I, G236P, G236V, E246K, L248C, L248S, S292L/L411P, S292T, T295S, L326C, L326M, L326N, L326S, L326T, Q329K, Q329R, D330E, W333A, W333D, W333G, W333H, W333R, T334E, T334R, T334S, D338T, L340A, L340G, L340M, L340M/G438V, L340R, L340S, S363C, E369G, E369M, E369N, Q370G, D372G, Q373H, Q373N, K383R, D400A/G401E/K402F, H425D, H425R, H425T, K427E, K427Q, M435A, M435C, M435E, M435G, M435K, M435Q, M435S, M435S/I503M, M435T, T437Q, T437S, N440E, N440V, E441N, S442G, E443T, A444R, S446E, S446P, Y459Q, D460G, K488S, M490E, M490H, M490R, M490V, M490W, K501A, K502G, K502R, 1503E, I503Q, I503R, I503V, F504N, F504R, F504W, and K506E.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1348 and one or more residue differences as compared to SEQ ID NO: 1348, selected from 3/175/213/313/325/340/457/481/485, 3/307/321/340/353/406/408/445, 148/175/201/457/485, 175/201/333/412/425/457/485, 175/325/397, 175/333, 175/333/369/481, 175/333/485, 175/485, 199/307/321/340/406/408/445/484, 201/213/333/344/397/425/481/485, 201/333/344/457/481, 201/333/481, 201/406/408/462/484/502, 213, 213/333/397, 307/333/340/408/445/462/502, 307/373/406/408/484, 321/333/340, 325/333/369/425, 325/425/457/481, 333, 333/344/369/397, 333/344/369/485, 340/484, 344/485, 353/406, 373, and 406/408.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1348 and one or more residue differences as compared to SEQ ID NO: 1348, selected from 3Q/175D/213S/313R/325T/340R/457V/481W/485D, 3Q/307M/321K/340R/353K/406G/408A/445N, 148T/175D/201A/457V/485D, 175D/201A/333A/412N/425D/457V/485D, 175D/325T/397Q, 175D/333A, 175D/333A/369N/481W, 175D/333A/485D, 175D/485D, 199H/307M/321K/340R/406G/408A/445N/484M, 201A/213S/333A/344V/397Q/425D/481W/485D, 201A/333A/344V/457V/481W, 201A/333A/481W, 201R/406G/408A/462L/484M/502G, 213S, 213S/333A/397Q, 307M/333G/340R/408A/445N/462L/502G, 307M/373H/406G/408A/484M, 321K/333G/340R, 325T/333A/369N/425D, 325T/425D/457V/481W, 333A/344V/369N/397Q, 333A/344V/369N/485D, 333G, 340R/484M, 344V/485D, 353K/406G, 373H, and 406G/408A. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1348 and one or more residue differences as compared to SEQ ID NO: 1348, selected from H3Q/N175D/C213S/A313R/S325T/L340R/C457V/R481W/H485D, H3Q/C307M/A321K/L340R/F353K/R406G/I408A/K445N, P148T/N175D/C201A/C457V/H485D, N175D/C201A/W333A/D412N/H425D/C457V/H485D, N175D/S325T/R397Q, N175D/W333A, N175D/W333A/E369N/R481W, N175D/W333A/H485D, N175D/H485D, D199H/C307M/A321K/L340R/R406G/I408A/K445N/T484M, C201A/C213S/W333A/T344V/R397Q/H425D/R481W/H485D, C201A/W333A/T344V/C457V/R481W, C201A/W333A/R481W, C201R/R406G/1408A/F462L/T484M/K502G, C213S, C213S/W333A/R397Q, C307M/W333G/L340R/1408A/K445N/F462L/K502G, C307M/Q373H/R406G/1408A/T484M, A321K/W333G/L340R, S325T/W333A/E369N/H425D, S325T/H425D/C457V/R481W, W333A/T344V/E369N/R397Q, W333A/T344V/E369N/H485D, W333G, L340R/T484M, T344V/H485D, F353K/R406G, Q373H, and R406G/I408A.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from 160/219/460/503/506, 219/307/326, 252, 252/333, 406/408, 406/408/442/446, 406/408/490, 408, and 446. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from 160M/219R/460G/503E/506E, 219R/307M/326T, 252K, 252K/333H, 406G/408A, 406G/408A/442G/446P, 406G/408A/490R, 408A, and 446P. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from T160M/V219R/D460G/I503E/K506E, V219R/C307M/L326T, A252K, A252K/A333H, R406G/I408A, R406G/I408A/S442G/S446P, R406G/I408A/M490R, 1408A, and S446P.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from 162, 163, 165, 171, 177, 179, 188, 200, 205, 208, 233, 252, 253, 260, 261, 277, 307, 325, 329, 330, 353, 371, 376, 382, 393, 400, 402, 405, 406, 407, 410, 413, 419, 441, 442, 460, 464, 484, 488, 490, 495, 506, 508, and 520. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from 162H, 163V, 165P, 171K, 177Y, 179K, 188M, 200A, 205R, 208A, 233D, 252E, 253I, 260K, 261A, 277E, 307L, 325L, 329K, 330E, 353Q, 371D, 376H, 382L, 393I, 400E, 402Q, 405N, 406P, 407A, 410Q, 413G, 419A, 441M, 442A, 460E, 464Y, 484E, 488N, 490L, 495G, 506S, 508D, and 520D. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from T162H, I163V, Q165P, R171K, H177Y, Q179K, L188M, T200A, M205R, I208A, E233D, A252E, V253I, Q260K, S261A, D277E, C307L, S325L, Q329K, D330E, F353Q, E371D, Q376H, W382L, L393I, D400E, K402Q, S405N, R406P, K407A, A410Q, H413G, L419A, E441M, S442A, D460E, F464Y, T484E, K488N, M490L, A495G, K506S, K508D, and E520D.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from 161, 162, 163, 165, 179, 188, 205, 208, 231, 233, 251, 252, 253, 261, 277, 306, 307, 321, 325, 327, 329, 353, 368, 370, 371, 376, 380, 393, 400, 402, 406, 410, 413, 414, 419, 426, 441, 442, 460, 464, 484, 488, 490, 495, 506, 508, and 518. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from 161R, 162H, 163V, 165P, 179K, 188M, 205R, 208A, 231T, 233D, 251K, 252E, 253I, 261A, 277E, 306F, 307L, 321K, 325L, 327I, 329K, 353Q, 368R, 370T, 371D, 376H, 380D, 393I, 400E, 402Q, 406P, 410Q, 413G, 414H, 419A, 426P, 441M, 442A, 460E, 464Y, 484E, 488N, 490L, 495G, 506S, 508D, and 518D. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from K161R, T162H, I163V, Q165P, Q179K, L188M, M205R, I208A, G231T, E233D, Q251K, A252E, V253I, S261A, D277E, L306F, C307L, A321K, S325L, L327I, Q329K, F353Q, K368R, Q370T, E371D, Q376H, N380D, L393I, D400E, K402Q, R406P, A410Q, H413G, F414H, L419A, H426P, E441M, S442A, D460E, F464Y, T484E, K488N, M490L, A495G, K506S, K508D, and G518D.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1654 and one or more residue differences as compared to SEQ ID NO: 1654, selected from 160/165/203/205/219/353/460/488, 160/165/205/219/441, 160/203/205/441, 160/219/330/484, 179/353, 205/307/441/460/488, and 441. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1654 and one or more residue differences as compared to SEQ ID NO: 1654, selected from 160M/165P/203E/205R/219R/353Q/460G/488N, 160M/165P/205R/219R/441M, 160M/203E/205R/441M, 160M/219R/330K/484E, 179K/353Q, 205R/307L/441M/460G/488N, and 441M. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1654 and one or more residue differences as compared to SEQ ID NO: 1654, selected from T160M/Q165P/T203E/M205R/V219R/F353Q/D460G/K488N, T160M/Q165P/M205R/V219R/E441M, T160M/T203E/M205R/E441M, T160M/V219R/D330K/T484E, Q179K/F353Q, M205R/C307L/E441M/D460G/K488N, and E441M.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1654 and one or more residue differences as compared to SEQ ID NO: 1654, selected from 3/160/205/208/219/307/353/371/376/413/414/441/488, 160, 160/165/179/203/205/208/219/353/376/413/414/441/488, 160/165/179/203/307/376/413/495, 160/165/203/205/219/353/460/488, 160/165/203/205/307/371/376/413/414, 160/165/203/208/371/376/413/414, 160/165/205, 160/165/205/208, 160/165/205/219/441, 160/165/205/414/441/495, 160/165/307/353/413/414/441/488/495, 160/179/203/205/208/219/371/414/484/506, 160/179/208/219/307/413/414/503/506, 160/179/208/307/371/376/414, 160/179/307/376/441/488/503, 160/203/205/208/219/414/460/506, 160/203/205/208/413/460/484/488, 160/203/205/441, 160/203/208/307/353/495, 160/203/208/371/413/414, 160/203/208/413/414/441/484, 160/203/326/353/413/414/484/495, 160/205/208/219/326/441/484/488/503, 160/208/326/376/414/441/484/488, 160/208/371/441/484/506, 160/208/414/441/452/480/488/495, 160/219/307/371/506, 160/219/330/484, 165/179/203/205/219/414/418/441/488/503, 165/179/203/205/484/503, 165/179/205/413/441, 165/179/208/353/413/414/503, 165/203/205, 165/203/205/307/414/441/484/495/503/506, 165/203/205/484/488, 165/203/208/326/376/503, 165/205, 165/208/326/413/414/484/495/506, 179, 179/203/205, 179/203/208/326/353/376/484, 179/205/208/353/414/441/460/484/488, 179/205/353, 179/208/353/460, 179/353, 203/205/208/307/330/353/441/460/503/506, 203/205/208/307/441, 203/205/208/353, 203/208/219/376/441, 203/208/219/441, 203/208/326/353, 203/413/503/506, 205/208/307/353/376/413, 205/208/414, 205/219/307/353, 205/307, 205/307/376/414/441/495, 205/307/441/460/488, 205/326/488/503/506, 208/488/506, 326/353/371/376/414, and 441. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1654 and one or more residue differences as compared to SEQ ID NO: 1654, selected from

### 3Q/160M/205R/208A/219R/307L/353Q/371D/376H/413G/414H/441M/488N, 160M,

### 160M/165P/179K/203E/205R/208A/219R/353Q/376H/413G/414H/441M/488N,

### 160M/165P/179K/203E/307L/376H/413G/495G, 160M/165P/203E/205R/219R/353Q/460G/488N,

### 160M/165P/203E/205R/307L/371D/376H/413G/414H, 160M/165P/203E/208A/371D/376H/413G/414H,

### 160M/165P/205R, 160M/165P/205R/208A, 160M/165P/205R/219R/441M,

### 160M/165P/205R/414H/441M/495G, 160M/165P/307L/353Q/413G/414H/441M/488N/495G,

### 160M/179K/203E/205R/208A/219R/371D/414H/484E/506E,

### 160M/179K/208A/219R/307L/413G/414H/503E/506E, 160M/179K/208A/307L/371D/376H/414H,

### 160M/179K/307L/376H/441M/488N/503E, 160M/203E/205R/208A/219R/414H/460G/506E,

### 160M/203E/205R/208A/413G/460G/484E/488N, 160M/203E/205R/441M,

### 160M/203E/208A/307L/353Q/495G, 160M/203E/208A/371D/413G/414H,

### 160M/203E/208A/413G/414H/441M/484E, 160M/203E/326T/353Q/413G/414H/484E/495G,

### 160M/205R/208A/219R/326T/441M/484E/488N/503E,

### 160M/208A/326T/376H/414H/441M/484E/488N, 160M/208A/371D/441M/484E/506E,

### 160M/208A/414H/441M/452I/480H/488N/495G, 160M/219R/307L/371D/506E,

### 160M/219R/330K/484E, 165P/179K/203E/205R/219R/414H/418I/441M/488N/503E,

### 165P/179K/203E/205R/484E/503E, 165P/179K/205R/413G/441M,

### 165P/179K/208A/353Q/413G/414H/503E, 165P/203E/205R,

### 165P/203E/205R/307L/414Y/441M/484E/495G/503E/506E, 165P/203E/205R/484E/488N,

### 165P/203E/208A/326T/376H/503E, 165P/205R, 165P/208A/326T/413G/414H/484E/495G/506E, 179K,

### 179K/203E/205R, 179K/203E/208A/326T/353Q/376H/484E,

### 179K/205R/208A/353Q/414H/441M/460G/484E/488N, 179K/205R/353Q, 179K/208A/353Q/460G,

### 179K/353Q, 203E/205R/208A/307L/330N/353Q/441M/460G/503E/506E, 203E/205R/208A/307L/441M,

### 203E/205R/208A/353Q, 203E/208A/219R/376H/441M, 203E/208A/219R/441M,

### 203E/208A/326T/353Q, 203E/413G/503E/506E, 205R/208A/307L/353Q/376H/413G, 205R/208A/414H,

### 205R/219R/307L/353Q, 205R/307L, 205R/307L/376H/414H/441M/495G,

### 205R/307L/441M/460G/488N, 205R/326T/488N/503E/506E, 208A/488N/506E,

326T/353Q/371D/376H/414H, and 441M. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1654 and one or more residue differences as compared to SEQ ID NO: 1654, selected from H3Q/T160M/M205R/I208A/V219R/C307L/F353Q/E371D/Q376H/H413G/F414H/E441M/K488N, T160M,

### T160M/Q165P/Q179K/T203E/M205R/I208A/V219R/F353Q/Q376H/H413G/F414H/E441M/K488N,

### T160M/Q165P/Q179K/T203E/C307L/Q376H/H413G/A495G,

### T160M/Q165P/T203E/M205R/V219R/F353Q/D460G/K488N,

### T160M/Q165P/T203E/M205R/C307L/E371D/Q376H/H413G/F414H,

### T160M/Q165P/T203E/1208A/E371D/Q376H/H413G/F414H, T160M/Q165P/M205R,

### T160M/Q165P/M205R/I208A, T160M/Q165P/M205R/V219R/E441M,

### T160M/Q165P/M205R/F414H/E441M/A495G,

### T160M/Q165P/C307L/F353Q/H413G/F414H/E441M/K488N/A495G,

### T160M/Q179K/T203E/M205R/I208A/V219R/E371D/F414H/T484E/K506E,

### T160M/Q179K/I208A/V219R/C307L/H413G/F414H/I503E/K506E,

### T160M/Q179K/I208A/C307L/E371D/Q376H/F414H,

### T160M/Q179K/C307L/Q376H/E441M/K488N/I503E,

### T160M/T203E/M205R/I208A/V219R/F414H/D460G/K506E,

### T160M/T203E/M205R/I208A/H413G/D460G/T484E/K488N, T160M/T203E/M205R/E441M,

### T160M/T203E/I208A/C307L/F353Q/A495G, T160M/T203E/I208A/E371D/H413G/F414H,

### T160M/T203E/I208A/H413G/F414H/E441M/T484E,

### T160M/T203E/L326T/F353Q/H413G/F414H/T484E/A495G,

### T160M/M205R/I208A/V219R/L326T/E441M/T484E/K488N/I503E,

### T160M/I208A/L326T/Q376H/F414H/E441M/T484E/K488N,

### T160M/I208A/E371D/E441M/T484E/K506E,

### T160M/I208A/F414H/E441M/V452I/R480H/K488N/A495G, T160M/V219R/C307L/E371D/K506E,

### T160M/V219R/D330K/T484E,

### Q165P/Q179K/T203E/M205R/V219R/F414H/F418I/E441M/K488N/I503E,

### Q165P/Q179K/T203E/M205R/T484E/I503E, Q165P/Q179K/M205R/H413G/E441M,

### Q165P/Q179K/I208A/F353Q/H413G/F414H/I503E, Q165P/T203E/M205R,

### Q165P/T203E/M205R/C307L/F414Y/E441M/T484E/A495G/I503E/K506E,

### Q165P/T203E/M205R/T484E/K488N, Q165P/T203E/I208A/L326T/Q376H/I503E, Q165P/M205R,

### Q165P/I208A/L326T/H413G/F414H/T484E/A495G/K506E, Q179K, Q179K/T203E/M205R,

### Q179K/T203E/I208A/L326T/F353Q/Q376H/T484E,

### Q179K/M205R/I208A/F353Q/F414H/E441M/D460G/T484E/K488N, Q179K/M205R/F353Q,

### Q179K/I208A/F353Q/D460G, Q179K/F353Q,

### T203E/M205R/I208A/C307L/D330N/F353Q/E441M/D460G/I503E/K506E,

### T203E/M205R/I208A/C307L/E441M, T203E/M205R/I208A/F353Q,

### T203E/I208A/V219R/Q376H/E441M, T203E/I208A/V219R/E441M, T203E/I208A/L326T/F353Q,

### T203E/H413G/I503E/K506E, M205R/I208A/C307L/F353Q/Q376H/H413G, M205R/I208A/F414H,

### M205R/V219R/C307L/F353Q, M205R/C307L, M205R/C307L/Q376H/F414H/E441M/A495G,

### M205R/C307L/E441M/D460G/K488N, M205R/L326T/K488N/I503E/K506E, I208A/K488N/K506E,

### L326T/F353Q/E371D/Q376H/F414H, and E441M.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1830 and one or more residue differences as compared to SEQ ID NO: 1830, selected from 163/179/277/338/340, 163/414/441, 171/200/334/406/490, 177, and 292/406. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1830 and one or more residue differences as compared to SEQ ID NO: 1830, selected from 163V/179K/277E/338T/340A, 163V/414H/441M, 171K/200V/334R/406P/490L, 177L, and 292T/406P.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1830 and one or more residue differences as compared to SEQ ID NO: 1830, selected from I163V/Q179K/D277E/D338T/L340A, I163V/F414H/E441M, R171K/T200V/T334R/G406P/M490L, H177L, and S292T/G406P.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1100 and one or more residue differences as compared to SEQ ID NO: 1100, selected from 101/137/264/476/525 and 264. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1100 and one or more residue differences as compared to SEQ ID NO: 1100, selected from 101E/137A/264R/476R/525F and 264R. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1100 and one or more residue differences as compared to SEQ ID NO: 1100, selected from T101E/M137A/L264R/E476R/S525F and L264R.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1654 and one or more residue differences as compared to SEQ ID NO: 1654, selected from 160/163/165/203/205/219/353/414/441/460/488 and 160/165/203/205/219/353/460/488. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1654 and one or more residue differences as compared to SEQ ID NO: 1654, selected from

### 160M/163V/165P/203E/205R/219R/353Q/414H/441M/460G/488N and

160M/165P/203E/205R/219R/353Q/460G/488N. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1654 and one or more residue differences as compared to SEQ ID NO: 1654, selected from

### T160M/I163V/Q165P/T203E/M205R/V219R/F353Q/F414H/E441M/D460G/K488N and

### T160M/Q165P/T203E/M205R/V219R/F353Q/D460G/K488N.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from 160/165/203/205/219/353/406/408/442/446/460/488, 160/165/205/219/406/408/441/442/446, 160/203/205/406/408/441/442/446, 160/219/330/406/408/442/446/484, 205/307/406/408/441/442/446/460/488, 406/408/441/442/446, and 406/408/442/446. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from

### 160M/165P/203E/205R/219R/353Q/406G/408A/442G/446P/460G/488N,

### 160M/165P/205R/219R/406G/408A/441M/442G/446P, 160M/203E/205R/406G/408A/441M/442G/446P,

160M/219R/330K/406G/408A/442G/446P/484E, 205R/307L/406G/408A/441M/442G/446P/460G/488N, 406G/408A/441M/442G/446P, and 406G/408A/442G/446P. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1596 and one or more residue differences as compared to SEQ ID NO: 1596, selected from

### T160M/Q165P/T203E/M205R/V219R/F353Q/R406G/I408A/S442G/S446P/D460G/K488N,

### T160M/Q165P/M205R/V219R/R406G/I408A/E441M/S442G/S446P,

### T160M/T203E/M205R/R406G/I408A/E441M/S442G/S446P,

### T160M/V219R/D330K/R406G/I408A/S442G/S446P/T484E,

### M205R/C307L/R406G/I408A/E441M/S442G/S446P/D460G/K488N,

### R406G/I408A/E441M/S442G/S446P, and R406G/I408A/S442G/S446P.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1950 and one or more residue differences as compared to SEQ ID NO: 1950, selected from 163/169, 185, 186, 192, 193, 194, 197, 198, 245, 251, 258, 259, 261, 263, 271, 274, 278, 280, 284, 286, 290, 291, 297, 304, 306, 308, 316, 347, 352, 359, 362, 378, 393, 396, 398, 399, 405, 407, 409/414, 410/414, 411/414, 413/414, 414, 414/415, 414/417, 415, 455, 465, 466, 468, 494, and 509. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1950 and one or more residue differences as compared to SEQ ID NO: 1950, selected from 163I/169E, 163/169R, 185C, 185F, 186C, 192C, 192G, 192I, 192L, 192R, 193R, 193S, 194A, 194C, 194D, 194G, 194M, 194W, 197G, 197L, 197R, 198A, 198L, 245A, 251R, 258E, 258G, 258K, 258L, 258Q, 258W, 259N, 259V, 261A, 261G, 263I, 263S, 271C, 274M, 274N, 274P, 274Q, 274T, 274V, 278C, 278L, 278N, 280L, 280S, 284I, 284M, 286N, 286S, 290A, 291M, 291W, 297L, 297P, 304L, 306I, 306M, 308N, 316A, 316C, 347Q, 352G, 352R, 359V, 362S, 362Y, 378L, 393R, 396T, 398W, 399C, 399D, 399F, 399G, 399T, 405G, 405L, 405Y, 407F, 407N, 407S, 409K/414F, 409Q/414F, 410F/414F, 410I/414F, 410S/414F, 410V/414F, 410Y/414F, 411A/414F, 411F/414F, 411G/414F, 411I/414F, 411Q/414F, 411R/414F, 411T/414F, 413A/414F, 413F/414F, 413G/414F, 413I/414F, 414F/415W, 414F/417W, 414G, 415F, 455I, 455L, 465E, 466M, 468M, 468Q, 468S, 468W, 494A, 494C, 494G, 494L, 494W, 509G, and 509K. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1950 and one or more residue differences as compared to SEQ ID NO: 1950, selected from V163I/Q169E, V163I/Q169R, L185C, L185F, E186C, Y192C, Y192G, Y192I, Y192L, Y192R, E193R, E193S, F194A, F194C, F194D, F194G, F194M, F194W, N197G, N197L, N197R, D198A, D198L, G245A, Q251R, R258E, R258G, R258K, R258L, R258Q, R258W, Y259N, Y259V, S261A, S261G, K263I, K263S, V271C, K274M, K274N, K274P, K274Q, K274T, K274V, K278C, K278L, K278N, Y280L, Y280S, L284I, L284M, T286N, T286S, V290A, R291M, R291W, K297L, K297P, A304L, L306I, L306M, Y308N, V316A, V316C, F347Q, P352G, P352R, L359V, T362S, T362Y, V378L, L393R, S396T, F398W, E399C, E399D, E399F, E399G, E399T, S405G, S405L, S405Y, K407F, K407N, K407S, D409K/H414F, D409Q/H414F, A410F/H414F, A410/1H414F, A410S/H414F, A410V/H414F, A410Y/H414F, L411A/H414F, L411F/H414F, L411G/H414F, L411I/H414F, L411Q/H414F, L411R/H414F, L411T/H414F, H413A/1H414F, H413F/H414F, H413G/H414F, H413I/H414F, H414F/A415W, H414F/C417W, H414G, A415F, V455I, V455L, A465E, L466M, G468M, G468Q, G468S, G468W, H494A, H494C, H494G, H494L, H494W, S509G, and S509K.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from 158/274/411/413/414, 185/274/413/414, 185/411/413/414, 263/411/413/414/468, 263/413/414, 274/286/411/413/417/468, 274/411/417/468, 274/411/468, 274/413/414/417/468, 274/468, 278/411/413/468, 411/413/417, 411/413/417/468, 411/413/468, 411/414, 413, 413/414, 413/414/468, 413/417/468, 413/468, 414, and 468. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from 158H/274V/411G/413A/414G, 185F/274V/413A/414G, 185F/411G/413A/414G, 263H/411G/413A/414G/468Q, 263H/413A/414G, 274V/286N/411G/413A/417W/468Q, 274V/411G/417W/468Q, 274V/411G/468Q, 274V/413A/414G/417W/468Q, 274V/468Q, 278N/411G/413A/468Q, 411G/413A/417W, 411G/413A/417W/468Q, 411G/413A/468Q, 411G/414G, 413A, 413A/414G, 413A/414G/468Q, 413A/417W/468Q, 413A/468Q, 414G, and 468Q. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from P158H/K274V/L411G/H413A/H414G, L185F/K274V/H413A/H414G, L185F/L411G/H413A/H414G, K263H/L411G/H413A/H414G/G468Q, K263H/H413A/H414G, K274V/T286N/L411G/H413A/C417W/G468Q, K274V/L411G/C417W/G468Q, K274V/L411G/G468Q, K274V/H413A/H414G/C417W/G468Q, K274V/G468Q, K278N/L411G/H413A/G468Q, L411G/H413A/C417W, L411G/H413A/C417W/G468Q, L411G/H413A/G468Q, L411G/H414G, H413A, H413A/H414G, H413A/H414G/G468Q, H413A/C417W/G468Q, H413A/G468Q, H414G, and G468Q.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from 3, 8, 11, 12, 15, 26, 26/27, 36, 39, 50, 58, 62, 76, 90, 116, 147, 151, 157, 246, 248, 249, 253, and 255. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from 3A, 3G, 8P, 11C, 12V, 15A, 26A/27R, 26G, 26I, 26Q, 26T, 36G, 36K, 39A, 50L, 58M, 58N, 58S, 62W, 76P, 90L, 90S, 116A, 147G, 151S, 157V, 246N, 248R, 248V, 249A, 249G, 249R, 253L, and 255G. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from H3A, H3G, G8P, G11C, S12V, I15A, R26A/Q27R, R26G, R26I, R26Q, R26T, S36G, S36K, Y39A, I50L, T58M, T58N, T58S, F62W, N76P, N90L, N90S, S116A, D147G, G151S, P157V, E246N, L248R, L248V, E249A, E249G, E249R, V253L, and N255G.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from 9, 11, 12, 15, 16, 26, 38, 39, 58, 70, 79, 81, 90, 151, 157, 158, and 249. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from 9A, 9T, 11S, 12A, 12N, 12Q, 15L, 15Q, 16S, 26S, 26W, 38L, 38T, 39G, 58A, 70A, 79T, 81E, 90M, 151I, 157L, 158A, and 249T.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from G9A, G9T, G11S, S12A, S12N, S12Q, I15L, I15Q, R16S, R26S, R26W, I38L, I38T, Y39G, T58A, K70A, S79T, S81E, N90M, G151I, P157L, P158A, and E249T.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from 189, 196, 205, 206, 262, 307, 314, 318, 324, 353, 397, 408, 410, 413, 469, 473, 480, 481, 491, and 493. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from 189E, 189Q, 189R, 196A, 196S, 196T, 196Y, 205E, 206N, 262V, 307E, 307H, 307L, 307S, 314A, 314M, 314Q, 318R, 324V, 353R, 397A, 408E, 408L, 408W, 410E, 413E, 413G, 413L, 413M, 413S, 469F, 469Y, 473A, 473D, 473G, 473P, 473Q, 473S, 480A, 480E, 480G, 480L, 480S, 480W, 481A, 481L, 481S, 481V, 491M, 493E, and 493Q. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2008 and one or more residue differences as compared to SEQ ID NO: 2008, selected from A189E, A189Q, A189R, R196A, R196S, R196T, R196Y, R205E, R206N, F262V, C307E, C307H, C307L, C307S, R314A, R314M, R314Q, K318R, 1324V, Q353R, Q397A, A408E, A408L, A408W, A410E, H413E, H413G, H413L, H413M, H413S, W469F, W469Y, R473A, R473D, R473G, R473P, R473Q, R473S, R480A, R480E, R480G, R480L, R480S, R480W, W481A, W481L, W481S, W481V, L491M, N493E, and N493Q.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2254 and one or more residue differences as compared to SEQ ID NO: 2254, selected from 197/407/455, 197/455, 284/398/466, 362/407/455, 396/398/410/466, 398/466, 399/411/416, and 466. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2254 and one or more residue differences as compared to SEQ ID NO: 2254, selected from 197R/407S/455L, 197R/455L, 284M/398W/466M, 362S/407S/455L, 396T/398W/410V/466M, 398W/466M, 399D/411Q/416Q, and 466M. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2254 and one or more residue differences as compared to SEQ ID NO: 2254, selected from N197R/K407S/V455L, N197R/V455L, L284M/F398W/L466M, T362S/K407S/V455L, S396T/F398W/A410V/L466M, F398W/L466M, E399D/G411Q/K416Q, and L466M.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2514 and one or more residue differences as compared to SEQ ID NO: 2514, selected from 26/90/94/248/261/266/362/455, 26/90/246, 26/90/246/248, 26/90/246/248/261, 26/90/246/248/362/455, 26/90/246/248/455, 26/90/246/266/362, 26/90/246/362, 26/90/246/455, 26/90/248, 26/90/248/266/455, 26/90/248/455, 26/90/248/455/459, 26/90/266, 26/90/362/455, 26/173/248, 26/246, 26/246/248/362, 26/246/248/362/455, 26/246/248/455, 26/248, 26/248/261/266, 26/248/261/266/362/455, 26/248/266/362, 26/248/362/455, 26/248/455, 26/362, 26/362/455, 58/197/249/407/410, 62/249, 90/246/248, 90/246/248/261/266/362/455, 90/246/248/266/362/455, 246/248, 246/248/362, 246/266/455, 248, 248/266/362, 248/362/455, 248/455, and 362. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2514 and one or more residue differences as compared to SEQ ID NO: 2514, selected from 26G/90L/94K/248R/261A/266V/362S/455L, 26G/90L/246N, 26G/90L/246N/248R, 26G/90L/246N/266V/362S, 26G/90L/248V/455L, 26G/90L/248V/455L/459H, 26G/90L/266V, 26G/90L/362S/455L, 26G/246N, 26G/246N/248R/362S, 26G/246N/248V/455L, 26G/248R, 26G/248V/261A/266V, 26I/90L/246N/248R, 26I/90L/248R, 26I/246N/248R/362S/455L, 26I/248R/266V/362S, 26I/248R/455L, 26Q/90L/246N/248R/261A, 26Q/90L/246N/248R/362S/455L, 26Q/90L/246N/248R/455L, 26Q/90L/246N/248V/362S/455L, 26Q/90L/246N/362S, 26Q/90L/246N/455L, 26Q/90L/248R/266V/455L, 26Q/173I/248R, 26Q/246N/248V/362S, 26Q/248R, 26Q/248R/261A/266V/362S/455L, 26Q/248R/362S/455L, 26Q/248R/455L, 26Q/248V, 26Q/248V/266V/362S, 26Q/248V/362S/455L, 26Q/362S, 26Q/362S/455L, 58N/197L/249R/407N/410S, 62W/249R, 90L/246N/248R, 90L/246N/248R/261A/266V/362S/455L, 90L/246N/248R/266V/362S/455L, 246N/248R, 246N/248R/362S, 246N/266V/455L, 248R, 248R/455L, 248V/266V/362S, 248V/362S/455L, and 362S. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2514 and one or more residue differences as compared to SEQ ID NO: 2514, selected from R26G/N90L/E94K/L248R/S261A/K266V/T362S/V455L, R26G/N90L/E246N, R26G/N90L/E246N/L248R, R26G/N90L/E246N/K266V/T362S, R26G/N90L/L248V/V455L, R26G/N90L/L248V/V455L/Y459H, R26G/N90L/K266V, R26G/N90L/T362S/V455L, R26G/E246N, R26G/E246N/L248R/T362S, R26G/E246N/L248V/V455L, R26G/L248R, R26G/L248V/S261A/K266V, R26I/N90L/E246N/L248R, R26I/N90L/L248R, R26I/E246N/L248R/T362S/V455L, R26I/L248R/K266V/T362S, R26I/L248R/V455L, R26Q/N90L/E246N/L248R/S261A, R26Q/N90L/E246N/L248R/T362S/V455L, R26Q/N90L/E246N/L248R/V455L, R26Q/N90L/E246N/L248V/T362S/V455L, R26Q/N90L/E246N/T362S, R26Q/N90L/E246N/V455L, R26Q/N90L/L248R/K266V/V455L, R26Q/T173I/L248R, R26Q/E246N/L248V/T362S, R26Q/L248R, R26Q/L248R/S261A/K266V/T362S/V455L, R26Q/L248R/T362S/V455L, R26Q/L248R/V455L, R26Q/L248V, R26Q/L248V/K266V/T362S, R26Q/L248V/T362S/V455L, R26Q/T362S, R26Q/T362S/V455L, T58N/N197L/E249R/K407N/V410S, F62W/E249R, N90L/E246N/L248R, N90L/E246N/L248R/S261A/K266V/T362S/V455L, N90L/E246N/L248R/K266V/T362S/V455L, E246N/L248R, E246N/L248R/T362S, E246N/K266V/V455L, L248R, L248R/V455L, L248V/K266V/T362S, L248V/T362S/V455L, and T362S.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2524 and one or more residue differences as compared to SEQ ID NO: 2524, selected from 9/16/62/157/246/249/362, 11/58/227/246, 12/16/158/246/248/249, 30/189/261/266/353/465/468, 30/189/266, 30/261/266/353/468, 30/266, 30/266/303, 30/266/353, 38, 38/81/318, 38/197, 39, 39/79, 58/157/158/362, 70/353, 79/81, 81, 189, 189/261, 189/353, 246/249, 261/353, 266/307/353/468, 266/353/468, and 266/468. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2524 and one or more residue differences as compared to SEQ ID NO: 2524, selected from 9T/16S/62W/157L/246E/249R/362T, 11S/58N/227M/246E, 12A/16S/158A/246E/248L/249R, 30E/189R/266V, 30G/261A/266V/353R/468S, 30G/266V, 30G/266V/353R, 30T/189R/261A/266V/353R/465E/468S, 30T/266V/303E, 38T, 38T/81E/318R, 38T/197L, 39G, 39G/79T, 58N/157L/158A/362T, 70A/353R, 79T/81E, 81E, 189R, 189R/261A, 189R/353R, 246E/249R, 261A/353R, 266V/307S/353R/468S, 266V/353R/468S, and 266V/468S. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2524 and one or more residue differences as compared to SEQ ID NO: 2524, selected from G9T/R16S/F62W/P157L/N246E/E249R/S362T, G11S/T58N/L227M/N246E, S12A/R16S/P158A/N246E/R248L/E249R, M30E/A189R/K266V, M30G/S261A/K266V/Q353R/Q468S, M30G/K266V, M30G/K266V/Q353R, M30T/A189R/S261A/K266V/Q353R/A465E/Q468S, M30T/K266V/K303E, I38T, I38T/S81E/K318R, I38T/N197L, Y39G, Y39G/S79T, T58N/P157L/P158A/S362T, K70A/Q353R, S79T/S81E, S81E, A189R, A189R/S261A, A189R/Q353R, N246E/E249R, S261A/Q353R, K266V/C307S/Q353R/Q468S, K266V/Q353R/Q468S, and K266V/Q468S.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2524 and one or more residue differences as compared to SEQ ID NO: 2524, selected from 47/326, 169, 191/413, 200, 292, 304/329, 327/406, 329, 340, 353/459, 373, 379, 382, 402, 403, 404, 427, 429, 459, 461, 484, 490, 495, 504, and 506. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2524 and one or more residue differences as compared to SEQ ID NO: 2524, selected from 47A/326R, 169M, 191V/413V, 200V, 292N, 304V/329R, 327M/406T, 329R, 340I, 340V, 353H/459V, 373G, 379I, 379L, 379M, 379V, 382F, 382L, 402G, 402S, 402V, 403A, 403E, 403P, 403R, 403S, 404D, 404S, 427L, 427M, 427R, 427W, 429R, 459I, 461S, 484A, 490R, 495C, 495G, 495S, 504K, and 506P. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2524 and one or more residue differences as compared to SEQ ID NO: 2524, selected from V47A/L326R, Q169M, N191V/A413V, T200V, S292N, A304V/Q329R, L327M/G406T, Q329R, L340I, L340V, Q353H/Y459V, Q373G, T379I, T379L, T379M, T379V, W382F, W382L, K402G, K402S, K402V, L403A, L403E, L403P, L403R, L403S, P404D, P404S, K427L, K427M, K427R, K427W, D429R, Y459I, G461S, T484A, M490R, A495C, A495G, A495S, F504K, and K506P.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2524 and one or more residue differences as compared to SEQ ID NO: 2524, selected from 175, 179, 189, 200, 203, 292, 293, 325, 340, 373, 379, 402, 403, 404, 406, 427, 459, 461, 484, 495, 506, and 508. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2524 and one or more residue differences as compared to SEQ ID NO: 2524, selected from 175L, 179L, 189V, 200N, 203D, 203M, 292N, 293Q, 325W, 340V, 373C, 373G, 379L, 379M, 402E, 402G, 402S, 403A, 403E, 403G, 403P, 404D, 404E, 404S, 406N, 427C, 427F, 427L, 427M, 427N, 427W, 427Y, 459I, 461S, 484A, 484H, 484M, 495G, 495S, 506P, 506S, 506T, 508S, and 508T. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2524 and one or more residue differences as compared to SEQ ID NO: 2524, selected from N175L, Q179L, A189V, T200N, E203D, E203M, S292N, S293Q, S325W, L340V, Q373C, Q373G, T379L, T379M, K402E, K402G, K402S, L403A, L403E, L403G, L403P, P404D, P404E, P404S, G406N, K427C, K427F, K427L, K427M, K427N, K427W, K427Y, Y459I, G461S, T484A, T484H, T484M, A495G, A495S, K506P, K506S, K506T, K508S, and K508T.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2638 and one or more residue differences as compared to SEQ ID NO: 2638, selected from 11/30/79/189/480, 30/58/79/189/307/480, 79/189/307/410, and 79/307. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2638 and one or more residue differences as compared to SEQ ID NO: 2638, selected from 11S/30G/79T/189R/480E, 30G/58N/79T/189R/307L/480E, 79T/189R/307L/410E, and 79T/307L. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2638 and one or more residue differences as compared to SEQ ID NO: 2638, selected from G11S/M30G/S79T/A189R/R480E, M30G/T58N/S79T/A189R/C307L/R480E, S79T/A189R/C307L/V410E, and S79T/C307L.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2804 and one or more residue differences as compared to SEQ ID NO: 2804, selected from 169/304/340/402/427, 169/304/340/427/429/504, 169/304/340/429/506, 169/304/340/504, 169/304/402/403/427/506, 169/304/403/427/504, 169/304/427/504/506, 169/304/504, 169/340/402/403/427/429/504, 169/340/402/403/427/504/506, 169/340/402/504, 169/340/427, 169/340/506, 169/402/403/504/506, 169/402/427/429/504, 169/402/504, 169/402/504/506, 169/403/427/506, 266/327/329/404/410, 292/327/329/468, 304, 304/340/402, 304/340/402/403/427/429/504/506, 304/340/402/403/504/506, 304/340/402/403/506, 304/340/402/427/504/506, 304/340/402/506, 304/340/403/427/429/504/506, 304/340/427, 304/402/403, 304/402/403/427/504, 304/402/403/429, 304/402/403/504/506, 304/402/403/506, 304/403/504, 304/504, 304/504/506, 327, 327/329, 327/329/379/404/406/410, 327/329/404/410, 327/329/465/484, 327/382/406/410/484, 327/410/484, 340, 340/402/403, 340/402/427/429/506, 340/402/429/504/506, 340/402/504, 340/403/504, 340/504, 340/506, 379/382/468, 379/404/410, 379/410, 379/465/468/484, 379/468, 402/403/427/504/506, 402/403/429/504, 402/427/504, 402/504, 403/427, 427, 484, 504, 504/506, and 506. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2804 and one or more residue differences as compared to SEQ ID NO: 2804, selected from 169M/304V/340I/429R/506P, 169M/304V/340I/504K, 169M/304V/340V/402G/427L, 169M/304V/340V/427L/429R/504K, 169M/304V/402G/403S/427L/506P, 169M/304V/403S/427L/504K, 169M/304V/427M/504K/506P, 169M/304V/504K, 169M/340I/402G/403A/427M/429R/504K, 169M/340I/427L, 169M/340I/506P, 169M/340V/402G/403P/427M/504K/506P, 169M/340V/402G/504K, 169M/402G/403A/504K/506P, 169M/402G/427L/429R/504K, 169M/402G/504K, 169M/402G/504K/506P, 169M/403A/427L/506P, 266V/327M/329R/404S/410V, 292N/327M/329R/468S, 304V, 304V/340I/402G, 304V/340I/402G/403A/427L/429R/504K/506P, 304V/340I/402G/403S/506P, 304V/340I/402G/506P, 304V/340I/403A/427L/429R/504K/506P, 304V/340I/427M, 304V/340V/402G/403S/504K/506P, 304V/340V/402G/427L/504K/506P, 304V/402G/403A/429R, 304V/402G/403A/504K/506P, 304V/402G/403P/427M/504K, 304V/402G/403P/506P, 304V/402G/403S, 304V/403S/504K, 304V/504K, 304V/504K/506P, 327M, 327M/329R, 327M/329R/379M/404D/406T/410V, 327M/329R/404D/410V, 327M/329R/465E/484A, 327M/382L/406T/410V/484A, 327M/410V/484A, 340I/402G/429R/504K/506P, 340I/402G/504K, 340I/403S/504K, 340I/506P, 340V, 340V/402G/403A, 340V/402G/427L/429R/506P, 340V/504K, 379L/465E/468S/484A, 379L/468S, 379M/382L/468S, 379M/404D/410V, 379M/410V, 379M/468S, 402G/403A/427M/504K/506P, 402G/403S/427M/504K/506P, 402G/403S/429R/504K, 402G/427L/504K, 402G/504K, 403S/427L, 427L, 427M, 484A, 504K, 504K/506P, and 506P. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2804 and one or more residue differences as compared to SEQ ID NO: 2804, selected from Q169M/A304V/L340I/D429R/K506P, Q169M/A304V/L340I/F504K, Q169M/A304V/L340V/K402G/K427L, Q169M/A304V/L340V/K427L/D429R/F504K, Q169M/A304V/K402G/L403S/K427L/K506P, Q169M/A304V/L403S/K427L/F504K, Q169M/A304V/K427M/F504K/K506P, Q169M/A304V/F504K, Q169M/L340I/K402G/L403A/K427M/D429R/F504K, Q169M/L340I/K427L, Q169M/L340I/K506P, Q169M/L340V/K402G/L403P/K427M/F504K/K506P, Q169M/L340V/K402G/F504K, Q169M/K402G/L403A/F504K/K506P, Q169M/K402G/K427L/D429R/F504K, Q169M/K402G/F504K, Q169M/K402G/F504K/K506P, Q169M/L403A/K427L/K506P, K266V/L327M/Q329R/P404S/E410V, S292N/L327M/Q329R/Q468S, A304V, A304V/L340I/K402G, A304V/L340I/K402G/L403A/K427L/D429R/F504K/K506P, A304V/L340I/K402G/L403S/K506P, A304V/L340I/K402G/K506P, A304V/L340I/L403A/K427L/D429R/F504K/K506P, A304V/L340I/K427M, A304V/L340V/K402G/L403S/F504K/K506P, A304V/L340V/K402G/K427L/F504K/K506P, A304V/K402G/L403A/D429R, A304V/K402G/L403A/F504K/K506P, A304V/K402G/L403P/K427M/F504K, A304V/K402G/L403P/K506P, A304V/K402G/L403S, A304V/L403S/F504K, A304V/F504K, A304V/F504K/K506P, L327M, L327M/Q329R, L327M/Q329R/T379M/P404D/G406T/E410V, L327M/Q329R/P404D/E410V, L327M/Q329R/A465E/T484A, L327M/W382L/G406T/E410V/T484A, L327M/E410V/T484A, L340I/K402G/D429R/F504K/K506P, L340I/K402G/F504K, L340I/L403S/F504K, L340I/K506P, L340V, L340V/K402G/L403A, L340V/K402G/K427L/D429R/K506P, L340V/F504K, T379L/A465E/Q468S/T484A, T379L/Q468S, T379M/W382L/Q468S, T379M/P404D/E410V, T379M/E410V, T379M/Q468S, K402G/L403A/K427M/F504K/K506P, K402G/L403S/K427M/F504K/K506P, K402G/L403S/D429R/F504K, K402G/K427L/F504K, K402G/F504K, L403S/K427L, K427L, K427M, T484A, F504K, F504K/K506P, and K506P.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2812 and one or more residue differences as compared to SEQ ID NO: 2812, selected from 30, 30/179/200/373/403, 30/179/373/379, 30/184/246/325/379/429/495, 30/184/246/327/329/459, 30/184/246/379/404, 30/184/246/459/461/495/500/504, 30/200/373, 30/200/373/379, 30/246/325/327/329/404/461, 30/246/325/379/404/427/429/461, 30/246/427/459/461, 30/325/327, 30/325/327/379/404/429/495, 30/327/404/504, 30/329/379, 30/373, 30/373/403, 30/379/429/459/461, 30/379/459/461/504, 30/403/441/460, 200/373/379, 246/325/329/379/461, 246/327/404/461/495, 327/329/379/504, 327/459/461/495, 353/403, 373/379, 373/379/403, 379/403/406/468, and 403/441. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2812 and one or more residue differences as compared to SEQ ID NO: 2812, selected from 30G, 30G/179A/200N/373G/403E, 30G/179A/373G/379M, 30G/184S/246E/325W/379M/429R/495S, 30G/184S/246E/327M/329R/459I, 30G/184S/246E/379M/404D, 30G/184S/246E/459I/461S/495S/500N/504Q, 30G/200N/373G, 30G/200N/373G/379M, 30G/246E/325W/327M/329R/404D/461S, 30G/246E/325W/379M/404D/427L/429R/461S, 30G/246E/427L/459I/461S, 30G/325W/327M, 30G/325W/327M/379M/404D/429R/495S, 30G/327M/404D/504S, 30G/329R/379M, 30G/373G, 30G/373G/403L, 30G/379M/429R/459I/461S, 30G/379M/459I/461S/504Q, 30G/403E/441K/460P, 200N/373G/379M, 246E/325W/329R/379M/461S, 246E/327M/404D/461S/495S, 327M/329R/379M/504S, 327M/459I/461S/495S, 353R/403E, 373G/379M, 373G/379M/403L, 379M/403L/406N/468S, and 403L/441K. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2812 and one or more residue differences as compared to SEQ ID NO: 2812, selected from M30G, M30G/Q179A/T200N/Q373G/S403E, M30G/Q179A/Q373G/T379M, M30G/A184S/N246E/S325W/T379M/D429R/A495S, M30G/A184S/N246E/L327M/Q329R/Y459I, M30G/A184S/N246E/T379M/P404D, M30G/A184S/N246E/Y459I/G461S/A495S/T500N/F504Q, M30G/T200N/Q373G, M30G/T200N/Q373G/T379M, M30G/N246E/S325W/L327M/Q329R/P404D/G461S, M30G/N246E/S325W/T379M/P404D/K427L/D429R/G461S, M30G/N246E/K427L/Y459I/G461S, M30G/S325W/L327M, M30G/S325W/L327M/T379M/P404D/D429R/A495S, M30G/L327M/P404D/F504S, M30G/Q329R/T379M, M30G/Q373G, M30G/Q373G/S403L, M30G/T379M/D429R/Y459I/G461S, M30G/T379M/Y459I/G461S/F504Q, M30G/S403E/M441K/G460P, T200N/Q373G/T379M, N246E/S325W/Q329R/T379M/G461S, N246E/L327M/P404D/G461S/A495S, L327M/Q329R/T379M/F504S, L327M/Y459I/G461S/A495S, Q353R/S403E, Q373G/T379M, Q373G/T379M/S403L, T379M/S403L/G406N/Q468S, and S403L/M441K.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2812 and one or more residue differences as compared to SEQ ID NO: 2812, selected from 198, 231, 233, 248, 253, 264, 266, 278, 326, 367, 370, 396, 414, 433, 435, 437, 444, 446, 485, 499, 503, 520, and 525. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2812 and one or more residue differences as compared to SEQ ID NO: 2812, selected from 198E, 231S, 233R, 248K, 248T, 253I, 264E, 264L, 264T, 264V, 266R, 278M, 326R, 367D, 370D, 370G, 370M, 370S, 396R, 414E, 433A, 433E, 433G, 433M, 433P, 433R, 433S, 435A, 435E, 435G, 435S, 437G, 437R, 444G, 444R, 446G, 485E, 499M, 499R, 503A, 503E, 503T, 503V, 520P, 525Q, 525R, and 525S. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2812 and one or more residue differences as compared to SEQ ID NO: 2812, selected from D198E, G231S, E233R, R248K, R248T, V253I, R264E, R264L, R264T, R264V, K266R, K278M, L326R, E367D, Q370D, Q370G, Q370M, Q370S, T396R, G414E, W433A, W433E, W433G, W433M, W433P, W433R, W433S, M435A, M435E, M435G, M435S, T437G, T437R, A444G, A444R, P446G, D485E, L499M, L499R, I503A, 1503E, I503T, I503V, E520P, F525Q, F525R, and F525S.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2812 and one or more residue differences as compared to SEQ ID NO: 2812, selected from 186, 188, 198, 231, 233, 235, 243, 248, 253, 264, 266, 287, 297/440, 366, 367, 368, 370, 414, 433, 435, 437, 439, 442, 444, 485, 501, and 515. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2812 and one or more residue differences as compared to SEQ ID NO: 2812, selected from 186D, 188I, 198E, 231S, 233A, 235R, 243L, 243R, 243S, 243T, 248S, 253I, 264A, 264T, 266R, 266T, 287I, 297S/440K, 366V, 367A, 368R, 370M, 370N, 370S, 414E, 433M, 433P, 433V, 435E, 435I, 435P, 437A, 437G, 437K, 437P, 439G, 439P, 442A, 444G, 444H, 485E, 485S, 501R, and 515E. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2812 and one or more residue differences as compared to SEQ ID NO: 2812, selected from E186D, L188I, D198E, G231S, E233A, K235R, E243L, E243R, E243S, E243T, R248S, V253I, R264A, R264T, K266R, K266T, L287I, K297S/N440K, A366V, E367A, K368R, Q370M, Q370N, Q370S, G414E, W433M, W433P, W433V, M435E, M435I, M435P, T437A, T437G, T437K, T437P, S439G, S439P, G442A, A444G, A444H, D485E, D485S, K501R, and A515E.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2956 and one or more residue differences as compared to SEQ ID NO: 2956, selected from 140/142/153/177/427/434/441/444/461/502, 140/142/153/427/484, 140/142/177, 140/142/177/441, 140/142/365/373/404/427/484, 140/142/373/427/484, 140/148/161/177/404, 140/150/153/365/373/427/484, 140/150/177/404/436/441/484/502, 140/161/177/404/427/484, 140/177/404, 140/177/404/484, 142/150/158/177/427/445, 142/150/177/404, 142/153/177/441/444, 142/177/373, 142/177/373/441, 142/461/484/502, 150/177, 153, 153/161/325/404/427/441/484, 153/484, 177, 177/365/427/434, 325/427, 427, and 484. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2956 and one or more residue differences as compared to SEQ ID NO: 2956, selected from 140V/142V/153V/177R/427L/434N/441V/444S/461S/502G, 140V/142V/153V/427L/484L, 140V/142V/177R, 140V/142V/177R/441V, 140V/142V/365A/373R/404D/427L/484L, 140V/142V/373R/427L/484L, 140V/148T/161H/177R/404D, 140V/150P/153V/365A/373R/427L/484L, 140V/150P/177R/404D/436S/441V/484L/502G, 140V/161H/177R/404D/427L/484L, 140V/177R/404D, 140V/177R/404D/484L, 142V/150P/158S/177R/427L/445N, 142V/150P/177R/404D, 142V/153V/177R/441V/444S, 142V/177R/373R, 142V/177R/373R/441V, 142V/461S/484L/502G, 150P/177R, 153V, 153V/161H/325L/404D/427L/441V/484L, 153V/484L, 177R, 177R/365A/427L/434N, 325L/427L, 427L, and 484L. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2956 and one or more residue differences as compared to SEQ ID NO: 2956, selected from G140V/M142V/E153V/H177R/K427L/E434N/M441V/A444S/G461S/K502G, G140V/M142V/E153V/K427L/T484L, G140V/M142V/H177R, G140V/M142V/H177R/M441V, G140V/M142V/G365A/Q373R/P404D/K427L/T484L, G140V/M142V/Q373R/K427L/T484L, G140V/P148T/K161H/H177R/P404D, G140V/E150P/E153V/G365A/Q373R/K427L/T484L, G140V/E150P/H177R/P404D/P436S/M441V/T484L/K502G, G140V/K161H/H177R/P404D/K427L/T484L, G140V/H177R/P404D, G140V/H177R/P404D/T484L, M142V/E150P/P158S/H177R/K427L/K445N, M142V/E150P/H177R/P404D, M142V/E153V/H177R/M441V/A444S, M142V/H177R/Q373R, M142V/H177R/Q373R/M441V, M142V/G461S/T484L/K502G, E150P/H177R, E153V, E153V/K161H/W325L/P404D/K427L/M441V/T484L, E153V/T484L, H177R, H177R/G365A/K427L/E434N, W325L/K427L, K427L, and T484L.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3174 and one or more residue differences as compared to SEQ ID NO: 3174, selected from 186/188/248/253/365/366/444/445, 186/231/248/253/484, 186/231/248/365/366/368/484, 186/231/248/366/368/444, 186/231/248/484, 186/231/368/416/441/442/444/484/485, 186/231/441/444/445, 186/484/485, 188/231/248/253/365/441/442/444/445/484, 198/243/264/431/441, 198/243/396/414/431/433/441/499, 198/243/396/414/433/437/441/515, 198/264/266/414, 198/264/396/414/433/441/515, 198/266/396/414/433/441/499/501, 198/266/414/433/441/515, 198/266/437/441/499, 198/414/431/441/499/520, 198/414/433/441/515/520, 231/248/441/484, 243/264/515/520, 243/396/414/433/441/515/520, 243/414/433/437/441, 243/414/437/441/515, 248/442/444/445/484, 264/266/396/414/433/441/515, 264/266/414/441/499/501/515/520, 264/414, 264/414/441, 264/433/441, 266/437/441/499/515/520, 365/366/441/484/485, 396/414/441, 396/414/441/515, and 414/441/520. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3174 and one or more residue differences as compared to SEQ ID NO: 3174, selected from 186D/188I/248K/253I/365A/366V/444H/445N, 186D/231S/248K/253I/484L, 186D/231S/248K/365A/366V/368R/484L, 186D/231S/248K/366V/368R/444H, 186D/231S/248K/484L, 186D/231S/368R/416N/441M/442K/444A/484L/485E, 186D/231S/441M/444H/445N, 186D/484L/485E, 188I/231S/248K/253I/365A/441M/442K/444A/445N/484L, 198E/243S/264A/431R/441M, 198E/243S/396S/414E/431R/433V/441M/499M, 198E/243S/396S/414E/433R/437R/441M/515E, 198E/264A/266T/414E, 198E/264A/396S/414E/433V/441M/515E, 198E/266T/396S/414E/433V/441M/499M/501R, 198E/266T/414E/433S/441M/515E, 198E/266T/437R/441M/499M, 198E/414E/431R/441M/499M/520D, 198E/414E/433V/441M/515E/520D, 231S/248K/441M/484L, 243S/264A/515E/520D, 243S/396S/414E/433V/441M/515E/520D, 243S/414E/433S/437R/441M, 243S/414E/437R/441M/515E, 248K/442K/444A/445N/484L, 264A/266T/396S/414E/433R/441M/515E, 264A/266T/414E/441M/499M/501R/515E/520D, 264A/414E, 264A/414E/441M, 264A/433S/441M, 266T/437R/441M/499M/515E/520D, 365A/366V/441M/484L/485E, 396S/414E/441M, 396S/414E/441M/515E, and 414E/441M/520D. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3174 and one or more residue differences as compared to SEQ ID NO: 3174, selected from E186D/L188I/R248K/V253I/G365A/A366V/S444H/K445N, E186D/G231S/R248K/V253I/T484L, E186D/G231S/R248K/G365A/A366V/K368R/T484L, E186D/G231S/R248K/A366V/K368R/S444H, E186D/G231S/R248K/T484L, E186D/G231S/K368R/K416N/V441M/G442K/S444A/T484L/D485E, E186D/G231S/V441M/S444H/K445N, E186D/T484L/D485E, L188I/G231S/R248K/V253I/G365A/V441M/G442K/S444A/K445N/T484L, D198E/E243S/R264A/S431R/V441M, D198E/E243S/T396S/G414E/S431R/W433V/V441M/L499M, D198E/E243S/T396S/G414E/W433R/T437R/V441M/A515E, D198E/R264A/K266T/G414E, D198E/R264A/T396S/G414E/W433V/V441M/A515E, D198E/K266T/T396S/G414E/W433V/V441M/L499M/K501R, D198E/K266T/G414E/W433S/V441M/A515E, D198E/K266T/T437R/V441M/L499M, D198E/G414E/S431R/V441M/L499M/E520D, D198E/G414E/W433V/V441M/A515E/E520D, G231S/R248K/V441M/T484L, E243S/R264A/A515E/E520D, E243S/T396S/G414E/W433V/V441M/A515E/E520D, E243S/G414E/W433S/T437R/V441M, E243S/G414E/T437R/V441M/A515E, R248K/G442K/S444A/K445N/T484L, R264A/K266T/T396S/G414E/W433R/V441M/A515E, R264A/K266T/G414E/V441M/L499M/K501R/A515E/E520D, R264A/G414E, R264A/G414E/V441M, R264A/W433S/V441M, K266T/T437R/V441M/L499M/A515E/E520D, G365A/A366V/V441M/T484L/D485E, T396S/G414E/V441M, T396S/G414E/V441M/A515E, and G414E/V441M/E520D.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3174 and one or more residue differences as compared to SEQ ID NO: 3174, selected from 122/473, 189, 190, 193, 194, 196, 263, 264, 266, 267, 268, 269, 273, 273/501, 274, 278, 279, 281, 297, 298, 299, 300, 301, 302, 304, 309, 312, 315, 347, 350, 352, 353, 359, 390, 392, 394, 407, 408, 410, 411, 413, 414, 416, 436, 454, 468, 472, 473, 477, 479, 480, and 493. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3174 and one or more residue differences as compared to SEQ ID NO: 3174, selected from 122I/473K, 189L, 190L, 190M, 190R, 193S, 193T, 194C, 194L, 194S, 194W, 196G, 196L, 196M, 196N, 196T, 263M, 263R, 264C, 266F, 266I, 266R, 266T, 266V, 266Y, 267A, 267C, 267H, 267V, 267Y, 268C, 268I, 268L, 268T, 269W, 273C, 273D, 273E, 273E/501N, 273F, 273G, 273I, 273L, 273Y, 274A, 274G, 274I, 274V, 278F, 278H, 278M, 278R, 278V, 279Y, 281K, 281L, 297C, 297L, 297M, 297R, 297T, 297V, 298I, 298M, 299A, 299L, 299M, 299N, 299Y, 300H, 301S, 301T, 301V, 302N, 302S, 304D, 304M, 304T, 309F, 309G, 309I, 309L, 309M, 309V, 309W, 312M, 312T, 312V, 315Q, 347I, 350L, 350R, 350W, 352V, 353A, 359G, 390C, 390I, 390L, 390V, 392V, 394A, 394F, 394G, 394M, 394V, 394Y, 407L, 407M, 407N, 407R, 407W, 408G, 408I, 408L, 408M, 408T, 408V, 410F, 410G, 410I, 411E, 411N, 413F, 413L, 413P, 413S, 413V, 414A, 416G, 436S, 454F, 454M, 454V, 468F, 468H, 468M, 468T, 472G, 473G, 477G, 479F, 479V, 480H, 480K, 493V, and 493Y. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3174 and one or more residue differences as compared to SEQ ID NO: 3174, selected from M122I/R473K, R189L, E190L, E190M, E190R, E193S, E193T, F194C, F194L, F194S, F194W, R196G, R196L, R196M, R196N, R196T, H263M, H263R, R264C, K266F, K266I, K266R, K266T, K266V, K266Y, S267A, S267C, S267H, S267V, S267Y, V268C, V268I, V268L, V268T, F269W, V273C, V273D, V273E, V273E/K501N, V273F, V273G, V273I, V273L, V273Y, K274A, K274G, K274I, K274V, K278F, K278H, K278M, K278R, K278V, W279Y, R281K, R281L, K297C, K297L, K297M, K297R, K297T, K297V, L298I, L298M, T299A, T299L, T299M, T299N, T299Y, P300H, M301S, M301T, M301V, Q302N, Q302S, V304D, V304M, V304T, Y309F, Y309G, Y309I, Y309L, Y309M, Y309V, Y309W, L312M, L312T, L312V, G315Q, F347I, G350L, G350R, G350W, P352V, Q353A, L359G, Y390C, Y390I, Y390L, Y390V, R392V, E394A, E394F, E394G, E394M, E394V, E394Y, K407L, K407M, K407N, K407R, K407W, A408G, A408I, A408L, A408M, A408T, A408V, E410F, E410G, E410I, G411E, G411N, A413F, A413L, A413P, A413S, A413V, G414A, K416G, P436S, L454F, L454M, L454V, Q468F, Q468H, Q468M, Q468T, S472G, R473G, R477G, L479F, L479V, R480H, R480K, N493V, and N493Y.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3174 and one or more residue differences as compared to SEQ ID NO: 3174, selected from 196, 263, 266, 268, 273, 281, 394, 416, 454, 468, 473, 477, and 493. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3174 and one or more residue differences as compared to SEQ ID NO: 3174, selected from 196G, 263R, 266I, 266T, 266V, 268I, 273I, 273L, 281K, 394A, 394F, 394G, 394M, 394V, 394Y, 416S, 454M, 468A, 468F, 468H, 468M, 468T, 473G, 477S, and 493V. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3174 and one or more residue differences as compared to SEQ ID NO: 3174, selected from R196G, H263R, K266I, K266T, K266V, V268I, V273I, V273L, R281K, E394A, E394F, E394G, E394M, E394V, E394Y, K416S, L454M, Q468A, Q468F, Q468H, Q468M, Q468T, R473G, R477S, and N493V.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1830 and one or more residue differences as compared to SEQ ID NO: 1830, selected from 194, 196, 266, 267, 268, 269, 273, 273/501, 274, 277, 278, 297, 298, 299, 301, 302, 309, 312, 347, 359, 390, 392, 394, 407, 408, 413, 416, 454, 468, 473, 477, 479, and 493. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1830 and one or more residue differences as compared to SEQ ID NO: 1830, selected from 194L, 194W, 196G, 196M, 196N, 266R, 266T, 266V, 267A, 268L, 268T, 269W, 273D, 273E, 273E/501N, 273F, 273G, 273I, 273L, 273Y, 274A, 274G, 274I, 274V, 277E, 278Y, 297R, 298M, 299A, 299N, 299S, 301S, 301T, 301V, 302S, 309F, 309L, 309M, 309W, 312V, 347I, 359G, 390C, 390V, 392V, 394Y, 407D, 407L, 407M, 407N, 407R, 408I, 413S, 416G, 416S, 454M, 454V, 468T, 473G, 477G, 479V, and 493V. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 1830 and one or more residue differences as compared to SEQ ID NO: 1830, selected from F194L, F194W, R196G, R196M, R196N, K266R, K266T, K266V, S267A, V268L, V268T, F269W, V273D, V273E, V273E/K501N, V273F, V273G, V273I, V273L, V273Y, K274A, K274G, K274I, K274V, D277E, K278Y, K297R, L298M, T299A, T299N, T299S, M301S, M301T, M301V, Q302S, Y309F, Y309L, Y309M, Y309W, L312V, F347I, L359G, Y390C, Y390V, R392V, E394Y, K407D, K407L, K407M, K407N, K407R, A408I, A413S, K416G, K416S, L454M, L454V, Q468T, R473G, R477G, L479V, and N493V.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3222 and one or more residue differences as compared to SEQ ID NO: 3222, selected from 186/194/248/396, 186/198/243/248/366/368/394/501, 186/231/243/368/394/485/499, 186/231/248/485, 186/231/366/368/394/485, 186/243, 186/243/248/366/368/394, 186/243/248/394/484/485, 186/243/484/520, 186/248, 186/365, 186/365/366/368/394/499, 186/365/366/394/485, 186/366/368/394/396/484, 186/366/368/394/396/484/485, 186/394/396/485, 194/198/243/366/368/499, 194/515/520, 198/231/243/248/485, 198/243/248/365/394/501, 198/248/394/396/484/485/499, 198/394/396/484/485/499, 198/394/396/499/515/520, 198/394/499/501, 231/365/368/394/499/520, 231/368/394, 231/484/485/499/501, 243/248/394/396/484/485, 243/484, 243/484/485/499, 248/365/366/368/394/484/520, 248/394/484, 365/366/368/394, 365/368/394/396/520, 394/396, and 394/499. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3222 and one or more residue differences as compared to SEQ ID NO: 3222, selected from 186D/194L/248K/396T, 186D/198E/243S/248K/366V/368R/394Y/501R, 186D/231S/243S/368R/394Y/485E/499M, 186D/231S/248K/485E, 186D/231S/366V/368R/394Y/485E, 186D/243S, 186D/243S/248K/366V/368R/394Y, 186D/243S/248K/394Y/484L/485E, 186D/243S/484L/520D, 186D/248K, 186D/365A, 186D/365A/366V/368R/394Y/499M, 186D/365A/366V/394Y/485E, 186D/366V/368R/394Y/396T/484L, 186D/366V/368R/394Y/396T/484L/485E, 186D/394Y/396T/485E, 194L/198E/243S/366V/368R/499M, 194L/515A/520D, 198E/231S/243S/248K/485E, 198E/243S/248K/365A/394Y/501R, 198E/248K/394Y/396T/484L/485E/499M, 198E/394Y/396T/484L/485E/499M, 198E/394Y/396T/499M/515A/520D, 198E/394Y/499M/501R, 231S/365A/368R/394Y/499M/520D, 231S/368R/394Y, 231S/484L/485E/499M/501R, 243S/248K/394Y/396T/484L/485E, 243S/484L, 243S/484L/485E/499M, 248K/365A/366V/368R/394Y/484L/520D, 248K/394Y/484L, 365A/366V/368R/394Y, 365A/368R/394Y/396T/520D, 394Y/396T, and 394Y/499M. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3222 and one or more residue differences as compared to SEQ ID NO: 3222, selected from E186D/F194L/R248K/S396T, E186D/D198E/E243S/R248K/A366V/K368R/E394Y/K501R, E186D/G231S/E243S/K368R/E394Y/D485E/L499M, E186D/G231S/R248K/D485E, E186D/G231S/A366V/K368R/E394Y/D485E, E186D/E243S, E186D/E243S/R248K/A366V/K368R/E394Y, E186D/E243S/R248K/E394Y/T484L/D485E, E186D/E243S/T484L/E520D, E186D/R248K, E186D/G365A, E186D/G365A/A366V/K368R/E394Y/L499M, E186D/G365A/A366V/E394Y/D485E, E186D/A366V/K368R/E394Y/S396T/T484L, E186D/A366V/K368R/E394Y/S396T/T484L/D485E, E186D/E394Y/S396T/D485E, F194L/D198E/E243S/A366V/K368R/L499M, F194L/E515A/E520D, D198E/G231S/E243S/R248K/D485E, D198E/E243S/R248K/G365A/E394Y/K501R, D198E/R248K/E394Y/S396T/T484L/D485E/L499M, D198E/E394Y/S396T/T484L/D485E/L499M, D198E/E394Y/S396T/L499M/E515A/E520D, D198E/E394Y/L499M/K501R, G231S/G365A/K368R/E394Y/L499M/E520D, G231S/K368R/E394Y, G231S/T484L/D485E/L499M/K501R, E243S/R248K/E394Y/S396T/T484L/D485E, E243S/T484L, E243S/T484L/D485E/L499M, R248K/G365A/A366V/K368R/E394Y/T484L/E520D, R248K/E394Y/T484L, G365A/A366V/K368R/E394Y, G365A/K368R/E394Y/S396T/E520D, E394Y/S396T, and E394Y/L499M.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from 273, 273/309/493/499, 273/493/499, 273/499, 274/299/408/416, 274/408/416, 274/416, 288/299/416, 298/299/416, 309, 309/499, 416, and 493/499. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from 273I, 273I/309M/493V/499L, 273I/493V/499L, 273I/499L, 274A/299N/408I/416S, 274A/408I/416S, 274I/416S, 288V/299N/416S, 298M/299N/416S, 309M, 309M/499L, 416S, and 493V/499L. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from V273I, V273I/Y309M/N493V/M499L, V273I/N493V/M499L, V273I/M499L, K274A/T299N/A408I/K416S, K274A/A408I/K416S, K274I/K416S, E288V/T299N/K416S, L298M/T299N/K416S, Y309M, Y309M/M499L, K416S, and N493V/M499L.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from 168, 198, 267, 301, 307, 308, 308/361, 313, 372, 392, 397, 415, 419, 451, 452, 456, 472, 473, 475, 493/499, and 528. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from 168S, 198S, 267M, 267R, 301G, 301Q, 301S, 307A, 307G, 307S, 308A, 308A/361T, 308G, 308H, 308K, 308S, 308V, 313M, 372E, 392V, 397F, 397W, 415L, 415W, 419G, 419M, 451K, 452L, 456P, 456T, 472A, 473A, 473S, 475V, 493R/499L, and 528L. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from C168S, D198S, S267M, S267R, M301G, M301Q, M301S, L307A, L307G, L307S, Y308A, Y308A/1361T, Y308G, Y308H, Y308K, Y308S, Y308V, A313M, D372E, R392V, Q397F, Q397W, A415L, A415W, L419G, L419M, R451K, V452L, R456P, R456T, S472A, R473A, R473S, F475V, N493R/M499L, and N528L.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from 303/396, 308, 473, and 493/499. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from 303H/396A, 308L, 473A, 473M, 473S, and 493V/499L. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from K303H/S396A, Y308L, R473A, R473M, R473S, and N493V/M499L.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from 273, 273/309/413/499, 273/309/493/499, 273/493, 273/493/499, 273/499, 274/299/408/416, 274/408/416, 274/416, 281/413/499, 288/299/416, 298/299/416, 309, 309/413, 413, 413/493/499, 413/499, 416, and 493/499. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from 273E/493V, 273I, 273I/309M/413S/499L, 273I/309M/493V/499L, 273I/493V/499L, 273I/499L, 273S/309M/413S/499L, 274A/299N/408I/416S, 274A/408I/416S, 274I/416S, 281K/413S/499L, 288V/299N/416S, 298M/299N/416S, 309M, 309M/413S, 413S, 413S/493V/499L, 413S/499L, 416S, and 493V/499L. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3670 and one or more residue differences as compared to SEQ ID NO: 3670, selected from V273E/N493V, V273I, V273I/Y309M/A413S/M499L, V273I/Y309M/N493V/M499L, V273I/N493V/M499L, V273I/M499L, V273S/Y309M/A413S/M499L, K274A/T299N/A408I/K416S, K274A/A408I/K416S, K274I/K416S, R281K/A413S/M499L, E288V/T299N/K416S, L298M/T299N/K416S, Y309M, Y309M/A413S, A413S, A413S/N493V/M499L, A413S/M499L, K416S, and N493V/M499L.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3674 and one or more residue differences as compared to SEQ ID NO: 3674, selected from 194/196/390, 194/196/390/394/460/480, 194/196/390/394/480, 194/196/390/454/480, 194/196/390/480, 194/196/394/454/480, 194/196/454, 194/390, 194/394, 194/394/454, 194/394/454/480, 196, 196/390, 196/390/394, 196/390/394/454, 196/390/394/454/480, 196/390/394/480, 196/390/454, 196/394, 196/394/454, 196/394/454/480, 196/394/480, 196/454, 297/470/473, 297/473/493, 390, 390/394, 390/394/454, 390/394/454/480, 390/394/480, 390/454, 390/480, 394, 394/480, and 454. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3674 and one or more residue differences as compared to SEQ ID NO: 3674, selected from 194L/196G/390C, 194L/196G/390C/394F/460V/480K, 194L/196G/390C/394F/480K, 194L/196G/390C/454M/480K, 194L/196G/390C/480K, 194L/196G/394F/454M/480K, 194L/196G/454M, 194L/390C, 194L/394F, 194L/394F/454M, 194L/394F/454M/480K, 196G, 196G/390C, 196G/390C/394F, 196G/390C/394F/454M, 196G/390C/394F/454M/480K, 196G/390C/394F/480K, 196G/390C/454M, 196G/394F, 196G/394F/454M, 196G/394F/454M/480K, 196G/394F/480K, 196G/454M, 297R/470S/473G, 297R/473G/493V, 390C, 390C/394F, 390C/394F/454M, 390C/394F/454M/480K, 390C/394F/480K, 390C/454M, 390C/480K, 394F, 394F/480K, and 454M. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3674 and one or more residue differences as compared to SEQ ID NO: 3674, selected from F194L/R196G/Y390C, F194L/R196G/Y390C/E394F/G460V/R480K, F194L/R196G/Y390C/E394F/R480K, F194L/R196G/Y390C/L454M/R480K, F194L/R196G/Y390C/R480K, F194L/R196G/E394F/L454M/R480K, F194L/R196G/L454M, F194L/Y390C, F194L/E394F, F194L/E394F/L454M, F194L/E394F/L454M/R480K, R196G, R196G/Y390C, R196G/Y390C/E394F, R196G/Y390C/E394F/L454M, R196G/Y390C/E394F/L454M/R480K, R196G/Y390C/E394F/R480K, R196G/Y390C/L454M, R196G/E394F, R196G/E394F/L454M, R196G/E394F/L454M/R480K, R196G/E394F/R480K, R196G/L454M, K297R/T470S/R473G, K297R/R473G/N493V, Y390C, Y390C/E394F, Y390C/E394F/L454M, Y390C/E394F/L454M/R480K, Y390C/E394F/R480K, Y390C/L454M, Y390C/R480K, E394F, E394F/R480K, and L454M.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3796 and one or more residue differences as compared to SEQ ID NO: 3796, selected from 198, 198/267/313/451/475/494/499, 198/267/314/451, 198/267/314/475, 198/267/409/451, 198/267/475, 198/451/493, 208/308, 208/308/461, 267, 267/314/328/451/494/499, 267/451, 267/451/494/499, 308, 314/328/451/499, 314/451, 328/409/451, 328/451, 409, 409/475/494, 451, 451/493/494, 451/493/499, 451/494, and 475. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3796 and one or more residue differences as compared to SEQ ID NO: 3796, selected from 198P, 198P/267Q/314A/451K, 198P/267Q/314A/475V, 198P/267Q/409L/451K, 198P/267Q/475V, 198P/267R/313L/451K/475V/494V/499L, 198P/451K/493V, 208V/308A/461N, 208V/308V, 267Q/451K/494V/499L, 267R, 267R/314A/328T/451K/494R/499L, 267R/451K, 308A, 308V, 314A/328T/451K/499L, 314A/451K, 328T/409L/451K, 328T/451K, 409L, 409L/475V/494V, 451K, 451K/493V/494V, 451K/493V/499L, 451K/494V, and 475V. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3796 and one or more residue differences as compared to SEQ ID NO: 3796, selected from D198P, D198P/S267Q/R314A/R451K, D198P/S267Q/R314A/F475V, D198P/S267Q/D409L/R451K, D198P/S267Q/F475V, D198P/S267R/A313L/R451K/F475V/H494V/M499L, D198P/R451K/N493V, 1208V/Y308A/S461N, 1208V/Y308V, S267Q/R451K/H494V/M499L, S267R, S267R/R314A/V328T/R451K/H494R/M499L, S267R/R451K, Y308A, Y308V, R314A/V328T/R451K/M499L, R314A/R451K, V328T/D409L/R451K, V328T/R451K, D409L, D409L/F475V/H494V, R451K, R451K/N493V/H494V, R451K/N493V/M499L, R451K/H494V, and F475V.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3870 and one or more residue differences as compared to SEQ ID NO: 3870 at a position or set of positions selected from 165, 169, 171, 173, 175, 176, 179, 183, 187, 191, 192, 195, 197, 199, 200, 203, 204, 210, 257, 259, 267, 291, 293, 295, 301, 319, 325, 340, 341, 342, 374, 387, 398, 399, 403, 404, 406, 429, 480, 481, 483, 484, 490, 491,493, 494, 495, 521, 507, 508, 509, and 522. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3870 and one or more residue differences as compared to SEQ ID NO: 3870 selected from P165A, P165C, P165R, P165T, Q169M, Q169T, R171G, T173S, N175L, N175M, N175V, N176V, Q179K, N175I, Q179M, Q179P, Q179S, Q179T, Q179V, D183C, D183W, I187R, N191F, N191M, N191Q, N191V, Y192A, Y192M, Y192T, K195R, N197C, N197E, N197F, N197R, N197T, D199G, D199R, D199S, D199V, D199W, T200K, T200M, E203F, E203G, E203M, E203V, F204I, F204V, V210M, E257P, E257S, Y259F, R267S, R291M, S293C, S293T, T295A, T295G, T295N, T295S, T295V, M301G, M301W A319E, W325G, W325L, I340S, V341C, E342Q, L374V, L387I, W398V, E399S, S403K, S403L, S403R, S403T, S403V, S403W, P404G, P404M, G406L, G406R, G406S, G406T, R429F, R429H, R429V, R480A, R480Q, W481Q, A483C, L484S, M490A, M490E, M490L, M490R, M490S, M490V, L491V, N493K, H494A, H494T, S495C, S495M, S495N, S495R, A507G, K508N, S509R, Y521V, and L522I.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3918 and one or more residue differences as compared to SEQ ID NO: 3918 at a position or set of positions selected from 3, 7/400/459/504, 164, 173/367/459/500, 184, 203, 203/367/459, 203/367/459/500/501, 203/400/459/501, 203/459, 203/459/499/504, 203/459/500, 215, 218, 294, 335, 335/402/481/484, 335/402/512, 335/481/484, 335/481/493, 335/481/512, 336, 338, 339, 367, 367/459/500, 370, 373, 376, 380, 384, 390, 395, 395/402, 395/402/481/484, 395/481, 395/481/512, 395/484, 400, 400/459, 400/459/499/500/504, 400/459/500/501, 400/459/501/504, 400/501, 400/504, 402, 402/481, 402/481/484, 402/481/484/512, 402/481/493, 402/484/493, 402/512, 458, 459, 459/501, 460, 481, 481/512, 484, 485, 493, 499, 500, 501, 504, 512, 515, and 516. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 3918 and one or more residue differences as compared to SEQ ID NO: 3918 selected from 3Q, H7-/D400W/Y459R/F504Q, S164G, T173I/E367S/Y459V/T500S, S184A, E203R, E203R/E367S/Y459R, E203R/E367S/Y459V/T500S/R501P, E203R/D400W/Y459R/R501P, E203R/Y459R/M499R/F504G, E203R/Y459R/T500S, E203R/Y459V, P215Q, V218L, K294D, F335V, F335V/G402K/W481R/L484E, F335V/G402K/W481R/L484R, F335V/G402K/E512R, F335V/G402R/W481R/L484R, F335V/W481D/L484R, F335V/W481R/L484R, F335V/W481R/N493T, F335V/W481R/E512R, D336Q, D338E, D338Q, A339R, E367R, E367S, E367S/Y459V/T500S, E367T, Q370E, Q373G, Q376S, N380G, K384S, C390A, W395A, W395E, W395N, W395N/G402K, W395N/W481R/E512R, W395N/L484R, W395T, W395T/G402K/W481R/L484R, W395T/W481R, D400W, D400W/Y459R/T500S/R501P, D400W/Y459V, D400W/Y459V/M499R/T500S/F504Q, D400W/Y459V/R501P/F504G, D400W/R501P, D400W/F504Q, G402K, G402K/W481D, G402K/W481D/L484R/E512R, G402K/W481D/N493T, G402K/W481R/L484R, G402K/E512R, G402R, G402R/L484R/N493T, G402S, P458R, Y459R, Y459V, Y459V/R501P, G460M, G460R, W481D, W481R, W481R/E512R, L484E, L484R, E485Q, N493T, M499P, M499R, T500S, R501N, R501P, F504G, F504Q, E512G, E512R, E515P, and H516T.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4266 and one or more residue differences as compared to SEQ ID NO: 4266 at a position or set of positions selected from 95/428/480, 163/190, 163/203/366, 163/328/363/480, 163/363/480/485, 172/174/178/340, 178, 188, 190/202/203/363/366/480/483/485, 190/202/203/480, 190/480/485, 192, 192/498/499/503, 202, 202/203/328/362/363/366/428/480/485/498/499/503, 202/203/485, 203/328/363/428/483, 203/328/428, 203/328/480/485, 203/362/366, 203/498/499/503, 272, 280, 280/498/499/503, 296, 297, 299, 299/498/499/503, 301, 301/503, 308, 308/503, 311, 328/428, 328/480/483, 328/485, 343, 346, 349, 358, 359/498/499/503, 362/363, 362/363/366/428/480/483/498/499/503, 392/498/499/503, 406, 407, 410, 411, 418/498/499/503, 418/503, 419/503, 465, 471, 472/498/499/503, 473, 480/483, 491, and 498/499/503. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4266 and one or more residue differences as compared to SEQ ID NO: 4266 selected from L95M/R428V/W480D, S163Q/N190M, S163Q/F203I/E366S, S163Q/Q328L/P363G/W480D, S163Q/P363G/W480D/E485Q, T172S/N174M/Q178K/V340C, Q178K, R188K, N190M/E202R/F203I/P363G/E366S/W480D/L483R/E485Q, N190M/E202R/F203I/W480D, N190M/W480D/E485Q, E192G, E192S/M498P/T499S/Q503G, E192T, E192V, E192Y, E192Y/M498P/T499S/Q503G, E202R, E202R/F203I/Q328A/S362T/P363G/E366S/R428V/W480D/E485Q/M498P/T499S/Q503G, E202R/F203I/E485Q, F203I/Q328A/P363G/R428V/L483R, F203I/Q328A/R428V, F203I/Q328A/W480D/E485Q, F203I/S362T/E366S, F203I/M498P/T499S/Q503G, V272P, R280G/M498P/T499S/Q503G, R280H, K296G, K296P, L297F, P299E, P299K/M498P/T499S/Q503G, P299S, P299T, Q301G, Q301S/Q503G, Y308H/Q503G, Y308K, Y308Q, Y308R, Y308T, L311R, Q328A/E485Q, Q328L/R428V, Q328L/W480D/L483R, V343C, V343L, F346Q, F346W, G349Q, L358G, L359I/M498P/T499S/Q503G, L392R/M498P/T499S/Q503G, S362T/P363G, S362T/P363R/E366S/R428V/W480D/L483R/M498P/T499S/Q503G, K406S, A407S, G410A, G410Q, D411E, L418G/M498P/T499S/Q503G, L418V/Q503G, I419V/Q503G, M465E, S471N, R472G/M498P/T499S/Q503G, R472S/M498P/T499S/Q503G, M473Q, W480D/L483R, D491G, D491R, and M498P/T499S/Q503G.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4558 and one or more residue differences as compared to SEQ ID NO: 4558 at a position or set of positions selected from 94/365/367, 172/174/178/401/403, 172/174/178/401/403/507, 172/174/178/402/508, 172/174/401/403/507, 172/178, 172/178/401, 174/178, 178/401/403, 178/402/403, 318/375/380, 324/379/405/483, 340, 340/394, 365, 365/367/428, 365/389/394, 367, 375/376, 375/379/483, 375/380, 375/380/400/483, 375/380/483, 376/483, 379/483, 389/394, 394, 401, 401/402/403/507, 402/403, 405/483, and 483. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4558 and one or more residue differences as compared to SEQ ID NO: 4558 selected from V94I/A365F/K367R, T172S/N174M/Q178K/S402L/S508R, T172S/N174M/Q178R/G401K/P403G, T172S/N174M/Q178R/G401K/P403G/K507R, T172S/N174M/G401K/P403G/K507R, T172S/Q178K, T172S/Q178K/G401K, N174M/Q178R, Q178K/G401K/P403G, Q178K/S402L/P403G, A318E/Q375G/L380V, W324L/N379T/G405L/L483R, V340C, V340C/W394T, A365F, A365F/K367R/R428C, A365H/C389A/W394E, K367R, Q375G/K376H, Q375G/N379T/L483R, Q375G/L380V, Q375G/L380V/G400P/L483R, Q375G/L380V/L483R, K376H/L483R, N379T/L483R, C389A/W394E, W394T, G401K, G401K/S402L/P403G/K507N, S402L/P403G, G405L/L483R, and L483R.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4442 and one or more residue differences as compared to SEQ ID NO: 4442 at a position or set of positions selected from 174/296/299, 182, 185/190, 189/190, 190, 190/193, 192/280, 192/402/507, 257, 259, 260, 281, 289, 296/299, 305, 306, 307, 308, 312, 313, 316, 318, 327, 374, 381, 394, 395, 402, 402/507, 404, 405, 414, 432, 451, 455, 460, 461, 476/480, 480, 480/481, 493, 494, and 522. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4442 and one or more residue differences as compared to SEQ ID NO: 4442 selected from N174M/K296P/P299E, D182A, D182C, D182N, D182R, E185G/N190M, E189A/N190M, E189H/N190M, E189R/N190M, E189V/N190M, N190M, N190M/L193F, E192T/R280H, E192T/S402L/K507R, W257K, W257R, Q259C, Q259K, Q259R, S260A, M281C, M281L, V289I, K296P/P299T, L306A, L306C, L305F, L306M, L306R, L306T, Y307N, Y308Q, A312H, A312K, A312Q, A312R, A312V, R313A, R313I, R313L, T316F, A318L, A318M, A318N, A318P, A318R, A318T, A318V, A318Y, V327A, L374M, W381F, W381M, W394L, W394M, W394Y, S395G, S402L, S402L/K507R, S404A, G405L, A414C, A414E, A414P, R432H, V451L, R455A, S460A, F461M, R476L/W480D, R476W/W480D, W480D, W480E, W480L, W480M, W480D/Y481W, H493K, S494Q, S494R, S494T, and E522T.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4654 and one or more residue differences as compared to SEQ ID NO: 4654 at a position or set of positions selected from 190, 190/197/308, 190/308/380/405, 190/375, 190/375/380, 190/380/405, 190/405/406, 272/301/393/394/480, 272/318/480/483, 301/394/480, 318, 375, 375/380, 375/405, 375/405/406, 380, 394, 394/480, and 480/483. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4654 and one or more residue differences as compared to SEQ ID NO: 4654 selected from N190M, N190M/D197N/Y308Q, N190M/Y308Q/L380V/G405L, N190M/Q375G, N190M/Q375G/L380V, N190M/L380V/G405L, N190M/G405L/K406S, V272A/Q301S/E393K/W394T/W480D, V272A/A318E/W480D/L483R, Q301S/W394T/W480D, A318E, Q375G, Q375G/L380V, Q375G/G405L, Q375G/G405L/K406S, L380V, W394T, W394T/W480D, and W480D/L483R.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4850 and one or more residue differences as compared to SEQ ID NO: 4850 at a position or set of positions selected from 189, 189/193/207/307/353, 190/322, 193, 193/307, 261/322/421, 297/298/300/392, 297/300, 297/300/328, 298/300/328, 298/300/328/395, 298/300/360, 298/300/392/395, 298/300/392/395/492, 298/300/395, 298/300/481, 300, 300/392/395, 319, 322, 392, 421, and 492. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4850 and one or more residue differences as compared to SEQ ID NO: 4850 selected from R189K, R189K/E193V/A207V/L307T/Q353R, E190R/D322N, E193V, E193V/L307T, S261A/D322N/L421V, K297P/L298F/P300T/R392S, K297P/P300E/V328A, K297P/P300S, L298F/P300E/V328A/W395L, L298F/P300E/R392S/W395L, L298F/P300E/W395L, L298F/P300E/D481W, L298F/P300S/V328A, L298F/P300S/R392S/W395L, L298F/P300S/R392S/W395L/D492E, L298F/P300T/L360V, P300E, P300E/R392S/W395L, P300T, A319P, D322N, R392S, L421V, and D492E.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4856 and one or more residue differences as compared to SEQ ID NO: 4856 at a position or set of positions selected from 10/413, 260, 268, 302/307, 317, 353, 354, 362, 364, 392, 393, 394, 395, 397, 402, 404, 412, 413, 419, 436/512, 460, 477, 486, 490, 495, and 518. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4856 and one or more residue differences as compared to SEQ ID NO: 4856 selected from S10N/A413S, Q260R, V268T, Q302A/T307L, Q302S/T307L, T317C, Q353R, Q353S, G354S, S362V, P364Q, R392G, R392H, R392K, L393R, L393V, E394A, E394V, W395Y, Q397G, Q397S, G402K, G402L, G402P, G402T, G402V, P404L, P404Q, D412G, D412M, A413G, L419G, L419V, P436S/E512D, G460A, G460P, G460S, R477Q, R477S, E486V, M490E, M490N, M490Q, M490S, M490T, M490V, S495Q, S495R, and G518S.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4904 and one or more residue differences as compared to SEQ ID NO: 4904 at a position or set of positions selected from 190, 190/287/300/302, 190/300/477/490, 194/300/302/413, 194/300/302/481, 297/298/308/392/395, 298/392/525, 300, 300/317, 300/490, and 395. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 4904 and one or more residue differences as compared to SEQ ID NO: 4904 selected from E190R, E190R/L287V/P300T/Q302A, E190R/P300T/R477Q/M490E, L194F/P300E/Q302S/D481M, L194F/P300T/Q302A/A413G, K297P/L298F/Y308N/R392K/W395Y, L298F/R392K/F525A, P300E, P300E/T317C, P300T/M490E, and W395Y.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 5002 and one or more residue differences as compared to SEQ ID NO: 5002 at a position or set of positions selected from 11/523, 190/194, 194, 194/198, 202, 208, 264, 273, 273/347/354, 274, 281, 290, 298/300/302, 298/302/392/393/394/433, 298/302/392/394, 298/392/393/394, 298/392/393/394/477, 298/392/394/490, 298/393/394/395/433/477, 298/393/394/477/495, 298/394/433, 300/302/303, 308/402/460, 309, 313, 314, 324, 352, 359, 360, 361, 392/393/394/433, 392/393/394/477, 392/393/394/477/495, 392/393/394/490, 392/394, 392/394/395, 392/394/433/477, 392/394/433/495, 392/394/477/495, 392/394/495, 393/394, 393/394/433/477/490, 394/477, 394/490, 405, 408/413, 411/413, 413, 460/525, 463, 466, 467, 472, 473, 477, 492, 523, and 526. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 5002 and one or more residue differences as compared to SEQ ID NO: 5002 selected from G11D/E523R, E190L/F194L, E190R/F194L, E190T/F194L, F194L, F194L/D198G, F194L/D198P, F194L/D198Q, F194L/D198V, F194W, F194Y, L202T, I208G, I208S, I208V, A264S, V273M/Q347F/S354G, V273Q, V273S, K274G, K274V, K274W, R281G, R281Q, V290A, L298F/A302S/R392K/L393V/E394A/R433H, L298F/A302S/R392K/E394A, L298F/R392K/L393V/E394A/R477L, L298F/R392K/L393V/E394V, L298F/R392K/E394V/M490E, L298F/L393V/E394V/W395Y/R433H/R477V, L298F/L393V/E394V/R477L/S495Q, L298F/E394V/R433H, L298I/T300P/A302Q, T300P/A302Q/K303V, Y308N/G402V/G460A, Y309R, A313S, R314L, I324M, I324T, P352T, L359C, L359V, L360V, I361L, R392H/L393V/E394A/R477L, R392H/L393V/E394V/M490E, R392H/E394A/R433H/R477L, R392H/E394V/R433H/S495Q, R392K/L393V/E394V/R433H, R392K/L393V/E394V/R477V/S495Q, R392K/E394A/W395Y, R392K/E394V, R392K/E394V/R477V/S495Q, R392K/E394V/S495Q, L393V/E394A, L393V/E394V/R433H/R477V/M490E, E394V/R477V, E394V/M490E, S405T, A408G/G413A, A408L/G413A, G411L/G413A, G413A, G460S/F525A, A463P, A463V, M466V, L467A, S472T, R473S, R477L, D492R, E523T, E526L, and E526Y.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 5028 and one or more residue differences as compared to SEQ ID NO: 5028 at a position or set of positions selected from 82/194/198/313, 194, 194/198, 194/198/208/313, 194/198/309, 194/198/313, 194/198/411, 194/208/411, 194/309, 194/313, 194/411, 198, 198/208, 198/208/309/411, 198/208/313/411, 273/274, 274, 274/281/526, 274/359/526, 274/523, 309, 309/313/411, 324/526, 411, 466, 466/526, 523, and 526. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 5028 and one or more residue differences as compared to SEQ ID NO: 5028 selected from V82I/F194L/D198G/A313S, F194L, F194L/D198Q, F194L/D198Q/I208V/A313S, F194L/D198V, F194L/D198V/Y309R, F194L/D198V/G411L, F194L/I208V/G411L, F194L/Y309R, F194W, F194W/D198Q, F194W/D198V, F194W/D198V/A313S, F194W/A313S, F194W/G411L, D198G, D198Q, D198Q/I208S/A313S/G411L, D198Q/I208V/Y309R/G411L, D198V/I208S, V273Q/K274G, V273S/K274W, K274G/R281Q/E526Y, K274M/E523T, K274V, K274V/L359V/E526Y, K274W, Y309R, Y309R/A313S/G411L, I324M/E526Y, G411L, M466V, M466V/E526Y, E523T, and E526Y.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 5192 and one or more residue differences as compared to SEQ ID NO: 5192 at a position or set of positions selected from 96/295, 169, 176, 177, 179, 184, 187, 193, 195, 197, 197/307, 198, 199, 200, 203, 292, 295, 300/394, 304, 325, 326, 326/380, 329, 373, 376, 377, 383, 394, 403, 409, 430, 485, 508, and 520/526. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 5192 and one or more residue differences as compared to SEQ ID NO: 5192 selected from L96S/T295N, Q169E, N176H, R177L, R177T, R177W, Q179G, Q179L, S184G, S184H, S184P, 1187V, V193C, V193F, V193R, V193S, K195L, N197F, N197M/T307A, D198L, D198S, D199G, D199R, D199S, T200A, T200E, T200K, T200Q, T200R, T200S, T200V, T200Y, E203A, E203C, E203G, E203L, S292L, S292M, S292Q, S292R, T295G, T295H, T295K, T295L, T300A/V394F, V3041, W325M, L326Q, L326R, L326T, L326M/N380R, Q329K, Q329L, Q373G, Q376R, K377N, K377R, K383H, K383R, K383S, V394F, V394H, V3941, V394L, V394Q, V394S, V394W, V394Y, L403R, L403V, D409S, K430R, E485L, K508A, K508S, and E520I/Y526E.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 5246 and one or more residue differences as compared to SEQ ID NO: 5246 at a position or set of positions selected from 177/198/200, 177/200/203, 177/200/203/295, 177/200/295/326, 180, 184/198/200/203/295, 184/200/295/326, 190/198/200/203, 190/200/203/295/380, 190/200/295, 197, 198, 198/200, 198/200/203, 198/200/203/295, 200/326, 200/380, 203, 203/380, 233, 252, 295, 336, 364, 365, 367, 381, 384, 441, 459, and 485. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 5246 and one or more residue differences as compared to SEQ ID NO: 5246 selected from R177W/D198L/T200K, R177W/T200K/E203G, R177W/T200K/E203G/T295N, R177W/T200K/T295N/L326M, R180K, S184H/D198L/T200K/E203G/T295N, S184H/T200K/T295N/L326M, E190M/D198L/T200K/E203G, E190M/T200K/E203G/T295N/N380R, E190M/T200K/T295N, N197Q, D198L, D198L/T200K, D198L/T200K/E203G, D198L/T200K/E203G/T295N, T200K/L326M, T200K/N380R, E203G, E203G/N380R, E233L, E233R, A252R, T295N, D336C, P364A, G365H, G365R, E367S, L381H, L381Y, K384A, M441E, R459M, R459W, and E485L.

In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2 or 660 and one or more residue differences as compared to SEQ ID NO: 2 or 660, selected from

26/30/38/79/81/90/92/94/101/108/137/140/141/142/153/155/160/163/165/177/184/189/194/196/201/203/205/213/219/231/248/258/263/264/266/267/301/304/307/314/318/325/333/340/344/353/362/379/390/392/394/395/397/398/402/403/406/408/410/4 11/413/414/416/425/427/429/433/434/441/442/444/446/451/455/460/461/466/468/476/481/484/485/488/495/499/501/502/506/515/525,
26/30/38/79/81/90/92/94/101/108/137/140/141/142/153/155/160/163/165/177/184/189/201/203/205/213/219/231/248/258/263/264/266/304/307/314/318/325/333/340/344/353/362/379/392/394/395/397/398/402/403/406/408/410/411/413/414/416/425/4 27/429/433/434/441/442/444/446/455/460/461/466/468/476/481/484/485/488/495/499/501/502/506/515/525,
26/38/79/81/90/92/94/101/108/137/141/155/160/163/165/189/201/203/205/213/219/246/248/258/263/264/304/307/314/318/333/340/344/353/362/392/394/395/396/397/398/402/403/406/408/410/411/413/414/425/441/442/446/455/460/461/466/468/476/ 481/485/488/506/525,
92/94/101/108/137/141/155/160/163/165/201/203/205/213/219/258/263/264/314/333/344/353/392/394/395/397/406/408/411/413/414/425/441/442/446/460/461/468/476/481/485/488/525, and
92/94/101/108/137/141/155/201/213/264/314/333/344/392/394/395/397/406/408/425/442/446/461/476/481/485/525. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2 or 660 and one or more residue differences as compared to SEQ ID NO: 2 or 660, selected from 26Q/30G/38T/79T/81E/90L/92R/94E/101E/108K/137A/140V/141E/142V/153V/155E/160M/163V/165P/177R/184S/189R/194L/196G/201A/203E/205R/213S/219R/231S/248R/258W/263H/264A/266T/267R/30 1W/304V/307L/314R/318R/325W/333A/340I/344V/353Q/362S/379M/390C/392R/394E/395W/397Q/39 8W/402G/403S/406G/408A/410E/411G/413A/414E/416S/425D/427L/429R/433R/434N/441M/442G/444 S/446P/451K/455L/460G/461S/466M/468Q/476R/481W/484L/485E/488N/495S/499M/501R/502G/506P/515E/525F,
26Q/30G/38T/79T/81E/90L/92R/94E/101E/108K/137A/140V/141E/142V/153V/155E/160M/163V/165P/177R/184S/189R/201A/203E/205R/213S/219R/231S/248R/258W/263H/264A/266T/304V/307L/314R/31 8R/325W/333A/340I/344V/353Q/362S/379M/392R/394E/395W/397Q/398W/402G/403S/406G/408A/41 0E/411G/413A/414E/416S/425D/427L/429R/433R/434N/441M/442G/444S/446P/455L/460G/461S/466M/468Q/476R/481W/484L/485E/488N/495S/499M/501R/502G/506P/515E/525F, 26Q/38T/79T/81E/90L/92R/94E/101E/108K/137A/141E/155E/160M/163V/165P/189R/201A/203E/205R/213S/219R/246N/248R/258W/263H/264R/304V/307L/314R/318R/333A/340I/344V/353Q/362S/392R/3 94E/395W/396T/397Q/398W/402G/403S/406G/408A/410E/411G/413A/414G/425D/441M/442G/446P/4 55L/460G/461G/466M/468Q/476R/481W/485D/488N/506P/525F,

### 92R/94E/101E/108K/137A/141E/155E/160M/163V/165P/201A/203E/205R/213S/219R/258W/263H/264R/314R/333A/344V/353Q/392R/394E/395W/397Q/406G/408A/411G/413A/414G/425D/441M/442G/446P/460G/461G/468Q/476R/481W/485D/488N/525F, and

92R/94E/101E/108K/137A/141E/155E/201A/213S/264R/314R/333A/344V/392R/394E/395W/397Q/406 G/408A/425D/442G/446P/461G/476R/481W/485D/525F. In some embodiments, the engineered TdT polypeptide comprises an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to reference sequence SEQ ID NO: 2 or 660 and one or more residue differences as compared to SEQ ID NO: 2 or 660, selected from
R26Q/M30G/I38T/S79T/S81E/N90L/G92R/D94E/T101E/T108K/M137A/G140V/V141E/M142V/E153V/I155E/T160M/I163V/Q165P/H177R/A184S/A189R/F194L/R196G/C201A/T203E/M205R/C213S/V219R/G231S/L248R/R258W/K263H/L264A/K266T/S267R/M301W/A304V/C307L/D314R/K318R/S325W/W333A/L340I/T344V/F353Q/T362S/T379M/Y390C/D392R/R394E/E395W/R397Q/F398W/K402G/L40 3S/R406G/I408A/A410E/L411G/H413A/F414E/K416S/H425D/K427L/D429R/W433R/E434N/E441M/S 442G/A444S/S446P/R451K/V455L/D460G/R461S/L466M/G468Q/E476R/R481W/T484L/H485E/K488N/A495S/L499M/K501R/K502G/K506P/A515E/S525F,
R26Q/M30G/I38T/S79T/S81E/N90L/G92R/D94E/T101E/T108K/M137A/G140V/V141E/M142V/E153V/I155E/T160M/I163V/Q165P/H177R/A184S/A189R/C201A/T203E/M205R/C213S/V219R/G231S/L248R/R258W/K263H/L264A/K266T/A304V/C307L/D314R/K318R/S325W/W333A/L340I/T344V/F353Q/T 362S/T379M/D392R/R394E/E395W/R397Q/F398W/K402G/L403S/R406G/I408A/A410E/L411G/H413 A/F414E/K416S/H425D/K427L/D429R/W433R/E434N/E441M/S442G/A444S/S446P/V455L/D460G/R 461S/L466M/G468Q/E476R/R481W/T484L/H485E/K488N/A495S/L499M/K501R/K502G/K506P/A515E/S525F,
R26Q/I38T/S79T/S81E/N90L/G92R/D94E/T101E/T108K/M137A/V141E/I155E/T160M/I163V/Q165P/A189R/C201A/T203E/M205R/C213S/V219R/E246N/L248R/R258W/K263H/L264R/A304V/C307L/D31 4R/K318R/W333A/L340I/T344V/F353Q/T362S/D392R/R394E/E395W/S396T/R397Q/F398W/K402G/L 403S/R406G/I408A/A410E/L411 G/H413A/F414G/H425D/E441M/S442G/S446P/V455L/D460G/R461G/L466M/G468Q/E476R/R481W/H485D/K488N/K506P/S525F,
G92R/D94E/T101E/T108K/M137A/V141E/I155E/T160M/I163V/Q165P/C201A/T203E/M205R/C213S/V219R/R258W/K263H/L264R/D314R/W333A/T344V/F353Q/D392R/R394E/E395W/R397Q/R406G/I4 08A/L411G/H413A/F414G/H425D/E441M/S442G/S446P/D460G/R461G/G468Q/E476R/R481W/H485 D/K488N/S525F, and

### G92R/D94E/T101E/T108K/M137A/V141E/I155E/C201A/C213S/L264R/D314R/W333A/T344V/D392R/R394E/E395W/R397Q/R406G/I408A/H425D/S442G/S446P/R461G/E476R/R481W/H485D/S525F.

As will be appreciated by the skilled artisan, in some embodiments, one or a combination of residue differences above that is selected can be kept constant (i.e., maintained) in the engineered TdT as a core feature, and additional residue differences at other residue positions incorporated into the sequence to generate additional engineered TdT polypeptides with improved properties. Accordingly, it is to be understood for any engineered TdT containing one or a subset of the residue differences above, the present invention contemplates other engineered TdTs that comprise the one or subset of the residue differences, and additionally one or more residue differences at the other residue positions disclosed herein.

As noted above, the engineered TdT polypeptides are also capable of converting substrates (e.g., NTP-3′-O-RBG or a natural or modified NTP and an oligo acceptor substrate) to products (e.g., an oligo acceptor substrate with an added nucleotide-3′-O-RBG). In some embodiments, the engineered TdT polypeptide is capable of converting the substrate compounds to the product compound with at least 1.2 fold, 1.5 fold, 2 fold, 3 fold, 4 fold, 5 fold, 10 fold, 20 fold, 30 fold, 40 fold, 50 fold, 60 fold, 70 fold, 80 fold, 90 fold, 100 fold, or more activity relative to the activity of the reference polypeptide of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192, and/or 5246.

In some embodiments, the engineered TdT capable of converting the substrate compounds to the product compounds with at least 2 fold the activity relative to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192, and/or 5246, comprises an amino acid sequence selected from the even-numbered sequences in SEQ ID NO: 4-1960, 2004-3920, 4048-5466, and 5476.

In some embodiments, the engineered TdT has an amino acid sequence comprising one or more residue differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192, and/or 5246, that increases soluble expression or isolated protein yield of the engineered TdT in a bacterial host cell, particularly in E. coli, as compared to a wild-type or engineered reference TdT, comprises an amino acid sequence selected from the even-numbered sequences in SEQ ID NO: 4-1960, 2004-3920, 4048-5466, and 5476.

In some embodiments, the engineered TdT has an amino acid sequence comprising one or more residue differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192, and/or 5246, that increases thermostability of the engineered TdT, as compared to a wild-type or engineered reference TdT, comprises an amino acid sequence selected from the even-numbered sequences of SEQ ID NO: 4-1960, 2004-3920, 4048-5466, and 5476.

In some embodiments, the engineered TdT has an amino acid sequence comprising one or more residue differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, that increases the activity of the engineered TdT at high temperatures (by way of example and not limitation, 40° C., 45° C., 50° C., 55° C., 60° C., or 65° C.), as compared to a wild-type or engineered reference TdT, comprises an amino acid sequence selected from the even-numbered sequences of SEQ ID NO: 4-1960, 2004-3920, 4048-5466, and 5476.

In some embodiments, the engineered TdT has an amino acid sequence comprising one or more residue differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, that reduces the by-product formation of the engineered TdT, as compared to a wild-type or engineered reference TdT, comprises an amino acid sequence selected from the even-numbered sequences of SEQ ID NO: 4-1960, 2004-3920, 4048-5466, and 5476.

In some embodiments, the engineered TdT has an amino acid sequence comprising one or more residue differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, that increases specific activity of the engineered TdT on one or more NTP-3′-O-RBG or a natural or modified NTP substrates, as compared to a wild-type or engineered reference TdT, comprises an amino acid sequence selected from the even-numbered sequences of SEQ ID NO: 4-1960, 2004-3920, 4048-5466, and 5476.

In some embodiments, the engineered TdT has an amino acid sequence comprising one or more residue differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, that increases specific activity of the engineered TdT on one or more oligo acceptor substrates, as compared to a wild-type or engineered reference TdT, comprises an amino acid sequence selected from the even-numbered sequences of SEQ ID NO: 4-1960, 2004-3920, 4048-5466, and 5476.

In some embodiments, the engineered TdT has an amino acid sequence comprising one or more residue differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, that increases incorporation efficiency in extension of an oligo acceptor substrate by addition of an NTP or NQP of greater than 60%, 65%, 70%, 75%, 80%, 85%, 90%, 91%, 92%, 93%, 94%, 95%, 96%, 97%, 98%, or 99%. when compared to the incorporation efficiency of a wild-type or engineered reference TdT, and comprises an amino acid sequence selected from the even-numbered sequences of SEQ ID NO: 4-1960, 2004-3920, 4048-5466, and 5476

In some embodiments, the engineered TdT with improved properties has an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to a reference sequence of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246 and at least one substitution or substitution set at amino acid positions selected from 26, 30, 38, 79, 81, 90, 92, 94, 101, 108, 137, 140 141, 142, 153, 155, 160, 163, 165, 177, 184, 189, 194, 196, 201, 203, 205, 213, 219, 231, 246, 248, 258, 263, 264, 266, 267, 301, 304, 307, 314, 318, 325, 333, 340, 344, 353, 362, 379, 390, 392, 394, 395, 396, 397, 398, 402, 403, 406, 408, 410, 411, 413, 414, 416, 425, 427, 429, 433, 434, 441, 442, 444, 446, 451, 455, 460, 461, 466, 468, 476, 481, 484, 485, 488, 495, 499, 501, 502, 506, 515, and 525, and/or any combinations thereof, wherein the amino acid positions are numbered with reference to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246.

In some embodiments, the engineered TdT with improved properties has an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to a reference sequence of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246 and at least one substitution at amino acid position 12, 13, 14, 15, 16, 17, 18, 20, 21, 22, 23, 24, 25, 26, 27, 28, 29, 30, 31, 32, 33, 34, 35, 36, 37, 38, 39, 41, 44, 45, 46, 47, 50, 53, 57, 58, 61, 62, 65, 70, 76, 77, 79, 80, 81, 85, 89, 90, 92, 93, 94, 97, 101, 102, 103, 104, 105, 106, 107, 108, 109, 110, 116, 119, 121, 122, 123, 124, 126, 129, 130, 131, 132, 133, 134, 135, 137, 138, 139, 140, 141, 142, 144, 145, 147, 148, 149, 150, 151, 152, 153, 154, 155, 156, 157, 158, 159, 160, 161, 162, 163, 165, 168, 169, 171, 173, 174, 175, 177, 179, 183, 184, 185, 186, 188, 189, 190, 191, 192, 193, 194, 195, 196, 197, 198, 199, 200, 201, 203, 204, 205, 206, 208, 213, 217, 219, 220, 223, 227, 231, 233, 235, 236, 243, 244, 245, 246, 248, 249, 251, 252, 253, 255, 258, 259, 260, 261, 262, 263, 264, 266, 267, 268, 269, 271, 272, 273, 274, 275, 276, 277, 278, 279, 280, 281, 282, 284, 286, 287, 288, 289, 290, 291, 292, 293, 294, 295, 297, 298, 299, 300, 301, 302, 303, 304, 306, 307, 308, 309, 310, 312, 313, 314, 315, 316, 317, 318, 319, 320, 321, 322, 324, 325, 326, 327, 328, 329, 330, 333, 334, 336, 338, 340, 342, 343, 344, 347, 350, 352, 353, 359, 360, 361, 362, 363, 365, 366, 367, 368, 369, 370, 371, 372, 373, 376, 378, 379, 380, 382, 383, 385, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 421, 425, 426, 427, 428, 429, 431, 433, 434, 435, 436, 437, 438, 439, 440, 441, 442, 443, 444, 445, 446, 448, 451, 452, 454, 455, 456, 457, 459, 460, 461, 462, 464, 465, 466, 468, 469, 470, 472, 473, 474, 475, 476, 477, 479, 480, 481, 484, 485, 488, 490, 491, 492, 493, 494, 495, 499, 500, 501, 502, 503, 504, 506, 508, 509, 515, 518, 520, 522, 523, 525, 526, or 528, or any combinations thereof, wherein the amino acid positions are numbered with reference to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246.

In some embodiments, the engineered TdT with improved properties has an amino acid sequence having at least 60%, 70%, 80%, 85%, 86%, 87%, 88%, 89%, 90%, 91%, 92%, 93%, 94%, 95%, 96%, 97%, 98%, 99% or more sequence identity to a reference sequence of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246 and at least a substitution or amino acid residue 12A/F/N/Q/S/V, 13E/G/K/R/S, 14G/N/Q/Y, 15A/F/L/Q, 16S/V, 17A/G, 18D/R/Y, 20H/P/T, 21E, 22G/L, 23C/E/P/T, 24A/G/M/P, 25N, 26A/G/I/Q/S/T/W, 27D/F/R, 28N/R, 29G/I/P/R, 30E/G/L/T/V, 31G/S/V, 32E, 33A/C/G/K/P, 34D/K/R/S, 35E/G/H/W, 36G/K, 37A/F/G/S/T/V, 38L/T, 39A/G, 41V, 44S, 45R, 46M, 47A, 50L, 53K, 53E, 57H/T, 58A/M/N/S, 61A/H/L, 62W, 65S, 70A, 76P, 77C/S, 79T, 80D, 81E, 85V, 89A, 90L/M/S, 92M/R/S/V/Y, 93V, 94E/K/N/R, 97D/T, 101E/G/V, 102L, 103M, 104G/I/P/V, 105N/W, 106D/G/H/K/S/V, 107R/W, 108D/G/K/M, 109M/N/T, 110M/V, 116A, 119F/Q, 121S, 122I, 123M/Q, 124E/G/I/M/S, 126C/V, 129G, 130A/M/Q/S, 131E/G/R/W, 132S, 133G/M/Q, 134M/W, 135C/E/H/K/R, 137A/E, 138A/Q, 139A/G, 140E/G/V, 141E/M/R, 142M/S/V, 144C, 145E, 147G/L, 148T, 149R, 150E/G/P, 151I/S, 152G/H/R, 153E/G/K/M/P/Q/V, 154G, 155E/T, 156D/E/M/Q, 157L/V, 158A/H/S, 159D, 160G/M/N/S/V, 161D/E/G/H/R/S, 162A/E/H, 163I/L/V, 165K/P, 168S, 169E/M/R, 171K, 173I/L, 174L, 175D/I/L/V, 177L/R/Y, 179A/K/L/R, 183R, 184S, 185C/F/L, 186A/C/D/G/L/T, 188I/M, 189E/L/Q/R/V, 190L/M/R, 190E, 191V, 192C/G/I/L/R, 193C/G/N/R/S/T/V, 194A/C/D/E/F/G/L/M/R/S/W, 196A/G/L/M/N/R/S/T/W/Y, 197G/L/M/R, 198A/E/L/P/S, 199A/E/G/H/I/M/Q/R/S/V, 200A/C/K/N/V, 201A/R/W, 203A/D/E/G/L/M/R/S, 204L, 205E/L/R, 206N, 208A/V, 213S, 217Q, 219L/R, 220R, 223Q, 227M, 231S/T, 233A/D/R, 235R, 236E/I/P/V, 243L/R/S/T, 244V, 245A, 246E/K/N, 248C/K/L/R/S/T/V, 249A/G/R/T, 251K/R, 252E/K, 253I/L, 255G, 258A/C/E/G/K/L/M/Q/S/W, 259N/V, 260K, 261A/G/R/V, 262I/V, 263H/I/M/R/S, 264A/C/E/L/R/S/T/V, 266F/I/K/R/T/V/Y, 267A/C/H/M/Q/R/V/Y, 268C/I/L/T, 269W, 271C, 272T, 273C/D/E/F/G/I/L/M/S/V/W/Y, 274A/G/I/M/N/P/Q/T/V, 275D/V, 276S, 277E, 278C/E/F/H/I/L/M/N/R/T/V/Y, 279Y, 280F/L/S, 281A/C/G/K/L/Q/S/T/V, 282C/G/H/Q/R/W, 284I/L/M, 286N/S, 287I, 288E/V, 289D, 290A/L/V, 291M/S/W, 292L/N/T, 293S/Q, 294T, 295S, 297C/D/F/L/M/P/Q/R/S/T/V, 298I/M, 299A/L/M/N/S/Y, 300H/P/R/T, 301G/Q/S/T/V/W, 302A/N/S, 303A/E/H/M/N/Q/S/V, 304D/L/M/T/V, 306F/I/M, 307A/E/G/H/K/L/M/S/V, 308A/F/G/H/K/L/N/S/V/W, 309F/G/I/L/M/V/W, 310A/G/H/R/S, 312M/T/V, 313A/I/L/M/Q/R/S, 314A/G/I/K/L/M/Q/R/V/Y, 315A/G/Q/S, 316A/C/I/L/T, 317G/T, 318E/R/S/T/V, 319G/R, 320N, 321C/K/S, 322A/K/Q, 324I/V, 325A/F/L/T/V/W, 326C/M/N/R/S/T, 327I/M, 328T, 329K/R, 330E/K/N, 333A/D/G/H/R, 334E/R/S, 336D, 338T, 340A/G/I/M/R/S/V, 342E/R/V/W, 343V, 344V, 347I/Q, 350L/R/S/W, 352G/P/R/V, 353A/H/K/M/Q/R/S, 354S, 359G/L/V, 360C/I/V, 361T, 362S/T/Y, 363C/I, 365A, 366V, 367A/D, 368R, 369G/M/N, 370D/G/M/N/S/T, 371D, 372E/G, 373C/G/H/N/R, 376H/V, 378L, 379C/I/L/M/V, 380D, 382F/L, 383R, 385R, 390C/I/L/V, 391G/L/R, 392A/C/K/R/V, 393I/R/V, 394A/E/F/G/L/M/R/S/T/V/Y, 395A/L/R/S/T/W/Y, 396A/R/S/T, 397A/D/F/Q/R/T/W, 398W, 399C/D/F/G/T, 400A/E/W, 401E/G, 402E/F/G/Q/S/V, 403A/E/F/G/L/P/R/S, 404D/E/F/S/W, 405G/L/N/Y, 406G/N/P/T/V, 407A/D/F/L/M/N/R/S/W, 408A/E/G/I/L/M/P/R/T/V/W, 409K/L/Q, 410E/F/G/I/Q/S/V/Y, 411A/E/F/G/I/N/P/Q/R/T, 412N, 413A/C/E/F/G/I/L/M/P/S/V, 414A/E/F/G/H/Y, 415A/F/L/S/W, 416G/N/Q/S, 417G/V/W, 418I, 419A/G/H/L/M, 421F/I/M, 425D/K/R/T, 426P, 427C/E/F/L/M/N/Q/R/W/Y, 428V, 429R, 431R/S, 433A/E/G/H/M/P/R/S/V, 434N, 435A/C/E/G/I/K/P/Q/S/T, 436S, 437A/G/K/P/Q/R/S, 438V, 439G/P, 440E/K/V, 441K/M/N/V, 442A/G/K, 443T, 444A/G/H/R/S, 445N, 446E/G/P, 448R, 451K, 452I/L, 454F/M/V, 455I/L, 456K/P/R/S/T, 457S/V, 459H/I/Q/R/V, 460E/G/P/V, 461A/G/N/Q/S/V, 462E/F/H/I/L/Q/R/W, 464Y, 465E, 466M, 468A/F/H/M/Q/S/T/W, 469F/Q/Y, 470S/T, 472A/G, 473A/D/G/K/M/P/Q/S/V, 474M, 475V, 476R/V, 477G/Q/S/T, 479F/V, 480A/E/G/H/K/L/S/W, 481A/D/E/L/M/S/T/V/W, 484A/E/H/L/M/R, 485D/E/S, 488N/S, 490E/H/L/R/V/W, 491I/M, 492S/T, 493E/Q/R/V/Y, 494A/C/G/L/R/V/W, 495C/G/S, 499L/M/R, 500N, 501A/N/R, 502G/R, 503A/E/M/Q/R/S/T/V, 504K/N/Q/R/S/W, 506E/P/S/T, 508D/S/T, 509G/K, 515E/V, 515A, 518D, 520D/P, 522L, 523E/H, 525F/H/Q/R/S, 526L/Y, or 528L, or any combinations thereof, wherein the amino acid positions are numbered with reference to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246.

In some embodiments, the engineered TdT with improved properties has an amino acid sequence comprising a sequence selected from the even-numbered sequences of SEQ ID NO: 4-1960, 2004-3920, 4048-5466, and 5476. In some embodiments, the engineered TdT with improved properties has an amino acid sequence comprising a sequence selected from SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246.

In some embodiments, the engineered TdT, comprises an amino acid sequence having at least 80%, 85%, 86%, 87%, 88%, 89%, 90%, 91%, 92%, 93%, 94%, 95%, 96%, 97%, 98%, or 99% identity to one of the sequences of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, as provided in the Examples.

In addition to the residue positions specified above, any of the engineered TdT polypeptides disclosed herein can further comprise other residue differences relative to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, at other residue positions (i.e., residue positions other than those included herein). Residue differences at these other residue positions can provide for additional variations in the amino acid sequence without adversely affecting the ability of the polypeptide to carry out the conversion of substrate to product. Accordingly, in some embodiments, in addition to the amino acid residue differences present in any one of the engineered TdTs polypeptides selected from the even-numbered sequences in the range of SEQ ID NO: 4-1960, 2004-3920, 4048-5466, and 5476, the sequence can further comprise 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-100, or 1-150 residue differences at other amino acid residue positions as compared to the SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246. In some embodiments, the number of amino acid residue differences as compared to the reference sequence can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, 50, 100, or 150 residue positions. In some embodiments, the number of amino acid residue differences as compared to the reference sequence can be 1, 2, 3, 4, 5, 6, 7, 8, 9, 10, 11, 12, 13, 14, 15, 16, 18, 20, 21, 22, 23, 24, or 25 residue positions. The residue differences at these other positions can be conservative changes or non-conservative changes. In some embodiments, the residue differences can comprise conservative substitutions and non-conservative substitutions as compared to the TdT polypeptide of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246.

In some embodiments, the present invention also provides engineered polypeptides that comprise a fragment of any of the engineered TdT polypeptides described herein that retains the functional activity and/or improved property of that engineered TdT. Accordingly, in some embodiments, the present invention provides a polypeptide fragment capable of converting substrate to product under suitable reaction conditions, wherein the fragment comprises at least about 90%, 95%, 96%, 97%, 98%, or 99% of a full-length or truncated amino acid sequence of an engineered TdT of the present invention, such as an exemplary TdT polypeptide selected from the even-numbered sequences in the range of SEQ ID NO: 4-1960, 2004-3920, 4048-5466, and 5476. In some embodiments, the engineered TdT can have an amino acid sequence comprising a deletion in any one of the TdT polypeptide sequences described herein, such as the exemplary engineered polypeptides of the even-numbered sequences in the range of SEQ ID NO: 4-1960, 2004-3920, 4048-5466, and 5476.

Thus, for each and every embodiment of the engineered TdT polypeptides of the invention, the amino acid sequence can comprise deletions of one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 20% of the total number of amino acids, or up to 30% of the total number of amino acids of the TdT polypeptides, where the associated functional activity and/or improved properties of the engineered TdT described herein are maintained. In some embodiments, the deletions can comprise 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1- 45, or 1-50 amino acid residues. In some embodiments, the number of deletions can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residues. In some embodiments, the deletions can comprise deletions of 1, 2, 3, 4, 5, 6, 7, 8, 9, 10, 11, 12, 13, 14, 15, 16, 18, 20, 21, 22, 23, 24, or 25 amino acid residues.

In some embodiments, the engineered TdT polypeptide herein can have an amino acid sequence comprising an insertion as compared to any one of the engineered TdT polypeptides described herein, such as the exemplary engineered polypeptides of the even-numbered sequences in the range of SEQ ID NO: 4-1960, 2004-3920, 4048-5466, and 5476. Thus, for each and every embodiment of the TdT polypeptides of the invention, the insertions can comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, 20 or more amino acids, 30 or more amino acids, 40 or more amino acids, or 50 or more amino acids, where the associated functional activity and/or improved properties of the engineered TdT described herein is maintained. The insertions can be to amino or carboxy terminus, or internal portions of the TdT polypeptide.

In some embodiments, the engineered TdT described herein can have an amino acid sequence comprising a sequence selected from the even-numbered sequences in the range of SEQ ID NO: 4-1960, 2004-3920, 4048-5466, and 5476, and optionally one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, 1-50, 1-75, 1-100, or 1-150 amino acid residue deletions, insertions and/or substitutions. In some embodiments, the amino acid sequence has optionally around 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, 50, 55, 60, 65, 70, 80, 90, 100, 110, 120, 130, 140, or 150 amino acid residue deletions, insertions and/or substitutions. In some embodiments, the substitutions can be conservative or non-conservative substitutions.

In the above embodiments, the suitable reaction conditions for the engineered polypeptides are provided in Tables 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, 35.1, 36.1, 37.1, 38.1, 39.1, 40.1, 41.1, 42.1, 43.1, 44.1, 45.1, 46.1, 47.1, 48.1, 49.1, 50.1, 51.1, 52.1, 53.1, 54.1, 55.1, 56.1, 59.1, 60.1, 61.1, 63.1, 64.1, 65.1, 66.1, 67.1, 68.1, 69.1, 70.1, 71.1, 72.1, 73.1, 74.1, 75.1, 76.1, 77.1, 78.1, 79.1, and 80.1, as described in the Examples herein.

In some embodiments, the polypeptides of the present invention are fusion polypeptides in which the engineered polypeptides are fused to other polypeptides, such as, by way of example and not limitation, antibody tags (e.g., myc epitope), purification sequences (e.g., His tags for binding to metals), cell localization signals (e.g., secretion signals), and polypeptides with enzymatic activity. Thus, the engineered polypeptides described herein can be used with or without fusions to other polypeptides.

In one embodiment of the engineered TdT polypeptides of the present invention, the polypeptide further comprises an N-terminal truncation of 1-156 amino acids of the polypeptide sequence relative to any even-numbered sequence set forth in SEQ ID NO: 4-1960, 2004-3920, 4048-5466, and 5476. For example, a 156 amino acid truncated version of a TdT variant polypeptide SEQ ID NO: 5028 is prepared and demonstrated to have TdT activity in Examples 81 and 82.

In some embodiments, the engineered TdT polypeptides of the invention can be fused to another second polypeptide, such as a polypeptide with a different enzymatic activity. In some embodiments, the present provides a fusion polypeptide comprising an engineered TdT polypeptide fused to a second polypeptide with inorganic pyrophosphatase (IPP) activity. For example, synthetic genes encoding an N-terminal and C-terminal hexahistidine tagged version of a wild-type (WT) inorganic pyrophosphatase (IPP) polypeptide (e.g., polypeptide of SEQ ID NO: 3942 or 3944) can be fused to gene encoding a TdT variant polypeptide. Typically, the polypeptides (e.g., IPP and TdT) are fused via a polypeptide linker (e.g., a GSGGTG linker) introduced in the construct between the genes encoding the polypeptides. Such fusion proteins can be constructed using well-established techniques (e.g., Gibson assembly cloning) and expressed in E. coli (e.g., a strain derived from W3110). Exemplary IPP-TdT polypeptide fusion constructs (e.g., the fusion constructs of SEQ ID NO: 5468, 5470, 5472, and 5474) are provided and demonstrated in Examples 81 and 82. Although the Examples demonstrate a fusion of a particular engineered TdT polypeptide of the present invention with a second polypeptide with IPP activity, it is contemplated that any of the embodiments an engineered TdT polypeptides of even-numbered sequence set forth in SEQ ID NO: 4-1960, 2004-3920, 4048-5466, and 5476 could be used in such a fusion with a second polypeptide.

It is to be understood that the polypeptides described herein are not restricted to the genetically encoded amino acids. In addition to the genetically encoded amino acids, the polypeptides described herein may be comprised, either in whole or in part, of naturally occurring and/or synthetic non-encoded amino acids. Certain commonly encountered non-encoded amino acids of which the polypeptides described herein may be comprised include, but are not limited to: the D-stereoisomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn); citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (NaI); 2-chlorophenylalanine (Ocf); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); P-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aGly); propargylglycine (pgGly); homoalanine (hAla); norvaline (nVal); homoleucine (hLeu), homovaline (hVal); homoisoleucine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the polypeptides described herein may be comprised will be apparent to those of skill in the art (See e.g., the various amino acids provided in Fasman, CRC Practical Handbook of Biochemistry and Molecular Biology, CRC Press, Boca Raton, FL, pp. 3-70 [1989], and the references cited therein, all of which are incorporated by reference). These amino acids may be in either the L- or D-configuration.

Those of skill in the art will recognize that amino acids or residues bearing side chain protecting groups may also comprise the polypeptides described herein. Non-limiting examples of such protected amino acids, which in this case belong to the aromatic category, include (protecting groups listed in parentheses), but are not limited to: Arg(tos), Cys(methylbenzyl), Cys (nitropyridinesulfenyl), Glu(S-benzylester), Gln(xanthyl), Asn(N—S-xanthyl), His(bom), His(benzyl), His(tos), Lys(fmoc), Lys(tos), Ser(O-benzyl), Thr (O-benzyl) and Tyr(O-benzyl).

Non-encoding amino acids that are conformationally constrained of which the polypeptides described herein may be composed include, but are not limited to, N-methyl amino acids (L-configuration); 1-aminocyclopent-(2 or 3)-ene-4-carboxylic acid; pipecolic acid; azetidine-3-carboxylic acid; homoproline (hPro); and 1-aminocyclopentane-3-carboxylic acid.

In some embodiments, the engineered polypeptides can be in various forms, for example, such as an isolated preparation, as a substantially purified enzyme, whole cells transformed with gene(s) encoding the enzyme, and/or as cell extracts and/or lysates of such cells. The enzymes can be lyophilized, spray-dried, precipitated or be in the form of a crude paste, as further discussed below.

In some embodiments, the engineered polypeptides can be in the form of a biocatalytic composition. In some embodiments, the biocatalytic composition comprises (a) a means for conversion of an NTP-3-O-RBG or natural or modified NTP substrate and an oligo acceptor compound to an oligo acceptor product extended by one nucleotide by contact with a TdT and (b) a suitable cofactor. The suitable cofactor may be cobalt, manganese, or any other suitable cofactor.

In some embodiments, the polypeptides described herein are provided in the form of kits. The enzymes in the kits may be present individually or as a plurality of enzymes. The kits can further include reagents for carrying out the enzymatic reactions, substrates for assessing the activity of enzymes, as well as reagents for detecting the products. The kits can also include reagent dispensers and instructions for use of the kits.

In some embodiments, the kits of the present invention include arrays comprising a plurality of different TdT polypeptides at different addressable position, wherein the different polypeptides are different variants of a reference sequence each having at least one different improved enzyme property. In some embodiments, a plurality of polypeptides immobilized on solid supports are configured on an array at various locations, addressable for robotic delivery of reagents, or by detection methods and/or instruments. The array can be used to test a variety of substrate compounds for conversion by the polypeptides. Such arrays comprising a plurality of engineered polypeptides and methods of their use are known in the art (See e.g., WO2009/008908A2).

Polynucleotides Encoding Engineered Terminal Deoxynucleotidyl Transferases, Expression Vectors and Host Cells

In another aspect, the present invention provides polynucleotides encoding the engineered TdT polypeptides described herein. The polynucleotides may be operatively linked to one or more heterologous regulatory sequences that control gene expression to create a recombinant polynucleotide capable of expressing the polypeptide. Expression constructs containing a heterologous polynucleotide encoding the engineered TdT are introduced into appropriate host cells to express the corresponding TdT polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the improved TdT enzymes. Thus, having knowledge of a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made encoding the polypeptides described herein by selecting combinations based on the possible codon choices, and all such variations are to be considered specifically disclosed for any polypeptide described herein, including the amino acid sequences presented in Tables 5.1, 6.2, 7.2, 8.2, 9.2, 10.2, 11.2, 12.2, 13.2, 14.2, 15.2, 16.2, 17.2, 18.2, 19.2, 20.2, 21.2, 22.2, 23.2, 24.2, 25.2, 26.2, 26.3, 26.4, 27.2, 27.3, 27.4, 27.5, 28.1, 28.2, 28.3, 29.2, 30.2, 31.2, 32.2, 33.2, 34.2, 35.2, 36.2, 37.2, 38.2, 39.2, 40.2, 41.2, 42.2, 43.2, 44.2, 45.2 46.2, 47.2, 48.2, 49.2, 50.2, 51.2, 52.2, 53.2, 54.2, 55.2, 56.2, 56.3, 56.4, 61.2, 63.2, 64.2, 65.2, 66.2, 67.2, 68.2, 69.2, 70.2, 71.2, 72.2, 73.2, 74.2, 75.2, 76.2, 77.2, 78.2, 79.2, and 80.1, and disclosed in the sequence listing incorporated by reference herein as the even-numbered sequences in the range of SEQ ID NO: 4-1960, 2004-3920, 4048-5466, and 5476.

In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. In some embodiments, all codons need not be replaced to optimize the codon usage of the TdT since the natural sequence will comprise preferred codons and because use of preferred codons may not be required for all amino acid residues. Consequently, codon optimized polynucleotides encoding the TdT enzymes may contain preferred codons at about 40%, 50%, 60%, 70%, 80%, or greater than 90% of codon positions of the full-length coding region.

In some embodiments, the polynucleotide comprises a codon optimized nucleotide sequence encoding the TdT polypeptide amino acid sequence, as represented by SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246. In some embodiments, the polynucleotide has a nucleic acid sequence comprising at least 80%, 85%, 90%, 91%, 92%, 93%, 94%, 95%, 96%, 97%, 98%, 99% or more identity to the codon optimized nucleic acid sequences encoding the even-numbered sequences in the range of SEQ ID NOs: 4-1960, 2004-3920, 4048-5466, and 5476. In some embodiments, the polynucleotide has a nucleic acid sequence comprising at least 80%, 85%, 90%, 91%, 92%, 93%, 94%, 95%, 96%, 97%, 98%, 99% or more identity to the codon optimized nucleic acid sequences in the odd-numbered sequences in the range of SEQ ID NOs: 3-1959, 2003-3919, 4047-5465, and 5475. In some embodiments, the codon optimized sequences of the odd-numbered sequences in the range of SEQ ID NOs: 3-1959, 2003-3919, 4047-5465, and 5475, enhance expression of the encoded TdT, providing preparations of enzyme capable of converting substrate to product.

In some embodiments, the polynucleotide sequence comprises at least 85%, 90%, 91%, 92%, 93%, 94%, 95%, 96%, 97%, 98%, 99%, or more sequence identity to the reference sequence of SEQ ID NOs: 1, 7, 15, 23, 35, 267, 647, 659, 881, 1099, 1335, 1347, 1595, 1653, 1829, 1949, 2007, 2253, 2513, 2523, 2637, 2803, 2811, 2955, 3173, 3221, 3669, 3673, 3795, 3869, 3917, 4265, 4441, 4653, 4849, 4855, 4903, 5001, 5027, 5191, and/or 5245 and/or or a functional fragment thereof, wherein said polynucleotide sequence encodes an engineered polypeptide comprising at least one substitution at one or more amino acid positions.

In some embodiments, the polynucleotide sequence encodes at least one engineered terminal deoxynucleotidyl transferase comprising a sequence having at least 85%, 90%, 91%, 92%, 93%, 94%, 95%, 96%, 97%, 98%, 99%, or more sequence identity to the reference sequence of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246.

In some embodiments, the polynucleotide sequence comprises SEQ ID NOs: 7, 15, 23, 35, 267, 647, 659, 881, 1099, 1335, 1347, 1595, 1653, 1829, 1949, 2007, 2253, 2513, 2523, 2637, 2803, 2811, 2955, 3173, 3221, 3669, 3673, 3795, 3869, 3917, 4265, 4441, 4653, 4849, 4855, 4903, 5001, 5027, 5191, and/or 5245.

In some embodiments, the polynucleotides are capable of hybridizing under highly stringent conditions to a reference sequence selected from the odd-numbered sequences in SEQ ID NOs: 3-1959, 2003-3919, 4047-5465, and 5475, or a complement thereof, and encode a TdT.

In some embodiments, as described above, the polynucleotide encodes an engineered TdT polypeptide with improved properties as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, wherein the polypeptide comprises an amino acid sequence having at least 80%, 85%, 86%, 87%, 88%, 89%, 90%, 91%, 92%, 93%, 94%, 95%, 96%, 97%, 98%, 99% or more identity to a reference sequence selected from SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, and one or more residue differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, wherein the sequence is selected from the even-numbered sequences in the range of SEQ ID NOs: 4-1960, 2004-3920, 4048-5466, and 5476. In some embodiments, the reference amino acid sequence is selected from the even-numbered sequences in the range of SEQ ID NOs: 4-1960, 2004-3920, 4048-5466, and 5476. In some embodiments, the reference amino acid sequence is SEQ ID NO: 2, while in some other embodiments, the reference sequence is SEQ ID NO: 8, while in some other embodiments, the reference sequence is SEQ ID NO: 16. In some embodiments, the reference amino acid sequence is SEQ ID NO: 24, while in some other embodiments, the reference sequence is SEQ ID NO: 36, while in some other embodiments, the reference sequence is SEQ ID NO: 268. In some embodiments, the reference amino acid sequence is SEQ ID NO: 648, while in some other embodiments, the reference sequence is SEQ ID NO: 660, while in some other embodiments, the reference sequence is SEQ ID NO: 882. In some embodiments, the reference amino acid sequence is SEQ ID NO: 1100, while in some other embodiments, the reference sequence is SEQ ID NO: 1336, while in some other embodiments, the reference sequence is SEQ ID NO: 1348. In some embodiments, the reference amino acid sequence is SEQ ID NO: 1596, while in some other embodiments, the reference sequence is SEQ ID NO: 1654, while in some other embodiments, the reference sequence is SEQ ID NO: 1830. In some embodiments, the reference amino acid sequence is SEQ ID NO: 1950, while in some other embodiments, the reference sequence is SEQ ID NO: 2008, while in some other embodiments, the reference sequence is SEQ ID NO: 2254. In some embodiments, the reference amino acid sequence is SEQ ID NO: 2514, while in some other embodiments, the reference sequence is SEQ ID NO: 2524, while in some other embodiments, the reference sequence is SEQ ID NO: 2638. In some embodiments, the reference amino acid sequence is SEQ ID NO: 2804, while in some other embodiments, the reference sequence is SEQ ID NO: 2812, while in some other embodiments, the reference sequence is SEQ ID NO: 2956. In some embodiments, the reference amino acid sequence is SEQ ID NO: 3174, while in some other embodiments, the reference sequence is SEQ ID NO: 3222, while in some other embodiments, the reference sequence is SEQ ID NO: 3670. In some embodiments, the reference amino acid sequence is SEQ ID NO: 3674, while in some other embodiments, the reference sequence is SEQ ID NO: 3796, while in some other embodiments, the reference sequence is SEQ ID NO: 3870.

In some embodiments, the polynucleotide encodes a TdT polypeptide capable of converting one or more substrates to product with improved properties as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, wherein the polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246.

In some embodiments, the polynucleotide encoding the engineered TdT comprises a polynucleotide sequence selected from the odd-numbered sequences in the range of SEQ ID NOs: 3-1959, 2003-3919, 4047-5465, and 5475.

In some embodiments, the polynucleotides are capable of hybridizing under highly stringent conditions to a reference polynucleotide sequence selected from the odd-numbered sequences in the range of SEQ ID NOs: 3-1959, 2003-3919, 4047-5465, and 5475 or a complement thereof, and encode a TdT polypeptide with one or more of the improved properties described herein. In some embodiments, the polynucleotide capable of hybridizing under highly stringent conditions encodes a TdT comprising an amino acid sequence having at least 80%, 85%, 86%, 87%, 88%, 89%, 90%, 91%, 92%, 93%, 94%, 95%, 96%, 97%, 98%, 99% or more identity to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, that has an amino acid sequence comprising one or more residue differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246, as described above and in the Examples, below.

In some embodiments, the polynucleotide capable of hybridizing under highly stringent conditions encodes an engineered TdT polypeptide with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246. In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered TdT. In some embodiments, the reference polynucleotide sequence is selected from SEQ ID NOs: 3-1959, 2003-3919, 4047-5465, and 5475.

In some embodiments, the polynucleotide capable of hybridizing under highly stringent conditions encodes an engineered TdT polypeptide with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246. In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered TdT. In some embodiments, the reference polynucleotide sequence is selected from SEQ ID NOs: 3-1959, 2003-3919, 4047-5465, and 5475.

In some embodiments, an isolated polynucleotide encoding any of the engineered TdT polypeptides provided herein is manipulated in a variety of ways to provide for expression of the polypeptide. In some embodiments, the polynucleotides encoding the polypeptides are provided as expression vectors where one or more control sequences is present to regulate the expression of the polynucleotides and/or polypeptides. Manipulation of the isolated polynucleotide prior to its insertion into a vector may be desirable or necessary depending on the expression vector. The techniques for modifying polynucleotides and nucleic acid sequences utilizing recombinant DNA methods are well known in the art.

In some embodiments, the control sequences include among other sequences, promoters, leader sequences, polyadenylation sequences, propeptide sequences, signal peptide sequences, and transcription terminators. As known in the art, suitable promoters can be selected based on the host cells used. For bacterial host cells, suitable promoters for directing transcription of the nucleic acid constructs of the present application, include, but are not limited to the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA 75: 3727-3731 [1978]), as well as the tac promoter (See e.g., DeBoer et al., Proc. Natl Acad. Sci. USA 80: 21-25 [1983]). Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488 [1992]).

In some embodiments, the control sequence is a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice finds use in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra).

In some embodiments, the control sequence is a suitable leader sequence, a non-translated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include but are not limited to those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells include but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Bio., 15:5983-5990 [1995]).

In some embodiments, the control sequence is a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region that directs the expressed polypeptide into the secretory pathway of a host cell of choice finds use for expression of the engineered TdT polypeptides provided herein. Effective signal peptide coding regions for bacterial host cells include but are not limited to the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 57:109-137 [1993]). Effective signal peptide coding regions for filamentous fungal host cells include but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include but are not limited to those from the genes for Saccharomyces cerevisiae alpha-factor and Saccharomyces cerevisiae invertase.

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen,” in some cases). A propolypeptide can be converted to a mature active polypeptide by catalytic or autocatalytic cleavage of the propeptide from the propolypeptide. The propeptide coding region includes but is not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (See e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

The present invention also provides recombinant expression vectors comprising a polynucleotide encoding an engineered TdT polypeptide, and one or more expression regulating regions such as a promoter and a terminator, a replication origin, etc., depending on the type of hosts into which they are to be introduced. In some embodiments, the various nucleic acid and control sequences described above are combined together to produce a recombinant expression vector which includes one or more convenient restriction sites to allow for insertion or substitution of the nucleic acid sequence encoding the variant TdT polypeptide at such sites. Alternatively, the polynucleotide sequence(s) of the present invention are expressed by inserting the polynucleotide sequence or a nucleic acid construct comprising the polynucleotide sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), that can be conveniently subjected to recombinant DNA procedures and can result in the expression of the variant TdT polynucleotide sequence. The choice of the vector will typically depend on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiments, the expression vector preferably contains one or more selectable markers, which permit easy selection of transformed cells. A “selectable marker” is a gene the product of which provides for biocide or viral resistance, resistance to heavy metals, prototrophy to auxotrophy, and the like. Examples of bacterial selectable markers include but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in a filamentous fungal host cell include, but are not limited to, amdS (acetamidase), argB (ornithine carbamoyltransferases), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. In another aspect, the present invention provides a host cell comprising a polynucleotide encoding at least one engineered TdT polypeptide of the present invention, the polynucleotide being operatively linked to one or more control sequences for expression of the engineered TdT enzyme(s) in the host cell. Host cells for use in expressing the polypeptides encoded by the expression vectors of the present invention are well known in the art and include but are not limited to, bacterial cells, such as E. coli, Vibriofluvialis, Streptomyces and Salmonella typhimurium cells; fungal cells, such as yeast cells (e.g., Saccharomyces cerevisiae and Pichia pastoris [ATCC Accession No. 201178]); insect cells such as Drosophila S2 and Spodoptera Sf9 cells; animal cells such as CHO, COS, BHK, 293, and Bowes melanoma cells; and plant cells. Exemplary host cells are Escherichia coli strains (e.g., W3110 (ΔfhuA) and BL21).

In some embodiments, the host cell strain comprises a knockout of one or more genes, in particular phosphatase genes. In some embodiments, the host cell comprises a knockout or single gene deletion of E. coli genes aphA, surE, phoA, and/or cpdB, as described below in the Examples. In some embodiments, the host cell comprising a knockout of one or more phosphatase genes has increased production of the product and/or decreased de-phosphorylation of the product or substrate.

Accordingly, in another aspect, the present invention provides methods for producing the engineered TdT polypeptides, where the methods comprise culturing a host cell capable of expressing a polynucleotide encoding the engineered TdT polypeptide under conditions suitable for expression of the polypeptide. In some embodiments, the methods further comprise the steps of isolating and/or purifying the TdT polypeptides, as described herein.

Appropriate culture media and growth conditions for the above-described host cells are well known in the art. Polynucleotides for expression of the TdT polypeptides may be introduced into cells by various methods known in the art. Techniques include, among others, electroporation, biolistic particle bombardment, liposome mediated transfection, calcium chloride transfection, and protoplast fusion.

The engineered TdTs with the properties disclosed herein can be obtained by subjecting the polynucleotide encoding the naturally occurring or engineered TdT polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA 91:10747-10751 [1994]; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 16:258-261 [1998]), mutagenic PCR (See e.g., Caldwell et al., PCR Methods Appl., 3:S136-S140 [1994]), and cassette mutagenesis (See e.g., Black et al., Proc. Natl. Acad. Sci. USA 93:3525-3529 [1996]).

For example, mutagenesis and directed evolution methods can be readily applied to polynucleotides to generate variant libraries that can be expressed, screened, and assayed. Mutagenesis and directed evolution methods are well known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, and all related US, as well as PCT and non-US counterparts; Ling et al., Anal. Biochem., 254(2):157-78 [1997]; Dale et al., Meth. Mol. Biol., 57:369-74 [1996]; Smith, Ann. Rev. Genet., 19:423-462 [1985]; Botstein et al., Science, 229:1193-1201 [1985]; Carter, Biochem. J., 237:1-7 [1986]; Kramer et al., Cell, 38:879-887 [1984]; Wells et al., Gene, 34:315-323 [1985]; Minshull et al., Curr. Op. Chem. Biol., 3:284-290 [1999]; Christians et al., Nat. Biotechnol., 17:259-264 [1999]; Crameri et al., Nature, 391:288-291 [1998]; Crameri, et al., Nat. Biotechnol., 15:436-438 [1997]; Zhang et al., Proc. Nat. Acad. Sci. U.S.A., 94:4504-4509 [1997]; Crameri et al., Nat. Biotechnol., 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; and WO 2009/152336, all of which are incorporated herein by reference).

In some embodiments, the enzyme clones obtained following mutagenesis treatment are screened by subjecting the enzymes to a defined temperature (or other assay conditions, such as testing the enzyme's activity over a broad range of substrates) and measuring the amount of enzyme activity remaining after heat treatments or other assay conditions. Clones containing a polynucleotide encoding a TdT polypeptide are then sequenced to identify the nucleotide sequence changes (if any) and used to express the enzyme in a host cell. Measuring enzyme activity from the expression libraries can be performed using any suitable method known in the art (e.g., standard biochemistry techniques, such as HPLC analysis).

In some embodiments, the clones obtained following mutagenesis treatment can be screened for engineered TdTs having one or more desired improved enzyme properties (e.g., improved regioselectivity). Measuring enzyme activity from the expression libraries can be performed using the standard biochemistry techniques, such as HPLC analysis, LC-MS analysis, RapidFire-MS analysis, and/or capillary electrophoresis analysis.

When the sequence of the engineered polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence. For example, polynucleotides and oligonucleotides encoding portions of the TdT can be prepared by chemical synthesis as known in the art (e.g., the classical phosphoramidite method of Beaucage et al., Tet. Lett. 22:1859-69 [1981], or the method described by Matthes et al., EMBO J. 3:801-05 [1984]) as typically practiced in automated synthetic methods. According to the phosphoramidite method, oligonucleotides are synthesized (e.g., in an automatic DNA synthesizer), purified, annealed, ligated and cloned in appropriate vectors. In addition, essentially any nucleic acid can be obtained from any of a variety of commercial sources. In some embodiments, additional variations can be created by synthesizing oligonucleotides containing deletions, insertions, and/or substitutions, and combining the oligonucleotides in various permutations to create engineered TdTs with improved properties.

Accordingly, in some embodiments, a method for preparing the engineered TdT polypeptide comprises: (a) synthesizing a polynucleotide encoding a polypeptide comprising an amino acid sequence having at least about 80%, 85%, 86%, 87%, 88%, 89%, 90%, 91%, 92%, 93%, 94%, 95%, 96%, 97%, 98%, or 99% or more sequence identity to an amino acid sequence selected from the even-numbered sequences of SEQ ID NOs: 4-1960, 2004-3920, 4048-5466, and 5476, and having one or more residue differences as compared to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246; and (b) expressing the TdT polypeptide encoded by the polynucleotide.

In some embodiments of the method, the polynucleotide encodes an engineered TdT that has optionally one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, 1-50, 1-75, 1-100, or 1-150 amino acid residue deletions, insertions and/or substitutions. In some embodiments, the amino acid sequence has optionally around 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, 50, 55, 60, 65, 70, 80, 90, 100, 110, 120, 130, 140, or 150 amino acid residue deletions, insertions and/or substitutions. In some embodiments, the substitutions can be conservative or non-conservative substitutions.

In some embodiments, any of the engineered TdT enzymes expressed in a host cell can be recovered from the cells and/or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available (e.g., CelLytic B™, Sigma-Aldrich, St. Louis MO).

Chromatographic techniques for isolation of the TdT polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved TdT enzymes. For affinity chromatography purification, any antibody which specifically binds the TdT polypeptide may be used. For the production of antibodies, various host animals, including but not limited to rabbits, mice, rats, etc., may be immunized by injection with a TdT polypeptide, or a fragment thereof. The TdT polypeptide or fragment may be attached to a suitable carrier, such as BSA, by means of a side chain functional group or linkers attached to a side chain functional group. In some embodiments, the affinity purification can use a specific ligand bound by the TdT or dye affinity column (See e.g., EP0641862; Stellwagen, “Dye Affinity Chromatography,” In Current Protocols in Protein Science, Unit 9.2-9.2.16 [2001]).

Methods of Using the Engineered TdT Enzymes

In some embodiments, the TdT enzymes described herein find use in processes for conversion of one or more suitable substrates to a product.

In some embodiments, the engineered TdT polypeptides disclosed herein can be used in a process for the conversion of the oligo acceptor substrate and an NTP-3′-O-RBG or natural or modified NTP substrate to a product comprising an oligo acceptor substrate extended by one nucleotide.

In the embodiments provided herein and illustrated in the Examples, various ranges of suitable reaction conditions that can be used in the processes, include but are not limited to, substrate loading, co-substrate loading, pH, temperature, buffer, solvent system, cofactor, polypeptide loading, and reaction time. Further suitable reaction conditions for carrying out the process for biocatalytic conversion of substrate compounds to product compounds using an engineered TdT described herein can be readily optimized in view of the guidance provided herein by routine experimentation that includes, but is not limited to, contacting the engineered TdT polypeptide and one or more substrate compounds under experimental reaction conditions of concentration, pH, temperature, and solvent conditions, and detecting the product compound.

The oligo acceptor substrate may be any nucleotide chain or similar moiety with an exposed 3′-OH. In some embodiments, the acceptor substrate may be single stranded. In yet other embodiments, the acceptor substrate may be double stranded or partially doubled stranded. In some embodiments, the acceptor substrate may comprise a nucleotide chain consisting of 1-10 nucleotides, 5-20 nucleotides, 15-50 nucleotides, 30-100 nucleotides, or greater than 100 nucleotides. In some embodiments, the oligo acceptor substrate may comprise a chemical moiety that is not a nucleotide chain but contains a free —OH capable of being recognized as a substrate by a wild-type or engineered TdT.

In some embodiments, the oligo acceptor substrate may comprise one or more nucleotides with a 2′ modification, as described herein. In some embodiments, the oligo acceptor substrate may comprise one or more nucleotides with a 2′ modification selected from 2′-OH, 2′-H, 2′-O-methyl, 2′-fluoro, or 2′-methoxyethyl, 2′-OCH₂CH₂OCH₃, 2′—CO₂R′ (where R′ is any alkyl or aryl), or another 2′ atom or chemical group. In some embodiments, the oligo acceptor substrate may comprise one or more additional modifications, such as a phosphothiorate linkage.

In some embodiments, the sugar may have other modifications at other positions, such as locked nucleotides or constrained ethyl nucleotides, as is known in the art. In some embodiments, “locked nucleoside” or “locked nucleotide” refers to nucleoside or nucleotide, respectively, in which the ribose moiety is modified with a bridge connecting the 2′ oxygen and 4′ carbon (see, e.g., Obika et al., Tetrahedron Letters, 1997, 38(50):8735-8738; Orum et al., Current Pharmaceutical Design, 2008, 14(11):1138-1142). Typically, the bridge is a methylene bridge. In some embodiments, the 3′-phosphate group of the NQP may act as a removable blocking group or protecting group that may be selectively unblocked or removed to allow further modifications, reactions, or incorporation of the NQP into a growing oligonucleotide chain during template-dependent or template-independent oligonucleotide synthesis

In some embodiments, the oligo acceptor substrate comprises a nucleotide chain of repeating nucleotides. In other embodiments, the oligo acceptor substrate comprises a nucleotide chain of varied nucleotides that do not repeat. In some embodiments, the oligo acceptor substrate comprises a nucleotide chain with an odd number of nucleotides. In some embodiments, the oligo acceptor substrate comprises a nucleotide with an even number of nucleotides.

In some embodiments, the oligo acceptor substate is secured to solid support. Suitable solid supports are known to those in the art and described, below, in this disclosure.

In some embodiments, the oligo acceptor substrate comprises one or more nucleotide sequences selected from the following 5′-6-FAM-T17ATCmC, 5′-6-FAM-T12AT*mC, 5′-6-FAM-T17ATC(2′dF)C, 5′-6-FAM-T12ATCAC*(2′dF)A, 5′-6-FAM-T12ATCAC*mC, 5′-6-FAM-T12ATCAC*mA, 5′-6-FAM-T15AmG*mC, 5′-6-FAM-T15AmG*mC, 5′-6-FAM-T12TATCAC*mC, 5′-6-FAM-T15AmU*mG, 5′-6-FAM-T15AmU*mG, 5′-6-FAM-T14ATCmC, 5′-6-FAM-T15AT*mG, 5′-6-FAM-T17mAmUmC, 5′-6-FAM-T17mUmUmC, 5′-6-FAM-T17mCmUmG, 5′-6-FAM-T15AT*mA, 5′-6-FAM-T15AT*mC, 5′-6-FAM-T15AT*mU, T14ATCmC, 5′-6-FAM-T15mUmGmA, 5′-6-FAM-T15mAmU*mG, 5′-6-FAM-T15 mC*mG*mA, 5′-6-FAM-T17mGmUmC, 5′-6-FAM-T12mAmUmA, 5′-6-FAM-T22mAmUmU, 5′-6-FAM-T27mAmUmG, 5′-6-FAM-T57mUmUmC, 5′-6-FAM-T32mAmCmC, 5′-6-FAM-T37mAmGmC, 5′-6-FAM-T42mAmAmC, 5′-6-FAM-T47mGmUmC, 5′-6-FAM-T52mCmUmC, 5′-6-FAM-T15mAmU(2′dF)G, 5′-6-FAM-T15mAmG(MOE)C, 5′-6-FAM-T15mGmAmC, 5′-6-FAM-T22*(2′dF)A(2′dF)GmA, 5′-6-FAM-T22(2′dF)C(2′dF)G(2′dF)A, 5′-6-FAM-T27(2′dF)GmA(2′dF)U, 5′-6-FAM-T15mGmAmC, 5′-6-FAM-T 11mCmGmA, 5′-6-FAM-T11 mC*mA*mG, 5′-6-FAM-T15 mA(2′dF)UmC, 5′-6-FAM-T15mCmUmG, 5′-6-FAM-T27(2′dF)C*(2′dF)G*(2′dF)A, 5′-6-FAM-T11 mU*(2′dF)A*(2′dF)A, 5′-6-FAM-T48 mG*mA*mC, 5′-6-FAM-T15mAmCmU, 5′-6-FAM-T17*(2′dF)A*(2′dF)A(2′dF)G, 5′-6-FAM-T15mAmU(2′dF)U, 5′-6-FAM-T15mAmU(2′dF)C, 5′-BiosG-T3(iFluorT)T9mAmUmA, 5′-BiosG-T3(iFluorT)T9mAmUmAmA, 5′-BiosG-T3(iFluorT)T9mAmUmAmAmG, 5′-BiosG-T3(iFluorT)T9mAmUmAmAmGmA, 5′-BiosG-T3(iFluorT)T9mAmUmAmAmGmA(2′dF)A, 5′-BiosG-T13mAmUmA, 5′-BiosG-T13mAmUmAmA, 5′-BiosG-T13mAmUmAmAmG, 5′-BiosG-T13mAmUmAmAmGmA, 5′-BiosG-T13mAmUmAmAmGmA(2′dF)A, as further described in the Examples (Table 4.1) and the accompanying sequence listing. These embodiments are intended to be non-limiting. Any suitable oligo acceptor substrate finds use in the present invention.

In some embodiments, the NTP-3′-O-RBG substrate comprises a deoxyribonucleoside triphosphate with a 3′-O-RBG. In other embodiments, the NTP-3′-O-RBG substrate may comprise a ribonucleoside triphosphate with a 3′-O-RBG. In yet other embodiments, the NTP-3′-O-RBG substrate may comprise a synthetic nucleoside triphosphate with a 3′-O-RBG. In some embodiments, the NTP-3′-O-RBG substrate may comprise a sugar ring with a number of carbons that is not five. A non-limiting example of this is a threose nucleoside triphosphate.

A range of 3′ removable blocking groups for the NTP-3′-O-RBG substrate useful in the present disclosure are known in the art and include but are not limited to, —O—NH₂, —O—NO₂, —O—PO₃. In some embodiments, the NTP-3′-O-RBG substrate with 3′ removable blocking group can be selected from the group consisting of NTP-3′-O—NH₂, NTP-3′-O—NO₂, or NTP-3′-O—PO₃. In some embodiments, the NTP-3′-O-RBG substrate comprises another blocking group that would sterically hinder addition of a second NTP-3′-O-RBG substrate to the 3′ end of the growing oligo acceptor substrate strand prior to removal of the removable blocking from the first round of addition.

In some embodiments, the deoxyribonucleoside triphosphate with a 3′-O-RBG or ribonucleoside triphosphate with a 3′-O-RBG further comprises a natural purine or pyrimidine base, such as adenine, guanine, cytosine, thymine, or uridine. In some embodiments, deoxyribonucleoside triphosphate with a 3′-O-RBG or ribonucleoside triphosphate with a 3′-O-RBG further comprises an unnatural base analog such as inosine, xanthine, hypoxanthine or another base analog, as is known in the art. In some embodiments, the deoxyribonucleoside triphosphate with a 3′-O-RBG or ribonucleoside triphosphate with a 3′-O-RBG further comprises a base with modifications, as is known in the art. In some embodiments, the deoxyribonucleoside triphosphate with a 3′-O-RBG or ribonucleoside triphosphate with a 3′-O-RBG further comprises a 2′ modification or substitution. In some embodiments, the deoxyribonucleoside triphosphate with a 3′-O-RBG or ribonucleoside triphosphate with a 3′-O-RBG further comprises substitution of an oxygen for a sulfur atom for the creation of phosphorothioate linkages. In some embodiments, the deoxyribonucleoside triphosphate with a 3′-O-RBG or ribonucleoside triphosphate with a 3′-O-RBG further comprises substitution of two oxygens for sulfurs for the creation of phosphorodithioate linkages.

The substrate compound(s) in the reaction mixtures can be varied, taking into consideration, for example, the desired amount of product compound, the effect of each substrate concentration on enzyme activity, stability of enzyme under reaction conditions, and the percent conversion of each substrate to product. In some embodiments, the suitable reaction conditions comprise a substrate compound loading for each oligo acceptor substrate of at least about 0.1 μM to 1 μM, 1 μM to 2 μM, 2 μM to 3 μM, 3 μM to 5 μM, 5 μM to 10 μM, or 10 μM or greater. In some embodiments, the suitable reaction conditions comprise a substrate compound loading for each oligo acceptor substrate of at least about 0.5 to about 25 g/L, 1 to about 25 g/L, 5 to about 25 g/L, about 10 to about 25 g/L, or 20 to about 25 g/L. In some embodiments, the suitable reaction conditions comprise a substrate compound loading for each oligo acceptor substrate of at least about 0.5 g/L, at least about 1 g/L, at least about 5 g/L, at least about 10 g/L, at least about 15 g/L, at least about 20 g/L, or at least about 30 g/L, or even greater.

In some embodiments, the suitable reaction conditions comprise a substrate compound loading for each NTP-3′-O-RBG or natural or modified NTP substrate of at least about 1 μM to 5 μM, 5 μM to 10 μM, 10 μM to 25 μM, 25 μM to 50 μM, 50 μM to 100 μM, 100 μM to 200 μM, 200 μM to 300 μM, or 300 μM to 500 μM. In some embodiments, the suitable reaction conditions comprise a substrate compound loading for each oligo acceptor substrate of at least about 0.5 g/L, at least about 1 g/L, at least about 5 g/L, at least about 10 g/L, at least about 15 g/L, at least about 20 g/L, or at least about 30 g/L, or even greater.

In carrying out the TdT-mediated synthesis processes described herein, the engineered polypeptide may be added to the reaction mixture in the form of a purified enzyme, partially purified enzyme, whole cells transformed with gene(s) encoding the enzyme, as cell extracts and/or lysates of such cells, and/or as an enzyme immobilized on a solid support. Whole cells transformed with gene(s) encoding the engineered TdT enzyme or cell extracts, lysates thereof, and isolated enzymes may be employed in a variety of different forms, including solid (e.g., lyophilized, spray-dried, and the like) or semisolid (e.g., a crude paste). The cell extracts or cell lysates may be partially purified by precipitation (ammonium sulfate, polyethyleneimine, heat treatment or the like, followed by a desalting procedure prior to lyophilization (e.g., ultrafiltration, dialysis, etc.). Any of the enzyme preparations (including whole cell preparations) may be stabilized by crosslinking using known crosslinking agents, such as, for example, glutaraldehyde or immobilization to a solid phase (e.g., Eupergit C, and the like).

The gene(s) encoding the engineered TdT polypeptides can be transformed into host cell separately or together into the same host cell. For example, in some embodiments one set of host cells can be transformed with gene(s) encoding one engineered TdT polypeptide, and another set can be transformed with gene(s) encoding another TdT. Both sets of transformed cells can be utilized together in the reaction mixture in the form of whole cells, or in the form of lysates or extracts derived therefrom. In other embodiments, a host cell can be transformed with gene(s) encoding multiple engineered TdT polypeptides. In some embodiments the engineered polypeptides can be expressed in the form of secreted polypeptides, and the culture medium containing the secreted polypeptides can be used for the TdT reaction.

In some embodiments, the improved activity of the engineered TdT polypeptides disclosed herein provides for processes wherein higher percentage conversion can be achieved with lower concentrations of the engineered polypeptide. In some embodiments of the process, the suitable reaction conditions comprise an engineered polypeptide amount of about 1% (w/w), 2% (w/w), 5% (w/w), 10% (w/w), 20% (w/w), 30% (w/w), 40% (w/w), 50% (w/w), 75% (w/w), 100% (w/w) or more of substrate compound loading.

In some embodiments, the engineered polypeptide is present at a molar ratio of engineered polypeptide to substrate of about 50 to 1, 25 to 1, 10 to 1, 5 to 1, 1 to 1, 1 to 5, 1 to 10, 1 to 25 or 1 to 50.

In some embodiments, the engineered polypeptide is present at a molar ratio of engineered polypeptide to substrate from a range of about 50 to 1 to a range of about 1 to 50.

In some embodiments, the engineered polypeptide is present at about 0.01 g/L to about 50 g/L; about 0.01 to about 0.1 g/L; about 0.05 g/L to about 50 g/L; about 0.1 g/L to about 40 g/L; about 1 g/L to about 40 g/L; about 2 g/L to about 40 g/L; about 5 g/L to about 40 g/L; about 5 g/L to about 30 g/L; about 0.1 g/L to about 10 g/L; about 0.5 g/L to about 10 g/L; about 1 g/L to about 10 g/L; about 0.1 g/L to about 5 g/L; about 0.5 g/L to about 5 g/L; or about 0.1 g/L to about 2 g/L. In some embodiments, the TdT polypeptide is present at about 0.01 g/L, 0.05 g/L, 0.1 g/L, 0.2 g/L, 0.5 g/L, 1, 2 g/L, 5 g/L, 10 g/L, 15 g/L, 20 g/L, 25 g/L, 30 g/L, 35 g/L, 40 g/L, or 50 g/L.

In some embodiments, the suitable reaction conditions comprise a divalent metal cofactor. In some embodiments, the divalent metal cofactor is cobalt. In some embodiments, the cobalt (II) chloride is present at concentrations of about 1 to 1000 μM; about 50 to 400 μM; about 100 to 300 μM; or about 200 to 600 μM; about 500 to 1000 μM. In some embodiments, the cobalt (II) chloride is present at concentrations of about 150 μM; about 200 μM; about 250 μM, about 500 μM; or about 1000 μM.

In some embodiments of the reaction, a phosphatase is used to degrade inorganic phosphate and shift the reaction equilibrium toward the oligo acceptor extension product. In some embodiments, the phosphatase is an E. coli pyrophosphatase. In some embodiments, the phosphatase is present at a concentration of about 0.0001 to 0.01 units/uL; about 0.001 to 0.005 units/uL; or about 0.002 to 0.003 units/uL. In some embodiments, the phosphatase is present at a concentration of about 0.001 units/uL; about 0.002 units/uL; or about 0.003 units/uL. In some embodiments, the phosphatase is from Geobacillus zalihae. In some embodiments, the phosphatase is present at a concentration of about 0.01 to 10 μM; about 0.01 to 0.1 μM; or about 0.1 to 1 μM; or about 0.1 to 10 μM. In some embodiments, the phosphatase is present at a concentration of about 0.05 μM; about 0.5 μM; or about 5 μM; or about 10 μM.

During the course of the reaction, the pH of the reaction mixture may change. The pH of the reaction mixture may be maintained at a desired pH or within a desired pH range. This may be done by the addition of an acid or a base, before and/or during the course of the reaction. Alternatively, the pH may be controlled by using a buffer. Accordingly, in some embodiments, the reaction condition comprises a buffer. Suitable buffers to maintain desired pH ranges are known in the art and include, by way of example and not limitation, borate, phosphate, 2-(N-morpholino)ethanesulfonic acid (MES), 3-(N-morpholino)propanesulfonic acid (MOPS), acetate, triethanolamine (TEoA), and 2-amino-2-hydroxymethyl-propane-1,3-diol (Tris), and the like. In some embodiments, the reaction conditions comprise water as a suitable solvent with no buffer present.

In the embodiments of the process, the reaction conditions comprise a suitable pH. The desired pH or desired pH range can be maintained by use of an acid or base, an appropriate buffer, or a combination of buffering and acid or base addition. The pH of the reaction mixture can be controlled before and/or during the course of the reaction. In some embodiments, the suitable reaction conditions comprise a solution pH from about 4 to about 10, pH from about 5 to about 10, pH from about 5 to about 9, pH from about 6 to about 9, pH from about 6 to about 8. In some embodiments, the reaction conditions comprise a solution pH of about 4, 4.5, 5, 5.5, 6, 6.5, 7, 7.5, 8, 8.5, 9, 9.5, or 10.

In the embodiments of the processes herein, a suitable temperature is used for the reaction conditions, for example, taking into consideration the increase in reaction rate at higher temperatures, and the activity of the enzyme during the reaction time period. Accordingly, in some embodiments, the suitable reaction conditions comprise a temperature of about 10° C. to about 95° C., about 10° C. to about 75° C., about 15° C. to about 95° C., about 20° C. to about 95° C., about 20° C. to about 65° C., about 25° C. to about 70° C., or about 50° C. to about 70° C. In some embodiments, the suitable reaction conditions comprise a temperature of about 10° C., 15° C., 20° C., 25° C., 30° C., 35° C., 40° C., 45° C., 50° C., 55° C., 60° C., 65° C., 70° C., 75° C., 80° C., 85° C., 90° C. or 95° C. In some embodiments, the temperature during the enzymatic reaction can be maintained at a specific temperature throughout the course of the reaction. In some embodiments, the temperature during the enzymatic reaction can be adjusted over a temperature profile during the course of the reaction.

In some embodiments, the processes of the invention are carried out in a solvent. Suitable solvents include water, aqueous buffer solutions, organic solvents, polymeric solvents, and/or co-solvent systems, which generally comprise aqueous solvents, organic solvents and/or polymeric solvents. The aqueous solvent (water or aqueous co-solvent system) may be pH-buffered or unbuffered. In some embodiments, the processes using the engineered TdT polypeptides can be carried out in an aqueous co-solvent system comprising an organic solvent (e.g., ethanol, isopropanol (IPA), dimethyl sulfoxide (DMSO), dimethylformamide (DMF) ethyl acetate, butyl acetate, 1-octanol, heptane, octane, methyl t butyl ether (MTBE), toluene, and the like), ionic or polar solvents (e.g., 1-ethyl 4 methylimidazolium tetrafluoroborate, 1-butyl-3-methylimidazolium tetrafluoroborate, 1-butyl 3 methylimidazolium hexafluorophosphate, glycerol, polyethylene glycols, and the like). In some embodiments, the co-solvent can be a polar solvent, such as a polyol, dimethylsulfoxide (DMSO), or lower alcohol. The non-aqueous co-solvent component of an aqueous co-solvent system may be miscible with the aqueous component, providing a single liquid phase, or may be partly miscible or immiscible with the aqueous component, providing two liquid phases. Exemplary aqueous co-solvent systems can comprise water and one or more co-solvents selected from an organic solvent, polar solvent, and polyol solvent. In general, the co-solvent component of an aqueous co-solvent system is chosen such that it does not adversely inactivate the TdT enzyme under the reaction conditions. Appropriate co-solvent systems can be readily identified by measuring the enzymatic activity of the specified engineered TdT enzyme with a defined substrate of interest in the candidate solvent system, utilizing an enzyme activity assay, such as those described herein.

In some embodiments of the process, the suitable reaction conditions comprise an aqueous co-solvent, where the co-solvent comprises DMSO at about 1% to about 50% (v/v), about 1 to about 40% (v/v), about 2% to about 40% (v/v), about 5% to about 30% (v/v), about 10% to about 30% (v/v), or about 10% to about 20% (v/v). In some embodiments of the process, the suitable reaction conditions can comprise an aqueous co-solvent comprising ethanol at about 1% (v/v), about 5% (v/v), about 10% (v/v), about 15% (v/v), about 20% (v/v), about 25% (v/v), about 30% (v/v), about 35% (v/v), about 40% (v/v), about 45% (v/v), or about 50% (v/v).

In some embodiments, the reaction conditions comprise a surfactant for stabilizing or enhancing the reaction. Surfactants can comprise non-ionic, cationic, anionic and/or amphiphilic surfactants. Exemplary surfactants, include by way of example and not limitation, nonyl phenoxypolyethoxylethanol (NP40), TRITON™ X-100 polyethylene glycol tert-octylphenyl ether, polyoxyethylene-stearylamine, cetyltrimethylammonium bromide, sodium oleylamidosulfate, polyoxyethylene-sorbitanmonostearate, hexadecyldimethylamine, etc. Any surfactant that may stabilize or enhance the reaction may be employed. The concentration of the surfactant to be employed in the reaction may be generally from 0.1 to 50 mg/mL, particularly from 1 to 20 mg/mL.

In some embodiments, the reaction conditions include an antifoam agent, which aids in reducing or preventing formation of foam in the reaction solution, such as when the reaction solutions are mixed or sparged. Anti-foam agents include non-polar oils (e.g., minerals, silicones, etc.), polar oils (e.g., fatty acids, alkyl amines, alkyl amides, alkyl sulfates, etc.), and hydrophobic (e.g., treated silica, polypropylene, etc.), some of which also function as surfactants. Exemplary anti-foam agents include Y-30® (Dow Corning), poly-glycol copolymers, oxy/ethoxylated alcohols, and polydimethylsiloxanes. In some embodiments, the anti-foam can be present at about 0.001% (v/v) to about 5% (v/v), about 0.01% (v/v) to about 5% (v/v), about 0.1% (v/v) to about 5% (v/v), or about 0.1% (v/v) to about 2% (v/v). In some embodiments, the anti-foam agent can be present at about 0.001% (v/v), about 0.01% (v/v), about 0.1% (v/v), about 0.5% (v/v), about 1% (v/v), about 2% (v/v), about 3% (v/v), about 4% (v/v), or about 5% (v/v) or more as desirable to promote the reaction.

The quantities of reactants used in the TdT reaction will generally vary depending on the quantities of product desired, and concomitantly the amount of substrates employed. Those having ordinary skill in the art will readily understand how to vary these quantities to tailor them to the desired level of productivity and scale of production.

In some embodiments, the order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the cofactor, co-substrate and substrate may be added first to the solvent.

The solid reactants (e.g., enzyme, salts, etc.) may be provided to the reaction in a variety of different forms, including powder (e.g., lyophilized, spray dried, and the like), solution, emulsion, suspension, and the like. The reactants can be readily lyophilized or spray dried using methods and equipment that are known to those having ordinary skill in the art. For example, the protein solution can be frozen at −80° C. in small aliquots, then added to a pre-chilled lyophilization chamber, followed by the application of a vacuum.

For improved mixing efficiency when an aqueous co-solvent system is used, the TdT, and co-substrate may be added and mixed into the aqueous phase first. The substrate may be added and mixed in, followed by the organic phase or the substrate may be dissolved in the organic phase and mixed in. Alternatively, the substrate may be premixed in the organic phase, prior to addition to the aqueous phase.

The processes of the present invention are generally allowed to proceed until further conversion of substrate to product does not change significantly with reaction time (e.g., less than 10% of substrate being converted, or less than 5% of substrate being converted). In some embodiments, the reaction is allowed to proceed until there is complete or near complete conversion of substrate to product. Transformation of substrate to product can be monitored using known methods by detecting substrate and/or product, with or without derivatization. Suitable analytical methods include gas chromatography, HPLC, MS, and the like. In some embodiments, after suitable conversion to product, the reactants are separated from the oligo acceptor substrate extension product and additional reactants are added to the oligo acceptor substrate extension product to further extend the growing polynucleotide chain. The processes of the present invention may be used to iteratively extend the oligo acceptor extension product until a polynucleotide of a defined sequence and length is synthesized.

Any of the processes disclosed herein using the engineered polypeptides for the preparation of products can be carried out under a range of suitable reaction conditions, including but not limited to ranges of substrates, temperature, pH, solvent system, substrate loading, polypeptide loading, cofactor loading, and reaction time. In one example, the suitable reaction conditions comprise: (a) oligo acceptor substrate loading of about 0.1-5000 μM of substrate compound; (b) NTP-3′-O-RBG substrate or NTP loading of about 1-10000 μM of substrate compound; (c) of about 0.01 g/L to 5 g/L engineered polypeptide; (d) 100 to 5000 μM cobalt (II) chloride; (e) 5 to 100 mM triethanolamine buffer; (f) 0.05 to 10 μM pyrophosphatase; (g) pH at 5-9; and (h) temperature of about 15° C. to 70° C. In some embodiments, the suitable reaction conditions comprise: (a) oligo acceptor substrate loading of about 400 μM of substrate compound; (b) NTP-3′-O-RBG or NTP substrate loading of about 800 μM of substrate compound; (c) of about 0.06 g/L engineered polypeptide; (d) 600 μM cobalt (II) chloride; (e) 100 mM triethanolamine buffer; (f) 5 μM pyrophosphatase; (g) pH at 7.8; and (h) temperature of about 50° C. In some embodiments, the enzyme loading is between 1-30% w/w. In some embodiments, additional reaction components or additional techniques carried out to supplement the reaction conditions. These can include taking measures to stabilize or prevent inactivation of the enzyme, reduce product inhibition, shift reaction equilibrium to formation of the desired product.

In some embodiments, the present disclosure provides an engineered TdT, wherein said engineered TdT has improved activity on NTP-3′-RBGs or modified NTPs, such that NTP-3′-RBGs are incorporated with equivalent efficiency to native NTPs, as compared to another wild-type or engineered TdT. In some embodiments, the engineered TdT with improved activity on dNTP-3′-O—PO₃, such that dNTP-3′-O—PO₃ is incorporated with equivalent efficiency to native dNTPs, is an engineered TdT polypeptide comprising an amino acid sequence having at least 80%, 85%, 86%, 87%, 88%, 89%, 90%, 91%, 92%, 93%, 94%, 95%, 96%, 97%, 98%, 99% or more identity to SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246.

Methods of Using Engineered TdTs for Template-Independent Oligonucleotide Synthesis

As described in the above embodiments, modifications of an engineered TdT allow for improved oligonucleotide (with either a phosphodiester or phosphorothioate linkage) and 3′O-blocked NTP (either with a triphosphate or an alpha-thio triphosphate) acceptance, which enable template-independent oligonucleotide synthesis. While these embodiments have several advantages over phosphoramidite chemistry, a preferred embodiment, described herein, dramatically reduces the amount of solid support and organic solvent used in the method, furthering enabling the production of high volumes of single stranded oligonucleotides necessary for siRNA therapeutics applications.

Traditionally, methods of oligonucleotide synthesis, including both phosphoramidite chemistry and newer methods of template-independent enzymatic synthesis, have relied upon immobilization of the growing oligonucleotide chain on a support, such as a solid support or a solution-based support. While this method allows addition and purification steps to proceed, a large volume of solid support is required and requires concomitant high volumes of NTPs and other reagents to drive the synthesis reaction. These methods generate substantial waste and are not feasible for the industrial scale production of kilograms of oligonucleotide necessary for siRNA therapeutics.

In some embodiments, the present disclosure provides a novel method of oligonucleotide synthesis wherein the engineered TdT or a template-independent polymerase is immobilized. Although enzyme immobilization is known in the art, template-independent oligonucleotide synthesis using an immobilized TdT or template-independent polymerase for iterative rounds of nucleotide addition required for oligonucleotide synthesis has not been reported. The described novel method includes various embodiments that overcome process challenges inherent in such a method.

In some embodiments, the immobilized TdT or template-independent polymerase is an engineered TdT, described above, comprising greater than 60% sequence identity to the even-numbered sequences of SEQ ID NOs: 4-1960, 2004-3920, 4048-5466, and 5476 and one or more substitutions or substitution sets in the amino acid sequence of the engineered TdT, as compared to a reference sequence. In some embodiments, the immobilized TdT or template-independent polymerase is another wild-type or engineered polymerase. Any suitable enzyme having template-independent polymerase activity may be used in these methods.

In one embodiment, the present disclosure provides a method for template-independent synthesis of an oligonucleotide, the method comprising: (a) providing at least one TdT or template-independent polymerase; (b) providing at least one oligo acceptor substrate, wherein the oligo acceptor substrate comprises a 3′-OH or equivalent; and (c) contacting the oligo acceptor substrate, the TdT or template-independent polymerase, and a nucleotide triphosphate, a modified nucleotide triphosphate, or a NTP-3′-O-RBG under conditions sufficient for the addition of the nucleotide, modified nucleotide, or nucleotide-3′-O-RBG to the 3′ end. In some embodiments, step (c) optionally includes contacting the oligo acceptor substrate, the TdT or template-independent polymerase, and a nucleotide triphosphate, a modified nucleotide triphosphate, or NTP-3′-O-RBG with a phosphatase, such as an inorganic pyrophosphatase to convert pyrophosphate to inorganic phosphate. In another embodiment, the method further comprises (d) deblocking the oligonucleotide formed in step (c) at the protected 3-O-position of the oligonucleotide product. In some embodiments, the method comprises (e) deactivating unreacted nucleotide triphosphates, modified nucleotide triphosphates, or NTP-3′-O-RBGs. In some embodiments, step (d) deblocking the oligonucleotide formed in step (c) at the protected 3-O-position of the nucleotide-3′-O-RBG and step (e) deactivating unreacted nucleotide triphosphates, modified nucleotide triphosphates, or NTP-3′-O-RBGs occur simultaneously. In some embodiments, step (d) deblocking the oligonucleotide formed in step (c) at the protected 3-O-position of the nucleotide-3′-O-RBG and step (e) deactivating unreacted nucleotide triphosphates, modified nucleotide triphosphates, or NTP-3′-O-RBGs occur simultaneously, wherein the NTP-3′-O-RBG comprises a 3′ phosphate and a phosphatase is used to deblock the nucelotide-3′-O-RBG while simultaneously deactivating unreacted NTP-3′-O-RBGs by removing the 5′ phosphates to leave nucleosides. In some embodiments, the method comprises an optional step (f) of removing excess nucleoside and/or excess inorganic phosphate and/or pyrophosphate from the reaction. In some embodiments, steps (a)-(c) or (a)-(d) or (a)-(e) or (a)-(f) are repeated until a desired oligonucleotide sequence is obtained. In another embodiment, the method further comprises (g) cleaving or releasing the growing or completed oligonucleotide chain from the oligo acceptor substrate.

In some embodiments of the described method for template-independent synthesis of an oligonucleotide, the oligo acceptor substrate and growing oligonucleotide chain are immobilized on a solid support. In some embodiments of the described method for template-independent synthesis of an oligonucleotide, the TdT or template-independent polymerase, oligo acceptor substrate and growing oligonucleotide chain are all in solution phase. The oligo substrate and growing oligo chain can be optionally substituted with a soluble tag that aids extended oligo product isolation and purification. In some embodiments of the described method for template-independent synthesis of an oligonucleotide, the TdT or template-independent polymerase is immobilized. In some embodiments of the described method for template-independent synthesis of an oligonucleotide, the TdT or template-independent polymerase is simultaneously purifed and immobilized on a solid support. In some embodiments, the immobilized TdT or template-independent polymerase is an engineered TdT with greater than 60% sequence identity to SEQ ID NOs: 4-1960, 2004-3920, 4048-5466, and 5476 and one or more substitutions or substitution sets in the amino acid sequence of the engineered TdT. In some embodiments, the immobilized TdT or template-independent polymerase is immobilized on a solid support.

In some embodiments, the engineered TdT polypeptides can be provided on a solid support, such as a membrane, resin, solid carrier, or other solid phase material. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location.

In some embodiments, the engineered TdT polypeptides of the present invention can be immobilized on a solid support such that they retain their improved activity, and/or other improved properties relative to the reference polypeptide of SEQ ID NOs: 2, 8, 16, 24, 36, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192 and/or 5246. In such embodiments, the immobilized polypeptides can facilitate the biocatalytic conversion of the substrate compounds or other suitable substrates to the product and after the reaction is complete are easily retained (e.g., by retaining beads on which polypeptide is immobilized) and then reused or recycled in subsequent reactions. Such immobilized enzyme processes allow for further efficiency and cost reduction. Accordingly, it is further contemplated that any of the methods of using the TdT polypeptides of the present invention can be carried out using the TdT polypeptides bound or immobilized on a solid support.

Methods of enzyme immobilization are well-known in the art. The engineered polypeptides can be bound non-covalently or covalently. Various methods for conjugation and immobilization of enzymes to solid supports (e.g., resins, membranes, beads, glass, etc.) are well known in the art (See e.g., Yi et al., Proc. Biochem., 42(5): 895-898 [2007]; Martin et al., Appl. Microbiol. Biotechnol., 76(4): 843-851 [2007]; Koszelewski et al., J. Mol. Cat. B: Enzymatic, 63: 39-44 [2010]; Truppo et al., Org. Proc. Res. Dev., published online: dx.doi.org/10.1021/op200157c; Hermanson, Bioconjugate Techniques, 2^{nd} ed., Academic Press, Cambridge, MA [2008]; Mateo et al., Biotechnol. Prog., 18(3):629-34 [2002]; and “Bioconjugation Protocols: Strategies and Methods,” In Methods in Molecular Biology, Niemeyer (ed.), Humana Press, New York, NY [2004]; the disclosures of each which are incorporated by reference herein). Solid supports useful for immobilizing the engineered TdT of the present invention include but are not limited to beads or resins comprising polymethacrylate with epoxide functional groups, polymethacrylate with amino epoxide functional groups, styrene/DVB copolymer or polymethacrylate with octadecyl functional groups. Exemplary solid supports useful for immobilizing the engineered TdT polypeptides of the present invention include, but are not limited to, EnginZyme (including, EziG-1, EziG-1, and EziG-3), chitosan beads, Eupergit C, and SEPABEADs (Mitsubishi) (including EC-EP, EC-HFA/S, EXA252, EXE119 and EXE120).

In some embodiments of the described method for template-independent synthesis of an oligonucleotide, the TdT or template-independent polymerase is immobilized, and the method comprises an aqueous liquid phase. In some embodiments, the method for template-independent synthesis of an oligonucleotide comprising an aqueous phase and an immobilized TdT or template-independent polymerase further comprises a column solid support. In some embodiments, the method for template-independent synthesis of an oligonucleotide comprising an aqueous phase and an immobilized TdT or template-independent polymerase further comprises a batch method with a solid support. In some embodiments, the oligo acceptor substrate and/or growing oligonucleotide chain are provided in an aqueous phase. In some embodiments, the nucleotide triphosphate, the modified nucleotide triphosphate, or the NTP-3′-O-RBG are provided in an aqueous phase. In some embodiments, the method further comprises removing unreacted nucleotide triphosphates, modified nucleotide triphosphates, or NTP-3′-O-RBGs from the oligo acceptor substrate and/or growing oligonucleotide chain. In some embodiments of the described method for template-independent synthesis of an oligonucleotide, the oligo acceptor substrate and oligonucleotide are immobilized. In some embodiments, neither the oligo acceptor substrate nor the TdT of template-independent polymerase are immobilized.

In some further embodiments, the method for template-independent synthesis of an oligonucleotide comprising an immobilized TdT or template-independent polymerase and an aqueous liquid phase further comprises the steps of (a) providing at least one TdT or template-independent polymerase on a solid support; (b) providing at least one oligo acceptor substrate in an aqueous phase, wherein the oligo acceptor substrate comprises a 3′-OH or equivalent; and (c) contacting the oligo acceptor substrate, the TdT or template-independent polymerase, and a nucleotide triphosphate, a modified nucleotide triphosphate, or NTP-3′-O-RBG under aqueous conditions sufficient for the addition of the nucleotide, modified nucleotide, or nucleotide-3′-O-RBG to the 3′ end. In some embodiments, step (c) optionally includes contacting the oligo acceptor substrate, the TdT or template-independent polymerase, and a nucleotide triphosphate, a modified nucleotide triphosphate, or NTP-3′-O-RBG with a phosphatase, such as an inorganic pyrophosphatase to convert pyrophosphate to inorganic phosphate. In another embodiment, the method further comprises (d) deblocking the oligonucleotide formed in step (c) at the protected 3-O-position of the oligonucleotide product. In another embodiment, the method further comprises (e) deactivating the unreacted nucleotide triphosphates, modified nucleotide triphosphates, or NTP-3′-O-RBGs. In some embodiments, step (d) deblocking the oligonucleotide formed in step (c) at the protected 3-O-position of the nucleotide-3′-O-RBG and step (e) deactivating unreacted nucleotide triphosphates, modified nucleotide triphosphates, or NTP-3′-O-RBGs occur simultaneously. In some embodiments, step (d) deblocking the oligonucleotide formed in step (c) at the protected 3-O-position of the nucleotide-3′-O-RBG and step (e) deactivating unreacted nucleotide triphosphates, modified nucleotide triphosphates, or NTP-3′-O-RBGs occur simultaneously, wherein the NTP-3′-O-RBG comprises a 3′ phosphate and a phosphatase is used to deblock the nucleotide-3′-O-RBG while simultaneously deactivating unreacted NTP-3′-O-RBGs by removing the 5′ phosphates to leave nucleosides. In some embodiments, the method comprises an optional step (f) of removing excess nucleoside and/or excess inorganic phosphate and/or pyrophosphate from the reaction. In some embodiments, steps (a)-(c) or (a)-(d) or (a)-(e) or (a)-(f) are repeated until a desired oligonucleotide sequence is obtained. In another embodiment, the method further comprises (g) cleaving or releasing the growing or completed oligonucleotide chain from the oligo acceptor substrate once a desired oligonucleotide sequence is obtained. In some embodiments, any of steps (a)-(g) are completed on a solid support. In some embodiments the solid support is a column. In some embodiments, any of steps (a)-(g) are completed in an aqueous phase passing over one or a series of in line columns. In some embodiments, the solid support is used in a batch method. In some embodiments, any of the above-described methods comprise synthesis of an RNA oligonucleotide or a modified RNA oligonucleotide. In some embodiments, any of the above-described methods comprise synthesis of a DNA oligonucleotide. In any of the embodiments described herein, the aqueous phase may comprise an aqueous co-solvent or aqueous co-solvent system, as further described herein.

In some further embodiments, the method for template-independent synthesis of an oligonucleotide comprising an immobilized TdT or template-independent polymerase and an aqueous liquid phase further comprises the steps of (a) providing at least one TdT on a solid support, wherein said TdT comprises a polypeptide sequence comprising at least 60% identity to any of the even-numbered sequences of SEQ ID NOs: 4-1960, 2004-3920, 4048-5466, and 5476 and at least one substitution or substitution set in said polypeptide sequence as compared to a reference sequence of any of the even-numbered sequences of SEQ ID NOs: 4-1960, 2004-3920, 4048-5466, and 5476; (b) providing at least one oligo acceptor substrate in an aqueous phase, wherein the oligo acceptor substrate comprises a 3′-OH or equivalent; and (c) contacting the oligo acceptor substrate, the TdT or template-independent polymerase, and a nucleotide triphosphate, a modified nucleotide triphosphate, or NTP-3′-O-RBG under aqueous conditions sufficient for the addition of the nucleotide, modified nucleotide, or nucleotide-3′-O-RBG to the 3′ end. In some embodiments, step (c) optionally includes contacting the oligo acceptor substrate, the TdT or template-independent polymerase, and a nucleotide triphosphate, a modified nucleotide triphosphate, or NTP-3′-O-RBG with a phosphatase, such as a pyrophosphatase to convert pyrophosphate to inorganic phosphate. In another embodiment, the method further comprises (d) deblocking the oligonucleotide formed in step (c) at the protected 3-O-position of the oligonucleotide product. In another embodiment, the method further comprises (e) deactivating the unreacted nucleotide triphosphates, modified nucleotide triphosphates, or NTP-3′-O-RBGs. In some embodiments, step (d) deblocking the oligonucleotide formed in step (c) at the protected 3-O-position of the oligonucleotide product and step (e) deactivating unreacted nucleotide triphosphates, modified nucleotide triphosphates, or NTP-3′-O-RBGs occur simultaneously. In some embodiments, step (d) deblocking the oligonucleotide formed in step (c) at the protected 3-O-position of the oligonucleotide product and step (e) deactivating unreacted nucleotide triphosphates, modified nucleotide triphosphates, or NTP-3′-O-RBGs occur simultaneously, wherein the NTP-3′-O-RBG comprises a 3′ phosphate and a phosphatase is used to deblock the nucleotide-3′-O-RBG while simultaneously deactivating unreacted NTP-3′-O-RBGs by removing the 5′ phosphates to leave nucleosides. In some embodiments, the method comprises an optional step (f) of removing excess nucleoside and/or excess inorganic phosphate and/or pyrophosphate from the reaction. In some embodiments, steps (a)-(c) or (a)-(d) or (a)-(e) or (a)-(f) are repeated until a desired oligonucleotide sequence is obtained. In another embodiment, the method further comprises (g) cleaving or releasing the growing or completed oligonucleotide chain from the oligo acceptor substrate once a desired nucleotide sequence is obtained. In some embodiments, any of steps (a)-(g) are completed on a solid support. In some embodiments the solid support is a column. In some embodiments, any of steps (a)-(g) are completed in an aqueous phase passing over one or a series of in line columns. In some embodiments, the solid support is used in a batch method. In some embodiments, any of the above-described methods comprise synthesis of an RNA oligonucleotide or modified RNA oligonucleotide. In some embodiments, any of the above-described methods comprise synthesis of a DNA oligonucleotide.

In some further embodiments, the method for template-independent synthesis of an oligonucleotide comprises a nucleotide triphosphate, a modified nucleotide triphosphate, or an NTP-3′-O-RBG. The nucleotide triphosphate may comprise a deoxyribonucleotide triphosphate, a dideoxy ribonucleotide triphosphate, a ribonucleotide triphosphate, or any other modified nucleotide triphosphate, as is known in the art. Modifications may be at the 3′ position, as is in the case of NTP-3′-O-RBG, or at the 2′ position. Modifications may be at other positions of the sugar or to the base. Modifications may also be present as substitutions of one or more of the phosphate groups of the nucleotide triphosphate and may be incorporated into the phospho backbone of the growing oligonucleotide change. Although specific examples of suitable modifications are provided herein, any modification to the nucleotide triphosphate may be used in the described methods. Various modifications may confer various desired properties to the oligonucleotide chain. For example, the use of phosphorothiate linkages and 2′ modifications in RNA synthesis for RNA therapeutics protects the RNA strand from degradation in the body and extends the half-life of the therapeutic. Various photolabile or cleavable tags may also be present as modifications and may aid in visualization or purification of the oligonucleotide during the synthesis method.

In some embodiments, the method for template-independent synthesis of an oligonucleotide comprises a nucleotide triphosphate, a modified nucleotide triphosphate, or an NTP-3′-O-RBG comprising a 3′ modification. In some embodiments, the 3′ modification comprises —NH₂, —NO₂, —(CH₂)₂-CN, or —PO₃. In some embodiments, the 3′ modification comprises carbonitriles, phosphates, carbonates, carbamates, esters, ethers, borates, nitrates, sugars, phosphoramidates, phenylsulfenates, and sulfates,

In some embodiments, the method for template-independent synthesis of an oligonucleotide comprises a nucleotide triphosphate, a modified nucleotide triphosphate, or an NTP-3′-O-RBG comprising a 2′ modification. In some embodiments, the 2′ modification comprises a 2′-F or 2′-O-alkyl. In some further embodiments, the 2′-F modified nucleotide comprises 2′-fluoro-2′-deoxyadenosine-5′-triphosphate, 2′-fluoro-2′-deoxycytidine-5′-triphosphate, 2′-fluoro-2′-deoxyguanosine-5′-triphosphate, and 2′-fluoro-2′-deoxyuridine-5′-triphosphate. In some further embodiments, the 2′-O-alkyl modified nucleotide comprises 2′-O-methyladenosine-5′-triphosphate, 2′-O-methylcytidine-5′-triphosphate, 2′-O-methylguanosine-5′-triphosphate, 2′-O-methyluridine-5′-triphosphate, and 2′-O-methylinosine-5′-triphosphate. In yet some further embodiments, any of the 2′-F modified nucleotides or 2′-O-alkyl modified nucleotides further comprise a 3′-O-removable blocking group. In yet some further embodiments, any of the 2′-F modified nucleotide triphosphates or 2′-O-alkyl modified nucleotide triphosphates further comprise a 3′-O-phosphate removable blocking group. In some embodiments, the modified nucleotide triphosphate comprises or further comprises a phosphorothioate group at the 5′ alpha position.

In some embodiments, the method for template-independent synthesis of an oligonucleotide comprises optionally contacting the oligo acceptor substrate, the TdT or template-independent polymerase, and a nucleotide triphosphate, a modified nucleotide triphosphate, or NTP-3′-O-RBG with a phosphatase to convert pyrophosphate to inorganic phosphate. In some embodiments, the production of inorganic phosphate from pyrophosphate drives the extension reaction toward the N+1 product. In some embodiments, the phosphatase is an inorganic pyrophosphatase. In some embodiments, the inorganic pyrophosphatase is derived from Thermocrinis ruber, Aquifex pyrophilus, Thermus oshimai, Sulfolobus sp. A20, Geobacillus zalihae, Bacillus thermozeamaize, or Bacillus smithii. In some embodiments, the inorganic pyrophosphatase comprises a sequence selected from SEQ ID NOs: 3936, 3938, 3940, 3942, 3944, 3946, or 3948. In some embodiments, the inorganic pyrophosphatase may be immobilized on a solid support.

In some embodiments, the method for template-independent synthesis of an oligonucleotide comprises a of step deblocking the oligonucleotide at the protected 3′-O-position of the oligonucleotide product and/or a step of deactivating unreacted nucleotide triphosphates, modified nucleotide triphosphates, or NTP-3′-O-RBGs to nucleosides using a phosphatase. In some embodiments, the phosphatase is an alkaline phosphatase. In some embodiments, the alkaline phosphatase is derived from Pyrococcus furiosus, Thermotoga maritima, Thermotoga sp. 50_64, Pseudothermotoga lettingae, Thermotoga neapolitana, Thermoflexibacter ruber, or Bacillus licheniformis. In some embodiments, the alkaline phosphatase comprises a sequence selected from SEQ ID NOs: 3922, 3924, 3926, 3928, 3930, 3932, or 3934. In some embodiments, the alkaline phosphatase may be immobilized on a solid support.

In some embodiments, the method for template-independent synthesis of an oligonucleotide comprises an optional step of removing excess inorganic phosphate or nucleoside from the reaction.

In some embodiments, the method for template-independent synthesis of an oligonucleotide comprises an optional step of cleaving or releasing the growing or completed oligonucleotide chain from the oligo acceptor substrate. In some embodiments, an exonuclease is used to cleave or release the growing or completed oligonucleotide chain from the oligo acceptor substrate.

In further embodiments, any of the above-described processes for the conversion of one or more substrate compounds to product compound can further comprise one or more steps selected from: extraction; isolation; purification; and crystallization of product compound. As is known to those skilled in the art, acidic compounds such as oligonucleotides, NTPs, modified NTPs, and NTP-3′-O-RBGs may exist in various salt forms that can be used interchangeably in the methods described herein. All such forms are specifically envisaged for use in the methods described herein. Methods, techniques, and protocols for extracting, isolating, purifying, and/or crystallizing the product from biocatalytic reaction mixtures produced by the above disclosed processes are known to the ordinary artisan and/or accessed through routine experimentation. Additionally, illustrative methods are provided in the Examples below.

Various features and embodiments of the invention are illustrated in the following representative examples, which are intended to be illustrative, and not limiting.

### EXAMPLES

The following Examples, including experiments and results achieved, are provided for illustrative purposes only and are not to be construed as limiting the present invention. Indeed, there are various suitable sources for many of the reagents and equipment described below. It is not intended that the present invention be limited to any particular source for any reagent or equipment item.

In the experimental disclosure below, the following abbreviations apply: M (molar); mM (millimolar), μM and uM (micromolar); nM (nanomolar); mol (moles); gm and g (gram); mg (milligrams); ug and μg (micrograms); L and 1 (liter); ml and mL (milliliter); cm (centimeters); mm (millimeters); μM and μη(micrometers); sec. (seconds); min(s) (minute(s)); h(s) and hr(s) (hour(s)); U (units); MW (molecular weight); rpm (rotations per minute); psi and PSI (pounds per square inch); ° C. (degrees Celsius); RT and rt (room temperature); CV (coefficient of variability); CAM and cam (chloramphenicol); PMBS (polymyxin B sulfate); IPTG (isopropyl β-D-1-thiogalactopyranoside); LB (lysogeny broth); TB (terrific broth); SFP (shake flask powder); CDS (coding sequence); DNA (deoxyribonucleic acid); RNA (ribonucleic acid); nt (nucleotide; polynucleotide); aa (amino acid; polypeptide); E. coli W3110 (commonly used laboratory E. coli strain, available from the Coli Genetic Stock Center [CGSC], New Haven, CT); HTP (high throughput); HPLC (high pressure liquid chromatography); HPLC-UV (HPLC-Ultraviolet Visible Detector); 1H NMR (proton nuclear magnetic resonance spectroscopy); FIOPC (fold improvements over positive control); Sigma and Sigma-Aldrich (Sigma-Aldrich, St. Louis, MO; Difco (Difco Laboratories, BD Diagnostic Systems, Detroit, MI); Microfluidics (Microfluidics, Westwood, MA); Life Technologies (Life Technologies, a part of Fisher Scientific, Waltham, MA); Amresco (Amresco, LLC, Solon, OH); Carbosynth (Carbosynth, Ltd., Berkshire, UK); Varian (Varian Medical Systems, Palo Alto, CA); Agilent (Agilent Technologies, Inc., Santa Clara, CA); Infors (Infors USA Inc., Annapolis Junction, MD); and Thermotron (Thermotron, Inc., Holland, MI).

Abbreviations for Modified Nucleotides

“*”: “There is a phosphorothioate bond between these base pairs.”,
‘5′-6-FAM-T’: “n is a 5′-6-carboxyfluorescein-thymidine”,
‘ddG’: “n is a 2′,3′-dideoxyguanosine”,
“(2′dF)A”: “n is a 2′-deoxyfluoroadenosine”,
“(2′dF)C”: “n is a 2′-deoxyfluorocytidine”,
“(2′dF)G”: “n is a 2′-deoxyfluoroguanosine”,
“(2′dF)U”: “n is a 2′-deoxyfluorouridine”,
“(2′dF)A-3′P”: “n is a 2′-deoxyfluoroadenosine with a 3′-PO₄ blocking
group”,
“(2′dF)C-3′P”: “n is a 2′-deoxyfluorocytidine with a 3′-PO₄ blocking
groupp”,
“(2′dF)G-3′P”: “n is a 2′-deoxyfluoroguanosine with a 3′-PO₄ blocking
group”,
“(2′dF)U-3′P”: “n is a 2′-O-methyluridine with a 3′-PO₄ blocking group”,
‘mA’: “n is a 2′-O-methyladenosine”,
‘mC’: “n is a 2′-O-methylcytidine”,
‘mG’: “n is a 2′-O-methylguanosine”,
‘mU’: “n is a 2′-O-methyluridine”,
“mA-3′P”: “n is a 2′-O-methyladenosine with a 3′-PO₄ blocking group”,
“mC-3′P”: “n is a 2′-O-methylcytidine with a 3′-PO₄ blocking group”,
“mG-3′P”: “n is a 2′-O-methylguanosine with a 3′-PO₄ blocking group”,
“mU-3′P”: “n is a 2′-O-methyluridine with a 3′-PO₄ blocking group”,
‘rA’: “n is an adenosine ribonucleotide”,
‘rG’: “n is a guanosine ribonucleotide”,
‘rG’: “n is a guanosine ribonucleotide”,
‘rU’: “n is a uridine ribonucleotide”
‘(MOE)A’: “n is a 2′-O-(2-methoxyethyl)adenosine”,
‘(MOE)C’: “n is a 2′-O-(2-methoxyethyl)cytidine”,
‘(MOE)G’: “n is a 2′-O-(2-methoxyethyl)guanosine”,
‘(MOE)U’: “n is a 2′-O-(2-methoxyethyl)uridine”,
‘5′-BiosG-T’: “n is a thymidine with a 5′-biotin modifition”,
‘(iFluorT)’: “n is a thymidine with a 5′-fluroscein modification”,

### Example 1

### Terminal Deoxynucleotidyl Transferase (TdT) Gene Acquisition and Construction of Expression Vectors

The wild-type (WT) terminal deoxynucleotidyl transferase (TdT) enzyme (SEQ ID NO:2) is a predicted splice variant encoded by the genome of species Monodelphis domestica. A synthetic gene (SEQ ID NO:1 encoding an N-terminal 6-histidine tagged version of the WT TdT was designed with codon optimization for E. coli expression, synthesized, and subcloned into the E. coli expression vector pCK100900i (See e.g., U.S. Pat. No. 7,629,157 and US Pat. Appln. Publn. 2016/0244787, both of which are hereby incorporated by reference). This plasmid construct was transformed into an E. coli strain derived from W31 10. Directed evolution techniques generally known by those skilled in the art were used to generate libraries of gene variants from these plasmids (See e.g., U.S. Pat. No. 8,383,346 and WO 2010/144103, both of which are hereby incorporated by reference). The substitutions in the enzyme variants described herein are indicated with reference to the N-terminal 6-histidine tagged version of the WT TdT enzyme (i.e., SEQ ID NO: 2) or variants thereof, as indicated.

### Example 2

### TdT Expression and Lysate Processing for High Throughput (HTP) Screening High Throughput (HTP) Growth of TdT Enzyme and Variants

Transformed E. coli cells were selected by plating onto LB agar plates containing 1% glucose and 30 μg/mL chloramphenicol. After overnight incubation at 37° C., colonies were placed into the wells of 96-well shallow flat bottom NUNC™ (Thermo-Scientific) plates filled with 180 μl/well LB medium supplemented with 1% glucose and 30 μg/mL chloramphenicol. The cultures were allowed to grow overnight for 18-20 hours in a shaker (200 rpm, 30° C., and 85% relative humidity; Kuhner). Overnight growth samples (20 μL) were transferred into Costar 96-well deep plates filled with 380 μL of Terrific Broth supplemented with 30 μg/mL chloramphenicol. The plates were incubated for 120 minutes in a shaker (250 rpm, 30 C, and 85% relative humidity; Kuhner) until the ODoo reached between 0.4-0.8. The cells were then induced with 40 μL of 10 mM IPTG in sterile water and incubated overnight for 18-20 hours in a shaker (250 rpm, 30 C, and 85% relative humidity; Kuhner). The cells were pelleted (4,000 rpm for 20 min), the supernatants were discarded, and the cells were frozen at −80° C. prior to analysis.

Method 1: Lysis of HTP Cell Pellets with Lysozyme (Examples 7-12)

For lysis, 400 μL lysis buffer containing 50 mM MOPS buffer, pH 7.4, and 0.2 g/L lysozyme were added to the cell pellet in each well. The cells were lysed at room temperature for 2 hours with shaking on a bench top shaker. The plate was then centrifuged for 15 min at 4,000 rpm and 4° C. The clear supernatants were then used in biocatalytic reactions to determine their activity levels.

Method 2: Thermal lysis of HTP Cell Pellets with Lysozyme (Examples 13-25)

For lysis, 400 μL lysis buffer containing 50 mM triethanolamine buffer, pH 7.5, and 0.1 g/L lysozyme were added to the cell pellet in each well. The cells were shaken vigorously at room temperature for 5 minutes on a bench top shaker. A 100-uL aliquot of the re-suspended cells was transferred to a 96-well format 200 μL BioRad PCR plate, then briefly spun-down prior to 1 h heat treatment at the temperature indicated, typically 48-60° C. Following heat-treatment, the cell debris was pelleted by centrifugation (4,000 rpm at 4° C. for 10 min), and clear supernatants were then used in biocatalytic reactions to determine their activity levels.

### Example 3

### Shake Flask Expression and Purification of TdT Shake Flask Expression

Selected HTP cultures grown as described above were plated onto LB agar plates with 1% glucose and 30 μg/mL chloramphenicol and grown overnight at 37° C. A single colony from each culture was transferred to 5 mL of LB broth with 1% glucose and 30 μg/mL chloramphenicol. The cultures were grown for 20 h at 30° C., 250 rpm, and subcultured at a dilution of approximately 1:50 into 250 mL of Terrific Broth with 30 μg/mL of chloramphenicol, to a final OD₆₀₀ of about 0.05. The cultures were incubated for approximately 195 min at 30° C., 250 rpm, to an OD₆₀₀ of about 0.6, and then induced with the addition of IPTG at a final concentration of 1 mM. The induced cultures were incubated for 20 h at 30° C., 250 rpm. Following this incubation period, the cultures were centrifuged at 4,000 rpm for 10 min. The culture supernatant was discarded, and the pellets were resuspended in 35 mL of 20 mM triethanolamine, pH 7.5. This cell suspension was chilled in an ice bath and lysed using a Microfluidizer cell disruptor (Microfluidics M-110L). The crude lysate was pelleted by centrifugation (11,000 rpm for 60 min at 4° C.), and the supernatant was then filtered through a 0.2 pm PES membrane to further clarify the lysate.

Purification of TdT from Shake Flask Lysates

TdT lysates were supplemented with 1/10^{th} volume of SF elution buffer (50 mM Tris-HCl, 500 mM NaCl, 250 mM imidazole, 0.02% v/v Triton X-100 reagent) per well. Lysates were then purified using an AKTA Start purification system and a 5 mL HisTrap FF column (GE Healthcare) using the AC Step HiF setting (the run parameters are provided below). The SF wash buffer comprised 50 mM Tris-HCl, 300 mM NaCl, 20 mM imidazole, 0.02% v/v Triton X-100 reagent.

TABLE 3.1
Purification Parameters

| Parameter | Volume | |
|---|---|---|
| Column volume | 5 | mL |
| Flow rate | 8 | mL/min |
| Pressure limit | 0.3 | MPa |
| Sample volume | 35 | mL |
| Equilibration volume | 5 column volume (CV) = 25 mL | |
| Wash Unbound volume | 20 CV = 100 mLs | |
| Elution | Isocratic (step) | |
| Elution volume | 5 CV = 25 mLs | |
| Fraction volume | 1.5 | mLs |

| | |
|---|---|
| RE-equilibration volume | 5 CV = 25 mLs |

Elution fractions containing protein were identified by UV absorption (A280) and pooled, then dialyzed overnight in dialysis buffer (20 mM Tris-HCl, pH 7.4, 100 mM KCl, 0.1 mM EDTA, and 50% glycerol) in a 3.5K Slide-A-Lyzer™ dialysis cassette (Thermo Fisher) for buffer exchange. TdT concentrations in the preparations were measured by absorption at 280 nm.

### Example 4

### Capillary Electrophoresis (CE) Analysis of Oligonucleotides

### Sample Preparation for Reaction Analysis Using CE:

For analysis of the reaction samples, capillary electrophoresis was performed using an ABI 3500 xl Genetic Analyzer (ThermoFisher). Reactions (20 μL) were quenched by the addition of 60 μL of 35 mM aqueous EDTA. Reactions (1 μL) were quenched by the addition of 99 μL of 1 mM aqueous EDTA. Quenched reactions were diluted in water to 1.25 nM oligonuclelotide, and a 2-μL aliquot of this solution was transferred to a new 96-well MicroAmp Optical PCR plate or 384-well MicroAmp Optical PCR plate containing 18 μL Hi-Di™ Formamide (ThermoFisher) containing an appropriate size standard (LIZ or Alexa633). The ABI3500 xl was configured with POP6 polymer, 50 cm capillaries, and a 55° C. oven temperature. Pre-run settings were 18 KV for 50 sec. Injection was 10 KV for 2 sec, and the run settings were 19 KV for 620 sec. FAM-labeled oligo substrates and products were identified by their sizes relative to the sizing ladder.

### List of Oligonucleotide Substrates and Products

Oligonucleotide used as substrates and detected as products are listed in Table 4.1 below.

TABLE 4.1
List of substrate and product oligonucleotides

| Structural Description | SEQ ID NO: |
|---|---|
| 5′-6-FAM-T17ATCmC | 1961 |
| 5′-6-FAM-T17AT*mC | 1962 |
| 5′-6-FAM-T17ATC(2′dF)C | 1963 |
| 5′-6-FAM-T12ATCAC*(2′dF)A | 1964 |
| 5′-6-FAM-T12ATCAC*mC | 1965 |
| 5′-6-FAM-T12ATCAC*mA | 1966 |
| 5′-6-FAM-T15AmG*mC | 1967 |
| 5′-6-FAM-T12TATCAC*mC | 1968 |
| 5′-6-FAM-T15AmU*mG | 1969 |
| 5′-6-FAM-T15AmU*mG | 1970 |
| 5′-6-FAM-T14ATCmC | 1971 |
| 5′-6-FAM-T15AT*mG | 1972 |
| 5′-6-FAM-T17mAmUmC | 1973 |
| 5′-6-FAM-T17mUmUmC | 1974 |
| 5′-6-FAM-T17mCmUmG | 1975 |
| 5′-6-FAM-T15AT*mA | 1976 |
| 5′-6-FAM-T15AT*mC | 1977 |
| 5′-6-FAM-T15AT*mU | 1978 |
| T14ATCmC | 1979 |
| 5′-6-FAM-T17ATCmCddG | 1980 |
| 5′-6-FAM-T17AT*mCddG | 1981 |
| 5′-6-FAM-T17ATC(2′dF)CmA | 1982 |
| 5′-6-FAM-T17ATC(2′dF)CddG | 1983 |
| 5′-6-FAM-T12ATCAC*(2′dF)A*rA | 1984 |
| 5′-6-FAM-T12ATCAC*mC*rG | 1985 |
| 5′-6-FAM-T12ATCAC*mA*rG | 1986 |
| 5′-6-FAM-T12ATCAC*mC*rU | 1987 |
| 5′-6-FAM-T15AmG*mC *rG | 1988 |
| 5′-6-FAM-T15AmG*mCmG | 1989 |
| 5′-6-FAM-T12TATCAC*mCmG | 1990 |
| 5′-6-FAM-T15AmU*mGmA | 1991 |
| 5′-6-FAM-T14ATCmC*ddG | 1992 |
| 5′-6-FAM-T15AT*mGmA-3′P | 1993 |
| 5′-6-FAM-T17mAmUmCmA | 1994 |
| 5′-6-FAM-T17mUmUmCmA | 1995 |
| 5′-6-FAM-T17mCmUmGmA | 1996 |
| 5′-6-FAM-T15AT*mAmA-3′P | 1997 |
| 5′-6-FAM-T15AT*mGmC-3′P | 1998 |
| 5′-6-FAM-T15AT*mGmU-3′P | 1999 |
| 5′-6-FAM-T14ATCmCddG | 2000 |
| T14ATCmCddG | 2001 |
| 5′-6-FAM-T15mUmGmA | 2002 |
| 5′-6-FAM-T15mAmU*mG | 3949 |
| 5′-6-FAM-T15mC*mG*mA | 3950 |
| 5′-6-FAM-T17mGmUmC | 3951 |
| 5′-6-FAM-T12mAmUmA | 3952 |
| 5′-6-FAM-T22mAmUmU | 3953 |
| 5′-6-FAM-T27mAmUmG | 3954 |
| 5′-6-FAM-T57mUmUmC | 3955 |
| 5′-6-FAM-T32mAmCmC | 3956 |
| 5′-6-FAM-T37mAmGmC | 3957 |
| 5′-6-FAM-T42mAmAmC | 3958 |
| 5′-6-FAM-T47mGmUmC | 3959 |
| 5′-6-FAM-T52mCmUmC | 3960 |
| 5′-6-FAM-T15mAmU(2′dF)G | 3961 |
| 5′-6-FAM-T15mAmG(MOE)C | 3962 |
| 5′-6-FAM-T15mGmAmC | 3963 |
| 5′-6-FAM-T22*(2′dF)A(2′dF)GmA | 3964 |
| 5′-6-FAM-T22(2′dF)C(2′dF)G(2′dF)A | 3965 |
| 5′-6-FAM-T27(2′dF)GmA(2′dF)U | 3966 |
| 5′-6-FAM-T15mGmAmC | 3967 |
| 5′-6-FAM-T11mCmGmA | 3968 |
| 5′-6-FAM-T11mC*mA*mG | 3969 |
| 5′-6-FAM-T15mA(2′dF)UmC | 3970 |
| 5′-6-FAM-T15mCmUmG | 3971 |
| 5′-6-FAM-T27(2′dF)C*(2′dF)G*(2′dF)A | 3972 |
| 5′-6-FAM-T11mU*(2′dF)A*(2′dF)A | 3973 |
| 5′-6-FAM-T48mG*mA*mC | 3974 |
| 5′-6-FAM-T15mAmCmU | 3975 |
| 5′-6-FAM-T17*(2′dF)A*(2′dF)A(2′dF)G | 3976 |
| 5′-6-FAM-T15mAmU(2′dF)U | 3977 |
| 5′-6-FAM-T15mAmU(2′dF)C | 3978 |
| 5′-BiosG-T3(iFluorT)T9mAmUmA | 3979 |
| 5′-BiosG-T3(iFluorT)T9mAmUmAmA | 3980 |
| 5′-BiosG-T3(iFluorT)T9mAmUmAmAmG | 3981 |
| 5′-BiosG-T3(iFluorT)T9mAmUmAmAmGmA | 3982 |
| 5′-BiosG-T3(iFluorT)T9mAmUmAmAmGmA(2′dF)A | 3983 |
| 5′-BiosG-T13mAmUmA | 3984 |
| 5′-BiosG-T13mAmUmAmA | 3985 |
| 5′-BiosG-T13mAmUmAmAmG | 3986 |
| 5′-BiosG-T13mAmUmAmAmGmA | 3987 |
| 5′-BiosG-T13mAmUmAmAmGmA(2′dF)A | 3988 |
| 5′-6-FAM-T15AT*mUmA-3′P | 3989 |
| 5′-6-FAM-T15AmU*mGmA-3′P | 3990 |
| 5′-6-FAM-T15mUmGmAmA | 3991 |
| 5′-6-FAM-T15AmG*mCmA-3′P | 3992 |
| 5′-6-FAM-T15mAmU*mGmA-3′P | 3993 |
| 5′-6-FAM-T15mC*mG*mAmA-3′P | 3994 |
| 5′-6-FAM-T17mGmUmCmA-3′P | 3995 |
| 5′-6-FAM-T12mAmUmAmA-3′P | 3996 |
| 5′-6-FAM-T17mAmUmCmA-3′P | 3997 |
| 5′-6-FAM-T22mAmUmUmA-3′P | 3998 |
| 5′-6-FAM-T27mAmUmGmA-3′P | 3999 |
| 5′-6-FAM-T57mUmUmCmA-3′P | 4000 |
| 5′-6-FAM-T32mAmCmCmA-3′P | 4001 |
| 5′-6-FAM-T37mAmGmCmA-3′P | 4002 |
| 5′-6-FAM-T42mAmAmCmA-3′P | 4003 |
| 5′-6-FAM-T47mGmUmCmA-3′P | 4004 |
| 5′-6-FAM-T52mCmUmCmA-3′P | 4005 |
| 5′-6-FAM-T15mAmU(2′dF)GmA-3′P | 4006 |
| 5′-6-FAM-T15mAmG(MOE)CmA-3′P | 4007 |
| 5′-6-FAM-T15mGmAmC-3′P | 4008 |
| 5′-6-FAM-T22*(2′dF)A(2′dF)GmA(2′dF)A-3′P | 4009 |
| 5′-6-FAM-T22(2′dF)C(2′dF)G(2′dF)A(2′dF)A-3′P | 4010 |
| 5′-6-FAM-T27(2′dF)GmA(2′dF)U(2′dF)A-3′P | 4011 |
| 5′-6-FAM-T15mGmAmC(2′dF)A-3′P | 4012 |
| 5′-6-FAM-T11mCmGmA(2′dF)A-3′P | 4013 |
| 5′-6-FAM-T15mUmGmAmA-3′P | 4014 |
| 5′-6-FAM-T11mC*mA*mG(2′dF)A-3′P | 4015 |
| 5′-6-FAM-T11mC*mA*mGmA-3′P | 4016 |
| 5′-6-FAM-T15mA(2′dF)UmCmA-3′P | 4017 |
| 5′-6-FAM-T15mCmUmGmA-3′P | 4018 |
| 5′-6-FAM-T27(2′dF)C*(2′dF)G*(2′dF)A(2′dF)A-3′P | 4019 |
| 5′-6-FAM-T11mU*(2′dF)A*(2′dF)A(2′dF)A-3′P | 4020 |
| 5′-6-FAM-T15mGmAmC(2′dF)U-3′P | 4021 |
| 5′-6-FAM-T48mG*mA*mCmA-3′P | 4022 |
| 5′-6-FAM-T15mAmCmU(2′dF)A-3′P | 4023 |
| 5′-6-FAM-T15mGmAmCmA-3′P | 4024 |
| 5′-6-FAM-T15mAmCmU(2′dF)U-3′P | 4025 |
| 5′-6-FAM-T15mAmCmUmC-3′P | 4026 |
| 5′-6-FAM-T15mAmCmUmG-3′P | 4027 |
| 5′-6-FAM-T15mAmCmUmA-3′P | 4028 |
| 5′-6-FAM-T17*(2′dF)A*(2′dF)A(2′dF)G(2′dF)A-3′P | 4029 |
| 5′-6-FAM-T15mGmAmCmC-3′P | 4030 |
| 5′-6-FAM-T15mAmU(2′dF)UmA-3′P | 4031 |
| 5′-6-FAM-T15mGmAmCmG-3′P | 4032 |
| 5′-6-FAM-T15mAmCmU*mU-3′P | 4033 |
| 5′-6-FAM-T15mGmAmC*mU-3′P | 4034 |
| 5′-6-FAM-T15mGmAmC*mG-3′P | 4035 |
| 5′-6-FAM-T15mAmCmU*mG-3′P | 4036 |
| 5′-6-FAM-T15mAmU(2′dF)CmA-3′P | 4037 |
| 5′-BiosG-T3(iFluorT)T9mAmUmAmA-3′P | 4038 |
| 5′-BiosG-T3(iFluorT)T9mAmUmAmAmG-3′P | 4039 |
| 5′-BiosG-T3(iFluorT)T9mAmUmAmAmGmA-3′P | 4040 |
| 5′-BiosG-T3(iFluorT)T9mAmUmAmAmGmA(2′dF)A-3′P | 4041 |
| 5′-BiosG-T13mAmUmAmA-3′P | 4042 |
| 5′-BiosG-T13mAmUmAmAmG-3′P | 4043 |
| 5′-BiosG-T13mAmUmAmAmGmA-3′P | 4044 |
| 5′-BiosG-T13mAmUmAmAmGmA(2′dF)A-3′P | 4045 |
| 5′-6-FAM-T15mAmU*mG | 4046 |
| S08437572 | 5475 |
| 5′-6-FAM-T15mAmUmCmU | 5477 |
| T15mAmUmCmU | 5478 |
| 5′-6-FAM-T11mAmAmA(2′dF)U | 5479 |
| 5′-6-FAM-T18mA(2′dF)GmA(2′dF)G | 5480 |
| 5′-6-FAM-T11mCmCmU(2′dF)A | 5481 |
| 5′-6-FAM-T16*(2′dF)C*mA(2′dF)A(2′dF)A | 5482 |
| 5′-6-FAM-T32mAmAmA(2′dF)G | 5483 |
| 5′-6-FAM-T17*mA*mGmA | 5484 |
| 5′-6-FAM-T26mU(2′dF)GmU(2′dF)C | 5485 |
| 5′-6-FAM-T31mU(2′dF)CmA(2′dF)U | 5486 |
| 5′-6-FAM-T21mU(2′dF)CmU(2′dF)C | 5487 |
| 5′-6-FAM-T47*mA(2′dF)A(2′dF)A | 5488 |
| 5′-6-FAM-T37mG*mA*mC | 5489 |
| 5′-6-FAM-T46mA(2′dF)GmU(2′dF)G | 5490 |
| 5′-6-FAM-T41mA(2′dF)U(2′dF)C(2′dF)C | 5491 |
| 5′-6-FAM-T31mAmAmA(2′dF)G | 5492 |
| 5′-6-FAM-T57mUmU(2′dF)C | 5493 |
| 5′-6-FAM-T42*mA*mCmU | 5494 |
| 5′-6-FAM-T51mU*(2′dF)A*(2′dF)A(2′dF)G | 5495 |
| 5′-6-FAM-T12mC*mA*mG | 5496 |
| 5′-6-FAM-T22*mGmAmA | 5497 |
| 5′-6-FAM-T27mAmAmA | 5498 |
| 5′-6-FAM-T47*mCmUmU | 5499 |
| 5′-6-FAM-T52mUmUmU | 5500 |
| 5′-6-FAM-T16mC*mA*mGmA | 5501 |
| 5′-6-FAM-T11AmC*mA*mG | 5502 |
| 5′-6-FAM-T21*mA*mGmAmA | 5503 |
| 5′-6-FAM-T26*mGmAmAmA | 5504 |
| 5′-6-FAM-T36mAmA(2′dF)GmA | 5505 |
| 5′-6-FAM-T41(2′dF)GmA(2′dF)GmU | 5506 |
| 5′-6-FAM-T51(2′dF)GmU(2′dF)GmU | 5507 |
| 5′-6-FAM-T56(2′dF)GmU(2′dF)CmU | 5508 |
| 5′-6-FAM-T10(2′dF)CmU(2′dF)CmA | n/a |
| 5′-6-FAM-T16mA(2′dF)GmA(2′dF)G | 5509 |
| 5′-6-FAM-T31mU(2′dF)CmAmU | 5510 |
| 5′-6-FAM-T36(2′dF)CmAmUmC | 5511 |
| 5′-6-FAM-T41mAmUmCmU | 5512 |
| 5′-6-FAM-T46mUmCmUmU | 5513 |
| 5′-6-FAM-T51mCmUmUmA | 5514 |
| 5′-6-FAM-T10mAmAmA(MOE)A | n/a |
| 5′-6-FAM-T12mAmG(MOE)C | 5515 |
| 5′-6-FAM-T26mCmCmU*mU | 5516 |
| 5′-6-FAM-T21*(2′dF)A*(2′dF)A(2′dF)GmA | 5517 |
| 5′-6-FAM-T15mAmU*mG(2′dF)A-3′P | 5518 |
| 5′-6-FAM-T15mAmUmCmU(2′dF)A-3′P | 5519 |
| T15mAmUmCmU(2′dF)A-3′P | 5520 |
| 5′-6-FAM-T17*(2′dF)A*(2′dF)A(2′dF)GmA-3′P | 5521 |
| 5′-6-FAM-T11mC*mA*mG*mU-3′P | 5522 |
| 5′-6-FAM-T11mU*(2′dF)A*(2′dF)AmA-3′P | 5523 |
| 5′-6-FAM-T11mAmAmA(2′dF)U(2′dF)U-3′P | 5524 |
| 5′-6-FAM-T18mA(2′dF)GmA(2′dF)GmA-3′P | 5525 |
| 5′-6-FAM-T11mCmCmU(2′dF)A(2′dF)U-3′P | 5526 |
| 5′-6-FAM-T16*(2′dF)C*mA(2′dF)A(2′dF)AmU-3′P | 5527 |
| 5′-6-FAM-T32mAmAmA(2′dF)GmA-3′P | 5528 |
| 5′-6-FAM-T17*mA*mGmA*mU-3′P | 5529 |
| 5′-6-FAM-T18mA(2′dF)GmA(2′dF)G(2′dF)U-3′P | 5530 |
| 5′-6-FAM-T26mU(2′dF)GmU(2′dF)CmA-3′P | 5531 |
| 5′-6-FAM-T31mU(2′dF)CmA(2′dF)U(2′dF)U-3′P | 5532 |
| 5′-6-FAM-T21mU(2′dF)CmU(2′dF)CmU-3′P | 5533 |
| 5′-6-FAM-T47*mA(2′dF)A(2′dF)AmA-3′P | 5534 |
| 5′-6-FAM-T37mG*mA*mC*mU-3′P | 5535 |
| 5′-6-FAM-T27(2′dF)GmA(2′dF)UmA-3′P | 5536 |
| 5′-6-FAM-T26mU(2′dF)GmU(2′dF)C(2′dF)U-3′P | 5537 |
| 5′-6-FAM-T46mA(2′dF)GmU(2′dF)GmA-3′P | 5538 |
| 5′-6-FAM-T41mA(2′dF)U(2′dF)C(2′dF)C(2′dF)U-3′P | 5539 |
| 5′-6-FAM-T31mAmAmA(2′dF)GmU-3′P | 5540 |
| 5′-6-FAM-T57mUmU(2′dF)CmA-3′P | 5541 |
| 5′-6-FAM-T42*mA*mCmU*mU-3′P | 5542 |
| 5′-6-FAM-T47*mA(2′dF)A(2′dF)A(2′dF)U-3′P | 5543 |
| 5′-6-FAM-T51mU*(2′dF)A*(2′dF)A(2′dF)GmA-3′P | 5544 |
| 5′-6-FAM-T51mU*(2′dF)A*(2′dF)A(2′dF)G(2′dF)U-3′P | 5545 |
| 5′-6-FAM-T46mA(2′dF)GmU(2′dF)GmU-3′P | 5546 |
| 5′-6-FAM-T11mC*mA*mGmC-3′P | 5547 |
| 5′-6-FAM-T11mC*mA*mG*mG-3′P | 5548 |
| 5′-6-FAM-T12mC*mA*mG*mG-3′P | 5549 |
| 5′-6-FAM-T57mUmU(2′dF)CmC-3′P | 5550 |
| 5′-6-FAM-T57mUmU(2′dF)C*mG-3′P | 5551 |
| 5′-6-FAM-T17*mA*mGmAmC-3′P | 5552 |
| 5′-6-FAM-T17*mA*mGmA*mG-3′P | 5553 |
| 5′-6-FAM-T22*mGmAmAmC-3′P | 5554 |
| 5′-6-FAM-T22*mGmAmA*mG-3′P | 5555 |
| 5′-6-FAM-T27mAmAmAmC-3′P | 5556 |
| 5′-6-FAM-T27mAmAmA*mG-3′P | 5557 |
| 5′-6-FAM-T32mAmAmA(2′dF)GmC-3′P | 5558 |
| 5′-6-FAM-T32mAmAmA(2′dF)G*mG-3′P | 5559 |
| 5′-6-FAM-T37mG*mA*mCmC-3′P | 5560 |
| 5′-6-FAM-T37mG*mA*mC*mG-3′P | 5561 |
| 5′-6-FAM-T42*mA*mCmUmC-3′P | 5562 |
| 5′-6-FAM-T42*mA*mCmU*mG-3′P | 5563 |
| 5′-6-FAM-T47*mCmUmUmC-3′P | 5564 |
| 5′-6-FAM-T47*mCmUmU*mG-3′P | 5565 |
| 5′-6-FAM-T52mUmUmUmC-3′P | 5566 |
| 5′-6-FAM-T52mUmUmU*mG-3′P | 5567 |
| 5′-6-FAM-T31mAmAmA(2′dF)GmA-3′P | 5568 |
| 5′-6-FAM-T16mC*mA*mGmAmA-3′P | 5569 |
| 5′-6-FAM-T11AmC*mA*mGmA-3′P | 5570 |
| 5′-6-FAM-T21*mA*mGmAmAmA-3′P | 5571 |
| 5′-6-FAM-T26*mGmAmAmAmA-3′P | 5572 |
| 5′-6-FAM-T36mAmA(2′dF)GmAmA-3′P | 5573 |
| 5′-6-FAM-T41(2′dF)GmA(2′dF)GmUmA-3′P | 5574 |
| 5′-6-FAM-T51(2′dF)GmU(2′dF)GmUmU-3′P | 5575 |
| 5′-6-FAM-T56(2′dF)GmU(2′dF)CmUmU-3′P | 5576 |
| 5′-6-FAM-T10(2′dF)CmU(2′dF)CmAmU-3′P | n/a |
| 5′-6-FAM-T16mA(2′dF)GmA(2′dF)GmU-3′P | 5577 |
| 5′-6-FAM-T26mU(2′dF)GmU(2′dF)CmU-3′P | 5578 |
| 5′-6-FAM-T31mU(2′dF)CmAmUmU-3′P | 5579 |
| 5′-6-FAM-T36(2′dF)CmAmUmCmU-3′P | 5580 |
| 5′-6-FAM-T41mAmUmCmU(2′dF)A-3′P | 5581 |
| 5′-6-FAM-T46mUmCmUmU(2′dF)A-3′P | 5582 |
| 5′-6-FAM-T51mCmUmUmA(2′dF)A-3′P | 5583 |
| 5′-6-FAM-T10mAmAmA(MOE)A(2′dF)A-3′P | n/a |
| 5′-6-FAM-T12mAmG(MOE)C(2′dF)A-3′P | 5584 |
| 5′-6-FAM-T26mCmCmU*mUmC-3′P | 5585 |
| 5′-6-FAM-T26mCmCmU*mU*mG-3′P | 5586 |
| 5′-6-FAM-T26mCmCmU*mU*mU-3′P | 5587 |
| 5′-6-FAM-T26mCmCmU*mU(2′dF)G-3′P | 5588 |
| 5′-6-FAM-T26mCmCmU*mU(2′dF)U-3′P | 5589 |
| 5′-6-FAM-T21*(2′dF)A*(2′dF)A(2′dF)GmAmC-3′P | 5590 |
| 5′-6-FAM-T21*(2′dF)A*(2′dF)A(2′dF)GmA*mG-3′P | 5591 |
| 5′-6-FAM-T21*(2′dF)A*(2′dF)A(2′dF)GmA*mU-3′P | 5592 |
| 5′-6-FAM-T21*(2′dF)A*(2′dF)A(2′dF)GmA(2′dF)G-3′P | 5593 |
| 5′-6-FAM-T21*(2′dF)A*(2′dF)A(2′dF)GmA(2′dF)U-3′P | 5594 |
| 5′-6-FAM-T10(2′dF)CmU(2′dF)CmAmA-3′P | n/a |
| 5′-6-FAM-T10(2′dF)CmU(2′dF)CmAmA | n/a |
| 5′-6-FAM-T10(2′dF)CmU(2′dF)CmAmA(2′dF)G-3′P | n/a |
| 5′-6-FAM-T10(2′dF)CmU(2′dF)CmAmA(2′dF)G | n/a |
| 5′-6-FAM-T10(2′dF)CmU(2′dF)CmAmA(2′dF)GmU-3′P | n/a |
| 5′-6-FAM-T10(2′dF)CmU(2′dF)CmAmA(2′dF)GmU | n/a |
| 5′-6-FAM-T10(2′dF)CmU(2′dF)CmAmA(2′dF)GmU(2′dF)G-3′P | n/a |
| 5′-6-FAM-T10(2′dF)CmU(2′dF)CmAmA(2′dF)GmU(2′dF)G | n/a |
| 5′-6-FAM-T10(2′dF)CmU(2′dF)CmAmA(2′dF)GmU(2′dF)GmU-3′P | n/a |
| 5′-6-FAM-T10(2′dF)CmU(2′dF)CmAmA(2′dF)GmU(2′dF)GmU | n/a |
| 5′-6-FAM-T10(2′dF)CmU(2′dF)CmAmA(2′dF)GmU(2′dF)GmU(2′dF)C-3′P | n/a |
| 5′-6-FAM-T10(2′dF)CmU(2′dF)CmAmA(2′dF)GmU(2′dF)GmU(2′dF)C | n/a |
| 5′-6-FAM-T10(2′dF)CmU(2′dF)CmAmA(2′dF)GmU(2′dF)GmU(2′dF)CmU-3′P | n/a |
| 5′-6-FAM-T10(2′dF)CmU(2′dF)CmAmA(2′dF)GmU(2′dF)GmU(2′dF)CmU | n/a |
| 5′-6-FAM-T10(2′dF)CmU(2′dF)CmAmA(2′dF)GmU(2′dF)GmU(2′dF)CmU(2′dF)C-3′P | n/a |
| 5′-6-FAM-T10(2′dF)CmU(2′dF)CmAmA(2′dF)GmU(2′dF)GmU(2′dF)CmU(2′dF)C | n/a |
| 5′-6-FAM-T10(2′dF)CmU(2′dF)CmAmA(2′dF)GmU(2′dF)GmU*mG-3′P | n/a |
| 5′-6-FAM-T10(2′dF)CmU(2′dF)CmAmA(2′dF)GmU(2′dF)GmU*mG | n/a |
| 5′-6-FAM-T10(2′dF)CmU(2′dF)CmAmA(2′dF)GmU(2′dF)GmU(2′dF)C*mG-3′P | n/a |
| 5′-6-FAM-T10(2′dF)CmU(2′dF)CmAmA(2′dF)GmU(2′dF)GmU(2′dF)C*mG | n/a |
| 5′-6-FAM-T15mGmAmC(2′dF)C-3′P | 5595 |
| 5′-6-FAM-T15mGmAmC(2′dF)C | 5596 |
| 5′-6-FAM-T15mGmAmC(2′dF)CmU-3′P | 5597 |
| 5′-6-FAM-T15mGmAmC(2′dF)CmU | 5598 |
| 5′-6-FAM-T15mGmAmC(2′dF)CmU(2′dF)C-3′P | 5599 |
| 5′-6-FAM-T15mGmAmC(2′dF)CmU(2′dF)C | 5600 |
| 5′-6-FAM-T15mGmAmC(2′dF)CmU(2′dF)CmA-3′P | 5601 |
| 5′-6-FAM-T15mGmAmC(2′dF)CmU(2′dF)CmA | 5602 |
| 5′-6-FAM-T15mGmAmC(2′dF)CmU(2′dF)CmAmU-3′P | 5603 |
| 5′-6-FAM-T15mGmAmC(2′dF)CmU(2′dF)CmAmU | 5604 |
| 5′-6-FAM-T15mGmAmCmG | 5605 |
| 5′-6-FAM-T15mAmU(2′dF)GmA | 5606 |
| 5′-6-FAM-T21mU(2′dF)CmU(2′dF)CmA-3′P | 5607 |
| 5′-6-FAM-T21mU(2′dF)CmU(2′dF)CmA | 5608 |
| A4mC*mA*mG | n/a |
| A4mC*mA*mGmA-3′P | n/a |
| A4mC*mA*mGmA | n/a |
| 5′-6-FAM-T15mAmG*mGmA-3′P | 5609 |
| 5′-6-FAM-T15mAmG*mGmA | 5610 |
| T15mAmG*mGmA-3′P | 5611 |
| T15mAmG*mGmA | 5612 |
| 5′-6-FAM-T17mAmCmCmA-3′P | 5613 |
| 5′-6-FAM-T17mAmCmCmA | 5614 |
| T15mAmU(2′dF)G | 5615 |

### Example 5

### Isolated Protein Yield Improvements Over SEQ ID NO: 2

### Relative Isolated Protein Yield of Shake-Flask Purified TdT Variants

TdT variants of SEQ ID NO: 2-38 were produced in shake flask and purified as described in Example 3. TdT concentrations were measured by absorption at 280 nm.

Protein recovery relative to SEQ ID NO: 2 was calculated as the ratio of mg/mL protein recovered after purification of the variant compared with SEQ ID NO: 2. The results are shown in Table 5.1.

TABLE 5.1

| | | Protein |
|---|---|---|
| | | recovery after |
| SEQ ID | | purification |
| NO: | Amino Acid Differences | Relative to |
| (nt/aa) | (Relative to SEQ ID NO: 2) | SEQ ID NO: 2 |
| 3/4 | N80D/C121S/I174L/F185L/K190E/D244V/F284L/N288E/K289D/I290V/ | +++ |
| | D293S/I313A/C315G/S317T/L336D/T342E/F359L/C391G/I394R/E428V/ | |
| | R431S/Y462F/S470T/Q474M/K499L/I522L/Q523E | |
| 5/6 | N80D/C121S/I174L/F185L/K190E/D244V/L273V/F284L/N288E/K289D/ | +++ |
| | I290V/D293S/I313A/C315G/S317T/L336D/T342E/F359L/C391G/I394R/ | |
| | E428V/R431S/Y462F/S470T/Q474M/K499L/I522L/Q523E | |
| 7/8 | N80D/C121S/F185L/K190E/D244V/K289D/I290V/D293S/I313A/C315G/ | +++ |
| | S317T/L336D/T342E/F359L/C391G/S470T/Q474M/K499L/I522L/Q523E | |
| 9/10 | N80D/C121S/I174L/F185L/K190E/E193G/E196Y/D244V/L273V/F284L/ | +++ |
| | N288E/K289D/I290V/D293S/K297R/I313A/C315G/S317T/V324I/L336D/ | |
| | T342E/E352P/F359L/Q376H/N380D/C391G/I394R/L401G/Q415S/L419A/ | |
| | E428V/R431S/M435T/E441M/Y462F/S470T/Q474M/K499L/I522L/Q523E | |
| 11/12 | N80D/C121S/F185L/K190E/K289D/I290V/D293S/I313A/C315G/L336D/ | +++ |
| | T342E/F359L/C391G/F414H/S470T/Q474M/K499L/I522L/Q523E | |
| 13/14 | N80D/C121S/I174L/F185L/K190E/D244V/F284L/N288E/K289D/I290V/ | +++ |
| | D293S/I313A/C315G/S317T/L336D/T342E/E352P/F359L/C391G/I394R/ | |
| | L419A/E428V/R431S/Y462F/S470T/Q474M/K499L/I522L/Q523E | |
| 15/16 | N80D/C121S/I174L/F185L/K190E/D244V/L273V/F284L/N288E/K289D/ | +++ |
| | I290V/D293S/I313A/C315G/S317T/L336D/T342E/E352P/F359L/C391G/ | |
| | I394R/L419A/E428V/R431S/Y462F/S470T/Q474M/K499L/I522L/Q523E | |
| 17/18 | N80D/C121S/I174L/F185L/K190E/E193G/D244V/L273V/F284L/N288E/ | +++ |
| | K289D/I290V/D293S/K297R/I313A/C315G/S317T/L336D/T342E/E352P/ | |
| | F359L/C391G/I394R/Q415S/L419A/E428V/R431S/Y462F/S470T/Q474M/ | |
| | K499L/I522L/Q523E | |
| 19/20 | N80D/C121S/I174L/Q179K/F185L/K190E/G236P/D244V/N288E/K289D/ | ++ |
| | I290V/D293S/I313A/C315G/S317T/L336D/T342E/F359L/S363I/C391G/ | |
| | I394R/I408P/H426P/Y462F/S470T/Q474M/K499L/I522L/Q523E | |
| 21/22 | N80D/C121S/I174L/F185L/K190E/D244V/F284L/N288E/K289D/I290V/ | ++ |
| | D293S/I313A/C315G/S317T/L336D/T342E/E352P/F359L/C391G/I394R/ | |
| | E428V/R431S/Y462F/S470T/Q474M/K499L/I522L/Q523E | |
| 23/24 | N80D/C121S/I174L/F185L/K190E/E196R/D244V/T266K/L273V/F284L/ | ++ |
| | N288E/K289D/I290V/D293S/I313A/C315G/S317T/V324I/L336D/T342E/ | |
| | E352P/F359L/C391G/I394R/T397R/L401G/L419A/E428V/R431S/ | |
| | Y462F/S470T/Q474M/K499L/I522L/Q523E | |
| 25/26 | N80D/C121S/F185L/K190E/D244V/K289D/I290V/D293S/K300R/I313A/ | ++ |
| | C315G/S317T/L336D/T342E/F359L/C391G/S470T/Q474M/K499L/I522L/ | |
| | Q523E | |
| 27/28 | N80D/C121S/F185L/K190E/D244V/L273V/K289D/I290V/D293S/I313A/ | ++ |
| | C315G/S317T/L336D/T342E/E352P/F359L/C391G/L419A/M435T/S470T/ | |
| | Q474M/K499L/I522L/Q523E | |
| 29/30 | N80D/C121S/F185L/K190E/D244V/K289D/I290V/D293S/I313A/C315G/ | ++ |
| | S317T/L336D/T342E/F359L/N380D/C391G/L401G/L419A/S470T/Q474M/ | |
| | K499L/I522L/Q523E | |
| 31/32 | N80D/C121S/I174L/F185L/K190E/G236P/D244V/L273V/M282R/F284L/ | ++ |
| | N288E/K289D/I290V/D293S/I313A/C315G/S317T/L336D/T342E/E352P/ | |
| | F359L/C391G/I394R/E395R/L419A/E428V/R431S/Y462F/S470T/Q474M/ | |
| | K499L/I522L/Q523E | |
| 33/34 | N80D/C121S/F185L/K190E/D244V/K289D/I290V/D293S/I313A/C315G/ | ++ |
| | S317T/L336D/T342E/F359L/C391G/E395W/S470T/Q474M/K499L/I522L/ | |
| | Q523E | |
| 35/36 | N80D/C121S/F185L/K190E/K289D/I290V/D293S/I313A/T342E/K499L | ++ |
| 37/38 | N80D/C121S/F185L/K190E/E196R/D244V/K289D/I290V/D293S/I313A/ | ++ |
| | C315G/S317T/L336D/T342E/F359L/C391G/S470T/Q474M/K499L/I522L/ | |
| | Q523E | |
| 39/40 | N80D/C121S/F185L/K190E/D244V/K289D/I290V/D293S/I313A/C315G/ | ++ |
| | S317T/L336D/T342E/F359L/C391G/D392R/S470T/Q474M/K499L/I522L/ | |
| | Q523E | |
| 41/42 | N80D/C121S/I174L/F185L/E186G/K190E/G236P/D244V/L273V/F284L/ | ++ |
| | N288E/K289D/I290V/D293S/I313A/C315G/S317T/L336D/T342E/E352P/ | |
| | F359L/C391G/I394R/E395R/L419A/E428V/R431S/Y462F/S470T/Q474M/ | |
| | K499L/I522L/Q523E | |
| 43/44 | N80D/C121S/K190E/K289D/I290V | + |
| 45/46 | N80D/K106D/C121S/F185L/K190E/M205R/K289D/I290V/D293S/I313A/ | + |
| | T342E/S470T/Q474M/K499L/Q523E | |
| 47/48 | N80D/C121S/F185L/K190E/K289D/I290V/D293S/I313A/L336D/T342E/ | + |
| | F359L/C391G/S470T/Q474M/K499L/I522L/Q523E | |
| 49/50 | C121S/F185L/K190E/C213S/K289D/I290V/D293S | + |
| 51/52 | N80D/C121S/F185L/K190E/D244V/K289D/I290V/D293S/I313A/L336D/ | + |
| | T342E/F359L/C391G/F414H/S470T/Q474M/K499L/I522L/Q523E | |
| 53/54 | N80D/C121S/F185L/C315G | + |
| 55/56 | N80D/C121S/K131E/F185L/K190E/K289D/I290V/D293S/I313A/T342E/ | + |
| | S470T/Q474M/K499L/I522L/Q523E | |
| 57/58 | N80D/F185L/G236P/K289D/D293S | + |
| 59/60 | N80D/K106D/C121S/F185L/K190E/M205R/K289D/I290V/D293S/I313A/ | + |
| | C315G/L336D/T342E/F359L/C391G/S470T/Q474M/K499L/I522L/Q523E | |
| 61/62 | N80D/C121S/K131E/F185L/K190E/M205R/D244V/K289D/I290V/D293S/ | + |
| | I313A/C315G/L336D/T342E/F359L/C391G/F414H/S470T/Q474M/K499L/ | |
| | I522L/Q523E | |
| 63/64 | N80D/C121S/K131E/F185L/K190E/M205R/D244V/K289D/I290V/D293S/ | + |
| | I313A/L336D/T342E/F359L/C391G/F414H/S470T/Q474M/K499L/I522L/ | |
| | Q523E | |
| 65/66 | F185L/K289D/I290V/D293S | + |
| 67/68 | N80D/K106D/C121S/F185L/K190E/D244V/K289D/I290V/D293S/C307K/ | + |
| | T342E/F359L/S470T/Q474M/K499L | |
| 69/70 | N80D/C121S/F185L/K190E/C201R/K289D/I290V/D293S/I313A/T342E/ | + |
| | S470T/Q474M/K499L/I522L | |

Levels of increased protein recovery relative to the reference polypeptide of SEQ ID NO: 2 and defined as follows: “+” 1.00 to 3.20-fold increased activity; “++” >3.20-fold increased activity; “+++” >6.50-fold increased activity.

### Example 6

### Thermal Stability Improvements Over SEQ ID NO: 36

### Relative Stability Measurements of Shake-Flask Purified TdT Variants

TdT variants SEQ ID NO: 4, 8, 16, 36, and 48 were produced in shake flask and purified as described in Example 3. TdT concentrations were measured by absorption at 280 nm.

Reactions were performed in 96-well format 200 μL BioRad PCR plates. Reactions included 1 μM oligonucleotide, 25 M nucleotide triphosphate, 1 μM TdT, 20 mM triethanolamine (pH 7.8), and 250 μM cobalt (II) chloride. The reactions were set up as follows: (i) all reaction components, except for NTP, were pre-mixed in a single solution, and 20 μL of this solution was aliquoted into each well of the 96-well plate; (ii) the plate was heated at 39.8° C. or 63.1° C. as indicated; (iii) 15 μL of the heat-treated solution was transferred into a 96-well plate containing 5 μL of NTP solution (4×concentration in water); (iii) the solution was mixed well, spun down, and reacted at 45 RC for 15 minutes. Reaction plates were heat-sealed with a peelable aluminum seal and incubated in a thermocycler at the indicated temperature, then held at 4° C. until the reaction was quenched. Reactions were quenched and diluted for analytical analysis by CE as described in Example 4.

TABLE 6.1
Reaction conditions

Reaction buffer-20 mM TEA, 0.25 mM CoCl₂, pH 7.8; Preincubation Temp (C.) - 39.8° C. or 63.1° C.;
Preincubation Time (min)- 15; Reaction temperature (C.)- 45° C.; Reaction Time (min)- 15; Reaction
volume (μL) - 20; TdT concentration (μM)- 1; NTP- ddGTP; NTP conc (μM)-1; Oligonucleotide-5′-6-
FAM-T14ATCmC; Oligonucleotide conc (μM)- 1; Product- 5′-6-FAM-T14ATCmCddG.

Relative activity for each variant was calculated as the percent product of the variant as measured after a 39.8° C. pre-incubation relative to the percent product measured after pre-incubation at 63.1° C. multiplied by 100. The results are shown in Table 6.2.

TABLE 6.2
(relative % activity)

| | | Stability FIOP percent |
|---|---|---|
| SEQ ID | | activity at 63.1 vs. |
| NO: | Amino Acid Differences | activity at 39.8 Relative |
| (nt/aa) | (Relative to SEQ ID NO: 36) | to SEQ ID NO: 36 |
| 15/16 | I174L/D244V/L273V/F284L/N288E/C315G/S317T/L336D/ | ++ |
| | E352P/F359L/C391G/I394R/L419A/E428V/R431S/Y462F/ | |
| | S470T/Q474M/I522L/Q523E | |
| 3/4 | I174L/D244V/F284L/N288E/C315G/S317T/L336D/F359L/ | ++ |
| | C391G/I394R/E428V/R431S/Y462F/S470T/Q474M/I522L/ | |
| | Q523E | |
| 47/48 | L336D/F359L/C391G/S470T/Q474M/I522L/Q523E | + |
| 7/8 | D244V/C315G/S317T/L336D/F359L/C391G/S470T/Q474M/ | + |
| | I522L/Q523E | |

Levels of increased activity were determined relative to the reference polypeptide of SEQ ID NO: 36 and defined as follows: “+” 0.50 to 6.05-fold increased activity; “++” >6.05-fold increased activity; “+++” >15-fold increased activity.

### Example 7

### Improvements Over SEQ ID NO: 8 in the Extension of Oligonucleotide Acceptor Molecules with Nucleotide Triphosphate Donors

### HTP Screening for Improved TdT Variants

SEQ ID NO: 8 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 7.1.

Reactions were performed in 96-well format 200 μL BioRad PCR plates. Reactions included 1-10 μM oligonucleotide, 25-50 AM nucleotide triphosphate, 20 mM buffer, and 250 M cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 7.1
Reaction conditions

Lysis buffer-MOPS (50 mM, pH 7.4), 0.2 g/L lysozyme; Reaction buffer-20 mM MOPS, 50 mM
KOAc, 0.25 mM CoCl₂, pH 7.2; Lysate concentration (vol %)-25; Preincubation Temp (C.)-50° C.;
Preincubation Time (min)-15; Reaction temperature (C.)-50° C.; Reaction Time (min)-90; Reaction
volume (μL) -20; NTP (μM)-ddGTP; NTP conc (μM)-25; Oligonucleotide-5′-6-FAM-T17ATCmC;
Substrate conc (μM)-1; Product -5′-6-FAM-T17ATCmCddG.

Activity relative to SEQ ID NO: 8 (Activity FIOP) was calculated as the percent product of the variant compared with the percent product observed by the reaction with SEQ ID NO: 8 (where the percent product may be set as the average of replicates or else the highest single sample as appropriate). The results are shown in Table 7.2.

TABLE 7.2

| SEQ ID NO: | Amino Acid Differences | FIOP Product Peak Area |
|---|---|---|
| (nt/aa) | (Relative to SEQ ID NO: 8) | Relative to SEQ ID NO: 8 |
| 33/34 | E395W | +++ |
| 39/40 | D392R | +++ |
| 71/72 | E395T | +++ |
| 25/26 | K300R | +++ |
| 73/74 | E395R | +++ |
| 37/38 | E196R | +++ |
| 75/76 | E395Y | +++ |
| 77/78 | E395S | +++ |
| 79/80 | V456R | +++ |
| 81/82 | L421I | +++ |
| 83/84 | D392C | +++ |
| 85/86 | Q415S | +++ |
| 87/88 | I343V | +++ |
| 89/90 | E196G | +++ |
| 91/92 | K297T | +++ |
| 93/94 | K318E | ++ |
| 95/96 | R481V | ++ |
| 97/98 | R477T | ++ |
| 99/100 | K300P | ++ |
| 101/102 | K195W | ++ |
| 103/104 | V456K | ++ |
| 105/106 | L421M | ++ |
| 107/108 | V316C | ++ |
| 109/110 | R281A | ++ |
| 111/112 | E186A | ++ |
| 113/114 | L411R | ++ |
| 115/116 | C417G | ++ |
| 117/118 | L360V | ++ |
| 119/120 | K303M | ++ |
| 121/122 | V316I | ++ |
| 123/124 | K297Q | ++ |
| 125/126 | K195R | ++ |
| 127/128 | L360C | ++ |
| 129/130 | K318S | ++ |
| 131/132 | D183R | ++ |
| 133/134 | M282R | ++ |
| 135/136 | E186T | + |
| 137/138 | K303E | + |
| 139/140 | E193V | + |
| 141/142 | L454V | + |
| 143/144 | Q415A | + |
| 145/146 | D392A | + |
| 147/148 | E186L | + |
| 149/150 | T397R | + |
| 151/152 | D129G/E196G | + |
| 153/154 | K318T | + |
| 155/156 | E395L | + |
| 157/158 | K318V | + |
| 159/160 | D492T | + |
| 161/162 | E193N | + |
| 163/164 | E186G | + |
| 165/166 | T173L | + |
| 167/168 | E193G | + |
| 169/170 | C417V | + |
| 171/172 | E395A | + |
| 173/174 | L411A | + |
| 175/176 | E196A | + |
| 177/178 | E196W | + |
| 179/180 | L411G | + |
| 181/182 | E320N | + |
| 183/184 | E196Y | + |
| 185/186 | E193C | + |
| 187/188 | V268C | + |
| 189/190 | K297R | + |
| 191/192 | R481E | + |
| 193/194 | V324I | + |
| 195/196 | K303A | + |
| 197/198 | K297F | + |
| 199/200 | V316T | + |
| 201/202 | T266K | + |
| 203/204 | K263R | + |
| 205/206 | M282Q | + |

Levels of increased activity were determined relative to the reference polypeptide of SEQ ID NO: 8 and defined as follows: “+” 1.01 to 1.50-fold increased activity; “++” >1.50-fold increased activity; “+++” >2.25-fold increased activity.

### Example 8

### Improvements Over SEQ ID NO: 16 in the Extension of Oligonucleotide Acceptor Molecules with Nucleotide Triphosphate Donors

### HTP Screening for Improved TdT Variants

SEQ ID NO: 16 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and reactions prepared as described in Table 8.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 8.1
Reaction conditions

Lysis buffer-MOPS (50 mM, pH 7.4), 0.2 g/L lysozyme; Reaction buffer-20 mM MOPS, 50 mM
KOAc, 0.25 mM CoCl₂, pH 7.2; Lysate concentration (vol %)-12.5; Preincubation Temp (C.)-50° C.;
Preincubation Time (min)-15; Reaction temperature (C.)-50° C.; Reaction Time (min)-15; Reaction
volume (μL) -20; NTP (μM)-ddGTP; NTP conc (μM)-25; Oligonucleotide-5′-6-FAM-T17AT*mC;
Substrate conc (μM)-1; Product -5′-6-FAM-T17AT*mCddG.

Activity relative to SEQ ID NO: 16 (Activity FIOP) was calculated as the percent product of the variant compared with the percent product observed by the reaction with SEQ ID NO: 16 (where the percent product may be set as the average of replicates or else the highest single sample as appropriate). The results are shown in Table 8.2.

TABLE 8.2

| | | FIOP Product |
|---|---|---|
| SEQ ID | | Peak Area |
| NO: | Amino Acid Differences | Relative to SEQ |
| (nt/aa) | (Relative to SEQ ID NO: 16) | ID NO: 16 |
| 9/10 | E193G/E196Y/K297R/V324I/Q376H/N380D/ | +++ |
| | L401G/Q415S/M435T/E441M | |
| 17/18 | E193G/K297R/Q415S | +++ |
| 41/42 | E186G/G236P/E395R | +++ |
| 23/24 | E196R/T266K/V324I/T397R/L401G | +++ |
| 31/32 | G236P/M282R/E395R | +++ |
| 207/208 | Q415S | +++ |
| 209/210 | K297R/N380D/L401G/E441M | ++ |
| 211/212 | E193N/E196Y/V324I | ++ |
| 213/214 | G236P/M282R | ++ |
| 215/216 | Q376H/L401G/E441M | ++ |
| 217/218 | M435T/E441M | ++ |
| 219/220 | E186G | ++ |
| 221/222 | E196Y/K297R/V324I/M435T | ++ |
| 223/224 | K297R/M435T | ++ |
| 225/226 | E193G/K297R/V324I/Q376H/M435T | ++ |
| 227/228 | T266K/K297R/N380D/T397R/L401G | ++ |
| 229/230 | K318S/E395R | + |
| 231/232 | E193G/E196Y | + |
| 233/234 | E193G/M435T | + |
| 235/236 | E186G/G236P/K318S | + |
| 237/238 | E186G/M282R/K318S | + |
| 239/240 | E193G/E196Y/T266K/V324I/Q376H/N380D | + |
| 241/242 | M282R/K318S | + |
| 243/244 | E193G/E196R/K297R | + |
| 245/246 | E193G/E196Y/Q376H/N380D | + |
| 247/248 | M282R/R481E | + |
| 249/250 | G236P/K318S/R481E | + |
| 251/252 | M282R | + |
| 253/254 | E193G/E196Y/V324I/T397R/L401G/E441M | + |
| 255/256 | E196R/T266K | + |
| 257/258 | E196R | + |
| 259/260 | E193N/K297R/V324I/N380D | + |

Levels of increased activity were determined relative to the reference polypeptide of SEQ ID NO: 16 and defined as follows: “+” 1.24 to 1.57-fold increased activity; “++” >1.57-fold increased activity; “+++” >2.34-fold increased activity.

### Example 9

### Improvements Over SEQ ID NO: 24 in the Extension of Oligonucleotide Acceptor Molecules with Nucleotide Triphosphate Donors

### HTP Screening for Improved TdT Variants

SEQ ID NO: 24 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 9.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 9.1
Reaction conditions

Lysis buffer-MOPS (50 mM, pH 7.4), 0.2 g/L lysozyme; Reaction buffer-20 mM MOPS, 50 mM
KOAc, 0.25 mM CoCl₂, pH 7.2; Lysate concentration (vol %)-12.5; Preincubation Temp (C.)-47° C.;
Preincubation Time (min)-15; Reaction temperature (C.)-47° C.; Reaction Time (min)-30; Reaction
volume (μL) -20; NTP (μM)-mATP; NTP conc (μM)-25; Oligonucleotide-5′-6-FAM-
T17ATC(2′dF)C; Substrate conc (μM)-1; Product -5′-6-FAM-T17ATC(2′dF)CmA.

Activity relative to SEQ ID NO: 24 (Activity FIOP) was calculated as the percent product of the variant compared with the percent product observed by the reaction with SEQ ID NO: 24 (where the percent product may be set as the average of replicates or else the highest single sample as appropriate).

The results are shown in Table 8.2.

TABLE 9.2

| SEQ ID NO: | Amino Acid Differences | FIOP Product Peak Area |
|---|---|---|
| (nt/aa) | (Relative to SEQ ID NO: 24) | Relative to SEQ ID NO: 24 |
| 261/262 | D21E | +++ |
| 263/264 | G92R | +++ |
| 265/266 | V124E | +++ |
| 267/268 | K53E | +++ |
| 269/270 | K24G | +++ |
| 271/272 | T101E | +++ |
| 273/274 | I155E | +++ |
| 275/276 | I34S | +++ |
| 277/278 | I34D | +++ |
| 279/280 | L153E | +++ |
| 281/282 | T58S | +++ |
| 283/284 | T108K | +++ |
| 285/286 | D142M | +++ |
| 287/288 | G92Y | +++ |
| 289/290 | K161E | +++ |
| 291/292 | M37F | +++ |
| 293/294 | M37A | +++ |
| 295/296 | A150E | +++ |
| 297/298 | T107R | +++ |
| 299/300 | T162E | +++ |
| 301/302 | V141E | +++ |
| 303/304 | L156D | +++ |
| 305/306 | K161S | +++ |
| 307/308 | L156E/K294T | +++ |
| 309/310 | H33K | ++ |
| 311/312 | M30G | ++ |
| 313/314 | T58A | ++ |
| 315/316 | M104I | ++ |
| 317/318 | I155T | ++ |
| 319/320 | K161D | ++ |
| 321/322 | L156Q | ++ |
| 323/324 | K106S | ++ |
| 325/326 | M12S | ++ |
| 327/328 | L156M | ++ |
| 329/330 | M104P | ++ |
| 331/332 | R14G | ++ |
| 333/334 | T152R | ++ |
| 335/336 | L153Q | ++ |
| 337/338 | M37T | ++ |
| 339/340 | N145E | ++ |
| 341/342 | D142S | ++ |
| 343/344 | S20T | ++ |
| 345/346 | R14Q | ++ |
| 347/348 | D94E | ++ |
| 349/350 | T160G | ++ |
| 351/352 | H13S | ++ |
| 353/354 | G92M | ++ |
| 355/356 | L153K | ++ |
| 357/358 | S35E | ++ |
| 359/360 | L153G | ++ |
| 361/362 | L153P | ++ |
| 363/364 | T159D | ++ |
| 365/366 | M37G | ++ |
| 367/368 | G92V | ++ |
| 369/370 | I18D | ++ |
| 371/372 | K106H | ++ |
| 373/374 | V141R | ++ |
| 375/376 | M37V | ++ |
| 377/378 | K133G | ++ |
| 379/380 | D31S | + |
| 381/382 | M30V | + |
| 383/384 | I34K | + |
| 385/386 | M30E | + |
| 387/388 | T17A | + |
| 389/390 | D147L | + |
| 391/392 | T152G | + |
| 393/394 | I41V | + |
| 395/396 | H13R | + |
| 397/398 | T149R | + |
| 399/400 | M137A | + |
| 401/402 | I34R | + |
| 403/404 | L153V | + |
| 405/406 | T61H | + |
| 407/408 | K29P | + |
| 409/410 | K103M | + |
| 411/412 | A97D | + |
| 413/414 | M104G | + |
| 415/416 | H13K | + |
| 417/418 | L153M | + |
| 419/420 | H33P | + |
| 421/422 | T108D | + |
| 423/424 | S139A | + |
| 425/426 | T17G | + |
| 427/428 | M37S | + |
| 429/430 | I18R | + |
| 431/432 | G23P | + |
| 433/434 | T107W | + |
| 435/436 | K126V | + |
| 437/438 | F22G | + |
| 439/440 | H33G | + |
| 441/442 | G92S | + |
| 443/444 | Q135R | + |
| 445/446 | I163V | + |
| 447/448 | K29R | + |
| 449/450 | S20H | + |
| 451/452 | K161R | + |
| 453/454 | D94R | + |
| 455/456 | S35G | + |
| 457/458 | R140E | + |
| 459/460 | A57H | + |
| 461/462 | T61L | + |
| 463/464 | K106G | + |
| 465/466 | F22L | + |
| 467/468 | Q27D | + |
| 469/470 | V124I | + |
| 471/472 | K161G | + |
| 473/474 | E138Q | + |
| 475/476 | G23E | + |
| 477/478 | V141M | + |
| 479/480 | H102L | + |
| 481/482 | E105N | + |
| 483/484 | A144C | + |
| 485/486 | A150G | + |
| 487/488 | R140G | + |
| 489/490 | R26G | + |
| 491/492 | I163L | + |
| 493/494 | N154G | + |
| 495/496 | H13G | + |

Levels of increased activity were determined relative to the reference polypeptide of SEQ ID NO: 24 and defined as follows: “+” >1.00-fold increased activity; “++” >1.30-fold increased activity; “+++” >1.75-fold increased activity.

### Example 10

### Improvements Over SEQ ID NO: 24 in the Extension of Oligonucleotide Acceptor Molecules with Nucleotide Triphosphate Donors

### HTP Screening for Improved TdT Variants

SEQ ID NO: 24 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 10.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 10.1-Reaction conditions

Lysis buffer-MOPS (50 mM, pH 7.4), 0.2 g/L lysozyme;
Reaction buffer-20 mM MOPS, 50 mM
KOAc, 0.25 mM CoCl₂, pH 7.2; Lysate
concentration (vol %)-25; Preincubation Temp (C.)-58° C.;
Preincubation Time (min)-15; Reaction temperature (C.)-58° C.;
Reaction Time (min)-30; Reaction
volume (μL) -20; NTP (μM)-ddGTP; NTP conc (μM)-25;
Oligonucleotide-5′-6-FAM-
T17ATC(2′dF)C; Substrate conc (μM)-1;
Product -5′-6-FAM-T17ATC(2′dF)CddG.

Activity relative to SEQ ID NO: 24 (Activity FIOP) was calculated as the percent product of the variant compared with the percent product observed by the reaction with SEQ ID NO: 24 (where the percent product may be set as the average of replicates or else the highest single sample as appropriate). The results are shown in Table 10.2.

TABLE 10.2

| | Amino Acid | FIOP Product |
|---|---|---|
| SEQ | Differences | Peak Area |
| ID NO: | (Relative to | Relative to |
| (nt/aa) | SEQ ID NO: 24) | SEQ ID NO: 24 |
| 271/272 | T101E | +++ |
| 265/266 | V124E | +++ |
| 497/498 | V124G | +++ |
| 499/500 | T101V | +++ |
| 501/502 | H7Y/Q135C | +++ |
| 361/362 | L153P | +++ |
| 503/504 | K29I | +++ |
| 505/506 | R16V | +++ |
| 507/508 | Q109M | +++ |
| 509/510 | I85V | +++ |
| 511/512 | K24A | +++ |
| 513/514 | K131W | +++ |
| 515/516 | Q135E | +++ |
| 517/518 | F110V | +++ |
| 519/520 | R14Y | +++ |
| 521/522 | S35W | +++ |
| 523/524 | T101G | +++ |
| 525/526 | K123M | +++ |
| 527/528 | F110M | ++ |
| 529/530 | V124M | ++ |
| 531/532 | G23C | ++ |
| 533/534 | K24M | ++ |
| 535/536 | R14N | ++ |
| 537/538 | H44S | ++ |
| 539/540 | H13E | ++ |
| 541/542 | D65S | ++ |
| 543/544 | T108M | ++ |
| 545/546 | K123Q | ++ |
| 547/548 | H33C | ++ |
| 549/550 | A57T | ++ |
| 551/552 | T130M | ++ |
| 553/554 | K133M | ++ |
| 555/556 | D94N | ++ |
| 475/476 | G23E | ++ |
| 557/558 | G23T | ++ |
| 559/560 | E105W | ++ |
| 561/562 | G132S | ++ |
| 563/564 | E77C | ++ |
| 565/566 | V124S | ++ |
| 567/568 | E77S | ++ |
| 569/570 | Q135K | ++ |
| 571/572 | S35H | ++ |
| 409/410 | K103M | ++ |
| 303/304 | L156D | ++ |
| 573/574 | Y134M | ++ |
| 575/576 | Q135H | ++ |
| 577/578 | M137E | ++ |
| 579/580 | N32E | + |
| 581/582 | K131G | + |
| 583/584 | N89A | + |
| 585/586 | K133Q | + |
| 587/588 | T108G | + |
| 589/590 | Q27F | + |
| 591/592 | I119Q | + |
| 593/594 | Y134W | + |
| 595/596 | K29G | + |
| 597/598 | K25N | + |
| 397/398 | T149R | + |
| 469/470 | V124I | + |
| 599/600 | K24P | + |
| 335/336 | L153Q | + |
| 279/280 | L153E | + |
| 377/378 | K133G | + |
| 601/602 | S139G | + |
| 603/604 | T130A | + |
| 605/606 | K126C | + |
| 607/608 | S20P | + |
| 609/610 | E45R | + |
| 467/468 | Q27D | + |
| 479/480 | H102L | + |
| 611/612 | I15L | + |
| 613/614 | T130Q | + |
| 615/616 | T130S | + |
| 481/482 | E105N | + |
| 617/618 | A97T | + |
| 619/620 | K28R | + |
| 489/490 | R26G | + |
| 621/622 | Q109N | + |
| 623/624 | F46M | + |
| 625/626 | K106V | + |
| 627/628 | I119F | + |
| 629/630 | I18Y | + |
| 631/632 | H33A | + |
| 331/332 | R14G | + |
| 633/634 | D31V | + |
| 359/360 | L153G | + |
| 635/636 | Q109T | + |
| 275/276 | I34S | + |
| 399/400 | M137A | + |
| 295/296 | A150E | + |
| 637/638 | E138A | + |
| 639/640 | S93V | + |
| 641/642 | T17A/K131R | + |
| 643/644 | M12F | + |

Levels of increased activity were determined relative to the reference polypeptide of SEQ ID NO: 24 and defined as follows: “+” 1.21 to 1.42-fold increased activity; “++” >1.42-fold increased activity; “+++” >1.69-fold increased activity.

### Example 11

### Improvements Over SEQ ID NO: 268 in the Extension of Oligonucleotide Acceptor Molecules with Nucleotide Triphosphate Donors

### HTP Screening for Improved TdT Variants

SEQ ID NO: 268 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 11.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 11.1-Reaction conditions

Lysis buffer-MOPS (50 mM, pH 7.4), 0.2 g/L lysozyme;
Reaction buffer-20 mM MOPS, 50 mM
KOAc, 0.25 mM CoCl₂, pH 7.2;
Lysate concentration (vol %)-12.5; Preincubation Temp (C.)-47° C.;
Preincubation Time (min)-15; Reaction temperature (C.)-47° C.;
Reaction Time (min)-30; Reaction
volume (μL) -20; NTP (μM)-mATP; NTP conc (μM)-25;
Oligonucleotide-5′-6-FAM-
T17ATC(2′dF)C; Substrate conc (μM)-1;
Product -5′-6-FAM-T17ATC(2′dF)CmA.

Activity relative to SEQ ID NO: 268 (Activity FIOP) was calculated as the percent product of the variant compared with the percent product observed by the reaction with SEQ ID NO: 268 (where the percent product may be set as the average of replicates or else the highest single sample as appropriate). The results are shown in Table 11.2.

TABLE 11.2

| | Amino Acid | FIOP Product |
|---|---|---|
| SEQ | Differences | Peak Area |
| ID NO: | (Relative to | Relative to |
| (nt/aa) | SEQ ID NO: 268) | SEQ ID NO: 268 |
| 645/646 | K300P/E395Y/A419L | ++ |
| 647/648 | K106V/K300P/Q415A/A419L/V456R | ++ |
| 649/650 | R140G | + |
| 651/652 | R14G/E53K/A419L | + |
| 653/654 | R14G/E53K/K300P | + |

Levels of increased activity were determined relative to the reference polypeptide of SEQ ID NO: 268 and defined as follows: “+” 1.02 to 1.70-fold increased activity; “++” >1.70-fold increased activity.

### Example 12

### Improvements Over SEQ ID NO: 648 in the Extension of Oligonucleotide Acceptor Molecules with Nucleotide Triphosphate Donors

### HTP Screening for Improved TdT Variants

SEQ ID NO: 648 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 12.1.

Reactions were performed in 96-well format 200 μL BioRad PCR plates. Reactions included 1-M oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 12.1-Reaction conditions

Lysis buffer-MOPS (50 mM, pH 7.4), 0.2 g/L lysozyme;
Reaction buffer-20 mM MOPS, 50 mM
KOAc, 0.25 mM CoCl₂, pH 7.2;
Lysate concentration (vol %)-3.125; Preincubation Temp (C.)-50° C.;
Preincubation Time (min)-15; Reaction temperature (C.)-50° C.;
Reaction Time (min)-30; Reaction
volume (μL) -20; NTP (μM)-ddGTP; NTP conc (μM)-25;
Oligonucleotide-5′-6-FAM-T17AT*mC;
Substrate conc (μM)-1;
Product -5′-6-FAM-T17AT*mCddG.

Activity relative to SEQ ID NO: 648 (Activity FIOP) was calculated as the percent product of the variant compared with the percent product observed by the reaction with SEQ ID NO: 648 (where the percent product may be set as the average of replicates or else the highest single sample as appropriate). The results are shown in Table 12.2.

TABLE 12.2

| | Amino Acid | FIOP Product |
|---|---|---|
| SEQ | Differences | Peak Area |
| ID NO: | (Relative to | Relative to |
| (nt/aa) | SEQ ID NO: 648) | SEQ ID NO: 648 |
| 655/656 | R14G/I34D/D94E/V106K/T108K/V141E | +++ |
| 657/658 | I34D/M37A/G92R | +++ |
| 659/660 | M12S/R14G/I34D/M37A/V106K/R140G/D142M/ | +++ |
| | A150E/T152R/L153E | |
| 661/662 | I34D/G92R/D94E/V141E/D142M | +++ |
| 663/664 | M12S/M37A/D94E/V141E/A150E/T152R/L153E | +++ |
| 665/666 | M12S/I34D/G92R/R140G | +++ |
| 667/668 | I34D/A150E/T152R/L153E | +++ |
| 669/670 | M12S/R14G/I34D/M37F/D94E/R140G/V141E/N145E | +++ |
| 671/672 | T101E/V106S/V124E/I155E | +++ |
| 673/674 | G92R/D94E/V106K/D142S/N145E | +++ |
| 675/676 | S20T/D21E/K24G/H33K/T58A/M104P/ | +++ |
| | V106S/V124E/I155E/L156E | |
| 677/678 | M12S/R14G/I34D/M37A/A150E | +++ |
| 679/680 | S20T/D21E/H33K/T58A/T101E/M104P/V106K/V124E/I155E | +++ |
| 681/682 | I34S/M37A/A150E/L153E | +++ |
| 683/684 | M12S/R14G/V106K/T108K/R140G/V141E/N145E/A150E | +++ |
| 685/686 | M12S/R14G/I34D/R140G/A150E | +++ |
| 687/688 | R14G/I34S/A150E/L153E | +++ |
| 689/690 | I34D/M37A | +++ |
| 691/692 | M30G/M104P/V106S/I155E/L156E | +++ |
| 693/694 | M12S/R14G/I34D/M37A/V141E/D142S | +++ |
| 695/696 | R14G/D31G/I34D/M37A/R140G/V141E/N145E/K161E/T162E | +++ |
| 697/698 | V106K/T108K/R140G/V141E/T152R/L153E | +++ |
| 699/700 | M30G/T101E/M104P/V106S/I155E/L156E | +++ |
| 701/702 | D21E/T101E/M104P/V106S/L156E | ++ |
| 703/704 | D21E/H33K/T58A/T101E/V106K | ++ |
| 705/706 | R14G/I34S/M37A | ++ |
| 707/708 | M104P/V106S | ++ |
| 709/710 | M12S/M37F/R140G/V141E/A150E/T162E | ++ |
| 711/712 | M12S/R14G/G92R/D94E | ++ |
| 713/714 | M12S/I34D/A150E/T152R | ++ |
| 715/716 | T58S/T101E/M104P/V106S/L156E | ++ |
| 717/718 | R14G/V141E/K161E | ++ |
| 719/720 | R14G/I34D/A150E/L153E | ++ |
| 721/722 | R14G/I34S/M37A/T152R | ++ |
| 723/724 | V106K/T108K/R140G/D142S/A150E/L153E | ++ |
| 725/726 | M12S/R14G/I34D/R140G/D142S | ++ |
| 727/728 | D21E/H33K/T101E/M104P/V106S | ++ |
| 729/730 | R14G/K161E | ++ |
| 731/732 | T58A/T101E/V106K/1155E | ++ |
| 733/734 | M12S/R14G/D94E/A150E/T152R | ++ |
| 735/736 | M12S/R14G/I34D/M37F/A150E/L153E | ++ |
| 737/738 | T101E/V106K/I155E/L156E | ++ |
| 739/740 | M104P/V106S/I155E | ++ |
| 741/742 | M30G/M104P/V106S/I155E | ++ |
| 743/744 | M12S/R14G/I34S/M37A/D142M/K161E | ++ |
| 745/746 | M30G/V106K/I155E | ++ |
| 747/748 | M12S/R14G/A150E/T152R/L153E | ++ |
| 749/750 | S20T/T58S/T101E/M104P/V106S/L156E | ++ |
| 751/752 | M30G/H33K/T58S/M104P/V106S/I155E/L156E | ++ |
| 753/754 | T101E/V106K | ++ |
| 755/756 | M104P/V106K/I155E | ++ |
| 757/758 | M30G/H33K/M104I/V106S/I155E/L156E | ++ |
| 759/760 | M12S/R14G/I34S/D142S/A150E/L153E | ++ |
| 761/762 | D21E/M104P/V106S | ++ |
| 763/764 | D21E/T58S/T101E/M104I/V106S/I155E | + |
| 765/766 | K161E | + |
| 767/768 | D21E/M104P/V106S/L156E | + |
| 769/770 | M12S/R14G/I34D | + |
| 771/772 | R14G/L153E | + |
| 773/774 | M12S/R14G/L153E | + |
| 775/776 | S20T/T58A/T101E/V106K | + |
| 777/778 | R140G/N145E | + |
| 779/780 | S20T/H33K/M104P/V106K/V124E/L156E | + |
| 781/782 | D21E/H33K/T58S/V106S/I155E/L156E | + |
| 783/784 | D21E/T58A/V106K/I155E/L156E | + |
| 785/786 | H33K/T58A/M104I/V106S | + |
| 787/788 | I34S/M37F/R140G/V141E/D142M/N145E | + |
| 789/790 | M12S/R14G/V141E/D142S | + |
| 791/792 | R140G/N145E/A150E/T152R | + |
| 793/794 | H33K/T101E/M104P/V106K/I155E | + |
| 795/796 | T101E/M104I/V106K/L156E | + |
| 797/798 | T101E/M104I/V106K | + |
| 799/800 | M104I/V106K | + |
| 801/802 | M12S/R14G/V106K/T108K/T152R | + |
| 803/804 | M12S/R14G/I34D/M37F/D142S/N145E | + |
| 805/806 | M37A/V141E/D142S | + |
| 807/808 | I34D/V141E/D142S/N145E | + |
| 809/810 | M104I/V106K/V124E | + |
| 811/812 | T101E/M104I/V106S/L156E | + |
| 813/814 | M104I/V106S/I155E/L156E | + |
| 815/816 | V141E/T152R | + |
| 817/818 | M30G/H33K/T101E/V106S/L156E | + |
| 819/820 | R14G/I34S/M37F/N145E | + |
| 821/822 | S20T/T101E/V106K/L156E | + |
| 823/824 | V141E | + |
| 825/826 | S20T/D21E/T58A/M104I/V106S/I155E/L156E | + |
| 827/828 | M12S/K161E | + |
| 829/830 | R14G | + |
| 831/832 | M37F/L153E | + |
| 833/834 | T101E/M104P/V106K/I155E/L156E | + |
| 835/836 | M104I/V106S | + |
| 837/838 | D21E/H33K/V106S | + |
| 839/840 | I34D/M37F/V141E/D142S | + |
| 841/842 | M104P/V106K/L156E | + |
| 843/844 | T58S/M104I/V106K/I155E/L156E | + |
| 845/846 | V106K/T107R/T108K/D142S/S220R | + |
| 847/848 | M37F/G92R/D142M | + |
| 849/850 | V106K/L156E | + |
| 851/852 | R14G/V106K | + |
| 853/854 | D21E/M104I/V106S/V124E | + |
| 855/856 | R14G/R140G | + |
| 857/858 | R14G/D142M | + |
| 859/860 | R140G/V141E/D142M | + |
| 861/862 | M104P/V106K | + |
| 863/864 | R14G/D142S/K161E/T162E | + |
| 865/866 | M12S/R14G/V106K/T107R/V141E/D142M | + |

Levels of increased activity were determined relative to the reference polypeptide of SEQ ID NO: 648 and defined as follows: “+” 1.19 to 3.50-fold increased activity; “++” >3.50-fold increased activity; “+++” >8.50-fold increased activity.

### Example 13

### Improvements Over SEQ ID NO: 660 in the Extension of Oligonucleotide Acceptor Molecules with Nucleotide Triphosphate Donors

### HTP Screening for Improved TdT Variants

SEQ ID NO: 660 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 13.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 13.1-Reaction conditions

Lysis buffer-TEA (50 mM, pH 7.5), 0.1 g/L lysozyme;
Reaction buffer-20 mM TEA, 0.25 mM CoCl₂,
pH 7.8; Lysate concentration (vol %)-25;
Preincubation Temp (C.)-50° C.; Preincubation Time (min)-
60; Reaction temperature (C.)-50° C.; Reaction Time (min)-30;
Reaction volume (μL) -20; NTP
(μM)-*rATP; NTP conc (μM)-25;
Oligonucleotide-5′-6-FAM-T12ATCAC*(2′dF)A; Substrate conc
(μM)-1; Product -5′-6-FAM-T12ATCAC*(2′dF)A*rA.

Activity relative to SEQ ID NO: 660 (Activity FIOP) was calculated as the percent product of the variant compared with the percent product observed by the reaction with SEQ ID NO: 660 (where the percent product may be set as the average of replicates or else the highest single sample as appropriate). The results are shown in Table 13.2.

TABLE 13.2

| | Amino Acid | FIOP Product |
|---|---|---|
| SEQ | Differences | Peak Area |
| ID NO: | (Relative to | Relative to |
| (nt/aa) | SEQ ID NO: 660) | SEQ ID NO: 660 |
| 867/868 | R16V/K29I/M30E/T101E/M104V | +++ |
| 869/870 | T101E/V141E/I155E/L156E | +++ |
| 871/872 | T101E/M137E/L155E | +++ |
| 873/874 | G92R/T101E/M137A/I155E/E476R | +++ |
| 875/876 | T101E/M104V | ++ |
| 877/878 | T101E | ++ |
| 879/880 | R16V/K29I/M30E | ++ |
| 881/882 | G92R/D94E/T108K/V141E/I155E/D392R | ++ |
| 883/884 | T58A | ++ |
| 885/886 | T108K | + |
| 887/888 | D94E/T101E/L156E/E476R | + |
| 889/890 | R16V/M30L/M104V | + |
| 891/892 | K29I/M30E | + |
| 893/894 | R16V/K29I/M30E/H33K/E153P | + |
| 895/896 | R16V/H33K | + |

Levels of increased activity were determined relative to the reference polypeptide of SEQ ID NO: 660 and defined as follows: “+” 1.20 to 1.56-fold increased activity; “++” >1.56-fold increased activity; “+++” >2.42-fold increased activity.

### Example 14

### Improvements Over SEQ ID NO: 660 in the Extension of Oligonucleotide Acceptor Molecules with Nucleotide Triphosphate Donors

### HTP Screening for Improved TdT Variants

SEQ ID NO: 660 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 14.1.

Reactions were performed in 96-well format 200 mL BioRad PCR plates. Reactions included 1-10 μM oligonucleotide, 25-50 5M nucleotide triphosphate, 20 mM buffer, and 250 nM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 14.1-Reaction conditions

Lysis buffer-TEA (50 mM, pH 7.5), 0.1 g/L lysozyme;
Reaction buffer-20 mM TEA, 0.25 mM CoCl2,
pH 7.8; Lysate concentration (vol %)-25;
Preincubation Temp (C.)-48° C.; Preincubation Time (min)-
60; Reaction temperature (C.)-48° C.;
Reaction Time (min)-30; Reaction volume (μL) -20; NTP
(μM)-*rGTP; NTP conc (μM)-25;
Oligonucleotide-5′-6-FAM-T12ATCAC*mA; Substrate conc
(μM)-1; Product -5′-6-FAM-T12ATCAC*mA*rG.

Activity relative to SEQ ID NO: 660 (Activity FIOP) was calculated as the percent product of the variant compared with the percent product observed by the reaction with SEQ ID NO: 660 (where the percent product may be set as the average of replicates or else the highest single sample as appropriate). The results are shown in Table 14.2.

TABLE 14.2

| | Amino Acid | FIOP Product |
|---|---|---|
| SEQ | Differences | Peak Area |
| ID NO: | (Relative to | Relative to |
| (nt/aa) | SEQ ID NO: 660) | SEQ ID NO: 660 |
| 897/898 | E342V | +++ |
| 899/900 | F204L/E342W | +++ |
| 901/902 | L264R | +++ |
| 903/904 | E310A | +++ |
| 905/906 | K303V | +++ |
| 907/908 | V290L | +++ |
| 909/910 | G391R | +++ |
| 911/912 | L421F | +++ |
| 913/914 | Y308F | +++ |
| 915/916 | L306F | +++ |
| 917/918 | L454M | +++ |
| 919/920 | K278V | +++ |
| 921/922 | K303A | +++ |
| 923/924 | K278E | +++ |
| 925/926 | V316L | +++ |
| 927/928 | R281T | +++ |
| 929/930 | E310R | +++ |
| 931/932 | K278C | +++ |
| 933/934 | R281C | +++ |
| 935/936 | K278I | ++ |
| 937/938 | K297V | ++ |
| 939/940 | R456S | ++ |
| 941/942 | S525F | ++ |
| 943/944 | L360I | ++ |
| 945/946 | G236E/K297L | ++ |
| 947/948 | Q385R | ++ |
| 949/950 | E310G | ++ |
| 951/952 | R281G | ++ |
| 953/954 | R281L | ++ |
| 955/956 | G391L | ++ |
| 957/958 | K303N | ++ |
| 959/960 | V268L | ++ |
| 961/962 | L419G | ++ |
| 963/964 | K448R | ++ |
| 965/966 | M282G | ++ |
| 967/968 | K278T | ++ |
| 969/970 | K297D | ++ |
| 971/972 | E342R | ++ |
| 973/974 | P300R | ++ |
| 975/976 | R281S | ++ |
| 977/978 | R281V | ++ |
| 979/980 | K303S | ++ |
| 981/982 | K297S | ++ |
| 983/984 | H413F | ++ |
| 985/986 | M282W | ++ |
| 987/988 | E476V | ++ |
| 989/990 | R281A | ++ |
| 991/992 | H413C | + |
| 993/994 | H413V | + |
| 995/996 | R258S | + |
| 997/998 | Y308W | + |
| 999/1000 | M205E | + |
| 1001/1002 | R258W | + |
| 1003/1004 | G315S | + |
| 1005/1006 | M282C | + |
| 1007/1008 | R291S | + |
| 1009/1010 | K297C | + |
| 1011/1012 | L264A | + |
| 1013/1014 | Y309F | + |
| 1015/1016 | L264E | + |
| 1017/1018 | R473V | + |
| 1019/1020 | A276S | + |
| 1021/1022 | F353K | + |
| 1023/1024 | T344V | + |
| 1025/1026 | R258G | + |
| 1027/1028 | L264S | + |
| 1029/1030 | R258A | + |
| 1031/1032 | V290A | + |
| 1033/1034 | R258L | + |
| 1035/1036 | E310S | + |
| 1037/1038 | M282H | + |
| 1039/1040 | G315A | + |
| 1041/1042 | V316A | + |
| 1043/1044 | R258M | + |
| 1045/1046 | S261V | + |
| 1047/1048 | A515V | + |
| 1049/1050 | N197M | + |
| 1051/1052 | L419H | + |
| 1053/1054 | K303Q | + |
| 1055/1056 | L312V | + |
| 1057/1058 | F353M | + |
| 1059/1060 | F353A | + |
| 1061/1062 | Y280F | + |
| 1063/1064 | S261R | + |
| 1065/1066 | M205L | + |
| 1067/1068 | E310H | + |
| 1069/1070 | F262I | + |
| 1071/1072 | S525H | + |
| 1073/1074 | K278R | + |
| 1075/1076 | R258C | + |
| 1077/1078 | S261G | + |
| 1079/1080 | F353R | + |
| 1081/1082 | A410E | + |
| 1083/1084 | K195E | + |
| 1085/1086 | F353S | + |
| 1087/1088 | F269W | + |

Levels of increased activity were determined relative to the reference polypeptide of SEQ ID NO: 660 and defined as follows: “+” 1.08 to 1.94-fold increased activity; “++” >1.94-fold increased activity; “+++” >3.10-fold increased activity.

### Example 15

### Improvements Over SEQ ID NO: 882 in the Extension of Oligonucleotide Acceptor Molecules with Nucleotide Triphosphate Donors

### HTP Screening for Improved TdT Variants

SEQ ID NO: 882 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 15.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 15.1-Reaction conditions

Lysis buffer-TEA (50 mM, pH 7.5), 0.1 g/L lysozyme;
Reaction buffer-20 mM TEA, 0.25 mM CoCl₂,
pH 7.8; Lysate concentration (vol %)-25;
Preincubation Temp (C.)-50° C.; Preincubation Time (min)-
60; Reaction temperature (C.)-50° C.;
Reaction Time (min)-30; Reaction volume (μL)-20; NTP (μM)-
*rGTP; NTP conc (μM)-25; Oligonucleotide-5′-6-FAM-T12ATCAC*mC;
Substrate conc (μM)-1;
Product -5′-6-FAM-T12ATCAC*mC*rG.

Activity relative to SEQ ID NO: 882 (Activity FIOP) was calculated as the percent product of the variant compared with the percent product observed by the reaction with SEQ ID NO: 882 (where the percent product may be set as the average of replicates or else the highest single sample as appropriate). The results are shown in Table 15.2.

TABLE 15.2

| | Amino Acid | FIOP Product |
|---|---|---|
| SEQ | Differences | Peak Area |
| ID NO: | (Relative to | Relative to |
| (nt/aa) | SEQ ID NO: 882) | SEQ ID NO: 882 |
| 1089/1090 | T203A | +++ |
| 1091/1092 | R394E | +++ |
| 1093/1094 | R394T | +++ |
| 1095/1096 | D314R | +++ |
| 1097/1098 | D199R | +++ |
| 1099/1100 | R461G | +++ |
| 1101/1102 | L403F/F462H | +++ |
| 1103/1104 | T484R | +++ |
| 1105/1106 | D199V | +++ |
| 1107/1108 | T379C | +++ |
| 1109/1110 | S325F | +++ |
| 1111/1112 | R481D | +++ |
| 1113/1114 | D314V | +++ |
| 1115/1116 | F462I | ++ |
| 1117/1118 | T484M | ++ |
| 1119/1120 | D314K | ++ |
| 1121/1122 | A321K | ++ |
| 1123/1124 | D314L | ++ |
| 1125/1126 | T203S | ++ |
| 1127/1128 | I208V | ++ |
| 1129/1130 | D199M | ++ |
| 1131/1132 | F462R | ++ |
| 1133/1134 | I408A | ++ |
| 1135/1136 | C457V | ++ |
| 1137/1138 | T203R | ++ |
| 1139/1140 | T203L | ++ |
| 1141/1142 | R477T | ++ |
| 1143/1144 | A321C | ++ |
| 1145/1146 | D314Y | ++ |
| 1147/1148 | F462W | ++ |
| 1149/1150 | A495S | + |
| 1151/1152 | Q329R/F462E | + |
| 1153/1154 | T317G | + |
| 1155/1156 | A321S | + |
| 1157/1158 | A313I | + |
| 1159/1160 | T275V | + |
| 1161/1162 | D314G | + |
| 1163/1164 | S325V | + |
| 1165/1166 | D199G | + |
| 1167/1168 | R406V | + |
| 1169/1170 | D199S | + |
| 1171/1172 | C457S | + |
| 1173/1174 | R406G | + |
| 1175/1176 | R196G | + |
| 1177/1178 | R481M | + |
| 1179/1180 | D199Q | + |
| 1181/1182 | R397T | + |
| 1183/1184 | S325T | + |
| 1185/1186 | R196Y | + |
| 1187/1188 | R397D | + |
| 1189/1190 | S325W | + |
| 1191/1192 | N175L | + |
| 1193/1194 | R481T | + |
| 1195/1196 | W469Q | + |
| 1197/1198 | T203G | + |
| 1199/1200 | R461V | + |
| 1201/1202 | I408T | + |
| 1203/1204 | D322A | + |

Levels of increased activity were determined relative to the reference polypeptide of SEQ ID NO: 882 and defined as follows: “+” 1.01 to 1.60-fold increased activity; “++” >1.60-fold increased activity; “+++” >2.85-fold increased activity.

### Example 16

### Improvements Over SEQ ID NO: 882 in the Extension of Oligonucleotide Acceptor Molecules with Nucleotide Triphosphate Donors

### HTP Screening for Improved TdT Variants

SEQ ID NO: 882 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 16.1.

Reactions were performed in 96-well format 200 μL BioRad PCR plates. Reactions included 1-10 μM oligonucleotide, 25-50 μM nucleotide triphosphate, 20 mM buffer, and 250 nM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 16.1-Reaction conditions

Lysis buffer-TEA (50 mM, pH 7.5), 0.1 g/L lysozyme;
Reaction buffer-20 mM TEA, 0.25 mM CoCl₂,
pH 7.8; Lysate concentration (vol %)-25;
Preincubation Temp (C.)-50° C.; Preincubation Time (min)-
60; Reaction temperature (C.)-50° C.;
Reaction Time (min)-30; Reaction volume (μL) -20; NTP
(μM)-*rUTP; NTP conc (μM)-25;
Oligonucleotide-5′-6-FAM-T12ATCAC*mC; Substrate conc
(μM)-1; Product -5′-6-FAM-T12ATCAC*mC*rU.

Activity relative to SEQ ID NO: 882 (Activity FIOP) was calculated as the percent product of the variant compared with the percent product observed by the reaction with SEQ ID NO: 882 (where the percent product may be set as the average of replicates or else the highest single sample as appropriate).

The results are shown in Table 16.2.

TABLE 16.2

| | Amino Acid | FIOP Product |
|---|---|---|
| SEQ | Differences | Peak Area |
| ID NO: | (Relative to | Relative to |
| (nt/aa) | SEQ ID NO: 882) | SEQ ID NO: 882 |
| 1205/1206 | R394L | +++ |
| 1207/1208 | R394T | +++ |
| 1209/1210 | R461S | +++ |
| 1211/1212 | R394M | +++ |
| 1213/1214 | R461G | +++ |
| 1215/1216 | R461A | +++ |
| 1217/1218 | F462L | +++ |
| 1219/1220 | R477Q | +++ |
| 1221/1222 | T203M | +++ |
| 1223/1224 | R394S | +++ |
| 1225/1226 | D199R | +++ |
| 1227/1228 | A495G | +++ |
| 1229/1230 | D199A | +++ |
| 1231/1232 | T484R | +++ |
| 1233/1234 | I408L | +++ |
| 1235/1236 | T484M | ++ |
| 1237/1238 | D492S | ++ |
| 1239/1240 | V273W | ++ |
| 1241/1242 | D314I | ++ |
| 1243/1244 | C201A | ++ |
| 1245/1246 | D199G | ++ |
| 1247/1248 | N175V | ++ |
| 1249/1250 | D492T | ++ |
| 1251/1252 | A313R | ++ |
| 1253/1254 | D199H | ++ |
| 1255/1256 | T275D | ++ |
| 1257/1258 | A495S | ++ |
| 1259/1260 | A313Q | ++ |
| 1261/1262 | D199Q | ++ |
| 1263/1264 | C307M | ++ |
| 1265/1266 | G350S | ++ |
| 1267/1268 | Q179R | ++ |
| 1269/1270 | S325V | ++ |
| 1271/1272 | D199I | + |
| 1273/1274 | R481W | + |
| 1275/1276 | D199V | + |
| 1277/1278 | D199E | + |
| 1279/1280 | C307V | + |
| 1281/1282 | T203R | + |
| 1283/1284 | A319R | + |
| 1285/1286 | G272T | + |
| 1287/1288 | A321K | + |
| 1289/1290 | N175D | + |
| 1291/1292 | S325A | + |
| 1293/1294 | V273M | + |
| 1295/1296 | D322K | + |
| 1297/1298 | F462Q | + |
| 1299/1300 | I408G | + |
| 1301/1302 | C201W | + |
| 1303/1304 | N175I | + |
| 1305/1306 | R196G | + |
| 1307/1308 | P404F | + |
| 1309/1310 | I408R | + |
| 1311/1312 | E523H | + |
| 1313/1314 | D322Q | + |
| 1315/1316 | D314G | + |
| 1317/1318 | A319G | + |
| 1319/1320 | A313S | + |
| 1321/1322 | R406V | + |
| 1323/1324 | L491I | + |
| 1325/1326 | R461Q | + |
| 1327/1328 | Q376V | + |
| 1329/1330 | A313M | + |
| 1331/1332 | P404W | + |
| 1333/1334 | I324V | + |

Levels of increased activity were determined relative to the reference polypeptide of SEQ ID NO: 882 and defined as follows: “+” 1.10 to 1.82-fold increased activity; “++” >1.82-fold increased activity; “+++” >3.14-fold increased activity.

### Example 17

### Improvements Over SEQ ID NO: 1336 in the Extension of Oligonucleotide Acceptor Molecules with Nucleotide Triphosphate Donors

### HTP Screening for Improved TdT Variants

SEQ ID NO: 1336 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 17.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 17.1-Reaction conditions

Lysis buffer-TEA (50 mM, pH 7.5), 0.1 g/L lysozyme;
Reaction buffer-20 mM TEA, 0.25 mM CoCl2,
pH 7.8; Lysate concentration (vol %)-25;
Preincubation Temp (C.)-48° C.; Preincubation Time (min)-
60; Reaction temperature (C.)-48° C.;
Reaction Time (min)-30; Reaction volume (μL) -1; NTP (μM)-
*rGTP; NTP conc (μM)-25; Oligonucleotide-5′-6-FAM-T15AmG*mC;
Substrate conc (μM)-1;
Product -5′-6-FAM-T15AmG*mC*rG.

Activity relative to SEQ ID NO: 1336 (Activity FIOP) was calculated as the percent product of the variant compared with the percent product observed by the reaction with SEQ ID NO: 1336 (where the percent product may be set as the average of replicates or else the highest single sample as appropriate). The results are shown in Table 17.2.

TABLE 17.2

| SEQ ID | Amino Acid Differences | FIOP Product Peak |
|---|---|---|
| NO: | (Relative to | Area Relative to |
| (nt/aa) | SEQ ID NO: 1336) | SEQ ID NO: 1336 |
| 1337/1338 | T203A/T344V/R394E | +++ |
| 1339/1340 | T203A/K297D/D314R/R394E/ | +++ |
| | E395W | |
| 1341/1342 | K195E/T203A/K278E/R394E/ | +++ |
| | E395W | |
| 1343/1344 | T344V/R394E/E395W | +++ |
| 1345/1346 | K195E/T203A/R394E | +++ |
| 1347/1348 | D314R/R394E/E395W | ++ |
| 1349/1350 | R394E | ++ |
| 1351/1352 | D199R/T203A/K297D/R394E/ | ++ |
| | E395W | |
| 1353/1354 | T203A/R394E | ++ |
| 1355/1356 | K195E/R394E/E395W | ++ |
| 1357/1358 | T203A/K278E/K297D/R394E | ++ |
| 1359/1360 | I15F/D199R/T203A/R394E | ++ |
| 1361/1362 | K195E/D199R/T203A/K278E/ | ++ |
| | D314R/F353K/R394E | |
| 1363/1364 | K195E/K278E/K297D/R394E/ | + |
| | E395W | |
| 1365/1366 | K297D/R394E | + |
| 1367/1368 | K195E/D199R/T203A/K278E/ | + |
| | K297D/R394E | |
| 1369/1370 | K195E/T203A/K297D/D314R/ | + |
| | R394E | |
| 1371/1372 | T203A/R394E/E395W | + |
| 1373/1374 | K195E/K278E/K297D/R394E | + |
| 1375/1376 | K195E/T203A/K297D/R394E/ | + |
| | L419M | |
| 1377/1378 | K195E/D314R/T344V | + |
| 1379/1380 | K28N/T344V/F353K/E395W | + |
| 1381/1382 | T203A/E310R/D314R/R394E/ | + |
| | E395W/L419M | |
| 1383/1384 | T203A/F353K | + |
| 1385/1386 | I15F/D199R/R394E | + |
| 1387/1388 | F353K | + |

Levels of increased activity were determined relative to the reference polypeptide of SEQ ID NO: 1336 and defined as follows:
“+” 1.15 to 3.70-fold increased activity;
“++” >3.70-fold increased activity;
“+++” >5.18-fold increased activity.

### Example 18

### Improvements Over SEQ ID NO: 1336 in the Extension of Oligonucleotide Acceptor Molecules with Nucleotide Triphosphate Donors

### HTP Screening for Improved TdT Variants

SEQ ID NO: 1336 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 18.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 18.1
Reaction conditions

Lysis buffer-TEA (50 mM, pH 7.5), 0.1 g/L lysozyme; Reaction buffer-20
mM TEA, 0.25 mM CoCl2, pH 7.8; Lysate concentration (vol %)-25;
Preincubation Temp (C.)-50° C.; Preincubation Time (min)-60; Reaction
temperature (C.)-48° C.; Reaction Time (min)-30; Reaction volume
(μL) -1; NTP (μM)-mGTP; NTP conc (μM)-50; Oligonucleotide-5′-6-
FAM-T12TATCAC*mC; Substrate conc (μM)-1; Product -5′-6-FAM-
T12TATCAC*mCmG.

Activity relative to SEQ ID NO: 1336 (Activity FIOP) was calculated as the percent product of the variant compared with the percent product observed by the reaction with SEQ ID NO: 1336 (where the percent product may be set as the average of replicates or else the highest single sample as appropriate). The results are shown in Table 18.2.

TABLE 18.2

| SEQ ID | Amino Acid Differences | FIOP Product Peak |
|---|---|---|
| NO: | (Relative to | Area Relative to |
| (nt/aa) | SEQ ID NO: 1336) | SEQ ID NO: 1336 |
| 1389/1390 | D330E | +++ |
| 1391/1392 | W333H | +++ |
| 1393/1394 | W333R | +++ |
| 1395/1396 | L340S | +++ |
| 1397/1398 | S292L/L411P | +++ |
| 1399/1400 | M490R | +++ |
| 1401/1402 | I503E | +++ |
| 1403/1404 | H177L | +++ |
| 1405/1406 | M435C | +++ |
| 1407/1408 | D460G | +++ |
| 1409/1410 | W333G | +++ |
| 1411/1412 | W333A | +++ |
| 1413/1414 | T200C/H425K | +++ |
| 1415/1416 | L326T | +++ |
| 1417/1418 | L340R | +++ |
| 1419/1420 | V219R | +++ |
| 1421/1422 | D338T | +++ |
| 1423/1424 | T160M | +++ |
| 1425/1426 | S446P | +++ |
| 1427/1428 | L340M/G438V | +++ |
| 1429/1430 | Q373H | +++ |
| 1431/1432 | T334R | ++ |
| 1433/1434 | L326N | ++ |
| 1435/1436 | T334S | ++ |
| 1437/1438 | L340A | ++ |
| 1439/1440 | S363C | ++ |
| 1441/1442 | I503Q | ++ |
| 1443/1444 | H425R | ++ |
| 1445/1446 | I503V | ++ |
| 1447/1448 | E369N | ++ |
| 1449/1450 | M435T | ++ |
| 1451/1452 | S292T | ++ |
| 1453/1454 | M435A | ++ |
| 1455/1456 | L340M | ++ |
| 1457/1458 | W333D | ++ |
| 1459/1460 | D400A/G401E/K402F | ++ |
| 1461/1462 | Y459Q | ++ |
| 1463/1464 | M435K | ++ |
| 1465/1466 | Q373N | ++ |
| 1467/1468 | T160S | ++ |
| 1469/1470 | E217Q | ++ |
| 1471/1472 | M490V | ++ |
| 1473/1474 | K427Q | ++ |
| 1475/1476 | E369M | ++ |
| 1477/1478 | M435E | ++ |
| 1479/1480 | M435S | ++ |
| 1481/1482 | T61A | ++ |
| 1483/1484 | T160N | ++ |
| 1485/1486 | F504W | ++ |
| 1487/1488 | A444R | ++ |
| 1489/1490 | T162A | ++ |
| 1491/1492 | C213S | + |
| 1493/1494 | L326C | + |
| 1495/1496 | M435G | + |
| 1497/1498 | S446E | + |
| 1499/1500 | Q329K | + |
| 1501/1502 | Q370G | + |
| 1503/1504 | I503R | + |
| 1505/1506 | D372G | + |
| 1507/1508 | D223Q | + |
| 1509/1510 | T200V | + |
| 1511/1512 | K502G | + |
| 1513/1514 | T160V | + |
| 1515/1516 | L326M | + |
| 1517/1518 | H425T | + |
| 1519/1520 | T295S | + |
| 1521/1522 | K383R | + |
| 1523/1524 | L248S | + |
| 1525/1526 | E441N | + |
| 1527/1528 | T437S | + |
| 1529/1530 | S442G | + |
| 1531/1532 | K506E | + |
| 1533/1534 | M490H | + |
| 1535/1536 | M490E | + |
| 1537/1538 | L340G | + |
| 1539/1540 | G236I | + |
| 1541/1542 | T334E | + |
| 1543/1544 | T437Q | + |
| 1545/1546 | M435Q | + |
| 1547/1548 | M435S/I503M | + |
| 1549/1550 | G236V | + |
| 1551/1552 | K427E | + |
| 1553/1554 | H425D | + |
| 1555/1556 | K501A | + |
| 1557/1558 | F504R | + |
| 1559/1560 | V219L | + |
| 1561/1562 | K502R | + |
| 1563/1564 | E246K | + |
| 1565/1566 | E443T | + |
| 1567/1568 | L248C | + |
| 1569/1570 | R152H/I503S | + |
| 1571/1572 | T200C | + |
| 1573/1574 | G236P | + |
| 1575/1576 | E369G | + |
| 1577/1578 | L326S | + |
| 1579/1580 | N440V | + |
| 1581/1582 | K488S | + |
| 1583/1584 | T200K | + |
| 1585/1586 | Q329R | + |
| 1587/1588 | N440E | + |
| 1589/1590 | Q165K | + |
| 1591/1592 | M490W | + |
| 1593/1594 | F504N | + |

Levels of increased activity were determined relative to the reference polypeptide of SEQ ID NO: 1336 and defined as follows:
“+” 1.00 to 1.31-fold increased activity;
“++” >1.31-fold increased activity;
“+++” >1.67-fold increased activity.

### Example 19

### Improvements Over SEQ ID NO: 1348 in the Extension of Oligonucleotide Acceptor Molecules with Nucleotide Triphosphate Donors

### HTP Screening for Improved TdT Variants

SEQ ID NO: 1348 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 19.1.

Reactions were performed in 96-well format 200 μL BioRad PCR plates. Reactions included 1-10 M oligonucleotide, 25-50 HM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 19.1
Reaction conditions

Lysis buffer-TEA (50 mM, pH 7.5), 0.1 g/L lysozyme; Reaction buffer-20
mM TEA, 0.25 mM CoCl2, pH 7.8; Lysate concentration (vol %)-25;
Preincubation Temp (C.)-50° C.; Preincubation Time (min)-60; Reaction
temperature (C.)-50° C.; Reaction Time (min)-30; Reaction volume
(μL) -1; NTP (μM)-mGTP; NTP conc (μM)-50; Oligonucleotide-5′-
6-FAM-T15AmG*mC; Substrate conc (μM)-1; Product -5′-6-FAM-
T15AmG*mCmG.

Activity relative to SEQ ID NO: 1348 (Activity FIOP) was calculated as the percent product of the variant compared with the percent product observed by the reaction with SEQ ID NO: 1348 (where the percent product may be set as the average of replicates or else the highest single sample as appropriate). The results are shown in Table 19.2.

TABLE 19.2

| SEQ ID | Amino Acid Differences | FIOP Product Peak |
|---|---|---|
| NO: | (Relative to | Area Relative to |
| (nt/aa) | SEQ ID NO: 1348) | SEQ ID NO: 1348 |
| 1595/1596 | C201A/C213S/W333A/T344V/R397Q/ | +++ |
| | H425D/R481W/H485D | |
| 1597/1598 | N175D/C201A/W333A/D412N/H425D/ | +++ |
| | C457V/H485D | |
| 1599/1600 | W333A/T344V/E369N/H485D | +++ |
| 1601/1602 | N175D/W333A/H485D | +++ |
| 1603/1604 | N175D/H485D | +++ |
| 1605/1606 | C201A/W333A/T344V/C457V/R481W | +++ |
| 1607/1608 | C201A/W333A/R481W | ++ |
| 1609/1610 | H3Q/N175D/C213S/A313R/S325T/ | ++ |
| | L340R/C457V/R481W/H485D | |
| 1611/1612 | H3Q/C307M/A321K/L340R/F353K/ | ++ |
| | R406G/I408A/K445N | |
| 1613/1614 | P148T/N175D/C201A/C457V/H485D | ++ |
| 1615/1616 | T344V/H485D | ++ |
| 1617/1618 | C307M/Q373H/R406G/I408A/T484M | ++ |
| 1619/1620 | W333G | ++ |
| 1621/1622 | D199H/C307M/A321K/L340R/R406G/ | ++ |
| | I408A/K445N/T484M | |
| 1623/1624 | L340R/T484M | ++ |
| 1625/1626 | N175D/W333A | + |
| 1627/1628 | A321K/W333G/L340R | + |
| 1629/1630 | C213S/W333A/R397Q | + |
| 1631/1632 | C307M/W333G/L340R/I408A/K445N/ | + |
| | F462L/K502G | |
| 1633/1634 | S325T/H425D/C457V/R481W | + |
| 1635/1636 | S325T/W333A/E369N/H425D | + |
| 1637/1638 | C201R/R406G/I408A/F462L/T484M/ | + |
| | K502G | |
| 1639/1640 | R406G/I408A | + |
| 1641/1642 | F353K/R406G | + |
| 1643/1644 | N175D/W333A/E369N/R481W | + |
| 1645/1646 | Q373H | + |
| 1647/1648 | W333A/T344V/E369N/R397Q | + |
| 1649/1650 | C213S | + |
| 1651/1652 | N175D/S325T/R397Q | + |

Levels of increased activity were determined relative to the reference polypeptide of SEQ ID NO: 1348 and defined as follows:
“+” 1.33 to 2.18-fold increased activity;
“++” >2.18-fold increased activity;
“+++” >3.12-fold increased activity.

### Example 20

### Improvements Over SEQ ID NO: 1596 in the Extension of Oligonucleotide Acceptor Molecules with Nucleotide Triphosphate Donors

### HTP Screening for Improved TdT Variants

SEQ ID NO: 1596 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 20.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 20.1
Reaction conditions

Lysis buffer-TEA (50 mM, pH 7.5), 0.1 g/L lysozyme; Reaction buffer-20
mM TEA, 0.25 mM CoCl2, pH 7.8; Lysate concentration (vol %)-25;
Preincubation Temp (C.)-50° C.; Preincubation Time (min)-60; Reaction
temperature (C.)-50° C.; Reaction Time (min)-30; Reaction volume
(μL) -1; NTP (μM)-mATP; NTP conc (μM)-50; Oligonucleotide-5′-
6-FAM-T15AmU*mG; Substrate conc (μM)-10; Product -5′-6-FAM-
T15AmU*mG mA.

Activity relative to SEQ ID NO: 1596 (Activity FIOP) was calculated as the percent product of the variant compared with the percent product observed by the reaction with SEQ ID NO: 1596 (where the percent product may be set as the average of replicates or else the highest single sample as appropriate). The results are shown in Table 20.2.

TABLE 20.2

| SEQ ID | Amino Acid Differences | FIOP Product Peak |
|---|---|---|
| NO: | (Relative to | Area Relative to |
| (nt/aa) | SEQ ID NO: 1596) | SEQ ID NO: 1596 |
| 1653/1654 | R406G/I408A/S442G/S446P | ++ |
| 1655/1656 | R406G/I408A | ++ |
| 1657/1658 | T160M/V219R/D460G/I503E/K506E | ++ |
| 1659/1660 | S446P | ++ |
| 1661/1662 | I408A | + |
| 1663/1664 | V219R/C307M/L326T | + |
| 1665/1666 | R406G/I408A/M490R | + |
| 1667/1668 | A252K/A333H | + |
| 1669/1670 | A252K | + |

Levels of increased activity were determined relative to the reference polypeptide of SEQ ID NO: 1596 and defined as follows:
“+” 1.12 to 1.50-fold increased activity;
“++” >1.50-fold increased activity.

### Example 21

### Improvements Over SEQ ID NO: 1596 in the Extension of Oligonucleotide Acceptor Molecules with Nucleotide Triphosphate Donors

### HTP Screening for Improved TdT Variants

SEQ ID NO: 1596 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 21.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 21.1
Reaction conditions

Lysis buffer-TEA (50 mM, pH 7.5), 0.1 g/L lysozyme; Reaction buffer-20
mM TEA, 0.25 mM CoCl2, pH 7.8; Lysate concentration (vol %)-25;
Preincubation Temp (C.)-50° C.; Preincubation Time (min)-60; Reaction
temperature (C.)-50° C.; Reaction Time (min)-30; Reaction volume
(μL) -1; NTP (μM)-mATP; NTP conc (μM)-50; Oligonucleotide-5′-
6-FAM-T15AmU*mG; Substrate conc (μM)-10; Product -5′-6-FAM-
T15AmU*mGmA.

Activity relative to SEQ ID NO: 1596 (Activity FIOP) was calculated as the percent product of the variant compared with the percent product observed by the reaction with SEQ ID NO: 1596 (where the percent product may be set as the average of replicates or else the highest single sample as appropriate). The results are shown in Table 21.2.

TABLE 21.2

| SEQ ID | Amino Acid Differences | FIOP Product Peak |
|---|---|---|
| NO: | (Relative to | Area Relative to |
| (nt/aa) | SEQ ID NO: 1596) | SEQ ID NO: 1596 |
| 1671/1672 | T484E | +++ |
| 1673/1674 | Q179K | +++ |
| 1675/1676 | F353Q | +++ |
| 1677/1678 | W382L | +++ |
| 1679/1680 | C307L | +++ |
| 1681/1682 | M205R | +++ |
| 1683/1684 | K402Q | +++ |
| 1685/1686 | K488N | +++ |
| 1687/1688 | H413G | ++ |
| 1689/1690 | Q165P | ++ |
| 1691/1692 | Q376H | ++ |
| 1693/1694 | M490L | ++ |
| 1695/1696 | Q329K | ++ |
| 1697/1698 | L393I | ++ |
| 1699/1700 | R406P | ++ |
| 1701/1702 | D400E | ++ |
| 1703/1704 | R171K | ++ |
| 1705/1706 | I163V | ++ |
| 1707/1708 | S325L | ++ |
| 1709/1710 | S261A | ++ |
| 1711/1712 | S442A | ++ |
| 1713/1714 | D330E | + |
| 1715/1716 | S405N | + |
| 1717/1718 | E371D | + |
| 1719/1720 | K508D | + |
| 1721/1722 | A410Q | + |
| 1723/1724 | A495G | + |
| 1725/1726 | A252E | + |
| 1727/1728 | L188M | + |
| 1729/1730 | I208A | + |
| 1731/1732 | E520D | + |
| 1733/1734 | K506S | + |
| 1735/1736 | V253I | + |
| 1737/1738 | T162H | + |
| 1739/1740 | L419A | + |
| 1741/1742 | D460E | + |
| 1743/1744 | K407A | + |
| 1745/1746 | E441M | + |
| 1747/1748 | E233D | + |
| 1749/1750 | H177Y | + |
| 1751/1752 | T200A | + |
| 1753/1754 | F464Y | + |
| 1755/1756 | D277E | + |
| 1757/1758 | Q260K | + |

Levels of increased activity were determined relative to the reference polypeptide of SEQ ID NO: 1596 and defined as follows:
“+” 1.00 to 1.31-fold increased activity;
“++” >1.31-fold increased activity;
“+++” >1.58-fold increased activity.

### Example 22

### Improvements Over SEQ ID NO: 1596 in the Extension of Oligonucleotide Acceptor Molecules with Nucleotide Triphosphate Donors

### HTP Screening for Improved TdT Variants

SEQ ID NO: 1596 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 22.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 22.1
Reaction conditions

Lysis buffer-TEA (50 mM, pH 7.5), 0.1 g/L lysozyme; Reaction buffer-20
mM TEA, 0.25 mM CoCl2, pH 7.8; Lysate concentration (vol %)-25;
Preincubation Temp (C.)-57° C.; Preincubation Time (min)-60; Reaction
temperature (C.)-57° C.; Reaction Time (min)-60; Reaction volume
(μL)-1; NTP (μM)-*ddGTP; NTP conc (μM)-50; Oligonucleotide-5′-6-
FAM-T14ATCmC; Substrate conc (μM)-1; Product -5′-6-FAM-
T14ATCmC*ddG.

Activity relative to SEQ ID NO: 1596 (Activity FIOP) was calculated as the percent product of the variant compared with the percent product observed by the reaction with SEQ ID NO: 1596 (where the percent product may be set as the average of replicates or else the highest single sample as appropriate). The results are shown in Table 22.2.

TABLE 22.2

| SEQ ID | Amino Acid Differences | FIOP Product Peak |
|---|---|---|
| NO: | (Relative to | Area Relative to |
| (nt/aa) | SEQ ID NO: 1596) | SEQ ID NO: 1596 |
| 1671/1672 | T484E | +++ |
| 1759/1760 | S325L | +++ |
| 1761/1762 | M205R | +++ |
| 1763/1764 | Q165P | +++ |
| 1765/1766 | M490L | +++ |
| 1675/1676 | F353Q | +++ |
| 1745/1746 | E441M | +++ |
| 1709/1710 | S261A | +++ |
| 1767/1768 | K488N | +++ |
| 1769/1770 | D460E | ++ |
| 1771/1772 | A252E | ++ |
| 1773/1774 | C307L | ++ |
| 1775/1776 | F414H | ++ |
| 1673/1674 | Q179K | ++ |
| 1719/1720 | K508D | ++ |
| 1729/1730 | I208A | ++ |
| 1777/1778 | R406P | ++ |
| 1779/1780 | G518D | ++ |
| 1705/1706 | I163V | ++ |
| 1755/1756 | D277E | ++ |
| 1781/1782 | Q370T | ++ |
| 1783/1784 | E371D | ++ |
| 1785/1786 | N380D | ++ |
| 1721/1722 | A410Q | ++ |
| 1787/1788 | K402Q | + |
| 1789/1790 | L306F | + |
| 1791/1792 | Q376H | + |
| 1793/1794 | K506S | + |
| 1695/1696 | Q329K | + |
| 1795/1796 | K368R | + |
| 1727/1728 | L188M | + |
| 1797/1798 | H413G | + |
| 1799/1800 | S442A | + |
| 1801/1802 | K161R | + |
| 1803/1804 | V253I | + |
| 1805/1806 | L419A | + |
| 1807/1808 | A321K | + |
| 1753/1754 | F464Y | + |
| 1809/1810 | Q251K | + |
| 1747/1748 | E233D | + |
| 1811/1812 | H426P | + |
| 1813/1814 | L393I | + |
| 1723/1724 | A495G | + |
| 1815/1816 | G231T | + |
| 1817/1818 | L327I | + |
| 1701/1702 | D400E | + |
| 1819/1820 | T162H | + |

Levels of increased activity were determined relative to the reference polypeptide of SEQ ID NO: 1596 and defined as follows:
“+” 1.00 to 1.84-fold increased activity;
“++” >1.84-fold increased activity;
“+++” >3.00-fold increased activity.

### Example 23

### Improvements Over SEQ ID NO: 1654 in the Extension of Oligonucleotide Acceptor Molecules with Nucleotide Triphosphate Donors

### HTP Screening for Improved TdT Variants

SEQ ID NO: 1654 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 23.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 23.1
Reaction conditions

Lysis buffer-TEA (50 mM, pH 7.5), 0.1 g/L lysozyme; Reaction buffer-20
mM TEA, 0.25 mM CoCl2, pH 7.8; Lysate concentration (vol %)-25;
Preincubation Temp (C.)-50° C.; Preincubation Time (min)-60; Reaction
temperature (C.)-50° C.; Reaction Time (min)-45; Reaction volume
(μL) -1; NTP (μM)-3′P-mATP; NTP conc (μM)-50; Oligonucleotide-5′-
6-FAM-T15AT*mG; Substrate conc (μM)-10; Product -5′-6-FAM-
T15AT*mGmA-3′P.

Activity relative to SEQ ID NO: 1654 (Activity FIOP) was calculated as the percent product of the variant compared with the percent product observed by the reaction with SEQ ID NO: 1654 (where the percent product may be set as the average of replicates or else the highest single sample as appropriate). The results are shown in Table 23.2.

TABLE 23.2

| SEQ ID | Amino Acid Differences | FIOP Product Peak |
|---|---|---|
| NO: | (Relative to | Area Relative to |
| (nt/aa) | SEQ ID NO: 1654) | SEQ ID NO: 1654 |
| 1821/1822 | T160M/Q165P/M205R/V219R/E441M | ++ |
| 1823/1824 | M205R/C307L/E441M/D460G/K488N | ++ |
| 1825/1826 | E441M | ++ |
| 1827/1828 | T160M/T203E/M205R/E441M | + |
| 1829/1830 | T160M/Q165P/T203E/M205R/V219R/ | + |
| | F353Q/D460G/K488N | |
| 1831/1832 | Q179K/F353Q | + |
| 1833/1834 | T160M/V219R/D330K/T484E | + |

Levels of increased activity were determined relative to the reference polypeptide of SEQ ID NO: 1654 and defined as follows:
“+” 1.47 to 1.83-fold increased activity;
“++” >1.83-fold increased activity.

### Example 24

### Improvements Over SEQ ID NO: 1654 in the Extension of Oligonucleotide Acceptor Molecules with Nucleotide Triphosphate Donors

### HTP Screening for Improved TdT Variants

SEQ ID NO: 1654 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 24.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 24.1
Reaction conditions

Lysis buffer-TEA (50 mM, pH 7.5), 0.1 g/L lysozyme; Reaction buffer-20
mM TEA, 0.25 mM CoCl₂, pH 7.8; Lysate concentration (vol %)-25;
Preincubation Temp (C.)-57° C.; Preincubation Time (min)-60; Reaction
temperature (C.)-57° C.; Reaction Time (min)-60; Reaction volume
(μL) -1; NTP (μM)-*ddGTP; NTP conc (μM)-50; Oligonucleotide-5′-
6-FAM-T14ATCmC; Substrate conc (μM)-1; Product -5′-6-FAM-
T14ATCmC*ddG.

Activity relative to SEQ ID NO: 1654 (Activity FIOP) was calculated as the percent product of the variant compared with the percent product observed by the reaction with SEQ ID NO: 1654 (where the percent product may be set as the average of replicates or else the highest single sample as appropriate). The results are shown in Table 24.2.

TABLE 24.2

| SEQ ID | Amino Acid Differences | FIOP Product Peak |
|---|---|---|
| NO: | (Relative to | Area Relative to |
| (nt/aa) | SEQ ID NO: 1654) | SEQ ID NO: 1654 |
| 1835/1836 | Q165P/T203E/M205R/C307L/F414Y/ | +++ |
| | E441M/T484E/A495G/I503E/K506E | |
| 1837/1838 | Q165P/Q179K/T203E/M205R/T484E/ | +++ |
| | I503E | |
| 1839/1840 | T160M/Q179K/T203E/M205R/I208A/ | +++ |
| | V219R/E371D/F414H/T484E/K506E | |
| 1841/1842 | T160M/M205R/I208A/V219R/L326T/ | +++ |
| | E441M/T484E/K488N/I503E | |
| 1843/1844 | T160M/I208A/E371D/E441M/T484E/ | +++ |
| | K506E | |
| 1845/1846 | T160M/Q179K/I208A/V219R/C307L/ | +++ |
| | H413G/F414H/I503E/K506E | |
| 1847/1848 | T160M/Q179K/C307L/Q376H/E441M/ | +++ |
| | K488N/I503E | |
| 1829/1830 | T160M/Q165P/T203E/M205R/V219R/ | +++ |
| | F353Q/D460G/K488N | |
| 1833/1834 | T160M/V219R/D330K/T484E | +++ |
| 1849/1850 | Q179K/M205R/I208A/F353Q/F414H/ | +++ |
| | E441M/D460G/T484E/K488N | |
| 1851/1852 | T160M/Q165P/C307L/F353Q/H413G/ | +++ |
| | F414H/E441M/K488N/A495G | |
| 1853/1854 | Q165P/Q179K/I208A/F353Q/H413G/ | +++ |
| | F414H/I503E | |
| 1821/1822 | T160M/Q165P/M205R/V219R/E441M | ++ |
| 1855/1856 | Q165P/T203E/M205R/T484E/K488N | ++ |
| 1857/1858 | T160M/T203E/M205R/I208A/H413G/ | ++ |
| | D460G/T484E/K488N | |
| 1859/1860 | M205R/L326T/K488N/I503E/K506E | ++ |
| 1861/1862 | T160M/I208A/L326T/Q376H/F414H/ | ++ |
| | E441M/T484E/K488N | |
| 1863/1864 | T160M/V219R/C307L/E371D/K506E | ++ |
| 1865/1866 | H3Q/T160M/M205R/I208A/V219R/ | ++ |
| | C307L/F353Q/E371D/Q376H/H413G/ | |
| | F414H/E441M/K488N | |
| 1867/1868 | M205R/V219R/C307L/F353Q | ++ |
| 1869/1870 | Q165P/I208A/L326T/H413G/F414H/ | ++ |
| | T484E/A495G/K506E | |
| 1823/1824 | M205R/C307L/E441M/D460G/K488N | ++ |
| 1871/1872 | T160M/Q165P/Q179K/T203E/M205R/ | ++ |
| | I208A/V219R/F353Q/Q376H/H413G/ | |
| | F414H/E441M/K488N | |
| 1873/1874 | M205R/C307L | ++ |
| 1875/1876 | T203E/I208A/V219R/E441M | ++ |
| 1877/1878 | T160M/T203E/L326T/F353Q/H413G/ | ++ |
| | F414H/T484E/A495G | |
| 1879/1880 | T160M/I208A/F414H/E441M/V452I/ | ++ |
| | R480H/K488N/A495G | |
| 1881/1882 | Q165P/M205R | ++ |
| 1831/1832 | Q179K/F353Q | ++ |
| 1883/1884 | T160M/T203E/M205R/I208A/V219R/ | ++ |
| | F414H/D460G/K506E | |
| 1885/1886 | T160M/T203E/I208A/H413G/F414H/ | ++ |
| | E441M/T484E | |
| 1887/1888 | Q165P/Q179K/T203E/M205R/V219R/ | + |
| | F414H/F418I/E441M/K488N/I503E | |
| 1889/1890 | T203E/M205R/I208A/F353Q | + |
| 1891/1892 | T160M/Q165P/M205R | + |
| 1893/1894 | T203E/I208A/V219R/Q376H/E441M | + |
| 1895/1896 | T160M/Q165P/Q179K/T203E/C307L/ | + |
| | Q376H/H413G/A495G | |
| 1897/1898 | Q179K/T203E/I208A/L326T/F353Q/ | + |
| | Q376H/T484E | |
| 1899/1900 | T160M/Q165P/M205R/F414H/E441M/ | + |
| | A495G | |
| 1901/1902 | Q165P/Q179K/M205R/H413G/E441M | + |
| 1903/1904 | I208A/K488N/K506E | + |
| 1905/1906 | Q179K/I208A/F353Q/D460G | + |
| 1907/1908 | T160M/Q165P/T203E/M205R/C307L/ | + |
| | E371D/Q376H/H413G/F414H | |
| 1909/1910 | T160M/T203E/I208A/E371D/H413G/ | + |
| | F414H | |
| 1911/1912 | T160M/T203E/I208A/C307L/F353Q/ | + |
| | A495G | |
| 1913/1914 | T160M | + |
| 1827/1828 | T160M/T203E/M205R/E441M | + |
| 1915/1916 | T203E/I208A/L326T/F353Q | + |
| 1917/1918 | M205R/I208A/C307L/F353Q/Q376H/ | + |
| | H413G | |
| 1919/1920 | T203E/M205R/I208A/C307L/E441M | + |
| 1921/1922 | Q179K/M205R/F353Q | + |
| 1923/1924 | T203E/H413G/I503E/K506E | + |
| 1925/1926 | T160M/Q165P/T203E/I208A/E371D/ | + |
| | Q376H/H413G/F414H | |
| 1927/1928 | M205R/I208A/F414H | + |
| 1929/1930 | Q165P/T203E/M205R | + |
| 1931/1932 | M205R/C307L/Q376H/F414H/E441M/ | + |
| | A495G | |
| 1933/1934 | T160M/Q179K/I208A/C307L/E371D/ | + |
| | Q376H/F414H | |
| 1935/1936 | T203E/M205R/I208A/C307L/D330N/ | + |
| | F353Q/E441M/D460G/I503E/K506E | |
| 1937/1938 | Q165P/T203E/I208A/L326T/Q376H/ | + |
| | I503E | |
| 1939/1940 | T160M/Q165P/M205R/I208A | + |
| 1941/1942 | Q179K/T203E/M205R | + |
| 1943/1944 | Q179K | + |
| 1945/1946 | L326T/F353Q/E371D/Q376H/F414H | + |
| 1825/1826 | E441M | + |

Levels of increased activity were determined relative to the reference polypeptide of SEQ ID NO: 1654 and defined as follows:
“+” 1.09 to 2.31-fold increased activity;
“++” >2.31-fold increased activity;
“+++” >3.89-fold increased activity.

### Example 25

### Improvements Over SEQ ID NO: 1830 in the Extension of Oligonucleotide Acceptor Molecules with Nucleotide Triphosphate Donors

### HTP Screening for Improved TdT Variants

SEQ ID NO: 1830 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 25.1.

Reactions were performed in 96-well format 200 μL BioRad PCR plates. Reactions included 1-10 μM oligonucleotide, 25-50 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 25.1
Reaction conditions

Lysis buffer-TEA (50 mM, pH 7.5), 0.1 g/L lysozyme; Reaction buffer-
20 mM TEA, 0.25 mM CoCl₂, pH 7.8; Lysate concentration (vol %)-25;
Preincubation Temp (C.)-50° C.; Preincubation Time (min)-60; Reaction
temperature (C.)-50° C.; Reaction Time (min)-60; Reaction volume
(μL) -1; NTP (μM)-3′P-mATP; NTP conc (μM)-50;
Oligonucleotide-5′-6-FAM-T15AT*mG; Substrate conc (μM)-10;
Product -5′-6-FAM-T15AT*mGmA-3′P.

Activity relative to SEQ ID NO: 1830 (Activity FIOP) was calculated as the percent product of the variant compared with the percent product observed by the reaction with SEQ ID NO: 1830 (where the percent product may be set as the average of replicates or else the highest single sample as appropriate). The results are shown in Table 25.2.

TABLE 25.2

| SEQ ID | Amino Acid Differences | FIOP Product Peak |
|---|---|---|
| NO: | (Relative to | Area Relative to |
| (nt/aa) | SEQ ID NO: 1830) | SEQ ID NO: 1830 |
| 1947/1948 | I163V/Q179K/D277E/D338T/ | ++ |
| | L340A | |
| 1949/1950 | I163V/F414H/E441M | ++ |
| 1951/1952 | H177L | + |
| 1953/1954 | R171K/T200V/T334R/G406P/ | + |
| | M490L | |
| 1955/1956 | S292T/G406P | + |

Levels of increased activity were determined relative to the reference polypeptide of SEQ ID NO: 1830 and defined as follows:
“+” 1.52 to 2.05-fold increased activity;
“++” >2.05-fold increased activity.

### Example 26

### Activity Improvement Over SEQ ID NO: 1100

Activity with 2′OMe Modified Oligonucleotides and Nucleotide Triphosphates of Shake-Flask Purified TdT Variants

TdT variants of SEQ ID NO: 1100, 1336, and 1958 were produced in shake flask and purified as described in Example 3.

Reactions were performed in 96-well format 200 μL BioRad PCR plates. Reactions included 1 μM oligonucleotide, 25 μM nucleotide triphosphate, 1 μM TdT, 20 mM triethanolamine (pH 7.8), and 250 M cobalt (II) chloride. The reactions were set up as follows: (i) all reaction components, except for TdT, were pre-mixed in a single solution and were aliquoted into each well of the 96-well plates (ii) TdT solution was then added into the wells to initiate the reaction. The reaction plate was heat-sealed with a peelable aluminum seal and incubated in a thermocycler at the indicated temperature and reaction time, then held at 4 mC until the reaction was quenched. Reactions were quenched and processed for CE analysis as described in Example 4.

TABLE 26.1
Reaction buffer-20 mM TEA, 0.25 mM CoCl₂, pH 7.8; Preincubation
Temp (C.) - none; Preincubation Time (min)- none; Reaction temperature
(C.)-50° C.; Reaction Time (min)- 60; Reaction volume (μL)-1; TdT
concentration (μM)- 4; NTP- mATP; NTP conc (μM)-25; Oligonucleotide-
5′-6-FAM-T17mAmUmC, 5′-6-FAM-T17mUmUmC, 5′-6-FAM-
T17mCmUmG; Oligonucleotide conc (μM)- 10; Product- 5′-6-FAM-
T17mAmUmCmA, 5′-6-FAM-T17mUmUmCmA, 5′-6-FAM-
T17mCmUmGmA .

Activity relative to SEQ ID NO: 1100 (Activity FIOP) was calculated as the percent product of the variant compared with the percent product observed by the reaction with SEQ ID NO: 1100 (where the percent product may be set as the average of replicates or else the highest single sample as appropriate). The results are shown in Tables 26.2, 26.3, and 26.4.

TABLE 26.2
Product 5′-6-FAM-T17mAmUmCmA

| SEQ ID | Amino Acid Differences | FIOP Product Peak |
|---|---|---|
| NO: | (Relative to | Area Relative to |
| (nt/aa) | SEQ ID NO: 1100) | SEQ ID NO: 1100 |
| 1957/1958 | L264R | + |
| 1335/1336 | T101E/M137A/L264R/E476R/ | ++ |
| | S525F | |

Levels of increased activity were determined relative to the reference polypeptide of SEQ ID NO: 1100 and defined as follows:
“+” 1.15 to 1.30-fold increased activity;
“++” >1.30-fold increased activity.

TABLE 26.3
Product 5′-6-FAM-T17mUmUmCmA

| SEQ ID | Amino Acid Differences | FIOP Product Peak |
|---|---|---|
| NO: | (Relative to | Area Relative to |
| (nt/aa) | SEQ ID NO: 1100) | SEQ ID NO: 1100 |
| 1957/1958 | L264R | + |
| 1335/1336 | T101E/M137A/L264R/E476R/ | ++ |
| | S525F | |

Levels of increased activity were determined relative to the reference polypeptide of SEQ ID NO: 1100 and defined as follows:
“+” 1.15 to 1.30-fold increased activity;
“++” >1.30-fold increased activity.

TABLE 26.4
Product 5′-6-FAM-T17mCmUmGmA

| SEQ ID | Amino Acid Differences | FIOP Product Peak |
|---|---|---|
| NO: | (Relative to | Area Relative to |
| (nt/aa) | SEQ ID NO: 1100) | SEQ ID NO: 1100 |
| 1957/1958 | L264R | + |
| 1335/1336 | T101E/M137A/L264R/E476R/ | ++ |
| | S525F | |

Levels of increased activity were determined relative to the reference polypeptide of SEQ ID NO: 1100 and defined as follows:
“+” 1.15 to 1.30-fold increased activity;
“++” >1.30-fold increased activity.

### Example 27

### Activity Improvement Over SEQ ID NO: 1654

Activity of Shake-Flask Purified TdT Variants with 3′0-Phosphorylated Nucleotide Triphosphates

TdT variants of SEQ ID NO: 1654, 1830, and 1950 were produced in shake flask and purified as described in Example 3.

Reactions were performed in 96-well format 200 μL BioRad PCR plates. Reactions included 1 μM oligonucleotide, 25 μM nucleotide triphosphate, 1 μM TdT, 20 mM triethanolamine (pH 7.8), and 250 M cobalt (II) chloride. The reactions were set up as follows: (i) all reaction components, except for TdT, were pre-mixed in a single solution and were aliquoted into each well of the 96-well plates (ii) TdT solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 27.1
Reaction buffer-20 mM TEA, 0.25 mM CoCl₂, pH 7.8; Preincubation
Temp (C.) - none; Preincubation Time (min)- none; Reaction temperature
(C.)- 50° C.; Reaction Time (min)- 60; Reaction volume (μL)-1; TdT
concentration (μM)- 4; NTP- 3′phos-mATP; NTP conc (μM)-50;
Oligonucleotide-5′-6-FAM-T15AT*mG, 5′-6-FAM-T15AT*mA, 5′-6-
FAM-T15AT*mC, 5′-6-FAM-T15AT*mU; Oligonucleotide conc (μM)-
25; Product- −5′-6-FAM-T15AT*mGmA-3′phos, 5′-6-FAM-
T15AT*mAmA-3′phos, 5′-6-FAM-T15AT*mCmA-3′phos, 5′-6-FAM-
T15AT*mUmA-3′phos.

Activity relative to SEQ ID NO: 1654 (Activity FIOP) was calculated as the percent product of the variant compared with the percent product observed by the reaction with SEQ ID NO: 1654 (where the percent product may be set as the average of replicates or else the highest single sample as appropriate). The results are shown in Tables 27.2, 27.3, 27.4, and 27.5.

TABLE 27.2
Product 5′-6-FAM-T15AT*mGmA-3′phos

| SEQ ID | Amino Acid Differences | FIOP Product Peak |
|---|---|---|
| NO: | (Relative to | Area Relative to |
| (nt/aa) | SEQ ID NO: 1654) | SEQ ID NO: 1654 |
| 1829/1830 | T160M/Q165P/T203E/M205R/ | + |
| | V219R/F353Q/D460G/K488N | |
| 1949/1950 | T160M/I163V/Q165P/T203E/ | ++ |
| | M205R/V219R/F353Q/F414H/ | |
| | E441M/D460G/K488N | |

Levels of increased activity were determined relative to the reference polypeptide of SEQ ID NO: 1654 and defined as follows:
“+” 1.46 to 2.20-fold increased activity;
“++” >2.20-fold increased activity;
“+++” >4.05-fold increased activity.

TABLE 27.3
Product 5′-6-FAM-T15AT*mAmA-3′phos

| SEQ ID | Amino Acid Differences | FIOP Product Peak |
|---|---|---|
| NO: | (Relative to | Area Relative to |
| (nt/aa) | SEQ ID NO: 1654) | SEQ ID NO: 1654 |
| 1829/1830 | T160M/Q165P/T203E/M205R/ | ++ |
| | V219R/F353Q/D460G/K488N | |
| 1949/1950 | T160M/I163V/Q165P/T203E/ | +++ |
| | M205R/V219R/F353Q/F414H/ | |
| | E441M/D460G/K488N | |

Levels of increased activity were determined relative to the reference polypeptide of SEQ ID NO: 1654 and defined as follows:
“+” 1.46 to 2.20-fold increased activity;
“++” >2.20-fold increased activity;
“+++” >4.05-fold increased activity.

TABLE 27.4
Product 5′-6-FAM-T15AT*mCmA-3′phos

| SEQ ID | Amino Acid Differences | FIOP Product Peak |
|---|---|---|
| NO: | (Relative to | Area Relative to |
| (nt/aa) | SEQ ID NO: 1654) | SEQ ID NO: 1654 |
| 1829/1830 | T160M/Q165P/T203E/M205R/ | ++ |
| | V219R/F353Q/D460G/K488N | |
| 1949/1950 | T160M/I163V/Q165P/T203E/ | ++ |
| | M205R/V219R/F353Q/F414H/ | |
| | E441M/D460G/K488N | |

Levels of increased activity were determined relative to the reference polypeptide of SEQ ID NO: 1654 and defined as follows:
“+” 1.46 to 2.20-fold increased activity;
“++” >2.20-fold increased activity;
“+++” >4.05-fold increased activity.

TABLE 27.5
5′-6-FAM-T15AT*mUmA-3′phos

| SEQ ID | Amino Acid Differences | FIOP Product Peak |
|---|---|---|
| NO: | (Relative to | Area Relative to |
| (nt/aa) | SEQ ID NO: 1654) | SEQ ID NO: 1654 |
| 1829/1830 | T160M/Q165P/T203E/M205R/ | + |
| | V219R/F353Q/D460G/K488N | |
| 1949/1950 | T160M/I163V/Q165P/T203E/ | ++ |
| | M205R/V219R/F353Q/F414H/ | |
| | E441M/D460G/K488N | |

Levels of increased activity were determined relative to the reference polypeptide of SEQ ID NO: 1654 and defined as follows:
“+” 1.46 to 2.20-fold increased activity;
“++” >2.20-fold increased activity;
“+++” >4.05-fold increased activity.

### Example 28

### Activity of Immobilized TdT Enzymes

### Activity of Shake-Flask Purified and Immobilized TdT Variants

TdT variants of SEQ ID NO: 1596, 1654, 1822, 1824, 1826, 1828, 1830, and 1834 were produced in shake flask and purified as described in Example 3.

Enzyme immobilization was performed in 1 mL round bottom costar 96-well plate. A slurry of 25 mg of controlled porosity glass (CPG) with either hydrophilic surface (EziG-1 and EziG-3, EnginZyme) or hydrophobic surface (EziG-2) was prepared in 1 mL of water. 1 mg of EziG (1, 2, or 3) (40 μL of slurry) was transferred to the plate, centrifuged and the supernatant was removed. 0.1 mg of TdT protein solutions (41 μM) were transferred to wells containing solid support. Enough storage buffer (20 mM Tris pH 7.4, 100 mM KC, 0.1 mM EDTA) was added to reach a 50 μL volume. The plate was sealed and gently shaken at room temperature. After 24 hours, the contents were centrifuged, and the supernatant was removed. The immobilized variants were washed twice with reaction buffer (20 mM triethanolamine pH 7.8), centrifuged, and the supernatant was removed. To each well was added 40 μL of reaction mixture including: 50 M 18-mer oligonucleotide (99% unlabeled T14-ATC-mC and 1% FAM-T14-ATC-mC), 100 μM 2′,3′-dideoxyguanosine-5′-triphosphate ddGTP, 0.5 mU/μL E. coli pyrophosphatase (New England Biolabs), 20 mM triethanolamine (pH 7.8), and 250 μM cobalt (II) chloride. The plate was sealed and shaken at 500 rpm at 50° C. for 90 minutes. Reactions were quenched by the addition of 120 μL of 35 mM EDTA. Quenched reactions were analyzed by capillary electrophoresis as described in Example 4.

Activity relative to SEQ ID NO: 1596 (Activity FIOP) was calculated as the percent product of the variant compared with the percent product observed by the reaction with SEQ ID NO: 1596 (where the percent product may be set as the average of replicates or else the highest single sample as appropriate). The results are shown in Tables 28.1, 28.2, and 28.3.

TABLE 28.1
Activity with EziG-1

| SEQ ID | Amino Acid Differences | FIOP Product Peak |
|---|---|---|
| NO: | (Relative to | Area Relative to |
| (nt/aa) | SEQ ID NO: 1596) | SEQ ID NO: 1596 |
| 1653/1654 | R406G/I408A/S442G/S446P | ++ |
| 1821/1822 | T160M/Q165P/M205R/V219R/ | +++ |
| | R406G/I408A/E441M/S442G/ | |
| | S446P | |
| 1833/1834 | T160M/V219R/D330K/R406G/ | +++ |
| | I408A/S442G/S446P/T484E | |
| 1827/1828 | T160M/T203E/M205R/R406G/ | ++ |
| | I408A/E441M/S442G/S446P | |
| 1829/1830 | T160M/Q165P/T203E/M205R/ | +++ |
| | V219R/F353Q/R406G/I408A/ | |
| | S442G/S446P/D460G/K488N | |
| 1825/1826 | R406G/I408A/E441M/S442G/ | ++ |
| | S446P | |
| 1823/1824 | M205R/C307L/R406G/I408A/ | ++ |
| | E441M/S442G/S446P/D460G/ | |
| | K488N | |

Levels of increased activity were determined relative to the reference polypeptide of SEQ ID NO: 1596 and defined as follows:
“+” 1.0 to 1.5-fold increased activity;
“++” >1.5-fold increased activity;
“+++” >2.5-fold increased activity.

TABLE 28.2
Activity with EziG-2

| SEQ ID | Amino Acid Differences | FIOP Product Peak |
|---|---|---|
| NO: | (Relative to | Area Relative to |
| (nt/aa) | SEQ ID NO: 1596) | SEQ ID NO: 1596 |
| 1821/1822 | T160M/Q165P/M205R/V219R/ | + |
| | R406G/I408A/E441M/S442G/ | |
| | S446P | |
| 1833/1834 | T160M/V219R/D330K/R406G/ | + |
| | I408A/S442G/S446P/T484E | |
| 1827/1828 | T160M/T203E/M205R/R406G/ | + |
| | I408A/E441M/S442G/S446P | |
| 1829/1830 | T160M/Q165P/T203E/M205R/ | + |
| | V219R/F353Q/R406G/I408A/ | |
| | S442G/S446P/D460G/K488N | |
| 1823/1824 | M205R/C307L/R406G/I408A/ | + |
| | E441M/S442G/S446P/D460G/ | |
| | K488N | |

Levels of increased activity were determined relative to the reference polypeptide of SEQ ID NO: 1596 and defined as follows:
“+” 1.0 to 1.5-fold increased activity;
“++” >1.5-fold increased activity;
“+++” >2.5-fold increased activity.

TABLE 28.3
Activity with EziG-3

| SEQ ID | Amino Acid Differences | FIOP Product Peak |
|---|---|---|
| NO: | (Relative to | Area Relative to |
| (nt/aa) | SEQ ID NO: 1596) | SEQ ID NO: 1596 |
| 1653/1654 | R406G/I408A/S442G/S446P | + |
| 1821/1822 | T160M/Q165P/M205R/V219R/ | ++ |
| | R406G/I408A/E441M/S442G/ | |
| | S446P | |
| 1833/1834 | T160M/V219R/D330K/R406G/ | ++ |
| | I408A/S442G/S446P/T484E | |
| 1827/1828 | T160M/T203E/M205R/R406G/ | ++ |
| | I408A/E441M/S442G/S446P | |
| 1829/1830 | T160M/Q165P/T203E/M205R/ | ++ |
| | V219R/F353Q/R406G/I408A/ | |
| | S442G/S446P/D460G/K488N | |
| 1825/1826 | R406G/I408A/E441M/S442G/ | ++ |
| | S446P | |
| 1823/1824 | M205R/C307L/R406G/I408A/ | + |
| | E441M/S442G/S446P/D460G/ | |
| | K488N | |

Levels of increased activity were determined relative to the reference polypeptide of SEQ ID NO: 1596 and defined as follows:
“+” 1.0 to 1.5-fold increased activity;
“++” >1.5-fold increased activity;
“+++” >2.5-fold increased activity.

### Improvements Over SEQ ID NO: 1950 in the Extension of Oligonucleotide Acceptor Molecules with Nucleotide Triphosphate Donors

### HTP Screening for Improved TdT Variants

SEQ ID NO: 1950 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 29.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 29.1
Reaction conditions

Lysis buffer-TEA (50 mM, pH 7.5), 0.1 g/L lysozyme; Reaction buffer-20
mM TEA, 0.25 mM CoCl2, pH 7.8; Lysate concentration (vol %)-0.25;
Preincubation Temp (C.)-50° C.; Preincubation Time (min)-60; Reaction
temperature (C.)-50° C.; Reaction Time (min)-60; Reaction volume (μL)-
1; NTP (μM)-mATP-3′P; NTP conc (μM)-100; Oligonucleotide-5′-6-FAM-
T15AT*mG; Substrate conc (μM)-10; Product -5′-6-FAM-
T15AT*mGmA-3′P.

Activity relative to SEQ ID NO: 1950 (Activity FIOP) was calculated as the percent product of the variant compared with the percent product observed by the reaction with SEQ ID NO: 1950 (where the percent product may be set as the average of replicates or else the highest single sample as appropriate). The results are shown in Table 29.2.

TABLE 29.2

| | Amino Acid Differences | FIOP % product |
|---|---|---|
| SEQ ID NO: | (Relative to SEQ | Relative to |
| (nt/aa) | ID NO: 1950) | SEQ ID NO: 1950 |
| 2003/2004 | V455L | +++ |
| 2005/2006 | T362S | +++ |
| 2007/2008 | R258W | +++ |
| 2009/2010 | G468Q | +++ |
| 2011/2012 | G468W | +++ |
| 2013/2014 | K274V | +++ |
| 2015/2016 | H414G | +++ |
| 2017/2018 | R258Q | +++ |
| 2019/2020 | K407S | +++ |
| 2021/2022 | S405Y | +++ |
| 2023/2024 | L411Q/H414F | +++ |
| 2025/2026 | E399G | +++ |
| 2027/2028 | Y192C | +++ |
| 2029/2030 | Y192R | +++ |
| 2031/2032 | H494A | +++ |
| 2033/2034 | K274M | +++ |
| 2035/2036 | T286N | +++ |
| 2037/2038 | E399D | +++ |
| 2039/2040 | H413A/H414F | +++ |
| 2041/2042 | E193S | +++ |
| 2043/2044 | D409Q/H414F | +++ |
| 2045/2046 | L411R/H414F | +++ |
| 2047/2048 | G245A | +++ |
| 2049/2050 | A410V/H414F | +++ |
| 2051/2052 | L466M | ++ |
| 2053/2054 | E399F | ++ |
| 2055/2056 | S405L | ++ |
| 2057/2058 | K407N | ++ |
| 2059/2060 | S509K | ++ |
| 2061/2062 | K278N | ++ |
| 2063/2064 | A410Y/H414F | ++ |
| 2065/2066 | F398W | ++ |
| 2067/2068 | F194M | ++ |
| 2069/2070 | L359V | ++ |
| 2071/2072 | K278C | ++ |
| 2073/2074 | H414F/C417W | ++ |
| 2075/2076 | H413I/H414F | ++ |
| 2077/2078 | D409K/H414F | ++ |
| 2079/2080 | K274Q | ++ |
| 2081/2082 | A410S/H414F | ++ |
| 2083/2084 | A410I/H414F | ++ |
| 2085/2086 | V316C | ++ |
| 2087/2088 | K278L | ++ |
| 2089/2090 | L185F | ++ |
| 2091/2092 | F194C | ++ |
| 2093/2094 | P352R | ++ |
| 2095/2096 | Y259N | ++ |
| 2097/2098 | A304L | ++ |
| 2099/2100 | F194W | ++ |
| 2101/2102 | R291M | ++ |
| 2103/2104 | R258K | ++ |
| 2105/2106 | K274N | ++ |
| 2107/2108 | S261G | ++ |
| 2109/2110 | G468S | ++ |
| 2111/2112 | P352G | ++ |
| 2113/2114 | S509G | ++ |
| 2115/2116 | L411G/H414F | ++ |
| 2117/2118 | L185C | ++ |
| 2119/2120 | K274P | ++ |
| 2121/2122 | V378L | ++ |
| 2123/2124 | K274T | ++ |
| 2125/2126 | R258G | + |
| 2127/2128 | R258L | + |
| 2129/2130 | K263S | + |
| 2131/2132 | F194A | + |
| 2133/2134 | R258E | + |
| 2135/2136 | Y280S | + |
| 2137/2138 | S405G | + |
| 2139/2140 | T362Y | + |
| 2141/2142 | Y308N | + |
| 2143/2144 | Y192L | + |
| 2145/2146 | H414F/A415W | + |
| 2147/2148 | L284I | + |
| 2149/2150 | G468M | + |
| 2151/2152 | V163I/Q169E | + |
| 2153/2154 | A465E | + |
| 2155/2156 | H494G | + |
| 2157/2158 | H494L | + |
| 2159/2160 | D198L | + |
| 2161/2162 | L411A/H414F | + |
| 2163/2164 | K263I | + |
| 2165/2166 | E399C | + |
| 2167/2168 | L306I | + |
| 2169/2170 | V271C | + |
| 2171/2172 | V455I | + |
| 2173/2174 | L411I/H414F | + |
| 2175/2176 | H413G/H414F | + |
| 2177/2178 | N197G | + |
| 2179/2180 | F194G | + |
| 2181/2182 | Y192I | + |
| 2183/2184 | Q251R | + |
| 2185/2186 | V316A | + |
| 2187/2188 | F194D | + |
| 2189/2190 | K297P | + |
| 2191/2192 | S396T | + |
| 2193/2194 | Y259V | + |
| 2195/2196 | N197R | + |
| 2197/2198 | H494C | + |
| 2199/2200 | F347Q | + |
| 2201/2202 | H494W | + |
| 2203/2204 | A410F/H414F | + |
| 2205/2206 | H413F/H414F | + |
| 2207/2208 | E186C | + |
| 2209/2210 | K297L | + |
| 2211/2212 | E399T | + |
| 2213/2214 | R291W | + |
| 2215/2216 | E193R | + |
| 2217/2218 | T286S | + |
| 2219/2220 | Y280L | + |
| 2221/2222 | L306M | + |
| 2223/2224 | Y192G | + |
| 2225/2226 | L411T/H414F | + |
| 2227/2228 | L284M | + |
| 2229/2230 | V163I/Q169R | + |
| 2231/2232 | N197L | + |
| 2233/2234 | K407F | + |
| 2235/2236 | V290A | + |
| 2237/2238 | D198A | + |
| 2239/2240 | A415F | + |
| 2241/2242 | S261A | + |
| 2243/2244 | L393R | + |
| 2245/2246 | L411F/H414F | + |

Levels of increased activity were determined relative to the reference polypeptide of SEQ ID NO: 1950 and defined as follows: “+” 1.21 to 1.91, “++” >1.91, “+++” >3.34.

### Example 30

### Improvements Over SEQ ID NO: 2008 in the Extension of Oligonucleotide Acceptor Molecules with Nucleotide Triphosphate Donors

### HTP Screening for Improved TdT Variants

SEQ ID NO: 2008 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 30.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 30.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
(min)-60; Reaction temperature (C.)-50° C.; Reaction Time (min)-60;
Reaction volume (μL)-1; NTP (μM)-mATP-3′P; NTP conc (μM)-100;
Oligonucleotide-5′-6-FAM-T15AT*mG; Substrate conc (μM)-10;
Product -5′-6-FAM-T15AT*mGmA-3′P.

Activity relative to SEQ ID NO: 2008 (Activity FIOP) was calculated as the percent product of the variant compared with the percent product observed by the reaction with SEQ ID NO: 2008 (where the percent product may be set as the average of replicates or else the highest single sample as appropriate). The results are shown in Table 30.2.

TABLE 30.2

| SEQ | | FIOP % Product |
|---|---|---|
| ID NO: | Amino Acid Differences | Relative to |
| (nt/aa) | (Relative to SEQ ID NO: 2008) | SEQ ID NO: 2008 |
| 2247/2248 | H413A/H414G/G468Q | +++ |
| 2249/2250 | H413A/H414G | +++ |
| 2251/2252 | L411G/H413A/G468Q | +++ |
| 2253/2254 | K263H/L411G/H413A/H414G/G468Q | +++ |
| 2255/2256 | H413A/G468Q | ++ |
| 2257/2258 | G468Q | ++ |
| 2259/2260 | P158H/K274V/L411G/H413A/H414G | ++ |
| 2261/2262 | K274V/L411G/G468Q | ++ |
| 2263/2264 | L411G/H414G | ++ |
| 2265/2266 | H413A | ++ |
| 2267/2268 | L185F/L411G/H413A/H414G | ++ |
| 2269/2270 | K274V/T286N/L411G/H413A/ | + |
| | C417W/G468Q | |
| 2271/2272 | H414G | + |
| 2273/2274 | K263H/H413A/H414G | + |
| 2275/2276 | K278N/L411G/H413A/G468Q | + |
| 2277/2278 | L185F/K274V/H413A/H414G | + |
| 2279/2280 | K274V/L411G/C417W/G468Q | + |
| 2281/2282 | L411G/H413A/C417W/G468Q | + |
| 2283/2284 | K274V/G468Q | + |
| 2285/2286 | L411G/H413A/C417W | + |
| 2287/2288 | H413A/C417W/G468Q | + |
| 2289/2290 | K274V/H413A/H414G/C417W/ | + |
| | G468Q | |

Levels of increased activity were determined relative to the reference polypeptide of SEQ ID NO: 2008 and defined as follows: “+” 1.05 to 1.28, “++” >1.28, “+++” >1.67.

### Example 31

### Improvements Over SEQ ID NO: 2008 in the Extension of Oligonucleotide Acceptor Molecules with Nucleotide Triphosphate Donors

### HTP Screening for Improved TdT Variants

SEQ ID NO: 2008 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 31.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 31.1
Reaction conditions

Lysis buffer-TEA (50 mM, pH 7.5), 0.1 g/L lysozyme; Reaction
buffer-20 mM TEA, 0.25 mM CoCl₂, pH 7.8; Lysate concentration
(vol %)-0.25; Preincubation Temp (C.)-50; Preincubation Time
(min)-60; Reaction temperature (C.)-50; Reaction Time (min)-60;
Reaction volume (μL)-1; NTP (μM)-mATP-3′P; NTP conc (μM)-100;
Oligonucleotide-5′-6-FAM-T15AT*mU; Substrate conc (μM)-10;
Product -5′-6-FAM-T15AT*mUmA-3′P.

Activity relative to SEQ ID NO: 2008 (Activity FIOP) was calculated as the percent product of the variant compared with the percent product observed by the reaction with SEQ ID NO: 2008 (where the percent product may be set as the average of replicates or else the highest single sample as appropriate). The results are shown in Table 31.2.

TABLE 31.2

| | Amino Acid Differences | FIOP % Product |
|---|---|---|
| SEQ ID NO: | (Relative to SEQ | Relative to |
| (nt/aa) | ID NO: 2008) | SEQ ID NO: 2008 |
| 2291/2292 | R26A/Q27R | +++ |
| 2293/2294 | R26G | +++ |
| 2295/2296 | E246N | +++ |
| 2297/2298 | L248R | +++ |
| 2299/2300 | R26I | +++ |
| 2301/2302 | G8P | +++ |
| 2303/2304 | H3G | +++ |
| 2305/2306 | S36G | ++ |
| 2307/2308 | R26Q | ++ |
| 2309/2310 | T58N | ++ |
| 2311/2312 | N76P | ++ |
| 2313/2314 | R26T | ++ |
| 2315/2316 | T58M | ++ |
| 2317/2318 | S36K | ++ |
| 2319/2320 | V253L | ++ |
| 2321/2322 | F62W | ++ |
| 2323/2324 | G151S | ++ |
| 2325/2326 | T58S | + |
| 2327/2328 | S116A | + |
| 2329/2330 | P157V | + |
| 2331/2332 | S12V | + |
| 2333/2334 | E249R | + |
| 2335/2336 | D147G | + |
| 2337/2338 | I50L | + |
| 2339/2340 | H3A | + |
| 2341/2342 | N90L | + |
| 2343/2344 | I15A | + |
| 2345/2346 | L248V | + |
| 2347/2348 | E249A | + |
| 2349/2350 | N255G | + |
| 2351/2352 | G11C | + |
| 2353/2354 | E249G | + |
| 2355/2356 | N90S | + |
| 2357/2358 | Y39A | + |

Levels of increased activity were determined relative to the reference polypeptide of SEQ ID NO: 2008 and defined as follows: “+” 1.16 to 1.26, “++” >1.26, “+++” >1.41.

### Example 32

### Improvements Over SEQ ID NO: 2008 in the Extension of Oligonucleotide Acceptor Molecules with Nucleotide Triphosphate Donors

### HTP Screening for Improved TdT Variants

SEQ ID NO: 2008 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 32.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 32.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
(min)-60; Reaction temperature (C.)-50° C.; Reaction Time (min)-60;
Reaction volume (μL)-1; NTP (μM)-mATP; NTP conc (μM)-100;
Oligonucleotide-5′-6-FAM-T15mUmGmA; Substrate conc (μM)-10;
Product -5′-6-FAM-T15mUmGmAmA.

Activity relative to SEQ ID NO: 2008 (Activity FIOP) was calculated as the percent product of the variant compared with the percent product observed by the reaction with SEQ ID NO: 2008 (where the percent product may be set as the average of replicates or else the highest single sample as appropriate). The results are shown in Table 32.2.

TABLE 32.2

| | Amino Acid Differences | FIOP % Product |
|---|---|---|
| SEQ ID NO: | (Relative to SEQ | Relative to |
| (nt/aa) | ID NO: 2008) | SEQ ID NO: 2008 |
| 2359/2360 | N90M | +++ |
| 2361/2362 | I15L | +++ |
| 2363/2364 | K70A | +++ |
| 2365/2366 | S12A | +++ |
| 2367/2368 | R26S | ++ |
| 2369/2370 | S81E | ++ |
| 2371/2372 | I38T | ++ |
| 2373/2374 | S79T | ++ |
| 2375/2376 | R16S | ++ |
| 2377/2378 | I38L | ++ |
| 2379/2380 | G9T | + |
| 2381/2382 | S12Q | ++ |
| 2383/2384 | G11S | + |
| 2385/2386 | P157L | + |
| 2387/2388 | I15Q | + |
| 2389/2390 | G9A | + |
| 2391/2392 | R26W | + |
| 2393/2394 | S12N | + |
| 2395/2396 | T58A | + |
| 2397/2398 | E249T | + |
| 2399/2400 | Y39G | + |
| 2401/2402 | P158A | + |
| 2403/2404 | G151I | + |

Levels of increased activity were determined relative to the reference polypeptide of SEQ ID NO: 2008 and defined as follows: “+” 1.03 to 1.07, “++” >1.07, “+++” >1.12.

### Example 33

### Improvements Over SEQ ID NO: 2008 in the Extension of Oligonucleotide Acceptor Molecules with Nucleotide Triphosphate Donors

### HTP Screening for Improved TdT Variants

SEQ ID NO: 2008 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 33.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 33.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
(min)-60; Reaction temperature (C.)-50° C.; Reaction Time (min)-60;
Reaction volume (μL)-1; NTP (μM)-mATP-3′P; NTP conc (μM)-100;
Oligonucleotide-5′-6-FAM-T15AT*mU; Substrate conc (μM)-10;
Product -5′-6-FAM-T15AT*mUmA-3′P.

Activity relative to SEQ ID NO: 2008 (Activity FIOP) was calculated as the percent product of the variant compared with the percent product observed by the reaction with SEQ ID NO: 2008 (where the percent product may be set as the average of replicates or else the highest single sample as appropriate). The results are shown in Table 33.2.

TABLE 33.2

| | Amino Acid Differences | FIOP % Product |
|---|---|---|
| SEQ ID NO: | (Relative to SEQ | Relative to |
| (nt/aa) | ID NO: 2008) | SEQ ID NO: 2008 |
| 2405/2406 | R473S | +++ |
| 2407/2408 | R473G | +++ |
| 2409/2410 | R473A | +++ |
| 2411/2412 | A189R | +++ |
| 2413/2414 | N493E | +++ |
| 2415/2416 | R480E | +++ |
| 2417/2418 | H413E | +++ |
| 2419/2420 | H413G | +++ |
| 2421/2422 | R473Q | +++ |
| 2423/2424 | A189Q | +++ |
| 2425/2426 | H413S | ++ |
| 2427/2428 | Q353R | ++ |
| 2429/2430 | R196T | ++ |
| 2431/2432 | R480S | ++ |
| 2433/2434 | R480G | ++ |
| 2435/2436 | R473P | ++ |
| 2437/2438 | R480A | ++ |
| 2439/2440 | A410E | ++ |
| 2441/2442 | R206N | ++ |
| 2443/2444 | C307S | ++ |
| 2445/2446 | R196S | ++ |
| 2447/2448 | C307H | ++ |
| 2449/2450 | R473D | ++ |
| 2451/2452 | A408E | ++ |
| 2453/2454 | C307E | ++ |
| 2455/2456 | Q397A | + |
| 2457/2458 | H413L | + |
| 2459/2460 | K318R | + |
| 2461/2462 | I324V | + |
| 2463/2464 | W481V | + |
| 2465/2466 | W469Y | + |
| 2467/2468 | R196A | + |
| 2469/2470 | H413M | + |
| 2471/2472 | C307L | + |
| 2473/2474 | A408W | + |
| 2475/2476 | N493Q | + |
| 2477/2478 | W481S | + |
| 2479/2480 | W469F | + |
| 2481/2482 | R196Y | + |
| 2483/2484 | R314M | + |
| 2485/2486 | R314A | + |
| 2487/2488 | A408L | + |
| 2489/2490 | F262V | + |
| 2491/2492 | A189E | + |
| 2493/2494 | W481L | + |
| 2495/2496 | R205E | + |
| 2497/2498 | R314Q | + |
| 2499/2500 | W481A | + |
| 2501/2502 | L491M | + |
| 2503/2504 | R480W | + |
| 2505/2506 | R480L | + |

Levels of increased activity were determined relative to the reference polypeptide of SEQ ID NO: 2008 and defined as follows: “+” 1.08 to 1.42, “++” >1.42, “+++” >1.71.

### Example 34

### Improvements Over SEQ ID NO: 2254 in the Extension of Oligonucleotide Acceptor Molecules with Nucleotide Triphosphate Donors

### HTP Screening for Improved TdT Variants

SEQ ID NO: 2254 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 34.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 34.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
(min)-60; Reaction temperature (C.)-50° C.; Reaction Time (min)-30;
Reaction volume (μL)-1; NTP (μM)-mATP-3′P; NTP conc (μM)-100;
Oligonucleotide-5′-6-FAM-T15AT*mU; Substrate conc (μM)-10;
Product -5′-6-FAM-T15AT*mUmA-3′P.

Activity relative to SEQ ID NO: 2254 (Activity FIOP) was calculated as the percent product of the variant compared with the percent product observed by the reaction with SEQ ID NO: 2254 (where the percent product may be set as the average of replicates or else the highest single sample as appropriate). The results are shown in Table 34.2.

TABLE 34.2

| SEQ ID NO: | Amino Acid Differences | FIOP % product Relative |
|---|---|---|
| (nt/aa) | (Relative to SEQ ID NO: 2254) | to SEQ ID NO: 2254 |
| 2507/2508 | F398W/L466M | +++ |
| 2509/2510 | E399D/G411Q/K416Q | +++ |
| 2511/2512 | T362S/K407S/V455L | ++ |
| 2513/2514 | S396T/F398W/A410V/L466M | ++ |
| 2515/2516 | L284M/F398W/L466M | + |
| 2517/2518 | N197R/V455L | + |
| 2519/2520 | N197R/K407S/V455L | + |
| 2521/2522 | L466M | + |

Levels of increased activity were determined relative to the reference polypeptide of SEQ ID NO: 2254 and defined as follows: “+” 1.13 to 1.20 (first 50%), “++” >1.20, “+++” >1.39.

### Example 35

### Improvements Over SEQ ID NO: 2514 in the Extension of Oligonucleotide Acceptor Molecules with Nucleotide Triphosphate Donors

### HTP Screening for Improved TdT Variants

SEQ ID NO: 2514 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 35.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 35.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
R(min)-60; eaction temperature (C.)-50° C.; Reaction Time (min)-60;
Reaction volume (μL)-1; NTP (μM)-mATP-3′P; NTP conc (μM)-100;
Oligonucleotide-5′-6-FAM-T15AmU*mG; Substrate conc (μM)-10;
Product -5′-6-FAM-T15AmU*mGmA-3′P.

Activity relative to SEQ ID NO: 2514 (Activity FIOP) was calculated as the percent product of the variant compared with the percent product observed by the reaction with SEQ ID NO: 2514 (where the percent product may be set as the average of replicates or else the highest single sample as appropriate). The results are shown in Table 35.2.

TABLE 35.2

| SEQ | Amino Acid Differences | FIOP % product |
|---|---|---|
| ID NO: | (Relative to SEQ | Relative to |
| (nt/aa) | ID NO: 2514) | SEQ ID NO: 2514 |
| 2523/2524 | R26Q/N90L/E246N/L248R/T362S/ | +++ |
| | V455L | |
| 2525/2526 | R26Q/N90L/E246N/L248R/V455L | +++ |
| 2527/2528 | R26I/L248R/V455L | +++ |
| 2529/2530 | R26G/N90L/L248V/V455L/Y459H | +++ |
| 2531/2532 | R26Q/N90L/E246N/V455L | +++ |
| 2533/2534 | R26Q/N90L/L248R/K266V/V455L | +++ |
| 2535/2536 | N90L/E246N/L248R/S261A/K266V/ | +++ |
| | T362S/V455L | |
| 2537/2538 | R26G/N90L/K266V | +++ |
| 2539/2540 | R26Q/T173I/L248R | +++ |
| 2541/2542 | R26Q/E246N/L248V/T362S | ++ |
| 2543/2544 | R26Q/L248V | ++ |
| 2545/2546 | R26Q/L248R | ++ |
| 2547/2548 | F62W/E249R | ++ |
| 2549/2550 | L248V/K266V/T362S | ++ |
| 2551/2552 | R26G/N90L/L248V/V455L | ++ |
| 2553/2554 | N90L/E246N/L248R/K266V/T362S/ | ++ |
| | V455L | |
| 2555/2556 | R26I/N90L/E246N/L248R | ++ |
| 2557/2558 | R26G/E246N/L248R/T362S | ++ |
| 2559/2560 | R26I/L248R/K266V/T362S | ++ |
| 2561/2562 | R26Q/N90L/E246N/L248R/S261A | ++ |
| 2563/2564 | R26G/E246N/L248V/V455L | ++ |
| 2565/2566 | R26Q/T362S | ++ |
| 2567/2568 | R26G/N90L/E246N/K266V/T362S | ++ |
| 2569/2570 | L248R | ++ |
| 2571/2572 | R26I/E246N/L248R/T362S/V455L | + |
| 2573/2574 | R26Q/L248R/S261A/K266V/T362S/ | + |
| | V455L | |
| 2575/2576 | L248R/V455L | |
| 2577/2578 | E246N/L248R/T362S | + |
| 2579/2580 | R26G/L248V/S261A/K266V | + |
| 2581/2582 | R26Q/L248R/T362S/V455L | + |
| 2583/2584 | R26Q/N90L/E246N/L248V/T362S/ | + |
| | V455L | |
| 2585/2586 | R26Q/L248V/T362S/V455L | + |
| 2587/2588 | R26G/N90L/T362S/V455L | + |
| 2589/2590 | R26Q/L248R/V455L | + |
| 2591/2592 | E246N/L248R | + |
| 2593/2594 | E246N/K266V/V455L | + |
| 2595/2596 | R26Q/N90L/E246N/T362S | |
| 2597/2598 | R26G/E246N | + |
| 2599/2600 | R26Q/L248V/K266V/T362S | + |
| 2601/2602 | T58N/N197L/E249R/K407N/V410S | + |
| 2603/2604 | R26G/N90L/E246N | + |
| 2605/2606 | R26Q/T362S/V455L | + |
| 2607/2608 | L248V/T362S/V455L | + |
| 2609/2610 | N90L/E246N/L248R | + |
| 2611/2612 | R26G/N90L/E94K/L248R/ | + |
| | S261A/K266V/T362S/V455L | |
| 2613/2614 | R26G/L248R | + |
| 2615/2616 | T362S | + |
| 2617/2618 | R26G/N90L/E246N/L248R | + |
| 2619/2620 | R26I/N90L/L248R | + |

Levels of increased activity were determined relative to the reference polypeptide of SEQ ID NO: 2514 and defined as follows: “+” 2.23 to 3.99, “++” >3.99, “+++” >6.49.

### Example 36

### Improvements Over SEQ ID NO: 2524 in the Extension of Oligonucleotide Acceptor Molecules with Nucleotide Triphosphate Donors

### HTP Screening for Improved TdT Variants

SEQ ID NO: 2524 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 36.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 36.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
(min)-60; Reaction temperature (C.)-50° C.; Reaction Time (min)-60;
Reaction volume (μL)-1; NTP (μM)-ATP-3′P; NTP conc (μM)-100;
Oligonucleotide-5′-6-FAM-T15AmU*mG; Substrate conc (μM)-10;
Product -5′-6-FAM-T15AmU*mGmA-3′P.

Activity relative to SEQ ID NO: 2524 (Activity FIOP) was calculated as the percent product of the variant compared with the percent product observed by the reaction with SEQ ID NO: 2524 (where the percent product may be set as the average of replicates or else the highest single sample as appropriate). The results are shown in Table 36.2.

TABLE 36.2

| SEQ | | FIOP % product |
|---|---|---|
| ID NO: | Amino Acid Differences | Relative to |
| (nt/aa) | (Relative to SEQ ID NO: 2524) | SEQ ID NO: 2524 |
| 2621/2622 | M30G/K266V/Q353R | +++ |
| 2623/2624 | M30G/S261A/K266V/Q353R/Q468S | +++ |
| 2625/2626 | K266V/Q353R/Q468S | +++ |
| 2627/2628 | M30E/A189R/K266V | +++ |
| 2629/2630 | K266V/Q468S | +++ |
| 2631/2632 | M30G/K266V | ++ |
| 2633/2634 | A189R/S261A | ++ |
| 2635/2636 | A189R/Q353R | ++ |
| 2637/2638 | I38T/S81E/K318R | ++ |
| 2639/2640 | K266V/C307S/Q353R/Q468S | ++ |
| 2641/2642 | G11S/T58N/L227M/N246E | ++ |
| 2643/2644 | M30T/K266V/K303E | ++ |
| 2645/2646 | S12A/R16S/P158A/N246E/R248L/ | ++ |
| | E249R | |
| 2647/2648 | M30T/A189R/S261A/K266V/Q353R/ | + |
| | A465E/Q468S | |
| 2649/2650 | N246E/E249R | + |
| 2651/2652 | G9T/R16S/F62W/P157L/N246E/ | + |
| | E249R/S362T | |
| 2653/2654 | S79T/S81E | + |
| 2655/2656 | Y39G/S79T | + |
| 2657/2658 | Y39G | + |
| 2659/2660 | S261A/Q353R | + |
| 2661/2662 | T58N/P157L/P158A/S362T | + |
| 2663/2664 | K70A/Q353R | + |
| 2665/2666 | I38T/N197L | + |
| 2667/2668 | S81E | + |
| 2669/2670 | A189R | + |
| 2671/2672 | I38T | + |

Levels of increased activity were determined relative to the reference polypeptide of SEQ ID NO: 2524 and defined as follows: “+” 1.09 to 1.33, “++” >1.33, “+++” >1.77.

### Example 37

### Improvements Over SEQ ID NO: 2524 in the Extension of Oligonucleotide Acceptor Molecules with Nucleotide Triphosphate Donors

### HTP Screening for Improved TdT Variants

SEQ ID NO: 2524 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 37.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 37.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
(min)-60; Reaction temperature (C.)-50° C.; Reaction Time (min)-60;
Reaction volume (μL)-1; NTP (μM)-mATP-3′P; NTP conc (μM)-100;
Oligonucleotide-5′-6-FAM-T15AmU*mG; Substrate conc (μM)-25;
Product -5′-6-FAM-T15AmU*mGmA-3′P.

Activity relative to SEQ ID NO: 2524 (Activity FIOP) was calculated as the percent product of the variant compared with the percent product observed by the reaction with SEQ ID NO: 2524 (where the percent product may be set as the average of replicates or else the highest single sample as appropriate). The results are shown in Table 37.2.

TABLE 37.2

| | Amino Acid Differences | |
|---|---|---|
| SEQ ID NO: | (Relative to SEQ | FIOP % product Relative |
| (nt/aa) | ID NO: 2524) | to SEQ ID NO: 2524 |
| 2673/2674 | T379L | +++ |
| 2675/2676 | T379M | +++ |
| 2677/2678 | K427L | +++ |
| 2679/2680 | K402G | +++ |
| 2681/2682 | K427M | +++ |
| 2683/2684 | L327M/G406T | +++ |
| 2685/2686 | W382L | +++ |
| 2687/2688 | A304V/Q329R | +++ |
| 2689/2690 | T379I | ++ |
| 2691/2692 | T484A | ++ |
| 2693/2694 | K506P | ++ |
| 2695/2696 | L340V | ++ |
| 2697/2698 | T379V | ++ |
| 2699/2700 | F504K | ++ |
| 2701/2702 | W382F | ++ |
| 2703/2704 | L403S | ++ |
| 2705/2706 | Q169M | ++ |
| 2707/2708 | L403A | ++ |
| 2709/2710 | S292N | ++ |
| 2711/2712 | K402V | ++ |
| 2713/2714 | K427W | ++ |
| 2715/2716 | L340I | + |
| 2717/2718 | L403P | + |
| 2719/2720 | P404S | + |
| 2721/2722 | K427R | + |
| 2723/2724 | Q373G | + |
| 2725/2726 | D429R | + |
| 2727/2728 | N191V/A413V | + |
| 2729/2730 | M490R | + |
| 2731/2732 | A495S | + |
| 2733/2734 | A495G | + |
| 2735/2736 | L403E | + |
| 2737/2738 | Q353H/Y459V | + |
| 2739/2740 | Y459I | + |
| 2741/2742 | A495C | + |
| 2743/2744 | K402S | + |
| 2745/2746 | G461S | + |
| 2747/2748 | P404D | + |
| 2749/2750 | Q329R | + |
| 2751/2752 | V47A/L326R | + |
| 2753/2754 | T200V | + |
| 2755/2756 | L403R | + |

Levels of increased activity were determined relative to the reference polypeptide of SEQ ID NO: 2524 and defined as follows: “+” 1.21 to 1.43, “++” >1.43, “+++” >1.80.

### Example 38

### Improvements Over SEQ ID NO: 2524 in the Extension of Oligonucleotide Acceptor Molecules with Nucleotide Triphosphate Donors

### HTP Screening for Improved TdT Variants

SEQ ID NO: 2524 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 38.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 38.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-60° C.; Preincubation Time
(min)-60; Reaction temperature (C.)-60° C.; Reaction Time (min)-60;
Reaction volume (μL)-1; NTP (μM)-mATP-3′P; NTP conc (μM)-100;
Oligonucleotide-5′-6-FAM-T15AT*mG; Substrate conc (μM)-25;
Product -5′-6-FAM-T15AT*mGmA-3′P.

Activity relative to SEQ ID NO: 2524 (Activity FIOP) was calculated as the percent product of the variant compared with the percent product observed by the reaction with SEQ ID NO: 2524 (where the percent product may be set as the average of replicates or else the highest single sample as appropriate). The results are shown in Table 38.2.

TABLE 38.2

| | Amino Acid Differences | |
|---|---|---|
| SEQ ID NO: | (Relative to SEQ | FIOP % product Relative |
| (nt/aa) | ID NO: 2524) | to SEQ ID NO: 2524 |
| 2675/2676 | T379M | +++ |
| 2693/2694 | K506P | +++ |
| 2681/2682 | K427M | +++ |
| 2757/2758 | S325W | +++ |
| 2673/2674 | T379L | +++ |
| 2691/2692 | T484A | +++ |
| 2677/2678 | K427L | +++ |
| 2713/2714 | K427W | +++ |
| 2759/2760 | K427F | +++ |
| 2679/2680 | K402G | ++ |
| 2761/2762 | K506T | ++ |
| 2763/2764 | T200N | ++ |
| 2765/2766 | K427C | ++ |
| 2723/2724 | Q373G | ++ |
| 2735/2736 | L403E | ++ |
| 2731/2732 | A495S | ++ |
| 2767/2768 | K508S | ++ |
| 2717/2718 | L403P | ++ |
| 2747/2748 | P404D | ++ |
| 2739/2740 | Y459I | ++ |
| 2769/2770 | K427Y | ++ |
| 2745/2746 | G461S | ++ |
| 2771/2772 | K506S | + |
| 2773/2774 | A189V | + |
| 2743/2744 | K402S | + |
| 2775/2776 | P404E | + |
| 2777/2778 | T484M | + |
| 2709/2710 | S292N | + |
| 2779/2780 | G406N | + |
| 2781/2782 | K508T | + |
| 2783/2784 | E203D | + |
| 2785/2786 | K427N | + |
| 2719/2720 | P404S | + |
| 2733/2734 | A495G | + |
| 2787/2788 | N175L | + |
| 2789/2790 | T484H | + |
| 2695/2696 | L340V | + |
| 2791/2792 | K402E | + |
| 2793/2794 | Q373C | + |
| 2795/2796 | L403G | + |
| 2707/2708 | L403A | + |
| 2797/2798 | E203M | + |
| 2799/2800 | Q179L | + |
| 2801/2802 | S293Q | + |

Levels of increased activity were determined relative to the reference polypeptide of SEQ ID NO: 2524 and defined as follows: “+” 1.19 to 1.44, “++” >1.44, “+++” >1.97.

### Example 39

### Improvements Over SEQ ID NO: 2638 in the Extension of Oligonucleotide Acceptor Molecules with Nucleotide Triphosphate Donors

### HTP Screening for Improved TdT Variants

SEQ ID NO: 2638 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 39.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 39.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
(min)-60; Reaction temperature (C.)-50° C.; Reaction Time (min)-60;
Reaction volume (μL)-1; NTP (μM)-mATP-3′P; NTP conc (μM)-100;
Oligonucleotide-5′-6-FAM-T15AmU*mG; Substrate conc (μM)-25;
Product -5′-6-FAM-T15AmU*mGmA-3′P.

Activity relative to SEQ ID NO: 2638 (Activity FIOP) was calculated as the percent product of the variant compared with the percent product observed by the reaction with SEQ ID NO: 2638 (where the percent product may be set as the average of replicates or else the highest single sample as appropriate). The results are shown in Table 39.2.

TABLE 39.2

| | | FIOP % product |
|---|---|---|
| SEQ ID | | Relative |
| NO: | Amino Acid Differences | to SEQ ID |
| (nt/aa) | (Relative to SEQ ID NO: 2638) | NO: 2638 |
| 2803/2804 | S79T/A189R/C307L/V410E | + |
| 2805/2806 | M30G/T58N/S79T/A189R/C307L/R480E | + |
| 2807/2808 | S79T/C307L | + |
| 2809/2810 | G11S/M30G/S79T/A189R/R480E | + |

Levels of increased activity were determined relative to the reference polypeptide of SEQ ID NO: 2638 and defined as follows: “+” from 1.29 to 1.94.

### Example 40

### Improvements Over SEQ ID NO: 2804 in the Extension of Oligonucleotide Acceptor Molecules with Nucleotide Triphosphate Donors

### HTP Screening for Improved TdT Variants

SEQ ID NO: 2804 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 40.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 40.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
(min)-60; Reaction temperature (C.)-50° C.; Reaction Time (min)-60;
Reaction volume (μL)-1; NTP (μM)-mATP-3′P; NTP conc (μM)-100;
Oligonucleotide-5′-6-FAM-T15AmG*mC; Substrate conc (μM)-25;
Product -5′-6-FAM-T15AmG*mCmA-3′P.

Activity relative to SEQ ID NO: 2804 (Activity FIOP) was calculated as the percent product of the variant compared with the percent product observed by the reaction with SEQ ID NO: 2804 (where the percent product may be set as the average of replicates or else the highest single sample as appropriate). The results are shown in Table 40.2.

TABLE 40.2

| | | FIOP % product |
|---|---|---|
| SEQ ID | | Relative to |
| NO: | Amino Acid Differences | SEQ ID |
| (nt/aa) | (Relative to SEQ ID NO: 2804) | NO: 2804 |
| 2811/2812 | A304V/L340I/K402G/L403S/K506P | +++ |
| 2813/2814 | A304V/L340I/K402G/K506P | +++ |
| 2815/2816 | A304V/L340V/K402G/L403S/F504K/ | +++ |
| | K506P | |
| 2817/2818 | Q169M/A304V/K402G/L403S/K427L/ | +++ |
| | K506P | |
| 2819/2820 | A304V/L340V/K402G/K427L/F504K/ | +++ |
| | K506P | |
| 2821/2822 | A304V/L340I/K402G/L403A/K427L/ | +++ |
| | D429R/F504K/K506P | |
| 2823/2824 | A304V/K402G/L403A/F504K/K506P | +++ |
| 2825/2826 | L340V/K402G/K427L/D429R/K506P | +++ |
| 2827/2828 | A304V/K402G/L403P/K506P | +++ |
| 2829/2830 | Q169M/A304V/L340I/D429R/K506P | +++ |
| 2831/2832 | Q169M/A304V/L340V/K402G/K427L | +++ |
| 2833/2834 | K402G/L403A/K427M/F504K/K506P | +++ |
| 2835/2836 | A304V/F504K/K506P | +++ |
| 2837/2838 | A304V/L340I/K427M | +++ |
| 2839/2840 | L340I/K402G/D429R/F504K/K506P | ++ |
| 2841/2842 | Q169M/L403A/K427L/K506P | ++ |
| 2843/2844 | Q169M/A304V/K427M/F504K/K506P | ++ |
| 2845/2846 | A304V/K402G/L403S | ++ |
| 2847/2848 | A304V/L403S/F504K | ++ |
| 2849/2850 | A304V/L340I/K402G | ++ |
| 2851/2852 | A304V/K402G/L403A/D429R | ++ |
| 2853/2854 | A304V/K402G/L403P/K427M/F504K | ++ |
| 2855/2856 | K402G/L403S/K427M/F504K/K506P | ++ |
| 2857/2858 | A304V/L340I/L403A/K427L/D429R/ | ++ |
| | F504K/K506P | |
| 2859/2860 | L403S/K427L | ++ |
| 2861/2862 | A304V/F504K | ++ |
| 2863/2864 | Q169M/A304V/L340I/F504K | ++ |
| 2865/2866 | Q169M/A304V/F504K | ++ |
| 2867/2868 | Q169M/K402G/F504K/K506P | ++ |
| 2869/2870 | Q169M/K402G/L403A/F504K/K506P | ++ |
| 2871/2872 | T379M/Q468S | ++ |
| 2873/2874 | K402G/K427L/F504K | ++ |
| 2875/2876 | K266V/L327M/Q329R/P404S/E410V | ++ |
| 2877/2878 | Q169M/K402G/K427L/D429R/F504K | ++ |
| 2879/2880 | K427L | ++ |
| 2881/2882 | L340V/K402G/L403A | + |
| 2883/2884 | Q169M/L340I/K427L | + |
| 2885/2886 | K402G/F504K | + |
| 2887/2888 | K402G/L403S/D429R/F504K | + |
| 2889/2890 | Q169M/L340V/K402G/F504K | + |
| 2891/2892 | L327M/Q329R | + |
| 2893/2894 | Q169M/L340I/K506P | + |
| 2895/2896 | K427M | + |
| 2897/2898 | L327M/Q329R/P404D/E410V | + |
| 2899/2900 | L340I/K402G/F504K | + |
| 2901/2902 | L340I/K506P | + |
| 2903/2904 | L327M/E410V/T484A | + |
| 2905/2906 | T379M/P404D/E410V | + |
| 2907/2908 | L327M/Q329R/T379M/P404D/G406T/ | + |
| | E410V | |
| 2909/2910 | Q169M/L340V/K402G/L403P/K427M/ | + |
| | F504K/K506P | |
| 2911/2912 | F504K/K506P | + |
| 2913/2914 | Q169M/A304V/L403S/K427L/F504K | + |
| 2915/2916 | T379L/Q468S | + |
| 2917/2918 | K506P | + |
| 2919/2920 | Q169M/L340I/K402G/L403A/K427M/ | + |
| | D429R/F504K | |
| 2921/2922 | Q169M/K402G/F504K | + |
| 2923/2924 | T379M/E410V | + |
| 2925/2926 | L327M | + |
| 2927/2928 | L340I/L403S/F504K | + |
| 2929/2930 | L340V | + |
| 2931/2932 | L327M/Q329R/A465E/T484A | + |
| 2933/2934 | T484A | + |
| 2935/2936 | Q169M/A304V/L340V/K427L/D429R/ | + |
| | F504K | |
| 2937/2938 | A304V | + |
| 2939/2940 | S292N/L327M/Q329R/Q468S | + |
| 2941/2942 | L340V/F504K | + |
| 2943/2944 | F504K | + |
| 2945/2946 | L327M/W382L/G406T/E410V/T484A | + |
| 2947/2948 | T379M/W382L/Q468S | + |
| 2949/2950 | T379L/A465E/Q468S/T484A | + |

Levels of increased activity were determined relative to the reference polypeptide of SEQ ID NO: 2804 and defined as follows: “+” 1.20 to 2.39, “++” >2.39, “+++” >4.43.

### Example 41

### Improvements Over SEQ ID NO: 2812 in the Extension of Oligonucleotide Acceptor Molecules with Nucleotide Triphosphate Donors

### HTP Screening for Improved TdT Variants

SEQ ID NO: 2812 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 41.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 41.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
(min)-60; Reaction temperature (C.)-50° C.; Reaction Time (min)-60;
Reaction volume (μL) -1; NTP (μM)-mATP-3′P; NTP conc (μM)-100;
Oligonucleotide-5′-6-FAM-T15mAmU*mG; Substrate conc (μM)-25;
Product -5′-6-FAM-T15mAmU*mGmA-3′P.

Activity relative to SEQ ID NO: 2812 (Activity FIOP) was calculated as the percent product of the variant compared with the percent product observed by the reaction with SEQ ID NO: 2812 (where the percent product may be set as the average of replicates or else the highest single sample as appropriate). The results are shown in Table 41.2.

TABLE 41.2

| SEQ ID | | FIOP % product |
|---|---|---|
| NO: | Amino Acid Differences | Relative to |
| (nt/aa) | (Relative to SEQ ID NO: 2812) | SEQ ID NO: 2812 |
| 2951/2952 | M30G/Q179A/Q373G/T379M | +++ |
| 2953/2954 | N246E/S325W/Q329R/T379M/G461S | +++ |
| 2955/2956 | M30G/A184S/N246E/S325W/T379M/ | +++ |
| | D429R/A495S | |
| 2957/2958 | M30G/N246E/S325W/L327M/Q329R/ | +++ |
| | P404D/G461S | |
| 2959/2960 | M30G/Q329R/T379M | +++ |
| 2961/2962 | M30G/N246E/S325W/T379M/P404D/ | +++ |
| | K427L/D429R/G461S | |
| 2963/2964 | M30G/A184S/N246E/T379M/P404D | ++ |
| 2965/2966 | Q373G/T379M | ++ |
| 2967/2968 | M30G/Q373G | ++ |
| 2969/2970 | N246E/L327M/P404D/G461S/A495S | ++ |
| 2971/2972 | M30G/T379M/D429R/Y459I/G461S | ++ |
| 2973/2974 | M30G/N246E/K427L/Y459I/G461S | ++ |
| 2975/2976 | M30G/T379M/Y459I/G461S/F504Q | ++ |
| 2977/2978 | M30G/T200N/Q373G/T379M | ++ |
| 2979/2980 | T379M/S403L/G406N/Q468S | ++ |
| 2981/2982 | S403L/M441K | + |
| 2983/2984 | M30G/Q179A/T200N/Q373G/S403E | + |
| 2985/2986 | M30G/A184S/N246E/L327M/Q329R/ | + |
| | Y459I | |
| 2987/2988 | M30G/A184S/N246E/Y459I/G461S/ | + |
| | A495S/T500N/F504Q | |
| 2989/2990 | M30G/T200N/Q373G | + |
| 2991/2992 | M30G/L327M/P404D/F504S | + |
| 2993/2994 | Q353R/S403E | + |
| 2995/2996 | L327M/Q329R/T379M/F504S | + |
| 2997/2998 | M30G/Q373G/S403L | + |
| 2999/3000 | M30G | + |
| 3001/3002 | M30G/S325W/L327M | + |
| 3003/3004 | L327M/Y459I/G461S/A495S | + |
| 3005/3006 | Q373G/T379M/S403L | + |
| 3007/3008 | M30G/S325W/L327M/T379M/P404D/ | + |
| | D429R/A495S | |
| 3009/3010 | M30G/S403E/M441K/G460P | + |
| 3011/3012 | T200N/Q373G/T379M | + |

Levels of increased activity were determined relative to the reference polypeptide of SEQ ID NO: 2812 and defined as follows: “+” 1.06 to 1.18 (first 50%), “++” >1.18 (next 30%), “+++” >1.43 (top 20%)

### Example 42

### Improvements Over SEQ ID NO: 2812 in the Extension of Oligonucleotide Acceptor Molecules with Nucleotide Triphosphate Donors

### HTP Screening for Improved TdT Variants

SEQ ID NO: 2812 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 42.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 42.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-50° C.; Preincubation Time
(min)-60; Reaction temperature (C.)-50° C.; Reaction Time (min)-60;
Reaction volume (μL)-1; NTP (μM)-mATP-3′P; NTP conc (μM)-100;
Oligonucleotide-5′-6-FAM-T15mAmU*mG; Substrate conc (μM)-25;
Product -5′-6-FAM-T15mAmU*mGmA-3′P.

Activity relative to SEQ ID NO: 2812 (Activity FIOP) was calculated as the percent product of the variant compared with the percent product observed by the reaction with SEQ ID NO: 2812 (where the percent product may be set as the average of replicates or else the highest single sample as appropriate). The results are shown in Table 42.2.

TABLE 42.2

| | Amino Acid Differences | |
|---|---|---|
| SEQ ID NO: | (Relative to SEQ ID | FIOP % product Relative |
| (nt/aa) | NO: 2812) | to SEQ ID NO: 2812 |
| 3013/3014 | Q370D | +++ |
| 3015/3016 | E367D | +++ |
| 3017/3018 | W433G | +++ |
| 3019/3020 | I503T | +++ |
| 3021/3022 | W433E | +++ |
| 3023/3024 | W433S | +++ |
| 3025/3026 | Q370S | +++ |
| 3027/3028 | R264L | +++ |
| 3029/3030 | W433A | +++ |
| 3031/3032 | K278M | ++ |
| 3033/3034 | K266R | ++ |
| 3035/3036 | E520P | ++ |
| 3037/3038 | R264V | ++ |
| 3039/3040 | L326R | ++ |
| 3041/3042 | M435G | ++ |
| 3043/3044 | I503E | ++ |
| 3045/3046 | L499M | ++ |
| 3047/3048 | F525S | ++ |
| 3049/3050 | I503V | ++ |
| 3051/3052 | G414E | ++ |
| 3053/3054 | R264E | ++ |
| 3055/3056 | M435A | ++ |
| 3057/3058 | Q370G | ++ |
| 3059/3060 | T437R | + |
| 3061/3062 | I503A | + |
| 3063/3064 | T396R | + |
| 3065/3066 | W433R | + |
| 3067/3068 | R248K | + |
| 3069/3070 | R264T | + |
| 3071/3072 | M435E | + |
| 3073/3074 | M435S | + |
| 3075/3076 | W433M | + |
| 3077/3078 | L499R | + |
| 3079/3080 | P446G | + |
| 3081/3082 | V253I | + |
| 3083/3084 | D485E | + |
| 3085/3086 | Q370M | + |
| 3087/3088 | W433P | + |
| 3089/3090 | A444R | + |
| 3091/3092 | F525R | + |
| 3093/3094 | G231S | + |
| 3095/3096 | D198E | + |
| 3097/3098 | A444G | + |
| 3099/3100 | T437G | + |
| 3101/3102 | E233R | + |
| 3103/3104 | F525Q | + |
| 3105/3106 | R248T | + |

Levels of increased activity were determined relative to the reference polypeptide of SEQ ID NO: 2812 and defined as follows: “+” 1.10 to 1.27, “++” >1.27, “+++” >1.48.

### Example 43

### Improvements Over SEQ ID NO: 2812 in the Extension of Oligonucleotide Acceptor Molecules with Nucleotide Triphosphate Donors

### HTP Screening for Improved TdT Variants

SEQ ID NO: 2812 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 43.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 43.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-62° C.; Preincubation Time
(min)-60; Reaction temperature (C.)-50° C.; Reaction Time (min)-60;
Reaction volume (μL)-1; NTP (M)-mATP-3′P; NTP conc (μM)-100;
Oligonucleotide-5′-6-FAM-T15AmU*mG; Substrate conc (μM)-25;
Product -5′-6-FAM-T15AmU*mGmA-3′P.

Activity relative to SEQ ID NO: 2812 (Activity FIOP) was calculated as the percent product of the variant compared with the percent product observed by the reaction with SEQ ID NO: 2812 (where the percent product may be set as the average of replicates or else the highest single sample as appropriate). The results are shown in Table 43.2.

TABLE 43.2

| | Amino Acid Differences | |
|---|---|---|
| SEQ ID NO: | (Relative to SEQ | FIOP % product Relative |
| (nt/aa) | ID NO: 2812) | to SEQ ID NO: 2812 |
| 3107/3108 | K266T | +++ |
| 3051/3052 | G414E | +++ |
| 3083/3084 | D485E | +++ |
| 3025/3026 | Q370S | +++ |
| 3109/3110 | A366V | +++ |
| 3093/3094 | G231S | +++ |
| 3111/3112 | E186D | +++ |
| 3113/3114 | K368R | +++ |
| 3115/3116 | Q370N | +++ |
| 3117/3118 | W433V | ++ |
| 3119/3120 | R264A | ++ |
| 3121/3122 | K501R | ++ |
| 3123/3124 | K235R | ++ |
| 3125/3126 | E367A | ++ |
| 3127/3128 | A444H | ++ |
| 3033/3034 | K266R | ++ |
| 3085/3086 | Q370M | ++ |
| 3129/3130 | L188I | ++ |
| 3131/3132 | M435P | ++ |
| 3133/3134 | M435I | ++ |
| 3095/3096 | D198E | ++ |
| 3135/3136 | T437P | ++ |
| 3099/3100 | T437G | + |
| 3137/3138 | A515E | + |
| 3139/3140 | E243S | + |
| 3141/3142 | T437A | + |
| 3143/3144 | G442A | + |
| 3145/3146 | L287I | + |
| 3147/3148 | E243L | + |
| 3149/3150 | K297S/N440K | + |
| 3151/3152 | E243T | + |
| 3087/3088 | W433P | + |
| 3081/3082 | V253I | + |
| 3153/3154 | S439G | + |
| 3155/3156 | E243R | + |
| 3157/3158 | D485S | + |
| 3159/3160 | S439P | + |
| 3161/3162 | E233A | + |
| 3163/3164 | T437K | + |
| 3069/3070 | R264T | + |
| 3075/3076 | W433M | + |
| 3097/3098 | A444G | + |
| 3071/3072 | M435E | + |
| 3165/3166 | R248S | + |

Levels of increased activity were determined relative to the reference polypeptide of SEQ ID NO: 2812 and defined as follows: “+” 1.11 to 1.29, “++” >1.29, “+++” >1.48.

### Example 44

### Improvements Over SEQ ID NO: 2956 in the Extension of Oligonucleotide Acceptor Molecules with Nucleotide Triphosphate Donors

### HTP Screening for Improved TdT Variants

SEQ ID NO: 2956 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 44.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 44.1
Reaction conditions

Lysis buffer-TEA (50 mM, pH 7.5), 0.1 g/L lysozyme; Reaction
buffer-20 mM TEA, 0.25 mM CoCl2, pH 7.8; Lysate concentration
(vol %)-0.25; Preincubation Temp (C.)-64° C.; Preincubation Time
(min)-60; Reaction temperature (C.)-50° C.; Reaction Time (min)-60;
Reaction volume (μL)-1; NTP (μM)-mATP-3′P; NTP conc (μM)-100;
Oligonucleotide-5′-6-FAM-T15AmU*mG; Substrate conc (μM)-25;
Product -5′-6-FAM-T15AmU*mGmA-3′P.

Activity relative to SEQ ID NO: 2956 (Activity FIOP) was calculated as the percent product of the variant compared with the percent product observed by the reaction with SEQ ID NO: 2956 (where the percent product may be set as the average of replicates or else the highest single sample as appropriate). The results are shown in Table 44.2.

TABLE 44.2

| SEQ | | FIOP % product |
|---|---|---|
| ID NO: | Amino Acid Differences | Relative to SEQ |
| (nt/aa) | (Relative to SEQ ID NO: 2956) | ID NO: 2956 |
| 3167/3168 | G140V/M142V/E153V/K427L/T484L | +++ |
| 3169/3170 | M142V/E150P/P158S/H177R/K427L/K445N | +++ |
| 3171/3172 | G140V/K161H/H177R/P404D/K427L/T484L | +++ |
| 3173/3174 | G140V/M142V/E153V/H177R/K427L/E434N/M441V/A444S/ | +++ |
| | G461S/K502G | |
| 3175/3176 | H177R/G365A/K427L/E434N | +++ |
| 3177/3178 | K427L | ++ |
| 3179/3180 | G140V/M142V/Q373R/K427L/T484L | ++ |
| 3181/3182 | G140V/E150P/E153V/G365A/Q373R/K427L/T484L | ++ |
| 3183/3184 | M142V/E153V/H177R/M441V/A444S | ++ |
| 3185/3186 | G140V/M142V/G365A/Q373R/P404D/K427L/T484L | ++ |
| 3187/3188 | H177R | ++ |
| 3189/3190 | G140V/H177R/P404D/T484L | ++ |
| 3191/3192 | E150P/H177R | ++ |
| 3193/3194 | M142V/E150P/H177R/P404D | + |
| 3195/3196 | M142V/G461S/T484L/K502G | + |
| 3197/3198 | E153V/T484L | + |
| 3199/3200 | W325L/K427L | + |
| 3201/3202 | G140V/M142V/H177R/M441V | + |
| 3203/3204 | G140V/H177R/P404D | + |
| 3205/3206 | T484L | + |
| 3207/3208 | E153V/K161H/W325L/P404D/K427L/M441V/T484L | + |
| 3209/3210 | M142V/H177R/Q373R/M441V | + |
| 3211/3212 | G140V/P148T/K161H/H177R/P404D | + |
| 3213/3214 | G140V/M142V/H177R | + |
| 3215/3216 | M142V/H177R/Q373R | + |
| 3217/3218 | E153V | + |
| 3219/3220 | G140V/E150P/H177R/P404D/P436S/M441V/T484L/K502G | + |

Levels of increased activity were determined relative to the reference polypeptide of SEQ ID NO: 2956 and defined as follows: “+” 1.24 to 1.76, “++” >1.76, “+++” >3.58.

### Example 45

### Improvements Over SEQ ID NO: 3174 in the Extension of Oligonucleotide Acceptor Molecules with Nucleotide Triphosphate Donors

### HTP Screening for Improved TdT Variants

SEQ ID NO: 3174 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 45.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 45.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-50° C.; Preincubation Time (min)-
60; Reaction temperature (C.)-50° C.; Reaction Time (min)-60; Reaction volume (μL)-1; NTP (μM)-
mATP-3′P; NTP conc (μM)-100; Oligonucleotide-5′-6-FAM-T15mAmU*mG; Substrate conc (μM)-
25; Product -5′-6-FAM-T15mAmU*mGmA-3′P.

Activity relative to SEQ ID NO: 3174 (Activity FIOP) was calculated as the percent product of the variant compared with the percent product observed by the reaction with SEQ ID NO: 3174 (where the percent product may be set as the average of replicates or else the highest single sample as appropriate). The results are shown in Table 45.2.

TABLE 45.2

| SEQ | | FIOP % product |
|---|---|---|
| ID NO: | Amino Acid Differences | Relative to SEQ |
| (nt/aa) | (Relative to SEQ ID NO: 3174) | ID NO: 3174 |
| 3221/3222 | R264A/K266T/T396S/G414E/W433R/V441M/A515E | +++ |
| 3223/3224 | D198E/R264A/K266T/G414E | +++ |
| 3225/3226 | R264A/K266T/G414E/V441M/L499M/K501R/A515E/E520D | +++ |
| 3227/3228 | E243S/T396S/G414E/W433V/V441M/A515E/E520D | +++ |
| 3229/3230 | D198E/R264A/T396S/G414E/W433V/V441M/A515E | +++ |
| 3231/3232 | D198E/E243S/T396S/G414E/S431R/W433V/V441M/L499M | +++ |
| 3233/3234 | T396S/G414E/V441M/A515E | ++ |
| 3235/3236 | D198E/E243S/T396S/G414E/W433R/T437R/V441M/A515E | ++ |
| 3237/3238 | D198E/K266T/G414E/W433S/V441M/A515E | ++ |
| 3239/3240 | D198E/G414E/W433V/V441M/A515E/E520D | ++ |
| 3241/3242 | E186D/G231S/R248K/T484L | ++ |
| 3243/3244 | T396S/G414E/V441M | ++ |
| 3245/3246 | E243S/G414E/T437R/V441M/A515E | ++ |
| 3247/3248 | D198E/G414E/S431R/V441M/L499M/E520D | ++ |
| 3249/3250 | R264A/G414E | ++ |
| 3251/3252 | E186D/G231S/R248K/G365A/A366V/K368R/T484L | ++ |
| 3253/3254 | E243S/G414E/W433S/T437R/V441M | ++ |
| 3255/3256 | E186D/G231S/V441M/S444H/K445N | + |
| 3257/3258 | K266T/T437R/V441M/L499M/A515E/E520D | + |
| 3259/3260 | E186D/T484L/D485E | + |
| 3261/3262 | G414E/V441M/E520D | + |
| 3263/3264 | R264A/G414E/V441M | + |
| 3265/3266 | E186D/G231S/R248K/V253I/T484L | + |
| 3267/3268 | D198E/E243S/R264A/S431R/V441M | + |
| 3269/3270 | E243S/R264A/A515E/E520D | + |
| 3271/3272 | R248K/G442K/S444A/K445N/T484L | + |
| 3273/3274 | E186D/G231S/K368R/K416N/V441M/G442K/S444A/T484L/D485E | + |
| 3275/3276 | G365A/A366V/V441M/T484L/D485E | + |
| 3277/3278 | E186D/G231S/R248K/A366V/K368R/S444H | + |
| 3279/3280 | D198E/K266T/T396S/G414E/W433V/V441M/L499M/K501R | + |
| 3281/3282 | L188I/G231S/R248K/V253I/G365A/V441M/G442K/S444A/K445N/ | + |
| | T484L | |
| 3283/3284 | G231S/R248K/V441M/T484L | + |
| 3285/3286 | D198E/K266T/T437R/V441M/L499M | + |
| 3287/3288 | R264A/W433S/V441M | + |
| 3289/3290 | E186D/L188I/R248K/V253I/G365A/A366V/S444H/K445N | + |

Levels of increased activity were determined relative to the reference polypeptide of SEQ ID NO: 3174 and defined as follows: “+” 1.11 to 1.54, “++” >1.54, “+++” >1.79.

### Example 46

### Improvements Over SEQ ID NO: 3174 in the Extension of Oligonucleotide Acceptor Molecules with Nucleotide Triphosphate Donors

### HTP Screening for Improved TdT Variants

SEQ ID NO: 3174 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 46.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 46.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48° C.; Preincubation Time (min)-
60; Reaction temperature (C.)-48° C.; Reaction Time (min)-60; Reaction volume (μL)-1; NTP (μM)-
mATP-3′P; NTP conc (μM)-100; Oligonucleotide-5′-6-FAM-T15mAmU*mG; Substrate conc (μM)-
25; Product -5′-6-FAM-T15mAmU*mGmA-3′P.

Activity relative to SEQ ID NO: 3174 (Activity FIOP) was calculated as the percent product of the variant compared with the percent product observed by the reaction with SEQ ID NO: 3174 (where the percent product may be set as the average of replicates or else the highest single sample as appropriate). The results are shown in Table 46.2.

TABLE 46.2

| SEQ ID NO: | Amino Acid Differences | FIOP % product Relative |
|---|---|---|
| (nt/aa) | (Relative to SEQ ID NO: 3174) | to SEQ ID NO: 3174 |
| 3291/3292 | E394Y | +++ |
| 3293/3294 | M301 | +++ |
| 3295/3296 | M301T | +++ |
| 3297/3298 | M301V | +++ |
| 3299/3300 | E394F | +++ |
| 3301/3302 | Y390C | +++ |
| 3303/3304 | F194L | +++ |
| 3305/3306 | A408I | +++ |
| 3307/3308 | V273F | +++ |
| 3309/3310 | V273L | +++ |
| 3311/3312 | K274I | +++ |
| 3313/3314 | V273D | +++ |
| 3315/3316 | R473G | +++ |
| 3317/3318 | K274G | +++ |
| 3319/3320 | V273I | +++ |
| 3321/3322 | R196G | +++ |
| 3323/3324 | V273E/K501N | +++ |
| 3325/3326 | V273Y | +++ |
| 3327/3328 | V273E | +++ |
| 3329/3330 | K278F | +++ |
| 3331/3332 | G350W | +++ |
| 3333/3334 | K266Y | +++ |
| 3335/3336 | K274A | +++ |
| 3337/3338 | F347I | +++ |
| 3339/3340 | L312M | +++ |
| 3341/3342 | V304M | +++ |
| 3343/3344 | K274V | +++ |
| 3345/3346 | V273G | +++ |
| 3347/3348 | F269W | +++ |
| 3349/3350 | F194W | ++ |
| 3351/3352 | K266I | ++ |
| 3353/3354 | K297C | ++ |
| 3355/3356 | Y309F | ++ |
| 3357/3358 | G414A | ++ |
| 3359/3360 | E394V | ++ |
| 3361/3362 | R196N | ++ |
| 3363/3364 | E394A | ++ |
| 3365/3366 | N493V | ++ |
| 3367/3368 | A413S | ++ |
| 3369/3370 | Q302S | ++ |
| 3371/3372 | E394M | ++ |
| 3373/3374 | K416G | ++ |
| 3375/3376 | K266V | ++ |
| 3377/3378 | E394G | ++ |
| 3379/3380 | V268C | ++ |
| 3381/3382 | T299L | ++ |
| 3383/3384 | T299M | ++ |
| 3385/3386 | K266T | ++ |
| 3387/3388 | L454F | ++ |
| 3389/3390 | R196L | ++ |
| 3391/3392 | Y309W | ++ |
| 3393/3394 | R477G | ++ |
| 3395/3396 | L298I | ++ |
| 3397/3398 | Y309V | ++ |
| 3399/3400 | K407L | ++ |
| 3401/3402 | K278R | ++ |
| 3403/3404 | L454M | ++ |
| 3405/3406 | A408G | ++ |
| 3407/3408 | K407N | ++ |
| 3409/3410 | K407R | ++ |
| 3411/3412 | V268I | ++ |
| 3413/3414 | Y390V | ++ |
| 3415/3416 | Y309L | ++ |
| 3417/3418 | K407M | ++ |
| 3419/3420 | Q353A | ++ |
| 3421/3422 | Y309G | ++ |
| 3423/3424 | Q302N | ++ |
| 3425/3426 | L312V | ++ |
| 3427/3428 | R196T | ++ |
| 3429/3430 | R480H | ++ |
| 3431/3432 | Y309M | ++ |
| 3433/3434 | S267A | ++ |
| 3435/3436 | Q468M | ++ |
| 3437/3438 | G411N | + |
| 3439/3440 | K278V | + |
| 3441/3442 | V268L | + |
| 3443/3444 | V304D | + |
| 3445/3446 | V273C | + |
| 3447/3448 | K266F | + |
| 3449/3450 | S267H | + |
| 3451/3452 | E190L | + |
| 3453/3454 | L359G | + |
| 3455/3456 | H263M | + |
| 3457/3458 | R196M | + |
| 3459/3460 | Q468H | + |
| 3461/3462 | A413L | + |
| 3463/3464 | A413P | + |
| 3465/3466 | G411E | + |
| 3467/3468 | Y390I | + |
| 3469/3470 | N493Y | + |
| 3471/3472 | F194C | + |
| 3473/3474 | T299N | + |
| 3475/3476 | V268T | + |
| 3477/3478 | T299A | + |
| 3479/3480 | S267Y | + |
| 3481/3482 | L298M | + |
| 3483/3484 | A408L | + |
| 3485/3486 | G350L | + |
| 3487/3488 | A413F | + |
| 3489/3490 | Y309I | + |
| 3491/3492 | A408V | + |
| 3493/3494 | P352V | + |
| 3495/3496 | R281L | + |
| 3497/3498 | R281K | + |
| 3499/3500 | L479V | + |
| 3501/3502 | L312T | + |
| 3503/3504 | V304T | + |
| 3505/3506 | E193S | + |
| 3507/3508 | W279Y | + |
| 3509/3510 | M122I/R473K | + |
| 3511/3512 | K297L | + |
| 3513/3514 | S267C | + |
| 3515/3516 | T299Y | + |
| 3517/3518 | A413V | + |
| 3519/3520 | L479F | + |
| 3521/3522 | H263R | + |
| 3523/3524 | K278M | + |
| 3525/3526 | Q468F | + |
| 3527/3528 | S267V | + |
| 3529/3530 | E410F | + |
| 3531/3532 | E193T | + |
| 3533/3534 | G315Q | + |
| 3535/3536 | G350R | + |
| 3537/3538 | R480K | + |
| 3539/3540 | A408T | + |
| 3541/3542 | F194S | + |
| 3543/3544 | Q468T | + |
| 3545/3546 | K297T | + |
| 3547/3548 | P436S | + |
| 3549/3550 | K297M | + |
| 3551/3552 | R264C | + |
| 3553/3554 | P300H | + |
| 3555/3556 | R189L | + |
| 3557/3558 | K266R | + |
| 3559/3560 | S472G | + |
| 3561/3562 | K297R | + |
| 3563/3564 | K297V | + |
| 3565/3566 | E410G | + |
| 3567/3568 | E410I | + |
| 3569/3570 | L454V | + |
| 3571/3572 | E190M | + |
| 3573/3574 | K407W | + |
| 3575/3576 | E190R | + |
| 3577/3578 | Y390L | + |
| 3579/3580 | A408M | + |
| 3581/3582 | K278H | + |
| 3583/3584 | R392V | + |

Levels of increased activity were determined relative to the reference polypeptide of SEQ ID NO: 3174 and defined as follows: “+” 1.05 to 1.35, “++” >1.35, “+++” >1.84.

### Example 47

### Improvements Over SEQ ID NO: 3174 in the Extension of Oligonucleotide Acceptor Molecules with Nucleotide Triphosphate Donors

### HTP Screening for Improved TdT Variants

SEQ ID NO: 3174 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 47.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 47.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-65° C.; Preincubation Time (min)-
60; Reaction temperature (C.)-50° C.; Reaction Time (min)-60; Reaction volume (μL)-1; NTP (μM)-
mATP-3′P; NTP conc (μM)-100; Oligonucleotide-5′-6-FAM-T15AmU*mG; Substrate conc (μM)-25;
Product -5′-6-FAM-T15AmU*mGmA-3′P.

Activity relative to SEQ ID NO: 3174 (Activity FIOP) was calculated as the percent product of the variant compared with the percent product observed by the reaction with SEQ ID NO: 3174 (where the percent product may be set as the average of replicates or else the highest single sample as appropriate). The results are shown in Table 47.2.

TABLE 47.2

| SEQ ID NO: | Amino Acid Differences | FIOP % product Relative |
|---|---|---|
| (nt/aa) | (Relative to SEQ ID NO: 3174) | to SEQ ID NO: 3174 |
| 3315/3316 | R473G | +++ |
| 3299/3300 | E394F | +++ |
| 3291/3292 | E394Y | +++ |
| 3385/3386 | K266T | +++ |
| 3371/3372 | E394M | ++ |
| 3321/3322 | R196G | ++ |
| 3363/3364 | E394A | ++ |
| 3359/3360 | E394V | ++ |
| 3585/3586 | Q468A | ++ |
| 3587/3588 | R477S | ++ |
| 3525/3526 | Q468F | ++ |
| 3365/3366 | N493V | ++ |
| 3375/3376 | K266V | + |
| 3377/3378 | E394G | + |
| 3351/3352 | K266I | + |
| 3459/3460 | Q468H | + |
| 3543/3544 | Q468T | + |
| 3435/3436 | Q468M | + |
| 3403/3404 | L454M | + |
| 3319/3320 | V273I | + |
| 3411/3412 | V268I | + |
| 3309/3310 | V273L | + |
| 3521/3522 | H263R | + |
| 3497/3498 | R281K | + |
| 3589/3590 | K416S | + |

Levels of increased activity were determined relative to the reference polypeptide of SEQ ID NO: 3174 and defined as follows: “+” 1.20 to 1.58, “++” >1.58, “+++” >2.20.

### Example 48

### Improvements Over SEQ ID NO: 3174 in the Extension of Oligonucleotide Acceptor Molecules with Nucleotide Triphosphate Donors

### HTP Screening for Improved TdT Variants

SEQ ID NO: 3174 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 48.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 48.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48° C.; Preincubation Time (min)-
60; Reaction temperature (C.)-48° C.; Reaction Time (min)-60; Reaction volume (μL)-1; NTP (μM)-
mATP-3′P; NTP conc (μM)-100; Oligonucleotide-5′-6-FAM-T15mC*mG*mA; Substrate conc (μM)-
25; Product -5′-6-FAM-T15mC*mG*mAmA-3′P.

Activity relative to SEQ ID NO: 3174 (Activity FIOP) was calculated as the percent product of the variant compared with the percent product observed by the reaction with SEQ ID NO: 3174 (where the percent product may be set as the average of replicates or else the highest single sample as appropriate). The results are shown in Table 48.2.

TABLE 48.2

| SEQ ID NO: | Amino Acid Differences | FIOP % product Relative |
|---|---|---|
| (nt/aa) | (Relative to SEQ ID NO: 1830) | to SEQ ID NO: 1830 |
| 3589/3590 | K416S | +++ |
| 3373/3374 | K416G | +++ |
| 3297/3298 | M301V | +++ |
| 3301/3302 | Y390C | +++ |
| 3293/3294 | M301S | +++ |
| 3315/3316 | R473G | +++ |
| 3295/3296 | M301T | +++ |
| 3349/3350 | F194W | +++ |
| 3343/3344 | K274V | +++ |
| 3311/3312 | K274I | +++ |
| 3325/3326 | V273Y | +++ |
| 3321/3322 | R196G | +++ |
| 3307/3308 | V273F | ++ |
| 3327/3328 | V273E | ++ |
| 3323/3324 | V273E/K501N | ++ |
| 3303/3304 | F194L | ++ |
| 3347/3348 | F269W | ++ |
| 3367/3368 | A413S | ++ |
| 3319/3320 | V273I | ++ |
| 3407/3408 | K407N | ++ |
| 3345/3346 | V273G | ++ |
| 3583/3584 | R392V | ++ |
| 3365/3366 | N493V | ++ |
| 3305/3306 | A408I | ++ |
| 3317/3318 | K274G | ++ |
| 3413/3414 | Y390V | ++ |
| 3361/3362 | R196N | ++ |
| 3385/3386 | K266T | ++ |
| 3391/3392 | Y309W | ++ |
| 3417/3418 | K407M | ++ |
| 3403/3404 | L454M | + |
| 3431/3432 | Y309M | ++ |
| 3591/3592 | K407D | + |
| 3335/3336 | K274A | + |
| 3477/3478 | T299A | + |
| 3593/3594 | T299S | + |
| 3313/3314 | V273D | + |
| 3415/3416 | Y309L | + |
| 3475/3476 | V268T | + |
| 3399/3400 | K407L | + |
| 3309/3310 | V273L | + |
| 3375/3376 | K266V | + |
| 3595/3596 | D277E | + |
| 3355/3356 | Y309F | + |
| 3337/3338 | F347I | + |
| 3453/3454 | L359G | + |
| 3561/3562 | K297R | + |
| 3481/3482 | L298M | + |
| 3569/3570 | L454V | + |
| 3291/3292 | E394Y | + |
| 3393/3394 | R477G | + |
| 3369/3370 | Q302S | + |
| 3557/3558 | K266R | + |
| 3543/3544 | Q468T | + |
| 3409/3410 | K407R | + |
| 3433/3434 | S267A | + |
| 3473/3474 | T299N | + |
| 3425/3426 | L312V | + |
| 3597/3598 | K278Y | + |
| 3457/3458 | R196M | + |
| 3441/3442 | V268L | + |
| 3499/3500 | L479V | + |

Levels of increased activity were determined relative to the reference polypeptide of SEQ ID NO: 3174 and defined as follows: “+” 1.19 to 1.43, “++” >1.43, “+++” >2.15.

### Example 49

### Improvements Over SEQ ID NO: 3222 in the Extension of Oligonucleotide Acceptor Molecules with Nucleotide Triphosphate Donors

### HTP Screening for Improved TdT Variants

SEQ ID NO: 3222 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 49.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 49.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-50° C.; Preincubation Time (min)-
60; Reaction temperature (C.)-50° C.; Reaction Time (min)-60; Reaction volume (μL)-1; NTP (μM)-
mATP-3′P; NTP conc (μM)-100; Oligonucleotide-5′-6-FAM-T15mC*mG*mA; Substrate conc (μM)-
25; Product -5′-6-FAM-T15mC*mG*mAmA-3′P.

Activity relative to SEQ ID NO: 3222 (Activity FIOP) was calculated as the percent product of the variant compared with the percent product observed by the reaction with SEQ ID NO: 3222 (where the percent product may be set as the average of replicates or else the highest single sample as appropriate). The results are shown in Table 49.2.

TABLE 49.2

| SEQ | | FIOP % product |
|---|---|---|
| ID NO: | Amino Acid Differences | Relative to SEQ |
| (nt/aa) | (Relative to SEQ ID NO: 3222) | ID NO: 3222 |
| 3599/3600 | E186D/E243S/R248K/E394Y/T484L/D485E | +++ |
| 3601/3602 | F194L/E515A/E520D | +++ |
| 3603/3604 | D198E/R248K/E394Y/S396T/T484L/D485E/L499M | +++ |
| 3605/3606 | E186D/G231S/A366V/K368R/E394Y/D485E | +++ |
| 3607/3608 | D198E/E394Y/S396T/T484L/D485E/L499M | +++ |
| 3609/3610 | R248K/E394Y/T484L | +++ |
| 3611/3612 | R248K/G365A/A366V/K368R/E394Y/T484L/E520D | +++ |
| 3613/3614 | G231S/K368R/E394Y | ++ |
| 3615/3616 | E186D/G231S/E243S/K368R/E394Y/D485E/L499M | ++ |
| 3617/3618 | E186D/F194L/R248K/S396T | ++ |
| 3619/3620 | E186D/E243S/R248K/A366V/K368R/E394Y | ++ |
| 3621/3622 | E186D/A366V/K368R/E394Y/S396T/T484L | ++ |
| 3623/3624 | E186D/E394Y/S396T/D485E | ++ |
| 3625/3626 | E394Y/S396T | ++ |
| 3627/3628 | E394Y/L499M | ++ |
| 3629/3630 | E186D/G365A/A366V/E394Y/D485E | ++ |
| 3631/3632 | E186D/A366V/K368R/E394Y/S396T/T484L/D485E | ++ |
| 3633/3634 | E243S/T484L | ++ |
| 3635/3636 | G365A/A366V/K368R/E394Y | + |
| 3637/3638 | E186D/R248K | + |
| 3639/3640 | G365A/K368R/E394Y/S396T/E520D | + |
| 3641/3642 | E186D/D198E/E243S/R248K/A366V/K368R/E394Y/K501R | + |
| 3643/3644 | D198E/E394Y/S396T/L499M/E515A/E520D | + |
| 3645/3646 | E243S/R248K/E394Y/S396T/T484L/D485E | + |
| 3647/3648 | F194L/D198E/E243S/A366V/K368R/L499M | + |
| 3649/3650 | G231S/G365A/K368R/E394Y/L499M/E520D | + |
| 3651/3652 | D198E/E394Y/L499M/K501R | + |
| 3653/3654 | E186D/G365A/A366V/K368R/E394Y/L499M | + |
| 3655/3656 | E186D/G365A | + |
| 3657/3658 | E243S/T484L/D485E/L499M | + |
| 3659/3660 | D198E/E243S/R248K/G365A/E394Y/K501R | + |
| 3661/3662 | E186D/E243S/T484L/E520D | + |
| 3663/3664 | E186D/E243S | + |
| 3665/3666 | D198E/G231S/E243S/R248K/D485E | + |
| 3667/3668 | E186D/G231S/R248K/D485E | + |
| 3669/3670 | G231S/T484L/D485E/L499M/K501R | + |

Levels of increased activity were determined relative to the reference polypeptide of SEQ ID NO: 3222 and defined as follows: “+” 1.00 to 1.20, “++” >1.20, “+++” >1.49.

### Example 50

### Improvements Over SEQ ID NO: 3670 in the Extension of Oligonucleotide Acceptor Molecules with Nucleotide Triphosphate Donors

### HTP Screening for Improved TdT Variants

SEQ ID NO: 3670 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 50.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 50.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48° C.; Preincubation Time (min)-
60; Reaction temperature (C.)-48° C.; Reaction Time (min)-180; Reaction volume (μL)-1; NTP (μM)-
mATP-3′P; NTP conc (μM)-100; Oligonucleotide-5′-6-FAM-T17mGmUmC; Substrate conc (μM)-50;
Product -5′-6-FAM-T17mGmUmCmA-3′P.

Activity relative to SEQ ID NO: 3670 (Activity FIOP) was calculated as the percent product of the variant compared with the percent product observed by the reaction with SEQ ID NO: 3670 (where the percent product may be set as the average of replicates or else the highest single sample as appropriate). The results are shown in Table 50.2.

TABLE 50.2

| SEQ ID NO: | Amino Acid Differences | FIOP % product Relative |
|---|---|---|
| (nt/aa) | (Relative to SEQ ID NO: 3670) | to SEQ ID NO: 3670 |
| 3671/3672 | K274I/K416S | +++ |
| 3673/3674 | K416S | +++ |
| 3675/3676 | K274A/A408I/K416S | ++ |
| 3677/3678 | K274A/T299N/A408I/K416S | ++ |
| 3679/3680 | E288V/T299N/K416S | ++ |
| 3681/3682 | V273I | ++ |
| 3683/3684 | L298M/T299N/K416S | + |
| 3685/3686 | V273I/M499L | + |
| 3687/3688 | V273I/N493V/M499L | + |
| 3689/3690 | V273I/Y309M/N493V/M499L | + |
| 3691/3692 | Y309M | + |
| 3693/3694 | N493V/M499L | + |
| 3695/3696 | Y309M/M499L | + |

Levels of increased activity were determined relative to the reference polypeptide of SEQ ID NO: 3670 and defined as follows: “+” 1.07 to 1.39, “++” >1.39, “+++” >1.92.

### Example 51

### Improvements Over SEQ ID NO: 3670 in the Extension of Oligonucleotide Acceptor Molecules with Nucleotide Triphosphate Donors

### HTP Screening for Improved TdT Variants

SEQ ID NO: 3670 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 51.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 51.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48° C.; Preincubation Time (min)-
60; Reaction temperature (C.)-48° C.; Reaction Time (min)-180; Reaction volume (μL)-1; NTP (μM)-
mATP-3′P; NTP conc (μM)-100; Oligonucleotide-5′-6-FAM-T12mAmUmA, 5′-6-FAM-
T17mAmUmC, 5′-6-FAM-T22mAmUmU, 5′-6-FAM-T27mAmUmG; Substrate conc (μM)-multiplex:
12.5 μM each of 4 oligo; Product -5′-6-FAM-T12mAmUmAmA-3′P, 5′-6-FAM-T17mAmUmCmA-
3′P, 5′-6-FAM-T22mAmUmUmA-3′P, 5′-6-FAM-T27mAmUmGmA-3′P.

Activity relative to SEQ ID NO: 3670 (Activity FIOP) was calculated as the percent product of the variant, defined as the sum of the area of products in the multiplexed assay divided by the sum of the total peak area, compared with the percent product observed by the reaction with SEQ ID NO: 3670 (where the percent product may be set as the average of replicates or else the highest single sample as appropriate). The results are shown in Table 51.2.

TABLE 51.2

| SEQ ID NO: | Amino Acid Differences | FIOP % product Relative |
|---|---|---|
| (nt/aa) | (Relative to SEQ ID NO: 3670) | to SEQ ID NO: 3670 |
| 3697/3698 | R473S | +++ |
| 3699/3700 | Y308V | +++ |
| 3701/3702 | Y308S | +++ |
| 3703/3704 | M301S | +++ |
| 3705/3706 | Y308A/I361T | +++ |
| 3707/3708 | Y308G | +++ |
| 3709/3710 | Y308A | +++ |
| 3711/3712 | M301G | ++ |
| 3713/3714 | L419G | ++ |
| 3715/3716 | R456T | ++ |
| 3717/3718 | A415L | ++ |
| 3719/3720 | R451K | ++ |
| 3721/3722 | Q397F | ++ |
| 3723/3724 | M301Q | ++ |
| 3725/3726 | A415W | ++ |
| 3727/3728 | R456P | ++ |
| 3729/3730 | R473A | ++ |
| 3731/3732 | S267R | ++ |
| 3733/3734 | S472A | + |
| 3735/3736 | A313M | + |
| 3737/3738 | N493R/M499L | + |
| 3739/3740 | L307G | + |
| 3741/3742 | L419M | + |
| 3743/3744 | L307A | + |
| 3745/3746 | F475V | + |
| 3747/3748 | V452L | + |
| 3749/3750 | Y308K | + |
| 3751/3752 | R392V | + |
| 3753/3754 | D372E | + |
| 3755/3756 | D198S | + |
| 3757/3758 | S267M | + |
| 3759/3760 | N528L | + |
| 3761/3762 | L307S | + |
| 3763/3764 | Q397W | + |
| 3765/3766 | Y308H | + |
| 3767/3768 | C168S | + |

Levels of increased activity were determined relative to the reference polypeptide of SEQ ID NO: 3670 and defined as follows: “+” 1.18 to 1.43, “++” >1.43, “+++” >2.07.

### Example 52

### Improvements Over SEQ ID NO: 3670 in the Extension of Oligonucleotide Acceptor Molecules with Nucleotide Triphosphate Donors

### HTP Screening for Improved TdT Variants

SEQ ID NO: 3670 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 52.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 52.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-68° C.; Preincubation Time (min)-
60; Reaction temperature (C.)-50° C.; Reaction Time (min)-60; Reaction volume (μL)-1; NTP (μM)-
mATP-3′P; NTP conc (μM)-100; Oligonucleotide-5′-6-FAM-T15AmU*mG; Substrate conc (μM)-50;
Product -5′-6-FAM-T15AmU*mGmA-3′P.

Activity relative to SEQ ID NO: 3670 (Activity FIOP) was calculated as the percent product of the variant compared with the percent product observed by the reaction with SEQ ID NO: 3670 (where the percent product may be set as the average of replicates or else the highest single sample as appropriate). The results are shown in Table 52.2.

TABLE 52.2

| SEQ ID NO: | Amino Acid Differences | FIOP % product Relative |
|---|---|---|
| (nt/aa) | (Relative to SEQ ID NO: 3670) | to SEQ ID NO: 3670 |
| 3697/3698 | R473S | +++ |
| 3769/3770 | R473A | ++ |
| 3771/3772 | N493V/M499L | ++ |
| 3773/3774 | Y308L | + |
| 3775/3776 | R473M | + |
| 3777/3778 | K303H/S396A | + |

Levels of increased activity were determined relative to the reference polypeptide of SEQ ID NO: 3670 and defined as follows: “+” 1.08 to 1.34, “++” >1.34, “+++” >1.78.

### Example 53

### Improvements Over SEQ ID NO: 3670 in the Extension of Oligonucleotide Acceptor Molecules with Nucleotide Triphosphate Donors

### HTP Screening for Improved TdT Variants

SEQ ID NO: 3670 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 53.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 53.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48° C.; Preincubation Time (min)-
60; Reaction temperature (C.)-48° C.; Reaction Time (min)-180; Reaction volume (μL)-1; NTP (μM)-
mATP-3′P; NTP conc (μM)-100; Oligonucleotide-5′-6-FAM-T12mAmUmA, 5′-6-FAM-
T17mAmUmC, 5′-6-FAM-T22mAmUmU, 5′-6-FAM-T27mAmUmG; Substrate conc (μM)-multiplex:
12.5 μM each of 4 oligo; Product -5′-6-FAM-T12mAmUmAmA-3′P, 5′-6-FAM-T17mAmUmCmA-
3′P, 5′-6-FAM-T22mAmUmUmA-3′P, 5′-6-FAM-T27mAmUmGmA-3′P.

Activity relative to SEQ ID NO: 3670 (Activity FIOP) was calculated as the percent product of the variant, defined as the sum of the area of products in the multiplexed assay divided by the sum of the total peak area, compared with the percent product observed by the reaction with SEQ ID NO: 3670 (where the percent product may be set as the average of replicates or else the highest single sample as appropriate). The results are shown in Table 53.2.

TABLE 53.2

| SEQ ID NO: | Amino Acid Differences | FIOP % product Relative |
|---|---|---|
| (nt/aa) | (Relative to SEQ ID NO: 3670) | to SEQ ID NO: 3670 |
| 3671/3672 | K274I/K416S | +++ |
| 3675/3676 | K274A/A408I/K416S | +++ |
| 3673/3674 | K416S | +++ |
| 3677/3678 | K274A/T299N/A408I/K416S | +++ |
| 3679/3680 | E288V/T299N/K416S | ++ |
| 3687/3688 | V273I/N493V/M499L | ++ |
| 3681/3682 | V273I | ++ |
| 3689/3690 | V273I/Y309M/N493V/M499L | ++ |
| 3685/3686 | V273I/M499L | ++ |
| 3693/3694 | N493V/M499L | ++ |
| 3779/3780 | V273E/N493V | + |
| 3781/3782 | V273I/Y309M/A413S/M499L | + |
| 3783/3784 | A413S | + |
| 3785/3786 | Y309M/A413S | + |
| 3787/3788 | R281K/A413S/M499L | + |
| 3691/3692 | Y309M | + |
| 3683/3684 | L298M/T299N/K416S | + |
| 3789/3790 | A413S/N493V/M499L | + |
| 3791/3792 | A413S/M499L | + |
| 3793/3794 | V273S/Y309M/A413S/M499L | + |

Levels of increased activity were determined relative to the reference polypeptide of SEQ ID NO: 3670 and defined as follows: “+” 1.05 to 1.18, “++” >1.18, “+++” >1.35.

### Example 54

### Improvements Over SEQ ID NO: 3674 in the Extension of Oligonucleotide Acceptor Molecules with Nucleotide Triphosphate Donors

### HTP Screening for Improved TdT Variants

SEQ ID NO: 3674 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 54.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 54.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48° C.; Preincubation Time (min)-
60; Reaction temperature (C.)-48° C.; Reaction Time (min)-180; Reaction volume (μL)-1; NTP (μM)-
mATP-3′P; NTP conc (μM)-100; Oligonucleotide-5′-6-FAM-T12mAmUmA, 5′-6-FAM-
T17mAmUmC, 5′-6-FAM-T22mAmUmU, 5′-6-FAM-T27mAmUmG ; Substrate conc (μM)-multiplex:
12.5 μM each of 4 oligo; Product -5′-6-FAM-T12mAmUmAmA-3′P, 5′-6-FAM-T17mAmUmCmA-
3′P, 5′-6-FAM-T22mAmUmUmA-3′P, 5′-6-FAM-T27mAmUmGmA-3′P.

Activity relative to SEQ ID NO: 3674 (Activity FIOP) was calculated as the percent product of the variant, defined as the sum of the area of products in the multiplexed assay divided by the sum of the total peak area, compared with the percent product observed by the reaction with SEQ ID NO: 3674 (where the percent product may be set as the average of replicates or else the highest single sample as appropriate). The results are shown in Table 54.2.

TABLE 54.2

| SEQ | | FIOP % product |
|---|---|---|
| ID NO: | Amino Acid Differences | Relative to SEQ |
| (nt/aa) | (Relative to SEQ ID NO: 3674) | ID NO: 3674 |
| 3795/3796 | F194L/R196G/Y390C | +++ |
| 3797/3798 | Y390C/E394F/R480K | +++ |
| 3799/3800 | R196G/Y390C/E394F | +++ |
| 3801/3802 | F194L/R196G/Y390C/R480K | +++ |
| 3803/3804 | F194L/R196G/Y390C/E394F/R480K | +++ |
| 3805/3806 | F194L/R196G/Y390C/L454M/R480K | +++ |
| 3807/3808 | Y390C/E394F | ++ |
| 3809/3810 | R196G/Y390C/E394F/R480K | ++ |
| 3811/3812 | F194L/Y390C | ++ |
| 3813/3814 | F194L/R196G/Y390C/E394F/G460V/ | ++ |
| | R480K | |
| 3815/3816 | R196G/Y390C/E394F/L454M/R480K | ++ |
| 3817/3818 | R196G/Y390C | ++ |
| 3819/3820 | Y390C | ++ |
| 3821/3822 | Y390C/E394F/L454M/R480K | ++ |
| 3823/3824 | R196G/Y390C/E394F/L454M | ++ |
| 3825/3826 | Y390C/E394F/L454M | ++ |
| 3827/3828 | Y390C/R480K | ++ |
| 3829/3830 | Y390C/L454M | + |
| 3831/3832 | R196G/Y390C/L454M | + |
| 3833/3834 | R196G/E394F | + |
| 3835/3836 | K297R/R473G/N493V | + |
| 3837/3838 | F194L/E394F | + |
| 3839/3840 | R196G/E394F/R480K | + |
| 3841/3842 | E394F/R480K | + |
| 3843/3844 | E394F | + |
| 3845/3846 | F194L/R196G/L454M | + |
| 3847/3848 | F194L/E394F/L454M/R480K | + |
| 3849/3850 | K297R/T470S/R473G | + |
| 3851/3852 | F194L/R196G/E394F/L454M/R480K | + |
| 3853/3854 | R196G/E394F/L454M/R480K | + |
| 3855/3856 | R196G/E394F/L454M | + |
| 3857/3858 | F194L/E394F/L454M | + |
| 3859/3860 | R196G/L454M | + |
| 3861/3862 | R196G | + |
| 3863/3864 | L454M | + |

Levels of increased activity were determined relative to the reference polypeptide of SEQ ID NO: 3674 and defined as follows: “+” 1.22 to 2.98, “++” >2.98, “+++” >3.79.

### Example 55

### Improvements Over SEQ ID NO: 3796 in the Extension of Oligonucleotide Acceptor Molecules with Nucleotide Triphosphate Donors

### HTP Screening for Improved TdT Variants

SEQ ID NO: 3796 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 55.1.

Reactions were performed in 96-well format 200 μL BioRad PCR plates. Reactions included 10-100 μM oligonucleotide, 100-200 μM nucleotide triphosphate, 20 mM buffer, and 250 μM cobalt (II) chloride. The reactions were set up as follows: (i) all reaction components, except for TdT, were pre-mixed in a single solution and were aliquoted into each well of the 96-well plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 55.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48° C.; Preincubation Time (min)-
60; Reaction temperature (C.)-48° C.; Reaction Time (min)-180; Reaction volume (μL)-1; NTP (μM)-
mATP-3′P; NTP conc (μM)-200; Oligonucleotide-5′-6-FAM-T12mAmUmA, 5′-6-FAM-
T17mAmUmC, 5′-6-FAM-T22mAmUmU, 5′-6-FAM-T27mAmUmG, 5′-6-FAM-T57mUmUmC, 5′-6-
FAM-T32mAmCmC, 5′-6-FAM-T37mAmGmC, 5′-6-FAM-T42mAmAmC, 5′-6-FAM-T47mGmUmC,
5′-6-FAM-T52mCmUmC; Substrate conc (μM)-multiplex: 10 μM each of 10 oligo; Product -5′-6-
FAM-T12mAmUmAmA-3′P, 5′-6-FAM-T17mAmUmCmA-3′P, 5′-6-FAM-T22mAmUmUmA-3′P, 5′-
6-FAM-T27mAmUmGmA-3′P, 5′-6-FAM-T57mUmUmCmA-3′P, 5′-6-FAM-T32mAmCmCmA-3′P,
5′-6-FAM-T37mAmGmCmA-3′P, 5′-6-FAM-T42mAmAmCmA-3′P, 5′-6-FAM-T47mGmUmCmA-
3′P, 5′-6-FAM-T52mCmUmCmA-3′P.

Activity relative to SEQ ID NO: 3796 (Activity FIOP) was calculated as the percent product of the variant, defined as the sum of the area of products in the multiplexed assay divided by the sum of the total peak area, compared with the percent product observed by the reaction with SEQ ID NO: 3796 (where the percent product may be set as the average of replicates or else the highest single sample as appropriate). The results are shown in Table 55.2.

TABLE 55.2

| SEQ | | FIOP % product |
|---|---|---|
| ID NO: | Amino Acid Differences | Relative to SEQ |
| (nt/aa) | (Relative to SEQ ID NO: 3796) | ID NO: 3796 |
| 3865/3866 | D198P/R451K/N493V | +++ |
| 3867/3868 | D198P/S267Q/R314A/R451K | +++ |
| 3869/3870 | S267R/R451K | +++ |
| 3871/3872 | R451K | +++ |
| 3873/3874 | D198P/S267Q/D409L/R451K | +++ |
| 3875/3876 | R451K/H494V | ++ |
| 3877/3878 | V328T/R451K | ++ |
| 3879/3880 | R314A/R451K | ++ |
| 3881/3882 | S267R/R314A/V328T/R451K/H494R/ | ++ |
| | M499L | |
| 3883/3884 | D198P | ++ |
| 3885/3886 | R451K/N493V/M499L | ++ |
| 3887/3888 | S267Q/R451K/H494V/M499L | ++ |
| 3889/3890 | R314A/V328T/R451K/M499L | ++ |
| 3891/3892 | I208V/Y308V | + |
| 3893/3894 | V328T/D409L/R451K | + |
| 3895/3896 | D198P/S267Q/R314A/F475V | + |
| 3897/3898 | D198P/S267R/A313L/R451K/F475V/ | + |
| | H494V/M499L | |
| 3899/3900 | S267R | + |
| 3901/3902 | D198P/S267Q/F475V | + |
| 3903/3904 | D409L | + |
| 3905/3906 | I208V/Y308A/S461N | + |
| 3907/3908 | Y308A | + |
| 3909/3910 | F475V | + |
| 3911/3912 | R451K/N493V/H494V | + |
| 3913/3914 | D409L/F475V/H494V | + |
| 3915/3916 | Y308V | + |

Levels of increased activity were determined relative to the reference polypeptide of SEQ ID NO: 3796 and defined as follows: “+” 1.12 to 1.33, “++” >1.33, “+++” >1.62.

### Example 56 Activity Improvement Over SEQ ID NO: 3870

Activity Improvement of Shake-Flask Purified TdT Variants with 3′Phosphate-Blocked Nucleotides and Modified RNA Oligonucleotide Substrates.

TdT variants of SEQ ID NO: 3870, 3918, and 3920 were produced in shake flask and purified as described in Example 3.

Reactions were performed in 96-well format 200 μL BioRad PCR plates. Reactions included 250 μM oligonucleotide, 300 μM nucleotide triphosphate, 4 μM TdT, 5 μM IPP, 20 mM triethanolamine (pH 7.8), and 250 μM cobalt (II) chloride. The reactions were set up as follows: (i) all reaction components, except for TdT, were pre-mixed in a single solution and were aliquoted into each well of the 96-well plates (ii) TdT solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 56.1
Reaction conditions

Reaction buffer-20 mM TEA, 0.25 mM CoCl₂, pH 7.8; IPP SEQ ID NO: 3942 (μM)- 0.5;
Preincubation Temp (C.) - none; Preincubation Time (min)- none; Reaction temperature (C.)- 50° C.;
Reaction Time (min)- 120; Reaction volume (μL)- 1; TdT concentration (μM)- 4; NTP- mATP; NTP
conc (μM)-300; Oligonucleotide-10x multiplex [5′-6-FAM-T12mAmUmA, 5′-6-FAM-T17mAmUmC,
5′-6-FAM-T22mAmUmU, 5′-6-FAM-T27mAmUmG, 5′-6-FAM-T57mUmUmC, 5′-6-FAM-
T32mAmCmC, 5′-6-FAM-T37mAmGmC, 5′-6-FAM-T42mAmAmC, 5′-6-FAM-T47mGmUmC, 5′-6-
FAM-T52mCmUmC], 5′-6-FAM-T15mAmU(2′dF)G, 5′-6-FAM-T15mAmG(MOE)C; Oligonucleotide
conc (μM)-250 or 25 each oligo for 10x multiplex; Product- 10x multiplex [5′-6-FAM-
T12mAmUmAmA-3′P, 5′-6-FAM-T17mAmUmCmA-3′P, 5′-6-FAM-T22mAmUmUmA-3′P, 5′-6-
FAM-T27mAmUmGmA-3′P, 5′-6-FAM-T57mUmUmCmA-3′P, 5′-6-FAM-T32mAmCmCmA-3′P, 5′-
6-FAM-T37mAmGmCmA-3′P, 5′-6-FAM-T42mAmAmCmA-3′P], 5′-6-FAM-T15mAmU(2′dF)GmA-
3′P, 5′-6-FAM-T15mAmG(MOE)CmA-3′P.

Activity relative to SEQ ID NO: 3870 (Activity FIOP) was calculated as the percent product of the variant, defined as the sum of the area of products in the multiplexed assay divided by the sum of the total peak area, compared with the percent product observed by the reaction with SEQ ID NO: 3870. The results are shown in Tables 56.2, 56.3, and 56.4.

TABLE 56.2
Products- 10x multiplex [5′-6-FAM-T12mAmUmAmA-
3′P, 5′-6-FAM-T17mAmUmCmA-3′P, 5′-6-
FAM-T22mAmUmUmA-3′P, 5′-6-FAM-T27mAmUmGmA-
3′P, 5′-6-FAM-T57mUmUmCmA-3′P, 5′-6-
FAM-T32mAmCmCmA-3′P, 5′-6-FAM-T37mAmGmCmA-
3′P, 5′-6-FAM-T42mAmAmCmA-3′P]

| SEQ ID NO: | Amino Acid Differences | FIOP % product Relative |
|---|---|---|
| (nt/aa) | (Relative to SEQ ID NO: 3870) | to SEQ ID NO: 3870 |
| 3917/3918 | M301W | + |

Levels of increased activity were determined relative to the reference polypeptide of SEQ ID NO: 3870 and defined as follows: “+” from 3.25 to 4.37.

TABLE 56.3
Product 5′-6-FAM-T15mAmU(2′dF)GmA-3′P

| SEQ ID NO: | Amino Acid Differences | FIOP % product Relative |
|---|---|---|
| (nt/aa) | (Relative to SEQ ID NO: 3870) | to SEQ ID NO: 3870 |
| 3917/3918 | M301W | + |

Levels of increased activity were determined relative to the reference polypeptide of SEQ ID NO: 3870 and defined as follows: “+” from 3.25 to 4.37.

TABLE 56.4
Product 5′-6-FAM-T15mAmG(MOE)CmA-3′P

| SEQ ID NO: | Amino Acid Differences | FIOP % product Relative |
|---|---|---|
| (nt/aa) | (Relative to SEQ ID NO: 3870) | to SEQ ID NO: 3870 |
| 3917/3918 | M301W | + |
| 3919/3920 | M301G | + |

Levels of increased activity were determined relative to the reference polypeptide of SEQ ID NO: 3870 and defined as follows: “+” from 3.25 to 4.37.

### Example 57

### Gene Acquisition and Expression of Wild-Type Alkaline Phosphatases

Synthetic genes encoding an N-terminal 6-histidine tagged version of multiple wild-type (WT) alkaline phosphatases were cloned into the pCK1 10900 vector system (See e.g., U.S. Pat. No. 9,714,437, which is hereby incorporated by reference in its entirety) and subsequently expressed in an E. coli strain derived from W3110.

Cells transformed with the alkaline phosphatase expression constructs were grown at shake-flask scale using IPTG induction as described in Example 3. Cells were then lysed, purified, and dialyzed into storage buffer (20 mM Tris-HCl, pH 7.4, 100 mM KCl, 0.1 mM EDTA, and 50% glycerol). After overnight dialysis, protein samples were removed, and enzyme concentrations were measured by absorption at 280 nm using a NanoDrop™ 1000 spectrophotometer. Soluble protein concentrations are summarized in Table 57.1 below, showing a fold improvement in soluble protein production following shake-flask purification relative to the alkaline phosphatase from Thermotoga neapolitana (SEQ ID NO: 3930).

TABLE 57.1
Soluble Enzyme Production of Variants
Relative to SEQ ID NO: 3922

| SEQ | | FIOP Soluble Enzyme |
|---|---|---|
| ID NO: | Source organism of | Production (Relative |
| (nt/aa) | AP gene sequence | to SEQ ID NO: 3930) |
| 3923/3924 | Pyrococcus furiosus | + |
| 3921/3922 | Thermotoga maritima | + |
| 3925/3926 | Thermotoga sp. 50_64 | ++ |
| 3927/3928 | Pseudothermotoga lettingae | + |
| 3929/3930 | Thermotoga neapolitana | + |
| 3931/3932 | Thermoflexibacter ruber | +++ |
| 3933/3934 | Bacillus licheniformis | ++ |

Levels of increased soluble enzyme production were relative to the reference polypeptide of SEQ ID NO: 3930 and defined as follows: “+” 1.00 to 1.30, “++” >1.30, “+++” >1.80.

### Example 58

### Gene Acquisition and Expression of Wild-Type Inorganic Pyrophosphatases

Synthetic genes encoding a C-terminal 6-histidine tagged version of multiple wild-type (WT) inorganic pyrophosphatase enzymes were cloned into the pCK110900 vector system (See e.g., U.S. Pat. No. 9,714,437, which is hereby incorporated by reference in its entirety) and subsequently expressed in an E. coli strain derived from W3110.

Cells transformed with the inorganic pyrophosphatase expression constructs were grown at shake-flask scale using IPTG induction as described in Example 3. Cells were then lysed, purified, and dialyzed into storage buffer (20 mM Tris-HCl, pH 7.4, 100 mM KCl, 0.1 mM EDTA, and 50% glycerol). After overnight dialysis, protein samples were removed, and enzyme concentrations were measured by absorption at 280 nm using a NanoDrop™ 1000 spectrophotometer. Soluble protein concentrations are summarized in Table 58.1 below, showing a fold improvement in soluble protein production following shake-flask purification relative to the inorganic pyrophosphatase from Thermocrinis ruber (SEQ ID NO: 3936).

TABLE 58.1
Soluble Enzyme Production of Variants
Relative to SEQ ID NO: 3936

| SEQ | | FIOP Soluble Enzyme |
|---|---|---|
| ID NO: | Source organism of | Production (Relative |
| (nt/aa) | IPP gene sequence | to SEQ ID NO: 3936) |
| 3935/3936 | Thermocrinis ruber | + |
| 3937/3938 | Aquifex pyrophilus | + |
| 3939/3940 | Thermus oshimai | + |
| 3941/3942 | Sulfolobus sp. A20 | +++ |
| 3943/3944 | Geobacillus zalihae | ++ |
| 3945/3946 | Bacillus thermozeamaize | ++ |
| 3947/3948 | Bacillus smithii | + |

Levels of increased soluble enzyme production were determined relative to the reference polypeptide of SEQ ID NO: 3936 and defined as follows: “+” 1.00 to 4.80, “++” >4.80, “+++” >6.10.

### Example 59

### 3′Phosphate Deblocking Activity of Shake-Flask Purified Wild-Type Alkaline Phosphatases

Wild-type (WT) alkaline phosphatases (APs) SEQ ID NO: 3924, 3922, 3926, 3928, 3930, 3932, and 3934 (as described in Example 56) were assayed for 3′dephosphorylation activity of oligonucleotide 5′-6-FAM-T15mGmAmC-3′P.

Reactions were performed in 96-well format 200 μL BioRad PCR plates. Reactions included 25 μM 3′blocked oligonucleotide, 1.2 μM AP, 20 mM triethanolamine (pH 7.8), and 250 μM cobalt (II) chloride. The reactions were set up as follows: (i) all reaction components, except for AP, were pre-mixed in a single solution and were aliquoted into each well of the 96-well plates (ii) AP solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 59.1
Reaction conditions

Reaction buffer-20 mM TEA, 0.25 mM CoCl₂, pH 7.8; Preincubation Temp (C.) - none; Preincubation
Time (min)- none; Reaction temperature (C.)- 37° C.; Reaction Time (min)- 60; Reaction volume (μL)-
1; AP concentration (μM)- 1.2; Oligonucleotide-5′-6-FAM-T15mGmAmC-3′P; Oligonucleotide conc
(μM)- 25; Product- 5′-6-FAM-T15mGmAmC.

Percent dephosphorylation was calculated as the product peak area divided by the sum of the total peak area of the electropherogram. The results are shown in Table 59.2.

TABLE 59.2
Dephosphorylation activity of alkaline phosphatases
with a 3′phosphorylated oligonucleotide

| SEQ ID NO: | Conversion to de-phosphorylated |
|---|---|
| (nt/aa) | oligonucleotide |
| 3923/3924 | + |
| 3921/3922 | + |
| 3925/3926 | + |
| 3927/3928 | ++ |
| 3929/3930 | +++ |
| 3931/3932 | ++ |
| 3933/3934 | + |

Percent dephosphorylation is defined as follows: “+” 97.11 to 98.29, “++” >98.29, +++” >98.39.

### Example 60

Suppression of by-Products Arising from TdT Reactions by Shake-Flask Purified Wild-Type Inorganic Pyrophosphatases.

Wild-type (WT) inorganic pyrophosphatases SEQ ID NO: 3936, 3938, 3940, 3942, 3944, 3946, and 3948 (as described in Example 59) were assayed for suppression of by-products generated in a reaction containing TdT SEQ ID NO: 3674.

Reactions were performed in 96-well format 200 μL BioRad PCR plates. Reactions included 80 μM oligonucleotide, 100 μM 3′phosphate blocked NTP, 10 μM TdT, 0.5 μM IPP, 20 mM triethanolamine (pH 7.8), and 250 μM cobalt (II) chloride. The reactions were set up as follows: (i) all reaction components, except for IPP, were pre-mixed in a single solution and were aliquoted into each well of the 96-well plates (ii) IPP solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 60.1
Reaction conditions

Reaction buffer-20 mM TEA, 0.25 mM CoCl₂, pH 7.8; Preincubation Temp (C.) - none; Preincubation
Time (min)- none; Reaction temperature (C.)- 50° C.; Reaction Time (min)- 120; Reaction volume (μL) −1;
IPP concentration (μM)- 0.5; TdT concentration (μM)- 10; NTP (μM)-3′P-mATP; NTP conc (μM)-
100; Oligonucleotide-5′-6-FAM-T15mGmAmC 5′-6-FAM-T15mGmAmC-3′P; Oligonucleotide conc
(μM)- 80; Product- 5′-6-FAM-T15mGmAmC-3′P; By-Product-unknown.

The relative suppression of by-products relative to a control without IPP was calculated as the percent by-product of the variant compared with the percent by-product observed by the control reaction with no IPP present. The results are shown in Table 60.2.

TABLE 60.2
Suppression of TdT reaction by-products by IPP

| SEQ ID NO: | |
|---|---|
| (nt/aa) | Relative suppression of by-products |
| 3935/3936 | + |
| 3937/3938 | ++ |
| 3939/3940 | +++ |
| 3941/3942 | + |
| 3943/3944 | ++ |
| 3945/3946 | + |
| 3947/3948 | + |

Levels of suppression of by-products are defined as follows: “+” 4.01 to 4.32, “++” >4.32, “+++” >4.41.

### Example 61 Activity Improvement Over SEQ ID NO: 660

Activity Improvement of Shake-Flask Purified TdT Variants with 3′Phosphate-Blocked Nucleotides and Modified RNA Oligonucleotide Substrates.

TdT variants of SEQ ID NO: 660, 1654, 2254, 2812, 3674 and 3674 were produced in shake flask and purified as described in Example 3.

Reactions were performed in 96-well format 200 μL BioRad PCR plates. Reactions included 25 μM oligonucleotide, 200 μM nucleotide triphosphate, 10 μM TdT, 0.5 μM IPP, 20 mM triethanolamine (pH 7.8), and 250 μM cobalt (II) chloride. The reactions were set up as follows: (i) all reaction components, except for TdT, were pre-mixed in a single solution and were aliquoted into each well of the 96-well plates (ii) TdT solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 61.1
Reaction conditions

Reaction buffer-20 mM TEA, 0.25 mM CoCl₂, pH 7.8; IPP SEQ ID NO: 3944 (μM)- 0.5;
Preincubation Temp (C.) - none; Preincubation Time (min)- none; Reaction temperature (C.)- 40° C.;
Reaction Time (min)- 999 min; Reaction volume (μL) −1; TdT concentration (μM)- 10; NTP- 3′P-mA,
3′P-(2′dF)A, 3′P-(2′dF)U, 3′P-mC, 3′P-mG, 3′P-mU; NTP conc (μM)-200; Oligonucleotide- 5′-6-FAM-
T15AT*mG, 5′-6-FAM-T22*(2′dF)A(2′dF)GmA, 5′-6-FAM-T15AmU*mG, 5′-6-FAM-
T22(2′dF)C(2′dF)G(2′dF)A, 5′-6-FAM-T27(2′dF)GmA(2′dF)U, 5′-6-FAM-T15mGmAmC, 5′-6-FAM-
T11mCmGmA, 5′-6-FAM-T15mUmGmA, 5′-6-FAM-T11mC*mA*mG, 5′-6-FAM-T15mAmU*mG,
5′-6-FAM-T15mA(2′dF)UmC, 5′-6-FAM-T15mCmUmG, 5′-6-FAM-T27(2′dF)C*(2′dF)G*(2′dF)A, 5′-
6-FAM-T11mU*(2′dF)A*(2′dF)A, 5′-6-FAM-T17mAmUmC, 5′-6-FAM-T48mG*mA*mC, 5′-6-
FAM-T15mAmCmU, 5′-6-FAM-T15mAmG(MOE)C, 5′-6-FAM-T17*(2′dF)A*(2′dF)A(2′dF)G, 5′-6-
FAM-T15mAmU(2′dF)G, 5′-6-FAM-T15mAmU(2′dF)U, 5′-6-FAM-T15mAmU(2′dF)C;
Oligonucleotide conc (μM)-25; Product- 5′-6-FAM-T15AT*mGmA-3′P, 5′-6-FAM-
T22*(2′dF)A(2′dF)GmA(2′dF)A-3′P, 5′-6-FAM-T15AmU*mGmA-3′P, 5′-6-FAM-
T22(2′dF)C(2′dF)G(2′dF)A(2′dF)A-3′P, 5′-6-FAM-T27(2′dF)GmA(2′dF)U(2′dF)A-3′P, 5′-6-FAM-
T15mGmAmC(2′dF)A-3′P, 5′-6-FAM-T11mCmGmA(2′dF)A-3′P, 5′-6-FAM-T15mUmGmAmA-3′P,
5′-6-FAM-T11mC*mA*mG(2′dF)A-3′P, 5′-6-FAM-T15mAmU*mGmA-3′P, 5′-6-FAM-
T11mC*mA*mGmA-3′P, 5′-6-FAM-T15mA(2′dF)UmCmA-3′P, 5′-6-FAM-T15mCmUmGmA-3′P, 5′-
6-FAM-T27(2′dF)C*(2′dF)G*(2′dF)A(2′dF)A-3′P, 5′-6-FAM-T11mU*(2′dF)A*(2′dF)A(2′dF)A-3′P,
5′-6-FAM-T17mAmUmCmA-3′P, 5′-6-FAM-T15mGmAmC(2′dF)U-3′P, 5′-6-FAM-
T48mG*mA*mCmA-3′P, 5′-6-FAM-T15mAmCmU(2′dF)A-3′P, 5′-6-FAM-T15mGmAmCmA-3′P, 5′-
6-FAM-T15mAmCmU(2′dF)U-3′P, 5′-6-FAM-T15mAmCmUmC-3′P, 5′-6-FAM-T15mAmCmUmG-
3′P, 5′-6-FAM-T15mAmCmUmA-3′P, 5′-6-FAM-T15mAmG(MOE)CmA-3′P, 5′-6-FAM-
T17*(2′dF)A*(2′dF)A(2′dF)G(2′dF)A-3′P, 5′-6-FAM-T15mGmAmCmC-3′P, 5′-6-FAM-
T15mAmU(2′dF)GmA-3′P, 5′-6-FAM-T15mAmU(2′dF)UmA-3′P, 5′-6-FAM-T15mGmAmCmG-3′P,
5′-6-FAM-T15mAmCmU*mU-3′P, 5′-6-FAM-T15mGmAmC*mU-3′P, 5′-6-FAM-
T15mGmAmC*mG-3′P, 5′-6-FAM-T15mAmCmU*mG-3′P, 5′-6-FAM-T15mAmU(2′dF)CmA-3′P.

SEQ ID NO: 660, 1654, 2254, 2812, 3674 and 3674, shown in Table 61.2, were evaluated in the experiments described in Tables 61.3-61.5.

TABLE 61.2
Sequences of variants used in tables 61.3-61.6

| SEQ ID | |
|---|---|
| NO: | Amino Acid Differences |
| (nt/aa) | (Relative to SEQ ID NO: 660) |
| 1653/1654 | G92R/D94E/T101E/T108K/M137A/V141E/I155E/C201A/C213S/L264R/D314R/W333A/ |
| | T344V/D392R/R394E/E395W/R397Q/R406G/1408A/H425D/S442G/S446P/R461G/E476R/ |
| | R481W/H485D/S525F |
| 2253/2254 | G92R/D94E/T101E/T108K/M137A/V141E/1155E/T160M/I163V/Q165P/C201A/T203E/ |
| | M205R/C213S/V219R/R258W/K263H/L264R/D314R/W333A/T344V/F353Q/D392R/R394E/ |
| | E395W/R397Q/R406G/1408A/L411G/H413A/F414G/H425D/E441M/S442G/S446P/D460G/ |
| | R461G/G468Q/E476R/R481W/H485D/K488N/S525F |
| 2811/2812 | R26Q/I38T/S79T/S81E/N90L/G92R/D94E/T101E/T108K/M137A/V141E/I155E/T160M/ |
| | I163V/Q165P/A189R/C201A/T203E/M205R/C213S/V219R/E246N/L248R/R258W/K263H/ |
| | L264R/A304V/C307L/D314R/K318R/W333A/L340I/T344V/F353Q/T362S/D392R/R394E/ |
| | E395W/S396T/R397Q/F398W/K402G/L403S/R406G/1408A/A410E/L411G/H413A/F414G/ |
| | H425D/E441M/S442G/S446P/V455L/D460G/R461G/L466M/G468Q/E476R/R481W/H485D/ |
| | K488N/K506P/S525F |
| 3673/3674 | R26Q/M30G/I38T/S79T/S81E/N90L/G92R/D94E/T101E/T108K/M137A/G140V/V141E/ |
| | M142V/E153V/I155E/T160M/I163V/Q165P/H177R/A184S/A189R/C201A/T203E/M205R/ |
| | C213S/V219R/G231S/L248R/R258W/K263H/L264A/K266T/A304V/C307L/D314R/K318R/ |
| | S325W/W333A/L340I/T344V/F353Q/T362S/T379M/D392R/R394E/E395W/R397Q/F398W/ |
| | K402G/L403S/R406G/I408A/A410E/L411G/H413A/F414E/K416S/H425D/K427L/D429R/ |
| | W433R/E434N/E441M/S442G/A444S/S446P/V455L/D460G/R461S/L466M/G468Q/E476R/ |
| | R481W/T484L/H485E/K488N/A495S/L499M/K501R/K502G/K506P/A515E/S525F |
| 3917/3918 | R26Q/M30G/I38T/S79T/S81E/N90L/G92R/D94E/T101E/T108K/M137A/G140V/V141E/ |
| | M142V/E153V/I155E/T160M/I163V/Q165P/H177R/A184S/A189R/F194L/R196G/C201A/ |
| | T203E/M205R/C213S/V219R/G231S/L248R/R258W/K263H/L264A/K266T/S267R/M301W/ |
| | A304V/C307L/D314R/K318R/S325W/W333A/L340I/T344V/F353Q/T362S/T379M/Y390C/ |
| | D392R/R394E/E395W/R397Q/F398W/K402G/L403S/R406G/1408A/A410E/L411G/H413A/ |
| | F414E/K416S/H425D/K427L/D429R/W433R/E434N/E441M/S442G/A444S/S446P/R451K/ |
| | V455L/D460G/R461S/L466M/G468Q/E476R/R481W/T484L/H485E/K488N/A495S/L499M/ |
| | K501R/K502G/K506P/A515E/S525F |

Activity relative to SEQ ID NO: 660 (Activity FIOP) was calculated as the percent product of the variant compared with the percent product observed by the reaction with SEQ ID NO: 660. The results are shown in Table 61.3.

TABLE 61.3
Product 5′-6-FAM-T15AT*mGmA-3′P

| SEQ ID NO: | FIOP Product % conversion Relative |
|---|---|
| (nt/aa) | to SEQ ID NO: 660 |
| 1653/1654 | + |
| 2253/2254 | ++ |
| 2811/2812 | +++ |
| 3673/3674 | + |
| 3917/3918 | ++ |

Levels of increased activity were determined relative to the reference polypeptide of SEQ ID NO: 660 and defined as follows: “+” 1.00 to 12.00, “++” >12.00, “+++” >19.00.

Activity relative to SEQ ID NO: 1654 (Activity FIOP) was calculated as the percent product of the variant compared with the percent product observed by the reaction with SEQ ID NO: 1654. The results are shown in Table 61.4.

TABLE 61.4
Products ^{a}5′-6-FAM-T22*(2′dF)A(2′dF)GmA(2′dF)A-3′P, ^{b}5′-6-FAM-
T15AmU*mGmA-3′P, ^{c}5′-6-FAM-T22(2′dF)C(2′dF)G(2′dF)A(2′dF)A-
3′P, ^{d}5′-6-FAM-T27(2′dF)GmA(2′dF)U(2′dF)A-3′P, ^{e}5′-
6-FAM-T15mGmAmC(2′dF)A-3′P, ^{f}5′-6-FAM-T11mCmGmA(2′dF)A-3′P,
^{g}5′-6-FAM-T15mUmGmAmA-3′P, ^{h}5′-6-FAM-T11mC*mA*mG(2′dF)A-
3′P, ⁱ5′-6-FAM-T15mAmU*mGmA-3′P, ^{j}5′-6-FAM-T11mC*mA*mGmA-
3′P, ^{k}5′-6-FAM-T15mA(2′dF)UmCmA-3′P.

| SEQ | | | | | | | | | | | |
|---|---|---|---|---|---|---|---|---|---|---|---|
| ID NO: | FIOP Product % conversion Relative to SEQ ID NO: 1654 | | | | | | | | | | |
| (nt/aa) | a | b | c | d | e | f | g | h | i | j | k |
| 2253/2254 | + | + | + | + | + | + | + | + | + | + | + |
| 2811/2812 | + | + | + | + | + | + | + | + | ++ | ++ | ++ |
| 3673/3674 | + | + | + | + | + | + | ++ | ++ | ++ | +++ | +++ |
| 3917/3918 | + | + | + | + | + | + | ++ | ++ | ++ | +++ | +++ |

Levels of increased activity were determined relative to the reference polypeptide of SEQ ID NO: 1654 and defined as follows: “+” from 1.00 to 100.00; “++” 100.01-200.00; “+++” 200.01-287.00”.

Activity relative to SEQ ID NO: 2254 (Activity FIOP) was calculated as the percent product of the variant compared with the percent product observed by the reaction with SEQ ID NO: 2254. The results are shown in Table 61.5.

TABLE 61.5
Products ^{a}5′-6-FAM-T15mCmUmGmA-3′P, ^{b}′5′-6-FAM-
T27(2′dF)C*(2′dF)G*(2′dF)A(2′dF)A-3′P, ^{c}5′-6-
FAM-T11mU*fA*fA(2′dF)A-3′P, ^{d}5′-6-FAM-T17mAmUmCmA-3′P,
^{e}5′-6-FAM-T15mGmAmC(2′dF)U-3′P, ^{f}5′-6-FAM-T48mG*mA*mCmA-
3′P, ^{g}5′-6-FAM-T15mAmCmU(2′dF)A-3′P, ^{h}5′-6-FAM-
T15mGmAmCmA-3′P, ⁱ5′-6-FAM-T15mAmCmU(2′dF)U-3′P,
^{j}5′-6-FAM-T15mAmCmUmC-3′P, ^{k}5′-6-FAM-T15mAmCmUmG-3′P,
^{l}5′-6-FAM-T15mAmCmUmA-3′P, ^{m}5′-6-FAM-T15mAmG(MOE)CmA-3′P.

| SEQ | | | | | | | | | | | | | |
|---|---|---|---|---|---|---|---|---|---|---|---|---|---|
| ID NO: | FIOP Product % conversion Relative to SEQ ID NO: 2254 | | | | | | | | | | | | |
| (nt/aa) | a | b | c | d | e | f | g | h | i | j | k | l | m |
| 2811/2812 | + | + | + | + | + | + | + | + | + | + | + | + | + |
| 3673/3674 | + | + | + | + | + | + | ++ | + | ++ | ++ | +++ | +++ | ++ |
| 3917/3918 | + | + | + | + | ++ | + | ++ | ++ | ++ | ++ | ++ | ++ | +++ |

Levels of increased activity were determined relative to the reference polypeptide of SEQ ID NO: 2254 and defined as follows: “+” from 1.00 to 50.00; “++” 50.01-100.00; “+++” 100.01-307.01”.

Activity relative to SEQ ID NO: 2812 (Activity FIOP) was calculated as the percent product of the variant compared with the percent product observed by the reaction with SEQ ID NO: 2812. The results are shown in Table 61.6.

TABLE 61.6
Products ^{a}5′-6-FAM-T17*fA*fAfG(2′dF)A-3′P,
^{b}5′-6-FAM-T15mGmAmCmC-3′P, ^{c}5′-6-FAM-T15mAmU(2′dF)GmA-
3′P, ^{d}5′-6-FAM-T15mAmU(2′dF)UmA-3′P, ^{e}5′-6-
FAM-T15mGmAmCmG-3′P.

| SEQ ID | | | | | |
|---|---|---|---|---|---|
| NO: | FIOP Product % conversion Relative to SEQ ID NO: 2812 | | | | |
| (nt/aa) | ^{a} | ^{b} | ^{c} | ^{d} | ^{e} |
| 3673/3674 | + | + | + | + | ++ |
| 3917/3918 | + | + | ++ | ++ | +++ |

Levels of increased activity were determined relative to the reference polypeptide of SEQ ID NO: 2812 and defined as follows: “+” from 1.00 to 20.00; “++” 20.01-50.00; “+++” 50.01-824.00”.

Activity relative to SEQ ID NO: 3674 (Activity FIOP) was calculated as the percent product of the variant compared with the percent product observed by the reaction with SEQ ID NO: 3674. The results are shown in Table 61.7.

TABLE 61.7
Products ^{a}5′-6-FAM-T15mAmCmU*mU-3′P, ^{b}5′-6-
FAM-T15mGmAmC*mU-3′P, ^{c}5′-6-FAM-T15mGmAmC*mG-3′P,
^{d}5′-6-FAM-T15mAmCmU*mG-3′P, ^{e}5′-6-FAM-
T15mAmU(2′dF)CmA-3′P.

| SEQ ID | | | | | |
|---|---|---|---|---|---|
| NO: | FIOP Product % conversion Relative to SEQ ID NO: 3674 | | | | |
| (nt/aa) | ^{a} | ^{b} | ^{c} | ^{d} | ^{e} |
| 3917/3918 | + | +++ | +++ | ++ | ++ |

Levels of increased activity were determined relative to the reference polypeptide of SEQ ID NO: 3674 and defined as follows: “+” from 1.00 to 25.00; “++” 25.01-60.00; “+++” 60.01-79.00”.

### Example 62

### Iterative Enzymatic Extension of RNA Oligomer Using Engineered TdT Variant SEQ ID NO: 3918

TdT enzyme variant SEQ ID NO: 3918 was produced in shake flask and purified as described in Example 3. Inorganic pyrophosphatase SEQ ID NO: 3944 was produced and purified, as described in Example 58. Alkaline phosphatase SEQ ID NO: 3934 was produced and purified, as described in Example 57.

### Solution Phase Extension

Fluorescently labeled RNA oligomer 5′-BiosG-T3(iFluorT)T9mAmUmA was reacted with TdT and mATP-3′P in a 1.1 mL Axygen 96 Deep Well plate. The reaction included 4 μM oligonucleotide, 1 μM inorganic pyrophosphatase, 8 μM TdT, 72 μM nucleotide triphosphate, and 250 μM CoCl₂ in 20 mM TEA-HCl at pH 7.8. The reaction was set up as follows: (i) all reaction components except for oligonucleotide were mixed in a 1.6 mL Eppendorf tube at a volume of 196 μL, (ii) a 4 μL aliquot of a 200 μM oligonucleotide stock was added to the Eppendorf tube, mixed well, then transferred to one well of an Axygen 96 DeepWell plate. The plate was then incubated at 40° C. for 18 h with 400 rpm agitation. A 4 μL aliquot was removed from the reaction plate, diluted to 1.25 nM final oligonucleotide concentration, then analyzed by capillary electrophoresis according to Example 4.

Next, 80 μL of Dynabeads MyOne Streptavidin C1 magnetic beads were washed with binding and wash buffer according to the Dynabeads protocol. After isolation, the beads were redispersed in 196 μL 2× binding and wash buffer and then combined with the 196 μL of crude extension reaction product containing 5′-BiosG-T3(iFluorT)T9mAmUmAmA-3′P in a 2 mL Eppendorf tube. The tube was placed on a slowly rotating tube rack in a 30° C. incubator for 45 min. Afterwards, the beads were isolated and washed three times with 200 μL binding and wash buffer followed by washing three times with 200 μL 20 mM TEA-HCl (pH 7.8).

### Immobilized Oligo Deprotection

Isolated beads bound to the biotinylated oligonucleotide were redispersed in 200 μL deprotection buffer containing 1 μM alkaline phosphatase, 250 μM CoCl₂, 20 mM TEA-HCl, and 6 v/v % formamide at pH 7.8. The reaction mixture was mixed well then transferred to one well of an Axygen 1.1 mL 96 DeepWell plate and incubated at 50° C. for 0.5 h with 650 rpm agitation.

Using the above procedure for the deprotection step, four sequential addition and deprotection steps were performed starting from biotiylated oligo 5′-BiosG-T3(iFluorT)T9mAmUmA: 5′-BiosG-T3(iFluorT)T9mAmUmAmA-3′P was reacted with alkaline phosphatase to give 5′-BiosG-T3(iFluorT)T9mAmUmAmA; 5′-BiosG-T3(iFluorT)T9mAmUmAmAmG-3′P was reacted with alkaline phosphatase to give 5′-BiosG-T3(iFluorT)T9mAmUmAmAmG; 5′-BiosG-T3(iFluorT)T9mAmUmAmAmGmA-3′P was reacted with alkaline phosphatase to give 5′-BiosG-T3(iFluorT)T9mAmUmAmAmGmA; and 5′-BiosG-T3(iFluorT)T9mAmUmAmAmGmAfA-3′P was reacted with alkaline phosphatase to give 5′-BiosG-T3(iFluorT)T9mAmUmAmAmGmAfA.

Afterward, a 5 μL aliquot was transferred to one well of a BioRad 96 well PCR plate which was placed on a magnetic plate holder to isolate the beads. The supernatant was removed, and the beads redispersed in 20 μL 95:5 formamide:water containing 10 mM EDTA. The mixture was then incubated at 90° C. for 2.5 min in a thermal cycler. The beads were then immediately isolated on a magnetic plate holder, and a 4 μL aliquot of the supernatant solution was diluted 50-fold in deionized water, which was subsequently analyzed by capillary electrophoresis according to Example 4.

The beads in the remaining 195 μL crude reaction product were isolated, then washed with three times with 200 μL of a solution containing 1 g/L bovine serum albumin, 1 mM EDTA, and 20 mM TEA-HCl at pH 7.8. The beads were then isolated and redispersed in 60 μL of a 0.1% sodium dodecyl sulfate (SDS) solution in nuclease free water. The mixture was mixed well, transferred to one well of a BioRad 96 well PCR plate, and incubated at 99° C. for 5 min in a thermal cycler. Immediately afterward the beads were isolated, and the supernatant was collected and diluted to 120 μL with deionized water.

A fresh 80 μL aliquot of Dynabeads MyOne Streptatvidin C1 were washed with binding and wash buffer according to the Dynabeads protocol and then redispersed in 120 μL 2× binding and wash buffer in a 1.6 mL Eppendorf tube. The dispersed beads were then combined with the 120 μL of free biotinylated oligonucleotide and placed on a slowly rotating tube holder in a 30° C. incubator for 30 min. The beads were then isolated and washed three times with 200 μL 20 mM TEA-HCl (pH 7.8).

TABLE 62.1
Dephosphorylation of Immobilized Oligonucleotide-3′P

| | | Conversion |
|---|---|---|
| Starting Oligonucleotide | Product Oligonucleotide | (%) |
| 5′-BiosG- | 5′-BiosG- | 100 |
| T3(iFluorT)T9mAmUmAmA-3′P | T3(iFluorT)T9mAmUmAmA | |
| 5′-BiosG- | 5′-BiosG- | 99 |
| T3(iFluorT)T9mAmUmAmAmG-3′P | T3(iFluorT)T9mAmUmAmAmG | |
| 5′-BiosG- | 5′-BiosG- | 95 |
| T3(iFluorT)T9mAmUmAmAmGmA-3′P | T3(iFluorT)T9mAmUmAmAmGmA | |
| 5′-BiosG- | 5′-BiosG- | 99 |
| T3(iFluorT)T9mAmUmAmAmGmAfA-3′P | T3(iFluorT)T9mAmUmAmAmGmAfA | |

Conversion of nucleotide was determined as percent of the product peak divided by the sum of the total fluorescence in the electropherogram.

### Immobilized Oligo Extension

The beads were isolated and then redispersed in 200 μL of reaction buffer containing 16 μM TdT, 1 μM inorganic pyrophosphatase, 72 μM nucleotide, 1 mM CoCl₂, 20 mM TEA-HCl, 6% v/v formamide, and 0.1% v/v PEG3350 at pH 7.8. After mixing well, the suspension was transferred to one well of an Axygen 1.1 mL 96 DeepWell plate and incubated at 50° C. for 3.5 h with 650 rpm agitation.

Using the above procedure for the addition step, four sequential addition and deprotection steps were performed starting from biotiylated oligo 5′-BiosG-T3(iFluorT)T9mAmUmA: 5′-BiosG-T3(iFluorT)T9mAmUmAmA was reacted with TdT and mGTP-3′P to give 5′-BiosG-T3(iFluorT)T9mAmUmAmAmG-3′P. 5′-BiosG-T3(iFluorT)T9mAmUmAmAmG was reacted with TdT and mATP-3′P to give 5′-BiosG-T3(iFluorT)T9mAmUmAmAmGmA. 5′-BiosG-T3(iFluorT)T9mAmUmAmAmGmA was reacted with TdT and fATP-3′P to give 5′-BiosG-T3(iFluorT)T9mAmUmAmAmGfA-3′P.

Afterward, a 5 μL aliquot was transferred to one well of a BioRad 96 well PCR plate which was placed on a magnetic plate holder to isolate the beads. The supernatant was removed, and the beads redispersed in 20 μL 95:5 formamide:water containing 10 mM EDTA. The mixture was then incubated at 90° C. for 2.5 min in a thermal cycler. The beads were then immediately isolated on a magnetic plate holder, and 4 μL aliquot of the supernant soluion was diluted 50-fold in deionized water, which was analyzed by capillary electrophoresis according to Example 4.

The beads in the remaining 195 μL crude reaction product were isolated and washed three times 200 μL solution containing 1 g/L bovine serum albumin, 1 mM EDTA, and 20 mM TEA-HCl at pH 7.8. Then, the beads were isolated and washed three times with 200 μL solution containing 1 g/L bovine serum albumin and 20 mM TEA-HCl at pH 7.8.

TABLE 62.2
TdT Catalyzed Extension of Oligonucleotide using Nucleotide-3′P

| Starting Oligonucleotide | Product Oligonucleotide | Conversion (%) |
|---|---|---|
| 5′-BiosG- | 5′-BiosG- | 86 |
| T3(iFluorT)T9mAmUmA | T3(iFluorT)T9mAmUmAmA-3′P | |
| 5′-BiosG- | 5′-BiosG- | 97 |
| T3(iFluorT)T9mAmUmAmA | T3(iFluorT)T9mAmUmAmAmG-3′P | |
| 5′-BiosG- | 5′-BiosG- | 91 |
| T3(iFluorT)T9mAmUmAmAmG | T3(iFluorT)T9mAmUmAmAmGmA-3′P | |
| 5′-BiosG- | 5′-BiosG- | 100 |
| T3(iFluorT)T9mAmUmAmAmGmA | T3(iFluorT)T9mAmUmAmAmGmAfA-3′P | |

Conversion of nucleotide was determined as percent of the product peak divided by the sum of the total fluorescence in the electropherogram.

### Example 63

### Improvements Over SEQ ID NO: 3870 in the Extension of Oligonucleotide Acceptor Molecules with Nucleotide Triphosphate Donors

### HTP Screening for Improved TdT Variants

SEQ ID NO: 3870 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 63.1.

Reactions were performed in 96-well or 384-well format BioRad PCR plates. Reactions included 10-500 NM oligonucleotide, 100-600 μM nucleotide triphosphate, 20-100 mM buffer, and 250-600 M cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 63.1
Reaction conditions

Lysis buffer-TEA (50 mM, pH 7.5), 0.1 g/L lysozyme; Reaction buffer-20 mM TEA, 0.25 mM CoCl₂,
pH 7.8; Lysate concentration (vol %)-0.25; Preincubation Temp (C.)-45; Preincubation Time (min)-60;
Reaction temperature (C.)-45; Reaction Time (min)-120; Reaction volume (μL) −1; NTP (μM) -mCTP-
3′P; NTP conc (μM)-200; Oligonucleotide-5′-6-FAM-T11mC*mA*mG, 5′-6-FAM-
T57mUmU(2′dF)C, 5′-6-FAM-T17*mA*mGmA, 5′-6-FAM-T22*mGmAmA, 5′-6-FAM-
T27mAmAmA, 5′-6-FAM-T32mAmAmA(2′dF)G, 5′-6-FAM-T37mG*mA*mC, 5′-6-FAM-
T42*mA*mCmU, 5′-6-FAM-T47*mCmUmU, 5′-6-FAM-T52mUmUmU; Substrate conc (μM)-10 μM
each of ten oligo; Product -5′-6-FAM-T11mC*mA*mGmC-3′P, 5′-6-FAM-T57mUmU(2′dF)CmC-3′P,
5′-6-FAM-T17*mA*mGmAmC-3′P, 5′-6-FAM-T22*mGmAmAmC-3′P, 5′-6-FAM-
T27mAmAmAmC-3′P, 5′-6-FAM-T32mAmAmA(2′dF)GmC-3′P, 5′-6-FAM-T37mG*mA*mCmC-
3′P, 5′-6-FAM-T42*mA*mCmUmC-3′P, 5′-6-FAM-T47*mCmUmUmC-3′P, 5′-6-FAM-
T52mUmUmUmC-3′P

Activity relative to SEQ ID NO: 3870 (Activity FOP) was calculated as the percent product of the variant, defined as the sum of the area of products in the multiplexed assay divided by the sum of the total peak area, compared with the percent product observed by the reaction with SEQ ID NO: 3870 (where the percent product may be set as the average of replicates or else the highest single sample as appropriate). The results are shown in Table 63.2.

TABLE 63.2

| | | FIOP Product Peak Area |
|---|---|---|
| SEQ ID NO: | Amino Acid Differences | Relative to SEQ ID NO: |
| (nt/aa) | (Relative to SEQ ID NO: 3870) | 3870 |
| 5475/5476 | T200K | +++ |
| 4047/4048 | F204I | +++ |
| 4049/4050 | G406L | +++ |
| 4051/4052 | G406S | +++ |
| 4053/4054 | E342Q | +++ |
| 4055/4056 | P404G | ++ |
| 4057/4058 | T295V | ++ |
| 4059/4060 | Q179M | ++ |
| 4061/4062 | Y192M | ++ |
| 4063/4064 | R171G | ++ |
| 4065/4066 | Q179P | ++ |
| 4067/4068 | M490R | ++ |
| 4069/4070 | Q169T | + |
| 4071/4072 | S403T | + |
| 4073/4074 | M490E | + |
| 4075/4076 | Y192T | + |
| 4077/4078 | F204V | + |
| 4079/4080 | S495C | + |
| 4081/4082 | T295S | + |
| 4083/4084 | S403L | + |
| 4085/4086 | M490L | + |
| 4087/4088 | L522I | + |
| 4089/4090 | D199G | + |
| 4091/4092 | T295G | + |
| 4093/4094 | P165A | + |
| 4095/4096 | N197E | + |
| 4097/4098 | I187R | + |
| 4099/4100 | V210M | + |
| 4101/4102 | Y521V | + |
| 4103/4104 | N197R | + |
| 4105/4106 | N493K | + |
| 4107/4108 | M490S | + |
| 4109/4110 | N175I | + |

Levels of increased activity were determined relative to the reference polypeptide of SEQ ID NO: 3870 and defined as follows: “+” 1.01 to 1.04, “++” 1.04 to 1.07, “+++” 1.07 to 1.12

### Example 64

### Improvements Over SEQ ID NO: 3870 in the Extension of Oligonucleotide Acceptor Molecules with Nucleotide Triphosphate Donors

### HTP Screening for Improved TdT Variants

SEQ ID NO: 3870 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 64.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 64.1
Reaction conditions

Lysis buffer-TEA (50 mM, pH 7.5), 0.1 g/L lysozyme; Reaction buffer-20 mM TEA, 0.25 mM CoCl₂,
pH 7.8; Lysate concentration (vol %)-25; Preincubation Temp (C.)-68; Preincubation Time (min)-60;
Reaction temperature (C.)-50; Reaction Time (min)-60; Reaction volume (μL) −1; NTP (μM)-fATP-3′P;
NTP conc (μM)-100; Oligonucleotide-5′-6-FAM-T15mAmU*mG; Substrate conc (μM)-50; Product -
5′-6-FAM-T15mAmU*mG(2′dF)A-3′P.

Activity relative to SEQ ID NO: 3870 (Activity FIOP) was calculated as the percent product of the variant, defined as the sum of the area of products assay divided by the sum of the total peak area, compared with the percent product observed by the reaction with SEQ ID NO: 3870 (where the percent product may be set as the average of replicates or else the highest single sample as appropriate). The results are shown in Table 64.2.

TABLE 64.2

| | | FIOP Product Peak Area |
|---|---|---|
| SEQ ID NO: | Amino Acid Differences | Relative to SEQ ID NO: |
| (nt/aa) | (Relative to SEQ ID NO: 3870) | 3870 |
| 4047/4048 | F204I | ++ |
| 4049/4050 | G406L | ++ |
| 4051/4052 | G406S | + |
| 4053/4054 | E342Q | + |
| 4055/4056 | P404G | +++ |
| 4059/4060 | Q179M | + |
| 4071/4072 | S403T | + |
| 4081/4082 | T295S | ++ |
| 4083/4084 | S403L | ++ |
| 4087/4088 | L522I | + |
| 4089/4090 | D199G | + |
| 4103/4104 | N197R | ++ |
| 4105/4106 | N493K | ++ |
| 4111/4112 | N175M | +++ |
| 4113/4114 | Q179K | +++ |
| 4115/4116 | N191M | +++ |
| 4117/4118 | V341C | +++ |
| 4119/4120 | E203F | +++ |
| 4121/4122 | T295N | +++ |
| 4123/4124 | L484S | +++ |
| 4125/4126 | D183C | ++ |
| 4127/4128 | K508N | ++ |
| 4129/4130 | T173S | ++ |
| 4131/4132 | N175L | ++ |
| 4133/4134 | P165C | ++ |
| 4135/4136 | N197C | ++ |
| 4137/4138 | G406R | ++ |
| 4139/4140 | N191Q | ++ |
| 4141/4142 | R429V | ++ |
| 4143/4144 | S509R | ++ |
| 4145/4146 | R267S | ++ |
| 4147/4148 | S403W | ++ |
| 4149/4150 | L491V | ++ |
| 4151/4152 | N197T | ++ |
| 4153/4154 | T295A | ++ |
| 4155/4156 | W325L | ++ |
| 4157/4158 | A507G | ++ |
| 4159/4160 | H494T | + |
| 4161/4162 | I340S | + |
| 4163/4164 | E203G | + |
| 4165/4166 | W481Q | + |
| 4167/4168 | K195R | + |
| 4169/4170 | D183W | + |
| 4171/4172 | P165R | + |
| 4173/4174 | T200M | + |
| 4175/4176 | A319E | + |
| 4177/4178 | E203M | + |
| 4179/4180 | N175V | + |
| 4181/4182 | Y192A | + |
| 4183/4184 | Q179S | + |
| 4185/4186 | W325G | + |
| 4187/4188 | Y259F | + |
| 4189/4190 | N176V | + |
| 4191/4192 | Q179T | + |
| 4193/4194 | R480A | + |
| 4195/4196 | R291M | + |
| 4197/4198 | S495R | + |
| 4199/4200 | G406T | + |
| 4201/4202 | L387I | + |
| 4203/4204 | S403K | + |
| 4205/4206 | P404M | + |
| 4207/4208 | E257S | + |
| 4209/4210 | S403R | + |
| 4211/4212 | D199R | + |
| 4213/4214 | A483C | + |
| 4215/4216 | S495M | + |
| 4217/4218 | M490V | + |
| 4219/4220 | N191V | + |
| 4221/4222 | E257P | + |
| 4223/4224 | W398V | + |
| 4225/4226 | H494A | + |
| 4227/4228 | L374V | + |
| 4229/4230 | R429F | + |
| 4231/4232 | Q169M | + |
| 4233/4234 | R429H | + |
| 4235/4236 | D199W | + |
| 4237/4238 | S403V | + |
| 4239/4240 | D199S | + |
| 4241/4242 | S293C | + |
| 4243/4244 | E399S | + |
| 4245/4246 | Q179V | + |
| 4247/4248 | N197F | + |
| 4249/4250 | R480Q | + |
| 4251/4252 | N191F | + |
| 4253/4254 | S495N | + |
| 4255/4256 | M490A | + |
| 4257/4258 | E203V | + |
| 4259/4260 | P165T | + |
| 4261/4262 | D199V | + |
| 4263/4264 | S293T | + |

“Levels of increased activity were determined relative to the reference polypeptide of SEQ ID NO: 3870 and defined as follows: “+” 1.10 to 1.50, “++” 1.50 to 1.97, “+++”, 1.97 to 4.12.

### Example 65

### Improvements Over SEQ ID NO: 3918 in the Extension of Oligonucleotide Acceptor Molecules with Nucleotide Triphosphate Donors

### HTP Screening for Improved TdT Variants

SEQ ID NO: 3918 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 65.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 65.1
Reaction conditions

Lysis buffer-TEA (50 mM, pH 7.5), 0.1 g/L lysozyme; Reaction buffer-20 mM TEA, 0.25 mM CoCl₂,
pH 7.8; Lysate concentration (vol %)-0.25; Preincubation Temp (C.)-48; Preincubation Time (min)-60;
Reaction temperature (C.)-48; Reaction Time (min)-120; Reaction volume (μL) −1; NTP (μM)-*mGTP-
3′P; NTP conc (μM)-200; Oligonucleotide-5′-6-FAM-T11mC*mA*mG, 5′-6-FAM-
T57mUmU(2′dF)C, 5′-6-FAM-T17*mA*mGmA, 5′-6-FAM-T22*mGmAmA, 5′-6-FAM-
T27mAmAmA, 5′-6-FAM-T32mAmAmA(2′dF)G, 5′-6-FAM-T37mG*mA*mC, 5′-6-FAM-
T42*mA*mCmU, 5′-6-FAM-T47*mCmUmU, 5′-6-FAM-T52mUmUmU; Substrate conc (μM)-10 μM
each of ten oligo; Product -5′-6-FAM-T11mC*mA*mG*mG-3′P, 5′-6-FAM-T57mUmU(2′dF)C*mG-
3′P, 5′-6-FAM-T17*mA*mGmA*mG-3′P, 5′-6-FAM-T22*mGmAmA*mG-3′P, 5′-6-FAM-
T27mAmAmA*mG-3′P, 5′-6-FAM-T32mAmAmA(2′dF)G*mG-3′P, 5′-6-FAM-
T37mG*mA*mC*mG-3′P, 5′-6-FAM-T42*mA*mCmU*mG-3′P, 5′-6-FAM-T47*mCmUmU*mG-
3′P, 5′-6-FAM-T52mUmUmU*mG-3′P.

Activity relative to SEQ ID NO: 3918 (Activity FIOP) was calculated as the percent product of the variant, defined as the sum of the area of products in the multiplexed assay divided by the sum of the total peak area, compared with the percent product observed by the reaction with SEQ ID NO: 3918 (where the percent product may be set as the average of replicates or else the highest single sample as appropriate). The results are shown in Table 65.2.

TABLE 65.2

| | | FIOP Product Peak Area |
|---|---|---|
| SEQ ID NO: | Amino Acid Differences | Relative to SEQ ID NO: |
| (nt/aa) | (Relative to SEQ ID NO: 3918) | 3918 |
| 4265/4266 | H7-/D400W/Y459R/F504Q | +++ |
| 4267/4268 | W481R | +++ |
| 4269/4270 | D400W/Y459V/M499R/T500S/F504Q | +++ |
| 4271/4272 | F335V/W481R/L484R | +++ |
| 4273/4274 | W481R/E512R | +++ |
| 4275/4276 | W395N/L484R | +++ |
| 4277/4278 | D400W/Y459V | +++ |
| 4279/4280 | E203R/Y459V | +++ |
| 4281/4282 | F335V/G402R/W481R/L484R | +++ |
| 4283/4284 | L484R | +++ |
| 4285/4286 | Y459R | +++ |
| 4287/4288 | D400W/Y459R/T500S/R501P | +++ |
| 4289/4290 | G402K/W481D/N493T | ++ |
| 4291/4292 | W395T/W481R | ++ |
| 4293/4294 | E203R/Y459R/T500S | ++ |
| 4295/4296 | V218L | ++ |
| 4297/4298 | G402R/L484R/N493T | ++ |
| 4299/4300 | D400W/Y459V/R501P/F504G | ++ |
| 4301/4302 | F335V/G402K/W481R/L484E | ++ |
| 4303/4304 | L484E | ++ |
| 4305/4306 | F335V/G402K/E512R | ++ |
| 4307/4308 | E203R/Y459R/M499R/F504G | ++ |
| 4309/4310 | F335V/W481R/N493T | ++ |
| 4311/4312 | W395N/G402K | ++ |
| 4313/4314 | Y459V | ++ |
| 4315/4316 | N493T | ++ |
| 4317/4318 | M499P | ++ |
| 4319/4320 | D400W | ++ |
| 4321/4322 | F335V/G402K/W481R/L484R | ++ |
| 4323/4324 | W481D | ++ |
| 4325/4326 | G402K/W481R/L484R | ++ |
| 4327/4328 | W395T | ++ |
| 4329/4330 | Y459V/R501P | ++ |
| 4331/4332 | W395N/W481R/E512R | ++ |
| 4333/4334 | E367S | ++ |
| 4335/4336 | F504G | + |
| 4337/4338 | R501P | + |
| 4339/4340 | D400W/F504Q | + |
| 4341/4342 | E367R | + |
| 4343/4344 | T173I/E367S/Y459V/T500S | + |
| 4345/4346 | H3Q | + |
| 4347/4348 | G402K | + |
| 4349/4350 | F504Q | + |
| 4351/4352 | F335V | + |
| 4353/4354 | F335V/W481R/E512R | + |
| 4355/4356 | M499R | + |
| 4357/4358 | E367S/Y459V/T500S | + |
| 4359/4360 | E203R | + |
| 4361/4362 | W395T/G402K/W481R/L484R | + |
| 4363/4364 | E203R/D400W/Y459R/R501P | + |
| 4365/4366 | G402R | + |
| 4367/4368 | Q373G | + |
| 4369/4370 | E485Q | + |
| 4371/4372 | W395N | + |
| 4373/4374 | G402K/W481D | + |
| 4375/4376 | D400W/R501P | + |
| 4377/4378 | Q376S | + |
| 4379/4380 | T500S | + |
| 4381/4382 | E203R/E367S/Y459V/T500S/R501P | + |
| 4383/4384 | P458R | + |
| 4385/4386 | F335V/W481D/L484R | + |
| 4387/4388 | G460M | + |
| 4389/4390 | D336Q | + |
| 4391/4392 | E367T | + |
| 4393/4394 | G460R | + |
| 4395/4396 | P215Q | + |
| 4397/4398 | C390A | + |
| 4399/4400 | Q370E | + |
| 4401/4402 | K384S | + |
| 4403/4404 | D338Q | + |
| 4405/4406 | E512R | + |
| 4407/4408 | G402S | + |
| 4409/4410 | K294D | + |
| 4411/4412 | G402K/W481D/L484R/E512R | + |
| 4413/4414 | E515P | + |
| 4415/4416 | G402K/E512R | + |
| 4417/4418 | A339R | + |
| 4419/4420 | S164G | + |
| 4421/4422 | W395E | + |
| 4423/4424 | E512G | + |
| 4425/4426 | D338E | + |
| 4427/4428 | R501N | + |
| 4429/4430 | W395A | + |
| 4431/4432 | N380G | + |
| 4433/4434 | H516T | + |
| 4435/4436 | E203R/E367S/Y459R | + |
| 4437/4438 | S184A | + |

“Levels of increased activity were determined relative to the reference polypeptide of SEQ ID NO: 3918 and defined as follows: ““+”” 1.08 to 1.20, ““++”” 1.20 to 1.32, ““+++”” 1.32 to 1.65.

### Example 66

### Improvements Over SEQ ID NO: 4266 in the Extension of Oligonucleotide Acceptor Molecules with Nucleotide Triphosphate Donors

### HTP Screening for Improved TdT Variants

SEQ ID NO: 4266 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 66.1.

Reactions were performed in 96-well or 384-well format BioRad PCR plates. Reactions included 10-500 μM oligonucleotide), 100-600 0M nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 66.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48; Preincubation Time (min)-60;
Reaction temperature (C.)-50; Reaction Time (min)-120; Reaction volume (μL) −1; NTP (μM)-*mGTP-
3P; NTP conc (μM)-200; Oligonucleotide-5′-6-FAM-T12mC*mA*mG, 5′-6-FAM-T57mUmU(2′dF)C,
5′-6-FAM-T17*mA*mGmA, 5′-6-FAM-T22*mGmAmA, 5′-6-FAM-T27mAmAmA, 5′-6-FAM-
T32mAmAmA(2′dF)G, 5′-6-FAM-T37mG*mA*mC, 5′-6-FAM-T42*mA*mCmU, 5′-6-FAM-
T47*mCmUmU, 5′-6-FAM-T52mUmUmU; Substrate conc (μM)-15 μM each of ten oligo; Product -5′-
6-FAM-T12mC*mA*mG*mG-3P, 5′-6-FAM-T57mUmU(2′dF)C*mG-3P, 5′-6-FAM-
T17*mA*mGmA*mG-3P, 5′-6-FAM-T22*mGmAmA*mG-3P, 5′-6-FAM-T27mAmAmA*mG-3P, 5′-
6-FAM-T32mAmAmA(2′dF)G*mG-3P, 5′-6-FAM-T37mG*mA*mC*mG-3P, 5′-6-FAM-
T42*mA*mCmU*mG-3P, 5′-6-FAM-T47mGmUmCmA-3P, 5′-6-FAM-T52mUmUmU*mG-3P.

Activity relative to SEQ ID NO: 4266 (Activity FIOP) was calculated as the percent product of the variant, defined as the sum of the area of products in the multiplexed assay divided by the sum of the total peak area, compared with the percent product observed by the reaction with SEQ ID NO: 4266 (where the percent product may be set as the average of replicates or else the highest single sample as appropriate). The results are shown in Table 66.2.

TABLE 66.2

| | | FIOP Product Peak Area |
|---|---|---|
| SEQ ID NO: | Amino Acid Differences | Relative to SEQ ID NO: |
| (nt/aa) | (Relative to SEQ ID NO: 4266) | 4266 |
| 4439/4440 | L311R | +++ |
| 4441/4442 | F346Q | +++ |
| 4443/4444 | R472S/M498P/T499S/Q503G | +++ |
| 4445/4446 | R472G/M498P/T499S/Q503G | +++ |
| 4447/4448 | S471N | ++ |
| 4449/4450 | Q301G | ++ |
| 4451/4452 | Y308Q | ++ |
| 4453/4454 | P299S | ++ |
| 4455/4456 | P299E | ++ |
| 4457/4458 | V343L | ++ |
| 4459/4460 | Q301S/Q503G | + |
| 4461/4462 | L418V/Q503G | + |
| 4463/4464 | E192T | + |
| 4465/4466 | P299T | + |
| 4467/4468 | P299K/M498P/T499S/Q503G | + |
| 4469/4470 | E192G | + |
| 4471/4472 | K296G | + |
| 4473/4474 | E192V | + |
| 4475/4476 | R280H | + |
| 4477/4478 | M473Q | + |
| 4479/4480 | L297F | + |
| 4481/4482 | K406S | + |
| 4483/4484 | Y308R | + |
| 4485/4486 | G349Q | + |
| 4487/4488 | L392R/M498P/T499S/Q503G | + |
| 4489/4490 | E192Y | + |
| 4491/4492 | Y308T | + |
| 4493/4494 | V343C | + |
| 4495/4496 | K296P | + |
| 4497/4498 | L359I/M498P/T499S/Q503G | + |
| 4499/4500 | M465E | + |
| 4501/4502 | D411E | + |
| 4503/4504 | R280G/M498P/T499S/Q503G | + |
| 4505/4506 | F346W | + |
| 4507/4508 | A407S | + |
| 4509/4510 | R188K | + |
| 4511/4512 | E192S/M498P/T499S/Q503G | + |
| 4513/4514 | D491R | + |
| 4515/4516 | G410Q | + |
| 4517/4518 | L358G | + |
| 4519/4520 | Y308K | + |
| 4521/4522 | G410A | + |
| 4523/4524 | Y308H/Q503G | + |
| 4525/4526 | L418G/M498P/T499S/Q503G | + |
| 4527/4528 | E192Y/M498P/T499S/Q503G | + |
| 4529/4530 | D491G | + |
| 4531/4532 | V272P | + |
| 4533/4534 | I419V/Q503G | + |

“Levels of increased activity were determined relative to the reference polypeptide of SEQ ID NO: 4266 and defined as follows: ““+”” 1.06 to 1.21, ““++”” 1.21 to 1.52, ““+++”” 1.52 to 3.08”

### Example 67

### Improvements Over SEQ ID NO: 4266 in the Extension of Oligonucleotide Acceptor Molecules with Nucleotide Triphosphate Donors

### HTP Screening for Improved TdT Variants

SEQ ID NO: 4266 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 67.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 67.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48; Preincubation Time (min)-60;
Reaction temperature (C.)-48; Reaction Time (min)-120; Reaction volume (μL) −1; NTP (μM)-*mGTP-
3P; NTP conc (μM)-200; Oligonucleotide-5′-6-FAM-T12mC*mA*mG, 5′-6-FAM-T57mUmU(2′dF)C,
5′-6-FAM-T17*mA*mGmA, 5′-6-FAM-T22*mGmAmA, 5′-6-FAM-T27mAmAmA, 5′-6-FAM-
T32mAmAmA(2′dF)G, 5′-6-FAM-T37mG*mA*mC, 5′-6-FAM-T42*mA*mCmU, 5′-6-FAM-
T47*mCmUmU, 5′-6-FAM-T52mUmUmU; Substrate conc (μM)-10 μM each of ten oligo; Product -5′-
6-FAM-T12mC*mA*mG*mG-3P, 5′-6-FAM-T57mUmU(2′dF)C*mG-3P, 5′-6-FAM-
T17*mA*mGmA*mG-3P, 5′-6-FAM-T22*mGmAmA*mG-3P, 5′-6-FAM-T27mAmAmA*mG-3P, 5′-
6-FAM-T32mAmAmA(2′dF)G*mG-3P, 5′-6-FAM-T37mG*mA*mC*mG-3P, 5′-6-FAM-
T42*mA*mCmU*mG-3P, 5′-6-FAM-T47*mCmUmU*mG-3P, 5′-6-FAM-T52mUmUmU*mG-3P.

Activity relative to SEQ ID NO: 4266 (Activity FIOP) was calculated as the percent product of the variant, defined as the sum of the area of products in the multiplexed assay divided by the sum of the total peak area, compared with the percent product observed by the reaction with SEQ ID NO: 4266 (where the percent product may be set as the average of replicates or else the highest single sample as appropriate). The results are shown in Table 67.2.

TABLE 67.2

| | | FIOP Product Peak Area |
|---|---|---|
| SEQ ID NO: | Amino Acid Differences | Relative to SEQ ID |
| (nt/aa) | (Relative to SEQ ID NO: 4266) | NO: 4266 |
| 4535/4536 | E202R | +++ |
| 4537/4538 | M498P/T499S/Q503G | +++ |
| 4539/4540 | S163Q/F203I/E366S | +++ |
| 4541/4542 | S163Q/Q328L/P363G/W480D | ++ |
| 4543/4544 | S163Q/P363G/W480D/E485Q | ++ |
| 4545/4546 | N190M/W480D/E485Q | ++ |
| 4547/4548 | S362T/P363G | ++ |
| 4549/4550 | F203I/S362T/E366S | ++ |
| 4551/4552 | F203I/M498P/T499S/Q503G | ++ |
| 4553/4554 | N190M/E202R/F203I/P363G/E366S/W480D/L483R/E485Q | ++ |
| 4555/4556 | S163Q/N190M | + |
| 4557/4558 | N190M/E202R/F203I/W480D | + |
| 4559/4560 | Q328L/R428V | + |
| 4561/4562 | Q328A/E485Q | + |
| 4563/4564 | E202R/F203I/E485Q | + |
| 4565/4566 | F203I/Q328A/R428V | + |
| 4567/4568 | T172S/N174M/Q178K/V340C | + |
| 4569/4570 | L95M/R428V/W480D | + |
| 4571/4572 | F203I/Q328A/W480D/E485Q | + |
| 4573/4574 | W480D/L483R | + |
| 4575/4576 | Q328L/W480D/L483R | + |
| 4577/4578 | S362T/P363R/E366S/R428V/W480D/L483R/M498P/T499S/ | + |
| | Q503G | |
| 4579/4580 | F203I/Q328A/P363G/R428V/L483R | + |
| 4581/4582 | E202R/F203I/Q328A/S362T/P363G/E366S/R428V/W480D/ | + |
| | E485Q/M498P/T499S/Q503G | |
| 4583/4584 | Q178K | + |

“Levels of increased activity were determined relative to the reference polypeptide of SEQ ID NO: 4266 and defined as follows: ““+”” 1.09 to 1.15, ““++”” 1.15 to 1.19, ““+++”” 1.19 to 1.29”

### Example 68

### Improvements Over SEQ ID NO: 4558 in the Extension of Oligonucleotide Acceptor Molecules with Nucleotide Triphosphate Donors

### HTP Screening for Improved TdT Variants

SEQ ID NO: 4558 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 68.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 68.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48; Preincubation Time (min)-60;
Reaction temperature (C.)-48; Reaction Time (min)-120; Reaction volume (μL) −1; NTP (μM)-mATP-
3P; NTP conc (μM)-25; Oligonucleotide-5′-6-FAM-T17*(2′dF)A*(2′dF)A(2′dF)G, 5′-6-FAM-
T32mAmAmA(2′dF)G, 5′-6-FAM-T47*mA(2′dF)A(2′dF)A, 5′-6-FAM-T57mUmU(2′dF)C; Substrate
conc (μM)-12.5 μM each of four oligo; Product -5′-6-FAM-T17*(2′dF)A*(2′dF)A(2′dF)GmA-3P, 5′-6-
FAM-T32mAmAmA(2′dF)GmA-3P, 5′-6-FAM-T47*mA(2′dF)A(2′dF)AmA-3P, 5′-6-FAM-
T57mUmU(2′dF)CmA-3P.

Activity relative to SEQ ID NO: 4558 (Activity FIOP) was calculated as the percent product of the variant, defined as the sum of the area of products in the multiplexed assay divided by the sum of the total peak area, compared with the percent product observed by the reaction with SEQ ID NO: 4558 (where the percent product may be set as the average of replicates or else the highest single sample as appropriate). The results are shown in Table 68.2.

TABLE 68.2

| | | FIOP Product Peak Area |
|---|---|---|
| SEQ ID NO: | Amino Acid Differences | Relative to SEQ ID NO: |
| (nt/aa) | (Relative to SEQ ID NO: 4558) | 4558 |
| 4585/4586 | T172S/Q178K/G401K | +++ |
| 4587/4588 | T172S/N174M/Q178R/G401K/P403G | +++ |
| 4589/4590 | W394T | +++ |
| 4591/4592 | V340C/W394T | +++ |
| 4593/4594 | G405L/L483R | +++ |
| 4595/4596 | S402L/P403G | ++ |
| 4597/4598 | T172S/N174M/Q178R/G401K/P403G/K507R | ++ |
| 4599/4600 | Q178K/G401K/P403G | ++ |
| 4601/4602 | G401K | ++ |
| 4603/4604 | N174M/Q178R | ++ |
| 4605/4606 | V94I/A365F/K367R | ++ |
| 4607/4608 | L483R | ++ |
| 4609/4610 | G401K/S402L/P403G/K507N | ++ |
| 4611/4612 | T172S/Q178K | ++ |
| 4613/4614 | C389A/W394E | + |
| 4615/4616 | W324L/N379T/G405L/L483R | + |
| 4617/4618 | T172S/N174M/Q178K/S402L/S508R | + |
| 4619/4620 | Q178K/S402L/P403G | + |
| 4621/4622 | T172S/N174M/G401K/P403G/K507R | + |
| 4623/4624 | V340C | + |
| 4625/4626 | A365F/K367R/R428C | + |
| 4627/4628 | A365H/C389A/W394E | + |
| 4629/4630 | Q375G/K376H | + |
| 4631/4632 | A365F | + |
| 4633/4634 | Q375G/L380V/L483R | + |
| 4635/4636 | K376H/L483R | + |
| 4637/4638 | Q375G/N379T/L483R | + |
| 4639/4640 | K367R | + |
| 4641/4642 | Q375G/L380V | + |
| 4643/4644 | N379T/L483R | + |
| 4645/4646 | Q375G/L380V/G400P/L483R | + |
| 4647/4648 | A318E/Q375G/L380V | + |

“Levels of increased activity were determined relative to the reference polypeptide of SEQ ID NO: 4558 and defined as follows: ““+”” 1.03 to 1.19, ““++”” 1.19 to 1.33, ““+++”” 1.33 to 1.56”

### Example 69

### Improvements Over SEQ ID NO: 4442 in the Extension of Oligonucleotide Acceptor Molecules with Nucleotide Triphosphate Donors

### HTP Screening for Improved TdT Variants

SEQ ID NO: 4442 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 69.1.

Reactions were performed in 96-well or 384-well format BioRad PCR plates. Reactions included 10-500 μM oligonucleotide, 100-600 μM nucleotide triphosphate, 20-100 mM buffer, and 250-600 M cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 69.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48; Preincubation Time (min)-60;
Reaction temperature (C.)-48; Reaction Time (min)-120; Reaction volume (μL) −1; NTP (μM)-mATP-
3P; NTP conc (μM)-200; Oligonucleotide-5′-6-FAM-T17*(2′dF)A*(2′dF)A(2′dF)G, 5′-6-FAM-
T32mAmAmA(2′dF)G, 5′-6-FAM-T47*mA(2′dF)A(2′dF)A, 5′-6-FAM-T57mUmU(2′dF)C; Substrate
conc (μM)-12.5 μM each of four oligo; Product -5′-6-FAM-T17*(2′dF)A*(2′dF)A(2′dF)GmA-3P, 5′-6-
FAM-T32mAmAmA(2′dF)GmA-3P, 5′-6-FAM-T47*mA(2′dF)A(2′dF)AmA-3P, 5′-6-FAM-
T57mUmU(2′dF)CmA-3P.

Activity relative to SEQ ID NO: 4442 (Activity FIOP) was calculated as the percent product of the variant, defined as the sum of the area of products in the multiplexed assay divided by the sum of the total peak area, compared with the percent product observed by the reaction with SEQ ID NO: 4442 (where the percent product may be set as the average of replicates or else the highest single sample as Table 69.2

TABLE 69.2

| | | FIOP Product Peak Area |
|---|---|---|
| SEQ ID NO: | Amino Acid Differences | Relative to SEQ ID NO: |
| (nt/aa) | (Relative to SEQ ID NO: 4442) | 4442 |
| 4649/4650 | K296P/P299T | +++ |
| 4651/4652 | E192T/S402L/K507R | +++ |
| 4653/4654 | S402L | +++ |
| 4655/4656 | E192T/R280H | +++ |
| 4657/4658 | E185G/N190M | +++ |
| 4659/4660 | M281L | ++ |
| 4661/4662 | E189R/N190M | ++ |
| 4663/4664 | W394M | ++ |
| 4665/4666 | R313A | ++ |
| 4667/4668 | A312Q | ++ |
| 4669/4670 | D182N | ++ |
| 4671/4672 | W480L | ++ |
| 4673/4674 | N174M/K296P/P299E | ++ |
| 4675/4676 | R313I | ++ |
| 4677/4678 | E189H/N190M | ++ |
| 4679/4680 | D182R | ++ |
| 4681/4682 | A318V | ++ |
| 4683/4684 | S402L/K507R | ++ |
| 4685/4686 | S460A | ++ |
| 4687/4688 | L306A | ++ |
| 4689/4690 | Y308Q | ++ |
| 4691/4692 | A318P | + |
| 4693/4694 | V327A | + |
| 4695/4696 | A318R | + |
| 4697/4698 | W257K | + |
| 4699/4700 | Y307N | + |
| 4701/4702 | L305F | + |
| 4703/4704 | Q259R | + |
| 4705/4706 | D182A | + |
| 4707/4708 | L306R | + |
| 4709/4710 | V451L | + |
| 4711/4712 | W394L | + |
| 4713/4714 | H493K | + |
| 4715/4716 | R313L | + |
| 4717/4718 | A318T | + |
| 4719/4720 | Q259C | + |
| 4721/4722 | S260A | + |
| 4723/4724 | W394Y | + |
| 4725/4726 | A318N | + |
| 4727/4728 | A312R | + |
| 4729/4730 | S494T | + |
| 4731/4732 | R476L/W480D | + |
| 4733/4734 | S494R | + |
| 4735/4736 | A318Y | + |
| 4737/4738 | E522T | + |
| 4739/4740 | N190M | + |
| 4741/4742 | A318L | + |
| 4743/4744 | E189A/N190M | + |
| 4745/4746 | A312H | + |
| 4747/4748 | A414C | + |
| 4749/4750 | L306M | + |
| 4751/4752 | A312K | + |
| 4753/4754 | D182C | + |
| 4755/4756 | T316F | + |
| 4757/4758 | W381F | + |
| 4759/4760 | S404A | + |
| 4761/4762 | A414P | + |
| 4763/4764 | G405L | + |
| 4765/4766 | L374M | + |
| 4767/4768 | E189V/N190M | + |
| 4769/4770 | S395G | + |
| 4771/4772 | N190M/L193F | + |
| 4773/4774 | V289I | + |
| 4775/4776 | A318M | + |
| 4777/4778 | W257R | + |
| 4779/4780 | W480D | + |
| 4781/4782 | A312V | + |

“Levels of increased activity were determined relative to the reference polypeptide of SEQ ID NO: 4442 and defined as follows: ““+”” 1.01 to 1.09, ““++”” 1.09 to 1.21, ““+++”” 1.21 to 1.62”

### Example 70

### Improvements Over SEQ ID NO: 4442 in the Extension of Oligonucleotide Acceptor Molecules with Nucleotide Triphosphate Donors

### HTP Screening for Improved TdT Variants

SEQ ID NO: 4442 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 70.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 70.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-25; Preincubation Temp (C.)-65; Preincubation Time (min)-60;
Reaction temperature (C.)-50; Reaction Time (min)-60; Reaction volume (μL) −1; NTP (μM)-fATP-3P;
NTP conc (μM)-100; Oligonucleotide-5′-6-FAM-T15mAmU*mG; Substrate conc (μM)-50; Product -
5′-6-FAM-T15mAmU*mG(2′dF)A-3P.

Activity relative to SEQ ID NO: 4442 (Activity FIOP) was calculated as the percent product of the variant, defined as the sum of the area of products divided by the sum of the total peak area, compared with the percent product observed by the reaction with SEQ ID NO: 4442 (where the percent product may be set as the average of replicates or else the highest single sample as appropriate). The results are shown in Table 70.2.

TABLE 70.2

| | | FIOP Product Peak Area |
|---|---|---|
| SEQ ID NO: | Amino Acid Differences | Relative to SEQ ID NO: |
| (nt/aa) | (Relative to SEQ ID NO: 4442) | 4442 |
| 4671/4672 | W480L | + |
| 4687/4688 | L306A | +++ |
| 4707/4708 | L306R | + |
| 4721/4722 | S260A | + |
| 4731/4732 | R476L/W480D | + |
| 4739/4740 | N190M | + |
| 4771/4772 | N190M/L193F | + |
| 4779/4780 | W480D | +++ |
| 4783/4784 | W480E | +++ |
| 4785/4786 | R476W/W480D | +++ |
| 4787/4788 | W480D/Y481W | ++ |
| 4789/4790 | L306T | ++ |
| 4791/4792 | A414E | ++ |
| 4793/4794 | Q259K | ++ |
| 4795/4796 | S494Q | + |
| 4797/4798 | W480M | + |
| 4799/4800 | F461M | + |
| 4801/4802 | L306C | + |
| 4803/4804 | R432H | + |
| 4805/4806 | W381M | + |
| 4807/4808 | M281C | + |
| 4809/4810 | R455A | + |

“Levels of increased activity were determined relative to the reference polypeptide of SEQ ID NO: 4442 and defined as follows: ““+”” 1.10 to 1.19, ““++”” 1.19 to 1.26 , ““+++”” 1.26 to 1.33”

### Example 71

### Improvements Over SEQ ID NO: 4654 in the Extension of Oligonucleotide Acceptor Molecules with Nucleotide Triphosphate Donors

### HTP Screening for Improved TdT Variants

SEQ ID NO: 4654 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 71.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 71.1
Reaction conditions

Lysis buffer-TEA (50 mM, pH 7.5), 0.1 g/L lysozyme; Reaction buffer-20 mM TEA, 0.25 mM CoCl₂,
pH 7.8; Lysate concentration (vol %)-25; Preincubation Temp (C.)-65; Preincubation Time (min)-60;
Reaction temperature (C.)-50; Reaction Time (min)-60; Reaction volume (μL) −1; NTP (μM)-mATP-
3P; NTP conc (μM)-100; Oligonucleotide-5′-6-FAM-T15mAmU*mG; Substrate conc (μM)-50;
Product -5′-6-FAM-T15mAmU*mGmA-3P.

Activity relative to SEQ ID NO: 4654 (Activity FIOP) was calculated as the percent product of the variant, defined as the sum of the area of products divided by the sum of the total peak area, compared with the percent product observed by the reaction with SEQ ID NO: 4654 (where the percent product may be set as the average of replicates or else the highest single sample as appropriate). The results are shown in Table 71.2.

TABLE 71.2

| | | FIOP Product Peak Area |
|---|---|---|
| SEQ ID NO: | Amino Acid Differences | Relative to SEQ ID NO: |
| (nt/aa) | (Relative to SEQ ID NO: 4654) | 4654 |
| 4811/4812 | Q375G/G405L/K406S | +++ |
| 4813/4814 | W480D/L483R | +++ |
| 4815/4816 | N190M/L380V/G405L | ++ |
| 4817/4818 | W394T/W480D | ++ |
| 4819/4820 | Q301S/W394T/W480D | ++ |
| 4821/4822 | N190M/G405L/K406S | ++ |
| 4823/4824 | Q375G | ++ |
| 4825/4826 | V272A/A318E/W480D/L483R | ++ |
| 4827/4828 | N190M | ++ |
| 4829/4830 | V272A/Q301S/E393K/W394T/W480D | + |
| 4831/4832 | Q375G/G405L | + |
| 4833/4834 | W394T | + |
| 4835/4836 | N190M/Q375G | + |
| 4837/4838 | N190M/Q375G/L380V | + |
| 4839/4840 | Q375G/L380V | + |
| 4841/4842 | N190M/D197N/Y308Q | + |
| 4843/4844 | N190M/Y308Q/L380V/G405L | + |
| 4845/4846 | L380V | + |
| 4847/4848 | A318E | + |

“Levels of increased activity were determined relative to the reference polypeptide of SEQ ID NO: 4654 and defined as follows: ““+”” 1.06 to 1.28, ““++”” 1.28 to 1.46, ““+++”” 1.46 to 1.67”

### Example 72

### Improvements Over SEQ ID NO: 4850 in the Extension of Oligonucleotide Acceptor Molecules with Nucleotide Triphosphate Donors

### HTP Screening for Improved TdT Variants

SEQ ID NO: 4814 was selected as the parent TdT enzyme and the gene was re-cloned into pCK900 to re-introduce a missing histidine in the N-terminal poly-histidine sequence, generating SEQ ID NO: 4850. Libraries of engineered genes were produced from the parent gene using various techniques (e.g., saturation mutagenesis and recombination of previously identified beneficial mutations). The polypeptides encoded by each gene were produced in HTP and prepared as described in Table 72.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 72.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48; Preincubation Time (min)-60;
Reaction temperature (C.)-48; Reaction Time (min)-60; Reaction volume (μL) −0.1; NTP (μM)-
*mUTP-3P; NTP conc (μM)-200; Oligonucleotide-5′-6-FAM-T11mC*mA*mG, 5′-6-FAM-
T17*mA*mGmA, 5′-6-FAM-T37mG*mA*mC, 5′-6-FAM-T42*mA*mCmU; Substrate conc (μM)-25
μM each of four oligo; Product -5′-6-FAM-T11mC*mA*mG*mU-3P, 5′-6-FAM-
T17*mA*mGmA*mU-3P, 5′-6-FAM-T37mG*mA*mC*mU-3P, 5′-6-FAM-T42*mA*mCmU*mU-3P.

Activity relative to SEQ ID NO: 4850 (Activity FIOP) was calculated as the percent product of the variant, defined as the sum of the area of products in the multiplexed assay divided by the sum of the total peak area, compared with the percent product observed by the reaction with SEQ ID NO: 4850 (where the percent product may be set as the average of replicates or else the highest single sample as appropriate). The results are shown in Table 72.2.

TABLE 72.2

| | | FIOP Product Peak Area |
|---|---|---|
| SEQ ID NO: | Amino Acid Differences | Relative to SEQ ID NO: |
| (nt/aa) | (Relative to SEQ ID NO: 4850) | 4850 |
| 4851/4852 | L298F/P300E/W395L | +++ |
| 4853/4854 | L298F/P300E/V328A/W395L | +++ |
| 4855/4856 | E193V/L307T | +++ |
| 4857/4858 | K297P/P300S | +++ |
| 4859/4860 | K297P/L298F/P300T/R392S | +++ |
| 4861/4862 | L298F/P300S/V328A | +++ |
| 4863/4864 | K297P/P300E/V328A | +++ |
| 4865/4866 | L298F/P300E/R392S/W395L | ++ |
| 4867/4868 | P300E/R392S/W395L | ++ |
| 4869/4870 | A319P | ++ |
| 4871/4872 | L298F/P300S/R392S/W395L/D492E | + |
| 4873/4874 | E190R/D322N | + |
| 4875/4876 | L298F/P300E/D481W | + |
| 4877/4878 | D492E | + |
| 4879/4880 | D322N | + |
| 4881/4882 | P300T | + |
| 4883/4884 | L298F/P300T/L360V | + |
| 4885/4886 | P300E | + |
| 4887/4888 | E193V | + |
| 4889/4890 | S261A/D322N/L421V | + |
| 4891/4892 | L298F/P300S/R392S/W395L | + |
| 4893/4894 | L421V | + |
| 4895/4896 | R392S | + |
| 4897/4898 | R189K/E193V/A207V/L307T/Q353R | + |
| 4899/4900 | R189K | + |

“Levels of increased activity were determined relative to the reference polypeptide of SEQ ID NO: 4850 and defined as follows: ““+”” 1.17 to 1.45, ““++”” 1.45 to 1.70, ““+++”” 1.70 to 1.89”

### Example 73

### Improvements Over SEQ ID NO: 4856 in the Extension of Oligonucleotide Acceptor Molecules with Nucleotide Triphosphate Donors

### HTP Screening for Improved TdT Variants

SEQ ID NO: 4856 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 73.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 73.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48; Preincubation Time (min)-60;
Reaction temperature (C.)-48; Reaction Time (min)-120; Reaction volume (μL) −0.1; NTP (μM)-
mATP-3P; NTP conc (μM)-200; Oligonucleotide-5′-6-FAM-T11mU*(2′dF)A*(2′dF)A, 5′-6-FAM-
T17*(2′dF)A*(2′dF)A(2′dF)G, 5′-6-FAM-T27(2′dF)GmA(2′dF)U, 5′-6-FAM-T47*mA(2′dF)A(2′dF)A;
Substrate conc (μM)-12.5 μM each of four oligo; Product -5′-6-FAM-T11mU*(2′dF)A*(2′dF)AmA-3P,
5′-6-FAM-T17*(2′dF)A*(2′dF)A(2′dF)GmA-3P, 5′-6-FAM-T27(2′dF)GmA(2′dF)UmA-3P, 5′-6-FAM-
T47*mA(2′dF)A(2′dF)AmA-3P.

Activity relative to SEQ ID NO: 4856 (Activity FIOP) was calculated as the percent product of the variant, defined as the sum of the area of products in the multiplexed assay divided by the sum of the total peak area, compared with the percent product observed by the reaction with SEQ ID NO: 4856 (where the percent product may be set as the average of replicates or else the highest single sample as appropriate). The results are shown in Table 73.2.

TABLE 73.2

| | | FIOP Product Peak Area |
|---|---|---|
| SEQ ID NO: | Amino Acid Differences | Relative to SEQ ID NO: |
| (nt/aa) | (Relative to SEQ ID NO: 4856) | 4856 |
| 4901/4902 | E394V | +++ |
| 4903/4904 | G354S | +++ |
| 4905/4906 | R392K | +++ |
| 4907/4908 | Q353S | +++ |
| 4909/4910 | E394A | +++ |
| 4911/4912 | Q302A/T307L | +++ |
| 4913/4914 | Q353R | +++ |
| 4915/4916 | A413G | ++ |
| 4917/4918 | L419G | ++ |
| 4919/4920 | M490E | ++ |
| 4921/4922 | L393V | ++ |
| 4923/4924 | Q302S/T307L | ++ |
| 4925/4926 | T317C | ++ |
| 4927/4928 | W395Y | ++ |
| 4929/4930 | R477Q | ++ |
| 4931/4932 | R392H | ++ |
| 4933/4934 | S495R | ++ |
| 4935/4936 | M490Q | ++ |
| 4937/4938 | L419V | + |
| 4939/4940 | S10N/A413S | + |
| 4941/4942 | Q397S | + |
| 4943/4944 | P436S/E512D | + |
| 4945/4946 | R477S | + |
| 4947/4948 | G402V | + |
| 4949/4950 | G460S | + |
| 4951/4952 | P364Q | + |
| 4953/4954 | S495Q | + |
| 4955/4956 | G460A | + |
| 4957/4958 | D412M | + |
| 4959/4960 | R392G | + |
| 4961/4962 | E486V | + |
| 4963/4964 | G460P | + |
| 4965/4966 | V268T | + |
| 4967/4968 | S362V | + |
| 4969/4970 | Q260R | + |
| 4971/4972 | D412G | + |
| 4973/4974 | G402T | + |
| 4975/4976 | P404Q | + |
| 4977/4978 | M490N | + |
| 4979/4980 | G518S | + |
| 4981/4982 | G402L | + |
| 4983/4984 | M490T | + |
| 4985/4986 | M490S | + |
| 4987/4988 | G402P | + |
| 4989/4990 | Q397G | + |
| 4991/4992 | G402K | + |
| 4993/4994 | M490V | + |
| 4995/4996 | P404L | + |
| 4997/4998 | L393R | + |

Levels of increased activity were determined relative to the reference polypeptide of SEQ ID NO: 4856 and defined as follows: “+” 1.02 to 1.22, “++” 1.22 to 1.44, “+++” 1.44 to 1.94

### Example 74

### Improvements Over SEQ ID NO: 4904 in the Extension of Oligonucleotide Acceptor Molecules with Nucleotide Triphosphate Donors

### HTP Screening for Improved TdT Variants

SEQ ID NO: 4904 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 74.1.

Reactions were performed in 96-well or 384-well format BioRad PCR plates. Reactions included 10-500 μM oligonucleotide, 100-600 M nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 74.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48; Preincubation Time (min)-60;
Reaction temperature (C.)-48; Reaction Time (min)-120; Reaction volume (μL) −1; NTP (μM)-fUTP-
3P; NTP conc (μM)-200; Oligonucleotide-5′-6-FAM-T11mAmAmA(2′dF)U, 5′-6-FAM-
T18mA(2′dF)GmA(2′dF)G, 5′-6-FAM-T26mU(2′dF)GmU(2′dF)C, 5′-6-FAM-T47*mA(2′dF)A(2′dF)A;
Substrate conc (μM)-37.5 μM each of four oligo; Product -5′-6-FAM-T11mAmAmA(2′dF)U(2′dF)U-
3P, 5′-6-FAM-T18mA(2′dF)GmA(2′dF)G(2′dF)U-3P, 5′-6-FAM-T26mU(2′dF)GmU(2′dF)C(2′dF)U-
3P, 5′-6-FAM-T47*mA(2′dF)A(2′dF)A(2′dF)U-3P.

Activity relative to SEQ ID NO: 4904 (Activity FIOP) was calculated as the percent product of the variant, defined as the sum of the area of products in the multiplexed assay divided by the sum of the total peak area, compared with the percent product observed by the reaction with SEQ ID NO: 4904 (where the percent product may be set as the average of replicates or else the highest single sample as appropriate). The results are shown in Table 74.2.

TABLE 74.2

| | | FIOP Product Peak Area |
|---|---|---|
| SEQ ID NO: | Amino Acid Differences | Relative to SEQ ID NO: |
| (nt/aa) | (Relative to SEQ ID NO: 4904) | 4904 |
| 4999/5000 | K297P/L298F/Y308N/R392K/W395Y | +++ |
| 5001/5002 | L194F/P300T/Q302A/A413G | +++ |
| 5003/5004 | P300E/T317C | +++ |
| 5005/5006 | W395Y | ++ |
| 5007/5008 | P300E | + |
| 5009/5010 | L298F/R392K/F525A | + |
| 5011/5012 | L194F/P300E/Q302S/D481M | + |
| 5013/5014 | E190R | + |
| 5015/5016 | P300T/M490E | + |
| 5017/5018 | E190R/P300T/R477Q/M490E | + |
| 5019/5020 | E190R/L287V/P300T/Q302A | + |

Levels of increased activity were determined relative to the reference polypeptide of SEQ ID NO: 4904 and defined as follows: “+” 1.01 to 1.15, “++” 1.15 to 1.32, “+++” 1.32 to 1.52

### Example 75

### Improvements Over SEQ ID NO: 5002 in the Extension of Oligonucleotide Acceptor Molecules with Nucleotide Triphosphate Donors

### HTP Screening for Improved TdT Variants

SEQ ID NO: 5002 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 75.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 75.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48; Preincubation Time (min)-60;
Reaction temperature (C.)-48; Reaction Time (min)-120; Reaction volume (μL) −1; NTP (μM)-mATP-
3P; NTP conc (μM)-300; Oligonucleotide-5′-6-FAM-T18mA(2′dF)GmA(2′dF)G, 5′-6-FAM-
T26mU(2′dF)GmU(2′dF)C, 5′-6-FAM-T46mA(2′dF)GmU(2′dF)G, 5′-6-FAM-
T51mU*(2′dF)A*(2′dF)A(2′dF)G; Substrate conc (μM)-62.5 μM each of four oligo; Product -5′-6-
FAM-T18mA(2′dF)GmA(2′dF)GmA-3P, 5′-6-FAM-T26mU(2′dF)GmU(2′dF)CmA-3P, 5′-6-FAM-
T46mA(2′dF)GmU(2′dF)GmA-3P, 5′-6-FAM-T51mU*(2′dF)A*(2′dF)A(2′dF)GmA-3P.

Activity relative to SEQ ID NO: 5002 (Activity FIOP) was calculated as the percent product of the variant, defined as the sum of the area of products in the multiplexed assay divided by the sum of the total peak area, compared with the percent product observed by the reaction with SEQ ID NO: 5002 (where the percent product may be set as the average of replicates or else the highest single sample as appropriate). The results are shown in Table 75.2.

TABLE 75.2

| | | FIOP Product Peak Area |
|---|---|---|
| SEQ ID NO: | Amino Acid Differences | Relative to SEQ ID NO: |
| (nt/aa) | (Relative to SEQ ID NO: 5002) | 5002 |
| 5021/5022 | L298F/R392K/E394V/M490E | +++ |
| 5023/5024 | R392K/E394V | +++ |
| 5025/5026 | L393V/E394V/R433H/R477V/M490E | +++ |
| 5027/5028 | R392K/L393V/E394V/R433H | +++ |
| 5029/5030 | R392K/E394V/R477V/S495Q | +++ |
| 5031/5032 | L298F/A302S/R392K/L393V/E394A/R433H | +++ |
| 5033/5034 | R392K/E394V/S495Q | +++ |
| 5035/5036 | E394V/M490E | +++ |
| 5037/5038 | L298F/L393V/E394V/W395Y/R433H/R477V | +++ |
| 5039/5040 | E394V/R477V | +++ |
| 5041/5042 | L298F/E394V/R433H | +++ |
| 5043/5044 | R392H/E394A/R433H/R477L | +++ |
| 5045/5046 | L298F/R392K/L393V/E394V | +++ |
| 5047/5048 | R392H/L393V/E394V/M490E | ++ |
| 5049/5050 | R392K/L393V/E394V/R477V/S495Q | ++ |
| 5051/5052 | F194W | ++ |
| 5053/5054 | I324M | ++ |
| 5055/5056 | Y309R | ++ |
| 5057/5058 | E190R/F194L | ++ |
| 5059/5060 | I208V | ++ |
| 5061/5062 | S472T | ++ |
| 5063/5064 | K274V | ++ |
| 5065/5066 | E526L | ++ |
| 5067/5068 | F194L | ++ |
| 5069/5070 | R392H/L393V/E394A/R477L | + |
| 5071/5072 | E526Y | + |
| 5073/5074 | L298F/R392K/L393V/E394A/R477L | + |
| 5075/5076 | K274G | + |
| 5077/5078 | K274W | + |
| 5079/5080 | R392H/E394V/R433H/S495Q | + |
| 5081/5082 | I208S | + |
| 5083/5084 | F194L/D198Q | + |
| 5085/5086 | M466V | + |
| 5087/5088 | F194L/D198V | + |
| 5089/5090 | G411L/G413A | + |
| 5091/5092 | L359C | + |
| 5093/5094 | F194L/D198G | + |
| 5095/5096 | E523T | + |
| 5097/5098 | A313S | + |
| 5099/5100 | V273S | + |
| 5101/5102 | L298F/A302S/R392K/E394A | + |
| 5103/5104 | V273Q | + |
| 5105/5106 | V273M/Q347F/S354G | + |
| 5107/5108 | R281Q | + |
| 5109/5110 | I361L | + |
| 5111/5112 | I324T | + |
| 5113/5114 | L360V | + |
| 5115/5116 | R473S | + |
| 5117/5118 | L359V | + |
| 5119/5120 | A463V | + |
| 5121/5122 | L202T | + |
| 5123/5124 | A408G/G413A | + |
| 5125/5126 | R477L | + |
| 5127/5128 | R392K/E394A/W395Y | + |
| 5129/5130 | R281G | + |
| 5131/5132 | L298I/T300P/A302Q | + |
| 5133/5134 | G460S/F525A | + |
| 5135/5136 | L298F/L393V/E394V/R477L/S495Q | + |
| 5137/5138 | F194L/D198P | + |
| 5139/5140 | V290A | + |
| 5141/5142 | L393V/E394A | + |
| 5143/5144 | R314L | + |
| 5145/5146 | A408L/G413A | + |
| 5147/5148 | P352T | + |
| 5149/5150 | Y308N/G402V/G460A | + |
| 5151/5152 | E190T/F194L | + |
| 5153/5154 | S405T | + |
| 5155/5156 | E190L/F194L | + |
| 5157/5158 | A463P | + |
| 5159/5160 | F194Y | + |
| 5161/5162 | G413A | + |
| 5163/5164 | T300P/A302Q/K303V | + |
| 5165/5166 | D492R | + |
| 5167/5168 | G11D/E523R | + |
| 5169/5170 | L467A | + |
| 5171/5172 | I208G | + |
| 5173/5174 | A264S | + |

“Levels of increased activity were determined relative to the reference polypeptide of SEQ ID NO: 5002 and defined as follows: ““+”” 1.02 to 1.37, ““++”” 1.37 to 1.75, ““+++”” 1.75 to 2.53”

### Example 76

### Improvements Over SEQ ID NO: 5028 in the Extension of Oligonucleotide Acceptor Molecules with Nucleotide Triphosphate Donors

### HTP Screening for Improved TdT Variants

SEQ ID NO: 5028 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 76.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4 FC until the reaction was quenched. Reactions were quenched and processed for CE analysis as described in Example 4.

TABLE 76.1
Reaction conditions

Lysis buffer-TEA (50 mM, pH 7.5), 0.1 g/L lysozyme; Reaction buffer-20 mM TEA, 0.25 mM CoCl2,
pH 7.8; Lysate concentration (vol %)-0.25; Preincubation Temp (C.)-48; Preincubation Time (min)-60;
Reaction temperature (C.)-48; Reaction Time (min)-120; Reaction volume (μL) −0.1; NTP (μM)-fUTP-
3P; NTP conc (μM)-300; Oligonucleotide-5′-6-FAM-T11mCmCmU(2′dF)A, 5′-6-FAM-
T31mU(2′dF)CmA(2′dF)U, 5′-6-FAM-T41mA(2′dF)U(2′dF)C(2′dF)C, 5′-6-FAM-
T51mU*(2′dF)A*(2′dF)A(2′dF)G; Substrate conc (μM)-62.5 uM each of four oligo; Product -5′-6-
FAM-T11mCmCmU(2′dF)A(2′dF)U-3P, 5′-6-FAM-T31mU(2′dF)CmA(2′dF)U(2′dF)U-3P, 5′-6-FAM-
T41mA(2′dF)U(2′dF)C(2′dF)C(2′dF)U-3P, 5′-6-FAM-T51mU*(2′dF)A*(2′dF)A(2′dF)G(2′dF)U-3P.

Activity relative to SEQ ID NO: 5028 (Activity FIOP) was calculated as the percent product of the variant, defined as the sum of the area of products in the multiplexed assay divided by the sum of the total peak area, compared with the percent product observed by the reaction with SEQ ID NO: 5028 (where the percent product may be set as the average of replicates or else the highest single sample as appropriate). The results are shown in Table 76.2.

TABLE 76.2

| | | FIOP Product Peak Area |
|---|---|---|
| SEQ ID NO: | Amino Acid Differences | Relative to SEQ ID NO: |
| (nt/aa) | (Relative to SEQ ID NO: 5028) | 5028 |
| 5175/5176 | K274W | +++ |
| 5177/5178 | F194L/I208V/G411L | +++ |
| 5179/5180 | F194L/Y309R | +++ |
| 5181/5182 | V273S/K274W | +++ |
| 5183/5184 | F194L/D198V | ++ |
| 5185/5186 | F194W | ++ |
| 5187/5188 | K274V/L359V/E526Y | ++ |
| 5189/5190 | K274V | ++ |
| 5191/5192 | K274G/R281Q/E526Y | ++ |
| 5193/5194 | D198V/I208S | ++ |
| 5195/5196 | Y309R/A313S/G411L | ++ |
| 5197/5198 | V82I/F194L/D198G/A313S | ++ |
| 5199/5200 | M466V/E526Y | ++ |
| 5201/5202 | Y309R | ++ |
| 5203/5204 | F194L/D198V/G411L | ++ |
| 5205/5206 | G411L | ++ |
| 5207/5208 | F194L/D198Q | + |
| 5209/5210 | K274M/E523T | + |
| 5211/5212 | F194W/A313S | + |
| 5213/5214 | D198Q/I208S/A313S/G411L | + |
| 5215/5216 | F194L | + |
| 5217/5218 | F194W/G411L | + |
| 5219/5220 | I324M/E526Y | + |
| 5221/5222 | F194W/D198V/A313S | + |
| 5223/5224 | M466V | + |
| 5225/5226 | F194L/D198V/Y309R | + |
| 5227/5228 | E523T | + |
| 5229/5230 | E526Y | + |
| 5231/5232 | D198Q/I208V/Y309R/G411L | + |
| 5233/5234 | D198Q | + |
| 5235/5236 | F194L/D198Q/I208V/A313S | + |
| 5237/5238 | V273Q/K274G | + |
| 5239/5240 | F194W/D198Q | + |
| 5241/5242 | F194W/D198V | + |
| 5243/5244 | D198G | + |

“Levels of increased activity were determined relative to the reference polypeptide of SEQ ID NO: 5028 and defined as follows: ““+”” 1.02 to 1.17, ““++”” 1.17 to 1.28, ““+++”” 1.28 to 1.56”

### Example 77

### Improvements Over SEQ ID NO: 5192 in the Extension of Oligonucleotide Acceptor Molecules with Nucleotide Triphosphate Donors

### HTP Screening for Improved TdT Variants

SEQ ID NO: 5192 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 77.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 77.1
Reaction conditions

Lysis buffer-TEA (50 mM, pH 7.5), 0.1 g/L lysozyme; Reaction buffer-100 mM TEA, 0.6 mM CoCl2,
pH 7.8; Lysate concentration (vol %)-0.25; Preincubation Temp (C.)-48; Preincubation Time (min)-60;
Reaction temperature (C.)-48; Reaction Time (min)-60; Reaction volume (μL) −0.1; NTP (μM)-mUTP-
3P; NTP conc (μM)-480; Oligonucleotide-5′-6-FAM-T16*(2′dF)C*mA(2′dF)A(2′dF)A, 5′-6-FAM-
T21mU(2′dF)CmU(2′dF)C, 5′-6-FAM-T31mAmAmA(2′dF)G, 5′-6-FAM-T46mA(2′dF)GmU(2′dF)G;
Substrate conc (μM)-100 μM each of four oligo; Product -5′-6-FAM-
T16*(2′dF)C*mA(2′dF)A(2′dF)AmU-3P, 5′-6-FAM-T21mU(2′dF)CmU(2′dF)CmU-3P, 5′-6-FAM-
T31mAmAmA(2′dF)GmU-3P, 5′-6-FAM-T46mA(2′dF)GmU(2′dF)GmU-3P.

Activity relative to SEQ ID NO: 5192 (Activity FIOP) was calculated as the percent product of the variant, defined as the sum of the area of products in the multiplexed assay divided by the sum of the total peak area, compared with the percent product observed by the reaction with SEQ ID NO: 5192 (where the percent product may be set as the average of replicates or else the highest single sample as appropriate). The results are shown in Table 77.2.

TABLE 77.2

| | | FIOP Product Peak Area |
|---|---|---|
| SEQ ID NO: | Amino Acid Differences | Relative to SEQ ID NO: |
| (nt/aa) | (Relative to SEQ ID NO: 5192) | 5192 |
| 5245/5246 | V394Y | +++ |
| 5247/5248 | V394I | +++ |
| 5249/5250 | T200S | +++ |
| 5251/5252 | R177L | +++ |
| 5253/5254 | Q179L | +++ |
| 5255/5256 | L326Q | +++ |
| 5257/5258 | K430R | ++ |
| 5259/5260 | K508S | ++ |
| 5261/5262 | L403V | ++ |
| 5263/5264 | V394F | ++ |
| 5265/5266 | E520I/Y526E | ++ |
| 5267/5268 | T200A | ++ |
| 5269/5270 | T200R | ++ |
| 5271/5272 | T295H | ++ |
| 5273/5274 | D198L | ++ |
| 5275/5276 | T200K | ++ |
| 5277/5278 | V193R | ++ |
| 5279/5280 | K383H | ++ |
| 5281/5282 | T200V | ++ |
| 5283/5284 | L326T | ++ |
| 5285/5286 | K508A | ++ |
| 5287/5288 | V394Q | ++ |
| 5289/5290 | Q329K | ++ |
| 5291/5292 | L96S/T295N | + |
| 5293/5294 | V394L | + |
| 5295/5296 | T295K | + |
| 5297/5298 | D199R | + |
| 5299/5300 | K383R | + |
| 5301/5302 | V394H | + |
| 5303/5304 | T200Y | + |
| 5305/5306 | E485L | + |
| 5307/5308 | V193F | + |
| 5309/5310 | D409S | + |
| 5311/5312 | V193S | + |
| 5313/5314 | L403R | + |
| 5315/5316 | T295G | + |
| 5317/5318 | N176H | + |
| 5319/5320 | T300A/V394F | + |
| 5321/5322 | I187V | + |
| 5323/5324 | K195L | + |
| 5325/5326 | D199S | + |
| 5327/5328 | L326M/N380R | + |
| 5329/5330 | E203A | + |
| 5331/5332 | E203C | + |
| 5333/5334 | V304I | + |
| 5335/5336 | E203L | + |
| 5337/5338 | T200E | + |
| 5339/5340 | Q376R | + |
| 5341/5342 | R177T | + |
| 5343/5344 | V193C | + |
| 5345/5346 | T200Q | + |
| 5347/5348 | Q329L | + |
| 5349/5350 | V394S | + |
| 5351/5352 | K377R | + |

“Levels of increased activity were determined relative to the reference polypeptide of SEQ ID NO: 5192 and defined as follows: ““+”” 1.02 to 1.14, ““++”” 1.14 to 1.23, ““+++”” 1.23 to 1.47”

### Example 78

### Improvements Over SEQ ID NO: 5192 in the Extension of Oligonucleotide Acceptor Molecules with Nucleotide Triphosphate Donors

### HTP Screening for Improved TdT Variants

SEQ ID NO: 5192 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 78.1.

Reactions were performed in 96-well or 384-well format BioRad PCR plates. Reactions included 10-500 μM oligonucleotide, 100-600 0M nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 78.1
Reaction conditions

Lysis buffer-TEA (50 mM, pH 7.5), 0.1 g/L lysozyme; Reaction buffer-100 mM TEA, 0.6 mM CoCl2,
pH 7.8; Lysate concentration (vol %)-25; Preincubation Temp (C.)-65; Preincubation Time (min)-60;
Reaction temperature (C.)-50; Reaction Time (min)-60; Reaction volume (μL) −1; NTP (μM)-fATP-3P;
NTP conc (μM)-200; Oligonucleotide-5′-6-FAM-T15mAmU*mG; Substrate conc (M)-100; Product -
5′-6-FAM-T15mAmU*mG(2′dF)A-3P.

Activity relative to SEQ ID NO: 5192 (Activity FIOP) was calculated as the percent product of the variant, defined as the sum of the area of products divided by the sum of the total peak area, compared with the percent product observed by the reaction with SEQ ID NO: 5192 (where the percent product may be set as the average of replicates or else the highest single sample as appropriate). The results are shown in Table 78.2.

TABLE 78.2

| | | FIOP Product Peak Area |
|---|---|---|
| SEQ ID NO: | Amino Acid Differences | Relative to SEQ ID NO: |
| (nt/aa) | (Relative to SEQ ID NO: 5192) | 5192 |
| 5353/5354 | L326R | +++ |
| 5291/5292 | L96S/T295N | +++ |
| 5327/5328 | L326M/N380R | +++ |
| 5355/5356 | E203G | +++ |
| 5357/5358 | S184H | +++ |
| 5359/5360 | Q169E | ++ |
| 5361/5362 | N197F | ++ |
| 5299/5300 | K383R | ++ |
| 5363/5364 | Q179G | ++ |
| 5347/5348 | Q329L | + |
| 5365/5366 | K383S | + |
| 5367/5368 | S292L | + |
| 5265/5266 | E520I/Y526E | + |
| 5369/5370 | D199G | + |
| 5371/5372 | T295L | + |
| 5261/5262 | L403V | + |
| 5373/5374 | W325M | + |
| 5293/5294 | V394L | + |
| 5345/5346 | T200Q | + |
| 5375/5376 | Q373G | + |
| 5377/5378 | V394W | + |
| 5287/5288 | V394Q | + |
| 5279/5280 | K383H | + |
| 5285/5286 | K508A | + |
| 5379/5380 | D198S | + |
| 5381/5382 | S184G | + |
| 5383/5384 | R177W | + |
| 5349/5350 | V394S | + |
| 5385/5386 | S292M | + |
| 5351/5352 | K377R | + |
| 5255/5256 | L326Q | + |
| 5387/5388 | S292Q | + |
| 5389/5390 | S184P | + |
| 5391/5392 | S292R | + |
| 5393/5394 | N197M/T307A | + |
| 5395/5396 | K377N | + |

“Levels of increased activity were determined relative to the reference polypeptide of SEQ ID NO: 5192 and defined as follows: ““+”” 1.08 to 2.16, ““++”” 2.16 to 3.64, ““+++”” 3.64 to 7.07”

### Example 79

### Improvements Over SEQ ID NO: 5246 in the Extension of Oligonucleotide Acceptor Molecules with Nucleotide Triphosphate Donors

### HTP Screening for Improved TdT Variants

SEQ ID NO: 5246 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 79.1.

Reactions were performed in 96-well or 384-well format BioRad PCR plates. Reactions included 10-500 μM oligonucleotide, 100-600 μM nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 79.1
Reaction conditions

Lysis buffer-TEA (50 mM, pH 7.5), 0.1 g/L lysozyme; Reaction buffer-100 mM TEA, 0.6 mM CoCl₂,
pH 7.8; Lysate concentration (vol %)-25; Preincubation Temp (C.)-58.5; Preincubation Time (min)-60;
Reaction temperature (C.)-50; Reaction Time (min)-60; Reaction volume (μL) −1; NTP (μM)-fATP-3′P;
NTP conc (μM)-480; Oligonucleotide-5′-6-FAM-T15mAmUmCmU, T15mAmUmCmU; Substrate
conc (μM)-100 μM FAM-labelled, 400 μM unlabelled; Product (detected)-5′-6-FAM-
T15mAmUmCmU(2′dF)A-3′P.

Activity relative to SEQ ID NO: 5246 (Activity FIOP) was calculated as the percent product of the variant, defined as the sum of the area of products divided by the sum of the total peak area, compared with the percent product observed by the reaction with SEQ ID NO: 5246 (where the percent product may be set as the average of replicates or else the highest single sample as appropriate). The results are shown in Table 79.2.

TABLE 79.2

| | | FIOP Product Peak Area |
|---|---|---|
| SEQ ID NO: | Amino Acid Differences | Relative to SEQ ID NO: |
| (nt/aa) | (Relative to SEQ ID NO: 5246) | 5246 |
| 5397/5398 | E233R | +++ |
| 5399/5400 | E485L | +++ |
| 5401/5402 | R459M | ++ |
| 5403/5404 | L381Y | ++ |
| 5405/5406 | G365H | ++ |
| 5407/5408 | L381H | ++ |
| 5409/5410 | E367S | + |
| 5411/5412 | N197Q | + |
| 5413/5414 | G365R | + |
| 5415/5416 | E233L | + |
| 5417/5418 | R180K | + |
| 5419/5420 | M441E | + |
| 5421/5422 | D336C | + |
| 5423/5424 | R459W | + |
| 5425/5426 | A252R | + |
| 5427/5428 | P364A | + |
| 5429/5430 | K384A | + |

“Levels of increased activity were determined relative to the reference polypeptide of SEQ ID NO: 5246 and defined as follows: ““+”” 1.02 to 1.28, ““++”” 1.28 to 1.52, ““+++”” 1.52 to 1.99”

### Example 80

### Improvements Over SEQ ID NO: 5246 in the Extension of Oligonucleotide Acceptor Molecules with Nucleotide Triphosphate Donors

### HTP Screening for Improved TdT Variants

SEQ ID NO: 5246 was selected as the parent TdT enzyme. Libraries of engineered genes were produced from the parent gene using various techniques (e.g., saturation mutagenesis and recombination of previously identified beneficial mutations). The polypeptides encoded by each gene were produced in HTP and prepared as described in Table 80.1.

Reactions were performed in 96-well or 384-well format BioRad PCR plates. Reactions included 10-500 μM oligonucleotide, 100-600 0M nucleotide triphosphate, 20-100 mM buffer, and 250-600 μM cobalt (II) chloride. The reactions were set up as follows: (i) all reaction components, except for TdT, were pre-mixed in a single solution, and was aliquoted into each well of the reaction plates (ii) TdT lysate solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 80.1
Reaction conditions

Lysis buffer-TEA (50 mM, pH 7.5), 0.1 g/L lysozyme; Reaction buffer-100 mM TEA, 0.6 mM CoCl2,
pH 7.8; Lysate concentration (vol %)-25; Preincubation Temp (C.)-58.5; Preincubation Time (min)-60;
Reaction temperature (C.)-50; Reaction Time (min)-60; Reaction volume (μL) −1; NTP (μM)-fATP-3′P;
NTP conc (μM)-200; Oligonucleotide-5′-6-FAM-T15mAmU*mG, FAM-T15mAmU*mG; Substrate
conc (μM)-50 μM FAM-labelled, 50 μM unlabelled; Product (detected)-5′-6-FAM-
T15mAmU*mG(2′dF)A-3′P.

Activity relative to SEQ ID NO: 5246 (Activity FIOP) was calculated as the percent product of the variant, defined as the sum of the area of products divided by the sum of the total peak area, compared with the percent product observed by the reaction with SEQ ID NO: 5246 (where the percent product may be set as the average of replicates or else the highest single sample as appropriate). The results are shown in Table 80.2.

TABLE 80.2

| | Amino Acid | FIOP Product |
|---|---|---|
| | Differences | Peak Area |
| SEQ ID NO: | (Relative to SEQ | Relative to SEQ |
| (nt/aa) | ID NO: 5246) | ID NO: 5246 |
| 5431/5432 | D198L/T200K/E203G/T295N | +++ |
| 5433/5434 | R177W/T200K/T295N/L326M | +++ |
| 5435/5436 | T200K/N380R | +++ |
| 5437/5438 | S184H/T200K/T295N/L326M | ++ |
| 5439/5440 | T200K/L326M | ++ |
| 5441/5442 | D198L/T200K/E203G | ++ |
| 5443/5444 | R177W/T200K/E203G/T295N | ++ |
| 5445/5446 | D198L/T200K | ++ |
| 5447/5448 | E190M/T200K/E203G/T295N/N380R | ++ |
| 5449/5450 | S184H/D198L/T200K/E203G/T295N | ++ |
| 5451/5452 | T295N | + |
| 5453/5454 | E203G | + |
| 5455/5456 | E190M/T200K/T295N | + |
| 5457/5458 | R177W/D198L/T200K | + |
| 5459/5460 | D198L | + |
| 5461/5462 | R177W/T200K/E203G | + |
| 5463/5464 | E190M/D198L/T200K/E203G | + |
| 5465/5466 | E203G/N380R | + |

“Levels of increased activity were determined relative to the reference polypeptide of SEQ ID NO: 5246 and defined as follows: ““+”” 1.22 to 1.55, ““++”” 1.55 to 1.75, ““+++”” 1.75 to 1.90”

### Example 81

### Expression of Inorganic Pyrophosphatase-TdT Fusion Proteins

Synthetic genes encoding an N-terminal and C-terminal hexahistidine tagged version of two wild-type (WT) inorganic pyrophosphatases (SEQ ID NO: 3942 and 3944) were fused to a truncated TdT variant generating four IPP-TdT fusion constructs (SEQ ID NO: 5468, 5470, 5472, and 5474). The TdT was derived from SEQ ID NO: 5028 by deleting the first 156 amino acids. A GSGGTG linker was introduced between IPP and TdT. The fused proteins were constructed using well-established techniques (e.g Gibson assembly cloning). Fused proteins were subsequently expressed in an E. coli strain derived from W3110.

Cells transformed with the fusion protein expression constructs were grown at shake-flask scale using IPTG induction as described in Example 3. Cells were then lysed, purified, and dialyzed into storage buffer (20 mM Tris-HCl, pH 7.4, 100 mM KCl, 0.1 mM EDTA, and 50% glycerol). After overnight dialysis, protein samples were removed, and enzyme concentrations were measured by absorption at 280 nm using a NanoDrop™ 1000 spectrophotometer. Soluble protein concentrations are summarized in Table 81.1 below, showing relative purified protein product levels following shake-flask purification relative to SEQ ID NO: 5468.

TABLE 81.1

| | | Levels of Soluble |
|---|---|---|
| | Description of | Enzyme Production |
| SEQ ID NO: | IPP-TdT | (Relative to SEQ |
| (nt/aa) | fusion variant | ID NO: 5468) |
| 5467/5468 | N-terminal 6xhis tagged SEQ ID NO: | + |
| | 3942 fused with truncated | |
| | SEQ ID NO: 5028 | |
| 5469/5470 | N-terminal 6xhis tagged SEQ ID NO: | ++ |
| | 3944 fused with truncated | |
| | SEQ ID NO: 5028 | |
| 5471/5472 | C-terminal 6xhis tagged SEQ ID NO: | + |
| | 3942 fused with truncated | |
| | SEQ ID NO: 5028 | |
| 5473/5474 | C-terminal 6xhis tagged SEQ ID NO: | ++ |
| | 3944 fused with truncated | |
| | SEQ ID NO: 5028 | |

Levels of increased soluble enzyme production were relative to the reference polypeptide of SEQ ID NO: 5468 and defined as follows: 1.31 to 1.41, ““++”” 1.41 to 1.49, ““+++”” 1.49 to 1.48”

### Example 82

### Suppression of by-Products by IPP-TdT Fusion Proteins

TdT-IPP fusion proteins (SEQ ID NO: 5468, 5470, 5472, and 5474) described in Example 81 were assayed for activity and suppression of by-products and compared to reactions containing TdT SEQ ID NO: 5028 with no inorganic pyrophosphatase (control) and to reactions with TdT SEQ ID NO: 5028 with inorganic pyrophosphatases SEQ ID NO: 3942 or 3944.

Reactions were performed in 96-well format 200 μL BioRad PCR plates. Reactions included 400 μM oligonucleotide, 480 μM 3′phosphate blocked NTP, 10 μM TdT or IPP-TdT fusion, 0 or 0.5 μM IPP, 100 mM triethanolamine (pH 7.8), and 600 μM cobalt (II) chloride. The reactions were set up as follows: (i) all reaction components, except for TdT or IPP-TdT fusion and IPP, were pre-mixed in a single solution, and were aliquoted into each well of the 96-well plates (ii) IPP was then added if present, (iii) TdT or IPP-TdT fusion was then added into the wells to initiate the reaction. The reaction plate was heat-sealed with a peelable aluminum seal and incubated in a thermocycler at the indicated temperature and reaction time, then held at 4 5C until the reaction was quenched. Reactions were quenched and processed for CE analysis as described in Example 4.

TABLE 82.1
Reaction buffer-100 mM TEA, 0.6 mM CoCl₂, pH 7.8; Reaction temperature (C.)- 50; Reaction Time
(min)- 60; Reaction volume (μL)- 1; IPP concentration (μM)- 0 or 0.05; TdT or IPP-TdT fusion
concentration (μM)- 10; NTP (μM)-3′P-mATP; NTP conc (μM)- 480; Oligonucleotide- 5′-6-FAM-
T31mAmAmA(2′dF)G, or 5′-6-FAM-T16mC*mA*mGmA; Oligonucleotide conc (μM)- 400; Product-
5′-6-FAM-T31mAmAmA(2′dF)GmA-3′P, or 5′-6-FAM-T16mC*mA*mGmAmA-3′P; By-Product-
unknown.

The ratio of product to by-product relative to a control with TdT SEQ ID NO: 5028 without IPP was calculated as the area of percent area product/percent area by-product ratio of the reaction compared with the percent area product/percent area by-product ratio observed by the control reaction with no IPP present. The results are shown in Table 82.2.

TABLE 82.2
Suppression of TdT reaction by-products by IPP

| | | Percent area product/percent area by-product | |
|---|---|---|---|
| SEQ ID NO: (nt/aa) | | compared relative to no IPP control | |
| TdT or | | Reaction with | Reaction with |
| IPP-TdT | | 5′-6-FAM- | 5′-6-FAM- |
| fusion | IPP | T31mAmAmA(2′dF)G | T16mC*mA*mGmA |
| 5027/5028 | none | + | + |
| 5027/5028 | 3941/3942 | + | + |
| 5027/5028 | 3943/3944 | ++ | + |
| 5467/5468 | none | ++ | ++ |
| 5469/5470 | none | ++ | ++ |
| 5471/5472 | none | ++ | + |
| 5473/5474 | none | ++ | +++ |

Levels of the percent area product/percent area by-product were determined relative to the reference polypeptide of SEQ ID NO: 5028 (control reaction without IPP) and defined as follows: ““+”” 2.44 to 6.12, ““++”” 6.12 to 7.99 , ““+++”” 7.99 to 7.51”

### Example 83

### Activity Improvements Over Multiple Rounds of Evolution

Activity Improvement of Shake-Flask Purified TdT Variants with 3′Phosphate-Blocked Nucleotides and Modified RNA Oligonucleotide Substrates.

TdT variants of SEQ ID NO: 36, 660, 1596, 2956, 3674, 3918, 4850 and 5246 were produced in shake flask and purified as described in Example 3.

Reactions were performed in 96-well format 200 μL BioRad PCR plates. Reactions included 400 M oligonucleotide, 800 μM nucleotide triphosphate, 10 μM TdT, 5 μM IPP, 100 mM triethanolamine (pH 7.8), and 600 μM cobalt (II) chloride. The reactions were set up as follows: (i) all reaction components, except for TdT, were pre-mixed in a single solution, and was aliquoted into each well of the 96-well plates (ii) TdT solution was then added into the wells to initiate the reaction. The reaction plate was heat-sealed with a peelable aluminum seal and incubated in a thermocycler at the indicated temperature and reaction time, then held at 4° C. until the reaction was quenched. Reactions were quenched and processed for CE analysis as described in Example 4.

TABLE 83.1
Reaction buffer-100 mM TEA, 0.6 mM CoCl₂, pH 7.8; IPP SEQ ID NO: 3944 (μM)- 5; Reaction
temperature (C.)- 50; Reaction Time (min)- 60 min; Reaction volume (μL)- 1; TdT concentration (μM)-
10; NTP- as listed in Table 83.2; NTP conc (μM)-800; Oligonucleotide- as listed in table 83.2;
Oligonucleotide conc (μM)-400; Products- 5′-6-FAM-T11AmC*mA*mGmA-3′P; 5′-6-FAM-
T16mC*mA*mGmAmA-3′P; 5′-6-FAM-T21*mA*mGmAmAmA-3′P; 5′-6-FAM-
T26*mGmAmAmAmA-3′P; 5′-6-FAM-T31mAmAmA(2′dF)GmA-3′P; 5′-6-FAM-
T36mAmA(2′dF)GmAmA-3′P; 5′-6-FAM-T41(2′dF)GmA(2′dF)GmUmA-3′P; 5′-6-FAM-
T46mA(2′dF)GmU(2′dF)GmA-3′P; 5′-6-FAM-T51(2′dF)GmU(2′dF)GmU mU-3′P; 5′-6-FAM-
T56(2′dF)GmU(2′dF)CmUmU-3′P; 5′-6-FAM-T10(2′dF)CmU(2′dF)CmAmU-3′P; 5′-6-FAM-
T16mA(2′dF)GmA(2′dF)GmU-3′P; 5′-6-FAM-T21mU(2′dF)CmU(2′dF)CmU-3′P; 5′-6-FAM-
T26mU(2′dF)GmU(2′dF)CmU-3′P; 5′-6-FAM-T31mU(2′dF)CmAmUmU-3′P; 5′-6-FAM-
T41mAmUmCmU(2′dF)A-3′P; 5′-6-FAM-T46mUmCmUmU(2′dF)A-3′P; 5′-6-FAM-
T51mCmUmUmA(2′dF)A-3′P; 5′-6-FAM-T10mAmAmA(MOE)A(2′dF)A-3′P; 5′-6-FAM-
T12mAmG(MOE)C(2′dF)A-3′P; 5′-6-FAM-T26mCmCmU*mUmC-3′P; 5′-6-FAM-
T26mCmCmU*mU*mG-3′P; 5′-6-FAM-T26mCmCmU*mU*mU-3′P; 5′-6-FAM-
T26mCmCmU*mU(2′dF)G-3′P; 5′-6-FAM-T26mCmCmU*mU(2′dF)U-3′P; 5′-6-FAM-
T21*(2′dF)A*(2′dF)A(2′dF)GmAmC-3′P; 5′-6-FAM-T21*(2′dF)A*(2′dF)A(2′dF)GmA*mG-3′P; 5′-6-
FAM-T21*(2′dF)A*(2′dF)A(2′dF)GmA*mU-3′P; 5′-6-FAM-T21*(2′dF)A*(2′dF)A(2′dF)GmA(2′dF)G-
3′P; 5′-6-FAM-T21*(2′dF)A*(2′dF)A(2′dF)GmA(2′dF)U-3′P.

Percent conversion was calculated as the percent product of the variant, defined as the sum of the area of products divided by the sum of the total peak area. The results are shown in Table 83.2.

TABLE 83

| Reaction substrates | | Percent conversion to product by SEQ ID NO: (aa) | | | | | | | |
|---|---|---|---|---|---|---|---|---|---|
| Oligonucleotide | | (see Table 83.1 for list of products detected) | | | | | | | |
| Description | Nucleotide | 36 | 660 | 1596 | 2956 | 3674 | 3918 | 4850 | 5246 |
| 5′-6-FAM- | mATP- | ~ | ~ | ~ | ++ | +++ | +++ | +++ | +++ |
| T11AmC*mA*mG | 3′P | | | | | | | | |
| 5′-6-FAM- | mATP- | ~ | ~ | ~ | ~ | + | ++ | ++ | +++ |
| T16mC*mA*mGmA | 3′P | | | | | | | | |
| 5′-6-FAM- | mATP- | ~ | ~ | ~ | ~ | ~ | ++ | ++ | +++ |
| T21*mA*mGmAmA | 3′P | | | | | | | | |
| 5′-6-FAM- | mATP- | ~ | ~ | ~ | ~ | ~ | ++ | +++ | +++ |
| T26*mGmAmAmA | 3′P | | | | | | | | |
| 5′-6-FAM- | mATP- | ~ | ~ | ~ | + | + | + | + | +++ |
| T31mAmAmA(2′dF)G | 3′P | | | | | | | | |
| 5′-6-FAM- | mATP- | ~ | ~ | ~ | ~ | + | +++ | +++ | +++ |
| T36mAmA(2′dF)GmA | 3′P | | | | | | | | |
| 5′-6-FAM- | mATP- | ~ | ~ | ~ | ~ | ++ | +++ | +++ | +++ |
| T41(2′dF)GmA(2′dF)GmU | 3′P | | | | | | | | |
| 5′-6-FAM- | mATP- | ~ | ~ | ~ | ~ | ~ | ~ | ~ | +++ |
| T46mA(2′dF)GmU(2′dF)G | 3′P | | | | | | | | |
| 5′-6-FAM- | mUTP- | ~ | ~ | ~ | ~ | ++ | +++ | +++ | +++ |
| T51(2′dF)GmU(2′dF)GmU | 3′P | | | | | | | | |
| 5′-6-FAM- | mUTP- | ~ | ~ | ~ | ~ | + | ++ | +++ | +++ |
| T56(2′dF)GmU(2′dF)CmU | 3′P | | | | | | | | |
| 5′-6-FAM- | mUTP- | ~ | ~ | ~ | + | ++ | ++ | +++ | +++ |
| T10(2′dF)CmU(2′dF)CmA | 3′P | | | | | | | | |
| 5′-6-FAM- | mUTP- | ~ | ~ | ~ | ~ | ~ | + | + | +++ |
| T21mU(2′dF)CmU(2′dF)C | 3′P | | | | | | | | |
| 5′-6-FAM- | mUTP- | ~ | ~ | ~ | ~ | ~ | ~ | + | +++ |
| T26mU(2′dF)GmU(2′dF)C | 3′P | | | | | | | | |
| 5′-6-FAM- | mUTP- | ~ | ~ | ~ | ~ | +++ | +++ | +++ | +++ |
| T31mU(2′dF)CmAmU | 3′P | | | | | | | | |
| 5′-6-FAM- | mUTP- | ~ | ~ | ~ | ~ | + | +++ | +++ | +++ |
| T36(2′dF)CmAmUmC | 3′P | | | | | | | | |
| 5′-6-FAM- | fATP- | ~ | ~ | ~ | ~ | + | +++ | +++ | +++ |
| T41mAmUmCmU | 3′P | | | | | | | | |
| 5′-6-FAM- | fATP- | ~ | ~ | ~ | ~ | + | +++ | +++ | +++ |
| T46mUmCmUmU | 3′P | | | | | | | | |
| 5′-6-FAM- | fATP- | ~ | ~ | ~ | + | + | +++ | +++ | +++ |
| T51mCmUmUmA | 3′P | | | | | | | | |
| 5′-6-FAM- | fATP- | ~ | ~ | ~ | + | + | ++ | +++ | +++ |
| T10mAmAmA(MOE)A | 3′P | | | | | | | | |
| 5′-6-FAM- | fATP- | ~ | ~ | ~ | ~ | ~ | ++ | +++ | +++ |
| T12mAmG(MOE)C | 3′P | | | | | | | | |
| 5′-6-FAM- | mCTP- | ~ | ~ | ~ | + | + | +++ | +++ | +++ |
| T26mCmCmU*mU | 3′P | | | | | | | | |
| 5′-6-FAM- | *mGTP- | ~ | ~ | ~ | ~ | ~ | ++ | +++ | +++ |
| T26mCmCmU*mU | 3′P | | | | | | | | |
| 5′-6-FAM- | *mUTP- | ~ | ~ | ~ | ~ | ~ | +++ | +++ | +++ |
| T26mCmCmU*mU | 3′P | | | | | | | | |
| 5′-6-FAM- | mGTP- | ~ | ~ | ~ | ~ | ++ | +++ | +++ | +++ |
| T26mCmCmU*mU | 3′P | | | | | | | | |
| 5′-6-FAM- | mUTP- | ~ | ~ | ~ | ~ | ++ | +++ | +++ | +++ |
| T26mCmCmU*mU | 3′P | | | | | | | | |
| 5′-6-FAM- | mCTP- | ~ | ~ | ~ | +++ | +++ | ++ | +++ | +++ |
| T21*(2′dF)A*(2′dF)A | 3′P | | | | | | | | |
| (2′dF)GmA | | | | | | | | | |
| 5′-6-FAM- | *mGTP- | ~ | ~ | ~ | ~ | + | + | ++ | ++ |
| T21*(2′dF)A*(2′dF)A | 3′P | | | | | | | | |
| (2′dF)GmA | | | | | | | | | |
| 5′-6-FAM- | *mUTP- | ~ | ~ | ~ | + | + | + | ++ | +++ |
| T21*(2′dF)A*(2′dF)A | 3′P | | | | | | | | |
| (2′dF)GmA | | | | | | | | | |
| 5′-6-FAM- | mGTP- | ~ | ~ | ~ | +++ | +++ | ++ | +++ | +++ |
| T21*(2′dF)A*(2′dF)A | 3′P | | | | | | | | |
| (2′dF)GmA | | | | | | | | | |
| 5′-6-FAM- | mUTP- | ~ | ~ | ~ | +++ | +++ | ++ | +++ | +++ |
| T21*(2′dF)A*(2′dF)A | 3′P | | | | | | | | |
| (2′dF)GmA | | | | | | | | | |

Levels of activity were determined by the % conversion to product and defined as follows: “~” from 0-1, “+” from 1-10, “++” from 10-25, “+++” from 25-64.

### Example 84

### High Performance Liquid Chromatography (HPLC) Analysis of Nucleotides

A 5 to 20 μL volume of crude reaction product is diluted to a final total nucleotide concentration of 250 μM using 75% MeOH in milli-Q water as diluent. The reaction container is then vortexed and centrifuged at 4000 rpm for 5 min at 4° C. 100 μL is transferred from the supernatant to a 96 well round bottom plate and then 10 μL is injected onto an Ultimate 3000 HPLC system using a PAL autosampler according to the method outlined in table 84.1.

TABLE 84.1
Achiral HPLC Parameters (Ion Pairing Gradient)

| Instrument | Ultimate 3000 HPLC | |
|---|---|---|
| | System with a PAL | |
| | autosampler | |
| Column | Agilent Zorbax RR StableBond Aq, | |
| | 150 × 3.0 mm × 3.5 μm | |
| Guard Column | Agilent Zorbax StabeBond Aq, | |
| | 5.0 × 3.0 mm, 1.8 μm | |
| Mobile Phases | A: 50 mM sodium phosphate, 2 mM | |
| | tetrabutylammonium bisulfate, pH 7.0 | |
| | B: Acetonitrile | |
| | C: Water | |
| LC Gradient | 0-4.0 min: 9.0% B, 48.5-16.2% C | |
| | 4.0-4.5 min: 9.0% B, 16.2-48.5% C | |
| | 4.5-5.5 min: 9.0% B, 48.5% C | |
| Flow rate | 1.2 | mL/min |
| Run time | 5.5 | min |
| Column temperature | 30° | C. |
| Injection volume | 10 | μL |
| UV Detector | 254 | nm |

### Example 85

### General Enzyme Immobilization Procedure on Affinity Resin

The desired His-tagged protein variant was first produced in shake flask and purified as described in Example 3. The storage buffer from an aliquot of the desired protein stock was then exchanged for TEA-HCl (20 mM, pH 7.8) by diluting 10-fold with TEA·HCl followed by concentration through a Sartorius VivaSpin 6 (10,000 MWCO) spin filter at 4000 rpm and 4° C. The reduced volume sample is then diluted another 2.5 fold with TEA·HCl (20 mM, pH 7.8) to restore the original volume.

Between 2 and 20 mg affinity resin was weighed out, either wet or dry, into a 2.0 mL Eppendorf tube. For larger preparations, 0.5 to 1.0 g affinity resin was weighed out into a 15 mL conical tube. The volume of protein stock containing the desired wt. % of protein vs resin mass was transferred to the tube containing resin. Using TEA·HCl (20 mM, pH 7.8) as diluent, the volume was diluted to 150 μL in each 2.0 mL Eppendorf tube, or 6.0 mL in each 15 mL conical tube.

The samples were then incubated at 4° C. for 24 to 48 h using either 500 rpm agitation or on a tube rotator operating at 10 rpm. Before subjecting to activity assays, the supernatant was removed, and the resin was washed three times. For smaller preparations in 2.0 mL Eppendorf tubes, 150 μL wash volumes of TEA·HCl (20 mM, pH 7.8) were used, allowing the mixture to agitate at room temperature and 500 rpm for 10 minutes during each wash. Between each wash the tubes were briefly centrifuged to collect resin at the bottom. In the case of a larger preparation in a 15 mL conical tube, the resin was collected by vacuum filtration and then washed three times with 100 mL of TEA·HCl (25 mM, pH 7.5), allowing the resin to mix well on a rotator for 10 minutes at room temperature during each wash.

### Example 86

### Immobilization of Purified TdT on Affinity Resin

The TdT enzyme variant of SEQ ID NO: 3918 was produced in shake flask and purified as described in Example 3. The storage buffer was then exchanged for TEA·HCl (20 mM, pH 7.8) as described in Example 85, resulting in a final protein concentration of 7.2 g/L as measured by UV-Vis absorbance at 280 nm (A280) on a nanodrop spectrophotometer.

Affinity resins were purchased from ChiralVision, Purolite, or EnginZyme pre-functionalized with a chelating agent and pre-loaded with a metal for binding his-tagged proteins by the supplier. The metals used can include but are not limited to Ni(II), Co(II), Cu(II), Fe(II), Fe(III), Zn(II) with chelators that include but are not limited to iminodiacetate (IDA), nitrilotriacetate (NTA), and ethylenediaminetetracetate (EDTA).

Resins were weighed out in 4.0 to 6.5 mg quantities in 2.0 mL Eppendorf tubes and charged with enzyme from the 7.2 g/L TdT stock solution, and immobilization was carried out as described in Example 85 with a 24 h incubation. The tubes containing ChiralVision resins were charged with the appropriate volume of TdT stock such that the mass of protein in the sample was 3.0% of the wet mass of resin. The same procedure was repeated for the Purolite Chromalite resins, but with a target of 2.5 wt. % vs the wet mass of resin. The same procedure was repeated for the EnginZyme resins but with a target of 2.5 wt. % vs dry mass of resin.

The samples were briefly centrifuged to collect the resin in the bottom of the Eppendorf tube and 60 μL was transferred from the supernatant to a Greiner 96 well clear bottom half-well plate. The A280 was measured on a plate-reader and compared to a negative control which contained TdT stock that had been incubated under the same conditions with no resin present. The difference in A280 was then used to calculate the percent of immobilization of the protein in solution:

$$Percent Immobilization = ( 1 - \frac{A 280}{A 28 {0}_{neg . ctrl .}} ) \times 100$$

TABLE 86.1
Immobilization of Purified TdT on Affinity Resin

| | | Percent Immobilization |
|---|---|---|
| Resin | Metal | of Added Protein |
| ChiralVision IB-HIS-1 | Ni(II) | ++ |
| ChiralVision IB-HIS-2 | Ni(II) | ++ |
| ChiralVision IB-HIS-2 | Co(II) | ++ |
| ChiralVision IB-HIS-3 | Ni(II) | + |
| ChiralVision IB-HIS-4 | Ni(II) | + |
| ChiralVision IB-HIS-5 | Ni(II) | +++ |
| ChiralVision IB-HIS-6 | Ni(II) | + |
| ChiralVision IB-HIS-7 | Ni(II) | + |
| ChiralVision IB-HIS-8 | Ni(II) | ++ |
| ChiralVision IB-HIS-9 | Ni(II) | ++ |
| ChiralVision IB-HIS-10 | Ni(II) | ++ |
| ChiralVision IB-HIS-11 | Ni(II) | ++ |
| ChiralVision IB-HIS-12 | Ni(II) | ++ |
| ChiralVision IB-HIS-13 | Ni(II) | +++ |
| ChiralVision IB-HIS-14 | Ni(II) | + |
| ChiralVision IB-HIS-15 | Ni(II) | + |
| ChiralVision IB-HIS-16 | Ni(II) | + |
| Purolite Chromalite | Co(II) | ++ |
| Purolite Chromalite | Cu(II) | ++ |
| Purolite Chromalite | Fe(II) | ++ |
| Purolite Chromalite | Ni(II) | ++ |
| Purolite Chromalite | Zn(II) | ++ |
| EnginZyme EziG¹ Opal | Fe(III) | +++ |
| EnginZyme EziG² Choral | Fe(III) | +++ |
| EnginZyme EziG³ Amber | Fe(III) | +++ |

Levels of immobilization are determined by ratio of A280 vs negative control and defined as follows: “+” 0 to 50%, “++” 50 to 80%, “+++” 80 to 100%

### Example 87

### Co-Immobilization of IPP and TdT on Affinity Resin and Enzymatic Single Extension of RNA Oligomer

The TdT enzyme variant of SEQ ID NO: 5028 was produced in shake flask and purified as described in Example 3.

The inorganic pyrophosphatase (IPP) of SEQ ID NO: 3944 was produced and purified, as described in Example 58.

For both the TdT and IPP, the storage buffer in 0.5 mL of stock was exchanged for TEA HCl (20 mM, pH 7.8) as described in Example 85, yielding solutions of IPP and TdT in 2.0 and 9.6 g/L concentrations, respectively.

ChiralVision IB-HIS-2 resin, loaded with CoCl₂, was weighed out in 4.5 to 9.0 mg quantities into 2.0 mL Eppendorf tubes and enzyme immobilization was carried out according to Example 85 using mixtures of the IPP and TdT stock solutions with a 24 h incubation. Each resin sample contained 1.0, 3.0, or 6.0 wt. % TdT vs wet weight of resin in combination with 0, 0.2, 0.8, or 1.6 wt. % IPP vs wet weight of resin.

The samples were removed from the rotator and briefly centrifuged to collect the resin the bottom of each tube. After removing the supernatant, each resin sample was washed three times with TEA HCl (20 mM, pH 7.8) according to Example 85.

To test the activity of the immobilized enzymes, a 1.6 mL reaction stock solution was prepared containing: mATP-3′P (30 μM), 5′-6-FAM-T15mAmUfG (0.2 μM), T15mAmUfG (18.8 μM), CoCl₂ (0.25 mM), TEA·HCl (20 mM, pH 7.8). The reaction stock was then added to each resin sample (20 μL stock per mg wet weight of resin). The reaction was then incubated at 50° C., 500 rpm. After 1 h, a 2 μL aliquot was removed from the supernatant of each reaction mixture and transferred into 98 μL 1 mM EDTA quench solution. Another 2 μL aliquot was removed from the quench solution and processed for CE analysis as described in Example 4. The conversion of 5′-6-FAM-T15mAmUfG to 5′-6-FAM-T15mAmUfGmA-3′P was measured as the % fluorescence of product vs total fluorescence in the CE electropherogram.

TABLE 87.1
Single RNA Extension Using Co-immobilized
IPP SEQ ID NO: 3944 and TdT SEQ ID NO: 5002

| Wt. % TdT | Wt. % IPP | % 5′-6-FAM-T15mAmUfGmA-3′P |
|---|---|---|
| 1 | 0 | + |
| 1 | 0.2 | + |
| 1 | 0.8 | ++ |
| 1 | 1.6 | ++ |
| 3 | 0 | ++ |
| 3 | 0.2 | +++ |
| 3 | 0.8 | +++ |
| 3 | 1.6 | +++ |
| 6 | 0 | +++ |
| 6 | 0.2 | +++ |
| 6 | 0.8 | +++ |
| 6 | 1.6 | +++ |

Conversion of oligonucleotide 5′-6-FAM-T15mAmUf to 5′-6-FAM-T15mAmUfGmA-3′P was determined as percent of total fluorescence in the CE trace and defined as follows:: “+” 0 to 50%, “++” 50 to 80%, “+++” 80 to 100%

### Example 88

### Immobilization of Alkaline Phosphatase on Affinity Resin and Enzymatic 3′-Dephosphorylation of RNA Oligomer

The alkaline phosphatase (AP) of SEQ ID NO: 3932 was produced in shake flask and purified as described in Example 3. The storage buffer in a 0.5 mL aliquot was exchanged for TEA HCl (20 mM, pH 7.8) as described in Example 85, giving a solution containing 7.1 g/L protein as measured by A280 on a nanodrop spectrophotometer.

Affinity resins from ChiralVision were weighed out in 2.5 to 3.0 mg quantities in Eppendorf tubes and charged with the appropriate amount of AP stock solution such that the amount of protein was 2.5 wt. % of the wet mass of resin in each tube. The immobilization was then carried out with a 24 h incubation according to Example 85.

The samples were then diluted to 150 μL using TEA HCl (20 mM, pH 7.8) as diluent and incubated at 5° C. for 24 h with 500 rpm agitation. The A280 of the resulting supernatant solution was compared to that of a solution of AP that had been incubated under the same conditions but without resin present. The percent immobilization was then calculated as:

$$( 1 - \frac{A 280}{A 28 {0}_{neg . ctrl .}} ) \times 100$$

TABLE 88.1
Immobilization of Alkaline Phosphatase
SEQ ID NO: 3932 on Affinity Resin

| | | Percent Immobilization |
|---|---|---|
| Resin | Metal | of Added Protein |
| ChiralVision IB-HIS-2 | Ni(II) | ++ |
| ChiralVision IB-HIS-7 | Ni(II) | + |
| ChiralVision IB-HIS-8 | Ni(II) | + |
| ChiralVision IB-HIS-12 | Ni(II) | ++ |

Levels of immobilization are determined by ratio of A280 vs negative control and defined as follows: “+” 0 to 50%, “++” >50%

To measure the 3′-dephosphorylation activity of the immobilized AP, a 1.8 mL reaction stock was prepared containing: 5′-6-FAM-T15mAmG*mGmA-3′P (0.125 μM) T15mAmG*mGmA-3′P (99.9 μM) COCl₂ (0.25 mM), and TEA HCl (20 mM, pH 7.8). 150 μL of reaction buffer was then transferred to each sample of resin and then briefly centrifuged to collect the resin in the bottom of the container. The reactions were then incubated at 50° C. with 500 rpm agitation.

After 0.5 h, a 2 μL aliquot was removed from the supernatant of each reaction mixture and transferred into 98 μL 1 mM EDTA quench solution. A subsequent 2 μL aliquot was removed from the quench solution and processed for CE analysis as described in Example 4. The conversion of 5′-6-FAM-T15mAmG*mGmA-3′P to 5′-6-FAM-T15mAmG*mGmA was measured as the % fluorescence of product vs total fluorescence in the CE electropherogram.

TABLE 88.2
Dephosphorylation of 5′-6-FAM-T15mAmG*mGmA-
3′P with Immobilized AP

| | Oligo/AP | % Conversion |
|---|---|---|
| Resin | Molar Ratio | of Substrate |
| ChiralVision IB-HIS-2 | 14 | + |
| ChiralVision IB-HIS-7 | 28 | + |
| ChiralVision IB-HIS-8 | 18 | + |
| ChiralVision IB-HIS-12 | 21 | + |

Levels of conversion of 5′-6-FAM-T15mAmG*mGmA-3′P to 5′-6-FAM-T15mAmG*mGmA determined as percent of total fluorescence in the CE trace and defined as follows: “+” >90%

### Example 89

### Immobilization of Alkaline Phosphatase on Affinity Resin and Enzymatic Dephosphorylation of ATP

The alkaline phosphatase (AP) of SEQ ID NO: 3932 was produced in shake flask and purified as described in Example 3. The storage buffer in a 0.5 mL aliquot was exchanged for TEA HCl (20 mM, pH 7.8) as described in Example 85, giving a solution containing 12.2 g/L protein as measured by A280 on a nanodrop spectrophotometer.

Affinity resins from ChiralVision were weighed out in 4.0 to 5.5 mg quantities in Eppendorf tubes and charged with the appropriate amount of AP stock solution such that the amount of protein was 5.0 wt. % of the wet mass of resin in each tube. The immobilization was then carried out with a 24 h incubation according to Example 85.

Each resin sample was washed according to the general procedure outline in Example 85. To measure the dephosphorylation activity of the immobilized AP, a reaction stock was prepared containing: ATP (1 mM), CoCl₂ (0.25 mM), and TEA HCl (20 mM, pH 7.8). The reaction stock was transferred to each resin sample (200 μL stock per mg resin). The reaction mixtures briefly centrifuged to collect the resin in the bottom of the container and then incubated at 50° C. with 500 rpm agitation.

After 1 h, a 5 μL aliquot was roved from each reaction supernatant and transferred to 115 μL 75% MeOH/milli-Q water quench solution in a 96 well round bottom plate. The plate was sealed, vortexed, and centrifuged at 4000 rpm, 4° C., for 5 min. 100 μL was then transferred out of each well into fresh wells on a separate 96 well round bottom plate and analyzed by HPLC according to Example 84. The percent conversion of ATP was measured as the area under curve (AUC) attributed to ATP vs the total AUC for the entire chromatogram.

TABLE 89.1
Reaction of Immobilized Alkaline Phosphatase
SEQ ID NO: 3932 with ATP

| | | Percent |
|---|---|---|
| | ATP/AP | Conversion |
| Resin | Molar Ratio | of ATP |
| ChiralVision IB-HIS-1 | 54 | + |
| ChiralVision IB-HIS-2 | 60 | ++ |
| ChiralVision IB-HIS-7 | 73 | ++ |
| ChiralVision IB-HIS-8 | 60 | ++ |
| ChiralVision IB-HIS-12 | 55 | ++ |
| ChiralVision IB-HIS-16 | 959 | ++ |

Levels of conversion ATP determined as percent of total AUC in chromatogram and defined as “+” 0 to 80%, “++” >80%.

### Example 90

### Immobilization of Alkaline Phosphatase on Epoxide Functionalized Resin

The alkaline phosphatase (AP) of SEQ ID NO: 3932 was produced in shake flask and purified as described in Example 3. An aliquot of the purified AP in storage buffer was diluted into MOPS buffer (20 mM, pH 7.0) to give two stocks, with concentrations of 1.0 and 0.1 g/L.

Epoxide functionalized resins purchased from ChiralVision, IB-COV-2 and IB-COV-7, were weighed out in 19 to 26 mg quantities in 2.0 mL Eppendorf tubes and briefly centrifuged to collect the resin in the bottom. The 1.0 and 0.1 g/L stocks of AP in MOPS (20 mM, pH 7.0) were then added to the resin samples such that two samples received 2.5 wt. % protein vs wet weight of resin, and the other two received 0.25 wt. % protein vs wet weight of resin. These samples were incubated 48 h at 4° C., with no agitation for the 0 to 24 h and then 10 rpm rotation for 24 to 48 h.

To measure the percent of AP that was immobilized, the supernatant was tested for residual enzymatic activity and compared to a sample of AP that had been subjected to the same immobilization conditions but with no resin present. First, 20 μL aliquots were transferred from the supernatant of each sample and diluted to 200 μL with MOPS (20 mM, pH 7.0) buffer. Three controls were prepared by serially diluting a 0.1 g/L AP stock into 200 μL MOPS (20 mM, pH 7.0) at 5, 20, and 40-fold dilutions.

In a separate 15 mL conical tube, 5.0 mL reaction buffer was prepared containing: p-nitrophenylphosphate (4 mM), CoCl₂ (0.5 mM), and TEA HCl (40 mM, pH 7.8). Then, in a 96 well clear flat-bottom plate, 100 μL from the 10× dilution of each sample supernatant and each control was combined with 100 μL from the p-nitrophenylphosphate reaction buffer. The reaction was monitored at 405 nm in a plate reader at 25° C. for 10 min, acquiring data every 10 seconds. The ratio of the reaction rates to the negative control rate gave the percent of AP immobilized:

$$( 1 - \frac{Rate}{{Rate}_{neg . ctrl .}} ) \times 100$$

TABLE 90.1
Covalent Immobilization of SEQ ID NO: 3932

| | Wt. % Added | Percent |
|---|---|---|
| | Protein vs | Immobilization |
| | Resin Wet | of |
| Resin | Weight | Protein |
| ChiralVision IB-COV-2 | 0.25% | ++ |
| ChiralVision IB-COV-2 | 2.50% | +++ |
| ChiralVision IB-COV-7 | 0.25% | ++ |
| ChiralVision IB-COV-7 | 2.50% | + |

Levels of immobilization on resin determined by relative activity in supernatant vs free enzyme when reacted with p-nitrophenyl phosphate, defined as: “+” 0 to 60%, “++” 60 to 90%, “+++” 90 to 100%.

### Example 91

### Immobilization of TdT-IPP Fusion Construct on Affinity Resin

The TdT-IPP fusion constructs of SEQ ID NO: 5470, SEQ ID NO: 5468, SEQ ID NO: 5472, and SEQ ID NO: 5474 were produced in shake flask and purified as described in Example 3. The enzyme stocks in storage buffer were used directly without exchanging the buffer.

Immobilization in 2.0 mL Eppendorf tubes was carried out according to Example 85 with 7.5 to 10 mg ChiralVision IB-HIS-2 resin (pre-loaded with CoCl₂) and 2.5 wt. % protein vs wet weight resin. A 48-hour incubation period at 4° C. was used.

After immobilization, the A280 of the resulting supernatant solution was compared to that of a solution of each enzyme that had been incubated under the same conditions but without resin present. The percent immobilization was then calculated as:

$$( 1 - \frac{A 280}{A 28 {0}_{neg . ctrl .}} ) \times 100$$

TABLE 91.1
Immobilization of Purified TdT-IPP
Fusion Construct on Affinity Resin

| | | | Percent |
|---|---|---|---|
| | | | Immobilization |
| | | | of Added |
| Resin | Metal | Enzyme | Protein |
| ChiralVision IB-HIS-2 | Co(II) | SEQ ID NO: 5470 | ++ |
| ChiralVision IB-HIS-2 | Co(II) | SEQ ID NO: 5468 | + |
| ChiralVision IB-HIS-2 | Co(II) | SEQ ID NO: 5472 | ++ |
| ChiralVision IB-HIS-2 | Co(II) | SEQ ID NO: 5474 | + |

Levels of immobilization are determined by ratio of A280 vs negative control and defined as follows: “+” 0 to 50%, “++” >50%

### Example 92

### Iterative Enzymatic Extension of RNA Oligomer

The TdT enzyme variant of SEQ ID NO: 5246 was produced in shake flask and purified as described in Example 3.

The inorganic pyrophosphatase (IPP) of SEQ ID NO: 3944 was produced and purified, as described in Example 58.

The alkaline phosphatase (AP) of SEQ ID NO: 3932 was produced and purified, as described in Example 57.

TdT and IPP were co-immobilized on ChiralVision IB-HIS-2 resin (0.5 g), pre-loaded with CoCl₂, at 2.5 and 0.2 wt. % vs wet weight of resin, respectively, according to Example 85.

AP was immobilized on a separate batch of Co(II)-loaded ChiralVision IB-HIS-2 resin (0.5 g) at 2.5 wt. % protein vs wet weight of resin, according to Example 85.

First Extension: 5′-6-FAM-T10(2′dF)CmU(2′dF)CmA+mATP-3′P to 5′-6-FAM-T10(2′dF)CmU(2′dF)CmAmA-3′P

To a 2.0 mL Eppendorf tube was added 23 mg IB-HIS-2 resin, charged with 2.5 wt. % TdT (SEQ ID NO: 5246) and 0.2 wt. % IPP (SEQ ID NO: 3944). The tube was briefly centrifuged to collect the resin in the bottom. In a separate Eppendorf tube, 450 μL reaction master mix was prepared containing: mATP-3′P (150 μM), 5′-6-FAM-T10(2′dF)CmU(2′dF)CmA (100 μM), CoCl₂ (0.25 mM), MOPS (20 mM, pH 8.0). The reaction was then incubated at 50° C. for 1.5 h at 500 rpm.

After 1.5 h, the supernatant was transferred into a separate Eppendorf tube containing a fresh 23 mg of IB-HIS-2 resin, charged with 2.5 wt. % TdT and 0.2 wt. % IPP. The reaction was incubated for a further 1.5 h at 50° C. with 500 rpm agitation.

Afterward, a 2 μL aliquot was removed for CE analysis according to Example 4, while the reaction sample was allowed to cool in a 4° C. fridge for 10 minutes. Then, the supernatant was separated from the resin and placed in a 1.5 mL conical Eppendorf tube and centrifuged for 3 min at 14,000 rpm to pellet residual resin particulate. The supernatant was then transferred into a new 2.0 mL Eppendorf tube and the general AP catalyzed dephosphorylation procedure outlined below was carried out.

Second Extension: 5′-6-FAM-T10(2′dF)CmU(2′dF)CmAmA+fGTP-3′P to 5′-6-FAM-T10(2′dF)CmU(2′dF)CmAmA(2′dF)G-3′P

To a 2.0 mL Eppendorf tube was added 15 mg IB-HIS-2 resin charged with 2.5 wt. % TdT (SEQ ID NO: 5246) and 0.2 wt. % IPP (SEQ ID NO: 3944). In a separate 2.0 mL Eppendorf tube, 100 μL from the de-phosphorylate product of the first extension was diluted with 1.5 μL fGTP-3′P (20 mM), 5 μL CoCl₂ (10 mM), 20 μL TEA-HCl (200 mM, pH 7.8), and 174 μL milli-Q water. The reaction solution was then transferred onto the resin and incubated at 50° C. for 1.5 h with 500 rpm agitation.

Afterward, a 2 μL aliquot was removed for CE analysis according to Example 4, while the reaction sample was allowed to cool in a 4° C. fridge for 10 minutes. Then, the supernatant was separated from the resin and placed in a 1.5 mL conical Eppendorf tube and centrifuged for 3 min at 14,000 rpm to pellet residual resin particulate. The supernatant was then transferred into a new 2.0 mL Eppendorf tube and the general AP catalyzed dephosphorylation procedure outlined below was carried out.

Third Extension: 5′-6-FAM-T10(2′dF)CmU(2′dF)CmAmA(2′dF)G+mUTP-3′P to 5′-6-FAM-T10(2′dF)CmU(2′dF)CmAmA(2′dF)GmU-3′ P

To a 2.0 mL Eppendorf tube was added 15 mg IB-HIS-2 resin charged with 2.5 wt. % TdT (SEQ ID NO: 5246) and 0.2 wt. % IPP (SEQ ID NO: 3944). In a separate tube, 100 μL from the de-phosphorylated product of the second extension was diluted with 7.5 μL mUTP-3′P (2 mM), 5 μL TEA-HCl (20 mM, pH 7.8), and 38 μL milli-Q water. The reaction solution was then transferred to the tube containing resin and incubated at 50° C. with 500 rpm agitation.

After 1.5 h, the supernatant was separated from the resin and transferred to an Eppendorf tube containing a fresh 15 mg of IB-HIS-2 resin charged with 2.5 wt. % TdT and 0.2 wt. % IPP. To this mixture was also added an additional 1.9 μL CoCl₂ (10 mM). The reaction was incubated at 50° C. with 500 rpm agitation for an additional 1.5 h.

Afterward, a 2 μL aliquot was removed for CE analysis according to Example 4, while the reaction sample was allowed to cool in a 4° C. fridge for 10 minutes. Then, the supernatant was separated from the resin and placed in a 1.5 mL conical Eppendorf tube and centrifuged for 3 min at 14,000 rpm to pellet residual resin particulate. The supernatant was then transferred into a new 2.0 mL Eppendorf tube and the general AP catalyzed dephosphorylation procedure outlined below was carried out.

Fourth Extension: 5′-6-FAM-T10(2′dF)CmU(2′dF)CmAmA(2′dF)GmU+fGTP-3′P to 5′-6-FAM-T10(2′dF)CmU(2′dF)CmAmA(2′dF)GmU(2′dF)G-3′P

To a 2.0 mL Eppendorf tube was added 8 mg IB-HIS-2 resin charged with 2.5 wt. % TdT (SEQ ID NO: 5246) and 0.2 wt. % IPP (SEQ ID NO: 3944). In a separate tube, 75 μL from the de-phosphorylated product of the third extension was diluted with 4.0 μL fGTP-3′P (2 mM). The reaction solution was then transferred to the tube containing resin and incubated at 50° C. with 500 rpm for 1.5 h.

Afterward, a 2 μL aliquot was removed for CE analysis according to Example 4, while the reaction sample was allowed to cool in a 4° C. fridge for 10 minutes. Then, the supernatant was separated from the resin and placed in a 1.5 mL conical Eppendorf tube and centrifuged for 3 min at 14,000 rpm to pellet residual resin particulate. The supernatant was then transferred into a new 2.0 mL Eppendorf tube and the general AP catalyzed dephosphorylation procedure outlined below was carried out.

Fifth Extension: 5′-6-FAM-T10(2′dF)CmU(2′dF)CmAmA(2′dF)GmU(2′dF)G+mUTP-3′P to 5′-6-FAM-T10(2′dF)CmU(2′dF)CmAmAfGmUfGmU-3′P

To a 2.0 mL Eppendorf tube was added 8 mg IB-HIS-2 resin charged with 2.5 wt. % TdT (SEQ ID NO: 5246) and 0.2 wt. % SEQ ID NO: 3944. In a separate tube, 75 μL from the de-phosphorylated product of the fourth extension was diluted with 4.0 μL mUTP-3′P (2 mM). The reaction solution was then transferred to the tube containing resin and incubated at 50° C. with 500 rpm for 1.5 h.

Afterward, another 8 mg IB-HIS-2 resin charged with 2.5 wt. % TdT (SEQ ID NO: 5246) and 0.2 wt. % SEQ ID NO: 3944 weighed out into a separate 2.0 mL Eppendorf tube. The reaction mixture was separated from the used resin and added to the tube containing fresh resin along with 1.0 μL CoCl₂ (10 mM). The reaction was then incubated for 1.5 h at 50° C. with 500 rpm agitation.

Afterward, another 8 mg IB-HIS-2 resin charged with 2.5 wt. % TdT (SEQ ID NO: 5246) and 0.2 wt. % SEQ ID NO: 3944 was weighed out into a separate 2.0 mL Eppendorf tube. The reaction mixture was separated from the used resin and added to the tube containing fresh resin along with 4.0 μL mUTP-3′P (2 mM). The reaction was then incubated for another 1.5 h at 50° C. with 500 rpm agitation.

Afterward, another 8 mg IB-HIS-2 resin charged with 2.5 wt. % SEQ ID NO: 5246 and 0.2 wt. % SEQ ID NO: 3944 was weighed out into a separate 2.0 mL Eppendorf tube. The reaction mixture was separated from the used resin and added to the tube containing fresh resin and incubated for another 1.5 h at 50° C. with 500 rpm agitation.

Afterward, a 2 μL aliquot was removed for CE analysis according to Example 4, while the reaction sample was allowed to cool in a 4° C. fridge for 10 minutes. Then, the supernatant was separated from the resin and placed in a 1.5 mL conical Eppendorf tube and centrifuged for 3 min at 14,000 rpm to pellet residual resin particulate.

Sixth Extension: 5′-6-FAM-T0(2′dF)CmU(2′dF)CmAmA(2′dF)GmU(2′dF)GmU+fCOP=>5′-6-FAM-T10(2′dF)CmU(2′dF)CmAmA(2′ dF)GmU(2′dF)GmU(2′dF)C-3′P

To a 2.0 mL Eppendorf tube was added 8 mg IB-HIS-2 resin charged with 2.5 wt. % TdT and 0.2 wt. % IPP. In a separate tube, 75 μL from the de-phosphorylated product of the fifth extension was diluted with 4.0 μL fCQP (2 mM). The reaction solution was then transferred to the tube containing resin and incubated at 50° C. with 500 rpm for 1.5 h.

Afterward, a 2 μL aliquot was removed for CE analysis according to Example 4, while the reaction sample was allowed to cool in a 4° C. fridge for 10 minutes. Then, the supernatant was separated from the resin and placed in a 1.5 mL conical Eppendorf tube and centrifuged for 3 min at 14,000 rpm to pellet residual resin particulate. The supernatant was then transferred into a new 2.0 mL Eppendorf tube and the general AP catalyzed dephosphorylation procedure outlined below was carried out.

TABLE 92.1
Extension of Oligonucleotides Catalyzed by Immobilized TdT

| | | Conversion |
|---|---|---|
| Starting Oligonucleotide | Product Oligonucleotide | (%) |
| 5′-6-FAM-T10(2′dF)CmU(2′dF)CmA | 5′-6-FAM- | + |
| | T10(2′dF)CmU(2′dF)CmAmA-3′P | |
| 5′-6-FAM-T10(2′dF)CmU(2′dF)CmAmA | 5′-6-FAM- | ++ |
| | T10(2′dF)CmU(2′dF)CmAmA(2′dF)G- | |
| | 3′P | |
| 5′-6-FAM- | 5′-6-FAM- | ++ |
| T10(2′dF)CmU(2′dF)CmAmA(2′dF)G | T10(2′dF)CmU(2′dF)CmAmA(2′dF)GmU-3′P | |
| 5′-6-FAM- | 5′-6-FAM- | ++ |
| T10(2′dF)CmU(2′dF)CmAmA(2′dF)GmU | T10(2′dF)CmU(2′dF)CmAmA(2′dF)GmU(2′dF)G- | |
| | 3′P | |
| 5′-6-FAM- | 5′-6-FAM- | + |
| T10(2′dF)CmU(2′dF)CmAmA(2′dF)GmU(2′dF)G | T10(2′dF)CmU(2′dF)CmAmA(2′dF)GmU(2′dF)GmU- | |
| | 3′P | |
| 5′-6-FAM- | 5′-6-FAM- | ++ |
| T10(2′dF)CmU(2′dF)CmAmA(2′dF)GmU(2′dF)GmU | T10(2′dF)CmU(2′dF)CmAmA(2′dF)GmU(2′dF)GmU(2′dF)C- | |
| | 3′P | |

Conversion of nucleotide was determined as percent of total fluorescence in the CE trace and defined as follows: “+” 80 to 90%, “++” >90%.

General AP Catalyzed 3′-Dephosphorylation of RNA Oligomer

The supernatant was removed and added to a 2.0 mL Eppendorf tube containing IB-HIS-2 resin charged with 2.5 wt. % AP (SEQ ID NO: 3932), 0.05 mg resin per μL reaction volume was used. The sample was then incubated at 50° C. for 20 minutes.

Afterward, a 2 μL aliquot was removed for CE analysis according to Example 4. Meanwhile, the supernatant was separated from the resin, and transferred into a fresh Eppendorf tube. An equal volume of 25:24:1 chloroform:phenol:isoamyl alcohol was then added and the sample sealed and vortexed. After briefly centrifuging, the top aqueous layer was collected and transferred to a fresh sample tube. Then, an equal volume of 49:1 chloroform:isoamyl alcohol was added and the sample vortexed. After briefly centrifuging, the bottom organic layer was removed and discarded. The 49:1 chloroform:isoamyl alcohol extraction procedure was then repeated two more times. After the third time, the aqueous top layer was removed and transferred to a fresh 2.0 mL Eppendorf tube.

The aqueous layer containing de-phosphorylated oligo product was then placed in a vaccufuge for 15 minutes to remove residual organic solvents. The total volume lost was typically around 50 μL and was accounted for when adding reagents during subsequent extensions.

TABLE 92.2
Dephosphorylation of 3′-phosphorylated Oligonucleotides by Immobilized Alkaline Phosphatase

| | | Conversion |
|---|---|---|
| Starting Oligonucleotide | Product Oligonucleotide | (%) |
| 5′-6-FAM-T10(2′dF)CmU(2′dF)CmAmA- | 5′-6-FAM-T10(2′dF)CmU(2′dF)CmAmA | + |
| 3′P | | |
| 5′-6-FAM- | 5′-6-FAM- | + |
| T10(2′dF)CmU(2′dF)CmAmA(2′dF)G- | T10(2′dF)CmU(2′dF)CmAmA(2′dF)G | |
| 3′P | | |
| 5′-6-FAM- | 5′-6-FAM- | + |
| T10(2′dF)CmU(2′dF)CmAmA(2′dF)GmU- | T10(2′dF)CmU(2′dF)CmAmA(2′dF)GmU | |
| 3′P | | |
| 5′-6-FAM- | 5′-6-FAM- | + |
| T10(2′dF)CmU(2′dF)CmAmA(2′dF)GmU(2′dF)G- | T10(2′dF)CmU(2′dF)CmAmA(2′dF)GmU(2′dF)G | |
| 3′P | | |
| 5′-6-FAM- | 5′-6-FAM- | + |
| T10(2′dF)CmU(2′dF)CmAmA(2′dF)GmU(2′dF)GmU- | T10(2′dF)CmU(2′dF)CmAmA(2′dF)GmU(2′dF)GmU | |
| 3′P | | |
| 5′-6-FAM- | 5′-6-FAM- | + |
| T10(2′dF)CmU(2′dF)CmAmA(2′dF)GmU(2′dF)GmU(2′dF)C- | T10(2′dF)CmU(2′dF)CmAmA(2′dF)GmU(2′dF)GmU(2′dF)C | |
| 3′P | | |

Conversion of nucleotide was determined as percent of total fluorescence in the CE trace and defined as follows: “+”, >95%.

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes in the United States of America, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

What is claimed is:

1. An engineered terminal deoxynucleotidyl transferase comprising a polypeptide sequence having at least 85%, 90%, 91%, 92%, 93%, 94%, 95%, 96%, 97%, 98%, 99%, or more sequence identity to a reference sequence selected from SEQ ID NO: 2, 8, 12, 16, 24, 36, 54, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192, 5246, or a functional fragment thereof, and at least one amino acid residue difference relative to the reference sequence.

2. The engineered terminal deoxynucleotidyl transferase of claim 1, wherein the polypeptide sequence comprises residue differences relative to the reference sequence of SEQ ID NO: 2 at a set of amino acid positions selected from:
80/121/185/315;
80/121/185/190/289/290/293/313/342/499;
80/121/185/190/289/290/293/313/315/336/342/359/391/414/470/474/499/522/523;
80/121/185/190/244/289/290/293/313/315/317/336/342/359/391/470/474/499/522/523;
80/121/174/185/190/196/244/266/273/284/288/289/290/293/313/315/317/324/336/342/352/359/391/394/3 97/401/419/428/431/462/470/474/499/522/523;
80/106/121/185/190/205/289/290/293/313/315/336/342/359/391/470/474/499/522/523;
80/106/121/185/190/205/289/290/293/313/342/470/474/499/523;
80/106/121/185/190/244/289/290/293/307/342/359/470/474/499;
80/121/131/185/190/205/244/289/290/293/313/315/336/342/359/391/414/470/474/499/522/523;
80/121/131/185/190/205/244/289/290/293/313/336/342/359/391/414/470/474/499/522/523;
80/121/131/185/190/289/290/293/313/342/470/474/499/522/523;
80/121/174/179/185/190/236/244/288/289/290/293/313/315/317/336/342/359/363/391/394/408/426/462/4 70/474/499/522/523;
80/121/174/185/186/190/236/244/273/284/288/289/290/293/313/315/317/336/342/352/359/391/394/395/4 19/428/431/462/470/474/499/522/523;
80/121/174/185/190/193/196/244/273/284/288/289/290/293/297/313/315/317/324/336/342/352/359/376/3 80/391/394/401/415/419/428/431/435/441/462/470/474/499/522/523;
80/121/174/185/190/193/244/273/284/288/289/290/293/297/313/315/317/336/342/352/359/391/394/415/4 19/428/431/462/470/474/499/522/523;
80/121/174/185/190/236/244/273/282/284/288/289/290/293/313/315/317/336/342/352/359/391/394/395/4 19/428/431/462/470/474/499/522/523;
80/121/174/185/190/244/273/284/288/289/290/293/313/315/317/336/342/352/359/391/394/419/428/431/4 62/470/474/499/522/523;
80/121/174/185/190/244/273/284/288/289/290/293/313/315/317/336/342/359/391/394/428/431/462/470/4 74/499/522/523;
80/121/174/185/190/244/284/288/289/290/293/313/315/317/336/342/352/359/391/394/419/428/431/462/4 70/474/499/522/523;
80/121/174/185/190/244/284/288/289/290/293/313/315/317/336/342/352/359/391/394/428/431/462/470/4 74/499/522/523;
80/121/174/185/190/244/284/288/289/290/293/313/315/317/336/342/359/391/394/428/431/462/470/474/4 99/522/523;
80/121/185/190/196/244/289/290/293/313/315/317/336/342/359/391/470/474/499/522/523;
80/121/185/190/201/289/290/293/313/342/470/474/499/522;
80/121/185/190/244/273/289/290/293/313/315/317/336/342/352/359/391/419/435/470/474/499/522/523;
80/121/185/190/244/289/290/293/300/313/315/317/336/342/359/391/470/474/499/522/523;
80/121/185/190/244/289/290/293/313/315/317/336/342/359/380/391/401/419/470/474/499/522/523;
80/121/185/190/244/289/290/293/313/315/317/336/342/359/391/392/470/474/499/522/523;
80/121/185/190/244/289/290/293/313/315/317/336/342/359/391/395/470/474/499/522/523;
80/121/185/190/244/289/290/293/313/336/342/359/391/414/470/474/499/522/523;
80/121/185/190/289/290/293/313/336/342/359/391/470/474/499/522/523;
80/121/190/289/290;
80/185/236/289/293;
121/185/190/213/289/290/293; and
185/289/290/293.

3. The engineered terminal deoxynucleotidyl transferase of claim 1, wherein the polypeptide sequence comprises at least one substitution set comprising substitutions as compared to the reference sequence of SEQ ID NO: 36 at amino acid positions selected from:
174/244/273/284/288/315/317/336/352/359/391/394/419/428/431/462/470/474/522/523;
174/244/284/288/315/317/336/359/391/394/428/431/462/470/474/522/523;
244/315/317/336/359/391/470/474/522/523; and
336/359/391/470/474/522/523; and/or any combination thereof.

4. The engineered terminal deoxynucleotidyl transferase of claim 1, wherein the polypeptide sequence comprises at least one substitution or substitution set comprising substitutions as compared to the reference sequence of SEQ ID NO: 8 at amino acid positions selected from:
129/196, 173, 183, 186, 193, 195, 196, 263, 266, 268, 281, 282, 297, 300, 303, 316, 318, 320, 324, 343, 360, 392, 395, 397, 411, 415, 417, 421, 454, 456, 477, 481, and 492, and/or any combinations thereof.

5. The engineered terminal deoxynucleotidyl transferase of claim 1, wherein the polypeptide sequence comprises at least one substitution or substitution set comprising substitutions as compared to the reference sequence of SEQ ID NO: 16 at amino acid positions selected from:
186, 186/236/318, 186/236/395, 186/282/318, 193/196, 193/196/266/324/376/380, 193/196/297, 193/196/297/324/376/380/401/415/435/441, 193/196/324, 193/196/324/397/401/441, 193/196/376/380, 193/297/324/376/435, 193/297/324/380, 193/297/415, 193/435, 196, 196/266, 196/266/324/397/401, 196/297/324/435, 236/282, 236/282/395, 236/318/481, 266/297/380/397/401, 282, 282/318, 282/481, 297/380/401/441, 297/435, 318/395, 376/401/441, 415, and 435/441, and/or any combinations thereof.

6. The engineered terminal deoxynucleotidyl transferase of claim 1, wherein the polypeptide sequence comprises at least one substitution or substitution set comprising substitutions as compared to the reference sequence of SEQ ID NO: 24 at amino acid positions selected from: 12, 13, 14, 17, 18, 20, 21, 22, 23, 24, 26, 27, 29, 30, 31, 33, 34, 35, 37, 41, 53, 57, 58, 61, 92, 94, 97, 101, 102, 103, 104, 105, 106, 107, 108, 124, 126, 133, 135, 137, 138, 139, 140, 141, 142, 144, 145, 147, 149, 150, 152, 153, 154, 155, 156, 156/294, 159, 160, 161, 162, 163, 7/135, 12, 13, 14, 15, 16, 17/131, 18, 20, 23, 24, 25, 26, 27, 28, 29, 31, 32, 33, 34, 35, 44, 45, 46, 57, 65, 77, 85, 89, 93, 94, 97, 101, 102, 103, 105, 106, 108, 109, 110, 119, 123, 124, 126, 130, 131, 132, 133, 134, 135, 137, 138, 139, 149, 150, 153, and 156, and/or any combinations thereof.

7. The engineered terminal deoxynucleotidyl transferase of claim 1, wherein the polypeptide sequence comprises at least one substitution or substitution set comprising substitutions as compared to the reference sequence of SEQ ID NO: 268 at amino acid positions selected from: 14/53/300, 14/53/419, 106/300/415/419/456, 140, and 300/395/419, and/or any combinations thereof.

8. The engineered terminal deoxynucleotidyl transferase of claim 1, wherein the polypeptide sequence comprises at least one substitution or substitution set comprising substitutions as compared to the reference sequence of SEQ ID NO: 648 at amino acid positions selected from: 12/14/34, 12/14/34/37/94/140/141/145, 12/14/34/37/106/140/142/150/152/153, 12/14/34/37/141/142, 12/14/34/37/142/145, 12/14/34/37/142/161, 12/14/34/37/150, 12/14/34/37/150/153, 12/14/34/140/142, 12/14/34/140/150, 12/14/34/142/150/153, 12/14/92/94, 12/14/94/150/152, 12/14/106/107/141/142, 12/14/106/108/140/141/145/150, 12/14/106/108/152, 12/14/141/142, 12/14/150/152/153, 12/14/153, 12/34/92/140, 12/34/150/152, 12/37/94/141/150/152/153, 12/37/140/141/150/162, 12/161, 14, 14/31/34/37/140/141/145/161/162, 14/34/37, 14/34/37/145, 14/34/37/152, 14/34/94/106/108/141, 14/34/150/153, 14/106, 14/140, 14/141/161, 14/142, 14/142/161/162, 14/153, 14/161, 20/21/24/33/58/104/106/124/155/156, 20/21/33/58/101/104/106/124/155, 20/21/58/104/106/155/156, 20/33/104/106/124/156, 20/58/101/104/106/156, 20/58/101/106, 20/101/106/156, 21/33/58/101/106, 21/33/58/106/155/156, 21/33/101/104/106, 21/33/106, 21/58/101/104/106/155, 21/58/106/155/156, 21/101/104/106/156, 21/104/106, 21/104/106/124, 21/104/106/156, 30/33/58/104/106/155/156, 30/33/101/106/156, 30/33/104/106/155/156, 30/101/104/106/155/156, 30/104/106/155, 30/104/106/155/156, 30/106/155, 33/58/104/106, 33/101/104/106/155, 34/37, 34/37/92, 34/37/140/141/142/145, 34/37/141/142, 34/37/150/153, 34/92/94/141/142, 34/141/142/145, 34/150/152/153, 37/92/142, 37/141/142, 37/153, 58/101/104/106/156, 58/101/106/155, 58/104/106/155/156, 92/94/106/142/145, 101/104/106, 101/104/106/155/156, 101/104/106/156, 101/106, 101/106/124/155, 101/106/155/156, 104/106, 104/106/124, 104/106/155, 104/106/155/156, 104/106/156, 106/107/108/142/220, 106/108/140/141/152/153, 106/108/140/142/150/153, 106/156, 140/141/142, 140/145, 140/145/150/152, 141, 141/152, and 161, and/or any combinations thereof.

9. The engineered terminal deoxynucleotidyl transferase of claim 1, wherein the polypeptide sequence comprises at least one substitution or substitution set comprising substitutions as compared to the reference sequence of SEQ ID NO: 660 at amino acid positions selected from: 16/29/30, 16/29/30/33/153, 16/29/30/101/104, 16/30/104, 16/33, 29/30, 58, 92/94/108/141/155/392, 92/101/137/155/476, 94/101/156/476, 101, 101/104, 101/137/155, 101/141/155/156, 108, 195, 197, 204/342, 205, 236/297, 258, 261, 262, 264, 268, 269, 276, 278, 280, 281, 282, 290, 291, 297, 300, 303, 306, 308, 309, 310, 312, 315, 316, 342, 344, 353, 360, 385, 391, 410, 413, 419, 421, 448, 454, 456, 473, 476, 515, 525, and/or any combinations thereof.

10. The engineered terminal deoxynucleotidyl transferase of claim 1, wherein the polypeptide sequence comprises at least one substitution or substitution set comprising substitutions as compared to the reference sequence of SEQ ID NO: 882 at amino acid positions selected from 175, 179, 196, 199, 201, 203, 208, 272, 273, 275, 313, 314, 317, 319, 321, 322, 324, 325, 329/462, 350, 376, 379, 394, 397, 403/462, 404, 406, 408, 457, 461, 462, 469, 477, 481, 484, 491, 492, and 495, and/or any combinations thereof, wherein the amino acid positions are numbered with reference to SEQ ID NO: 882.

11. The engineered terminal deoxynucleotidyl transferase of claim 1, wherein the polypeptide sequence comprises at least one substitution or substitution set comprising substitutions as compared to the reference sequence of SEQ ID NO: 1100 at amino acid positions selected from 101/137/264/476/525 and 264, and/or any combinations thereof.

12. The engineered terminal deoxynucleotidyl transferase of claim 1, wherein the polypeptide sequence comprises at least one substitution or substitution set comprising substitutions as compared to the reference sequence of SEQ ID NO: 1336 at amino acid positions selected from 61, 152/503, 160, 162, 165, 177, 200, 200/425, 213, 217, 219, 223, 236, 246, 248, 292, 292/411, 295, 326, 329, 330, 333, 334, 338, 340, 340/438, 363, 369, 370, 372, 373, 383, 400/401/402, 425, 427, 435, 435/503, 437, 440, 441, 442, 443, 444, 446, 459, 460, 488, 490, 501, 502, 503, 504, and 506, 15/199/203/394, 15/199/394, 28/344/353/395, 195/199/203/278/297/394, 195/199/203/278/314/353/394, 195/203/278/394/395, 195/203/297/314/394, 195/203/297/394/419, 195/203/394, 195/278/297/394, 195/278/297/394/395, 195/314/344, 195/394/395, 199/203/297/394/395, 203/278/297/394, 203/297/314/394/395, 203/310/314/394/395/419, 203/344/394, 203/353, 203/394, 203/394/395, 297/394, 314/394/395, 344/394/395, 353, and 394, and/or any combinations thereof.

13. The engineered terminal deoxynucleotidyl transferase of claim 1, wherein the polypeptide sequence comprises at least one substitution or substitution set comprising substitutions as compared to the reference sequence of SEQ ID NO: 1348 at amino acid positions selected from 3/175/213/313/325/340/457/481/485, 3/307/321/340/353/406/408/445, 148/175/201/457/485, 175/201/333/412/425/457/485, 175/325/397, 175/333, 175/333/369/481, 175/333/485, 175/485, 199/307/321/340/406/408/445/484, 201/213/333/344/397/425/481/485, 201/333/344/457/481, 201/333/481, 201/406/408/462/484/502, 213, 213/333/397, 307/333/340/408/445/462/502, 307/373/406/408/484, 321/333/340, 325/333/369/425, 325/425/457/481, 333, 333/344/369/397, 333/344/369/485, 340/484, 344/485, 353/406, 373, and 406/408, and/or any combinations thereof.

14. The engineered terminal deoxynucleotidyl transferase of claim 1, wherein the polypeptide sequence comprises at least one substitution or substitution set comprising substitutions as compared to the reference sequence of SEQ ID NO: 1596 at amino acid positions selected from 161, 162, 163, 165, 171, 177, 179, 188, 200, 205, 208, 231, 233, 251, 252, 253, 260, 261, 277, 306, 307, 321, 325, 327, 329, 330, 353, 368, 370, 371, 376, 380, 382, 393, 400, 402, 405, 406, 407, 408, 410, 413, 414, 419, 426, 441, 442, 446, 460, 464, 484, 488, 490, 495, 506, 508, 518, 520, 160/219/460/503/506, 160/165/203/205/219/353/406/408/442/446/460/488, 160/165/205/219/406/408/441/442/446, 160/203/205/406/408/441/442/446, 160/219/330/406/408/442/446/484, 205/307/406/408/441/442/446/460/488, 252/333, 406/408/441/442/446, 406/408/442/446219/307/326, 406/408, 406/408/442/446, and 406/408/490, and/or any combinations thereof.

15. The engineered terminal deoxynucleotidyl transferase of claim 1, wherein the polypeptide sequence comprises at least one substitution or substitution set comprising substitutions as compared to the reference sequence of SEQ ID NO: 1654 at amino acid positions selected from 14/53/300, 14/53/419, 106/300/415/419/456, 140, 160, 160/163/165/203/205/219/353/414/441/460/488, 160/165/203/205/219/353/460/488, 160/165/179/203/205/208/219/353/376/413/414/441/488, 160/165/179/203/307/376/413/495, 160/165/203/205/219/353/460/488, 160/165/203/205/307/371/376/413/414, 160/165/203/208/371/376/413/414, 160/165/205, 160/165/205/208, 160/165/205/219/441, 160/165/205/414/441/495, 160/165/307/353/413/414/441/488/495, 160/179/203/205/208/219/371/414/484/506, 160/179/208/219/307/413/414/503/506, 160/179/208/307/371/376/414, 160/179/307/376/441/488/503, 160/203/205/208/219/414/460/506, 160/203/205/208/413/460/484/488, 160/203/205/441, 160/203/208/307/353/495, 160/203/208/371/413/414, 160/203/208/413/414/441/484, 160/203/326/353/413/414/484/495, 160/205/208/219/326/441/484/488/503, 160/208/326/376/414/441/484/488, 160/208/371/441/484/506, 160/208/414/441/452/480/488/495, 160/219/307/371/506, 160/219/330/484, 165/179/203/205/219/414/418/441/488/503, 165/179/203/205/484/503, 165/179/205/413/441, 165/179/208/353/413/414/503, 165/203/205, 165/203/205/307/414/441/484/495/503/506, 165/203/205/484/488, 165/203/208/326/376/503, 165/205, 165/208/326/413/414/484/495/506, 179, 179/203/205, 179/203/208/326/353/376/484, 179/205/208/353/414/441/460/484/488, 179/205/353, 179/208/353/460, 179/353, 203/205/208/307/330/353/441/460/503/506, 203/205/208/307/441, 203/205/208/353, 203/208/219/376/441, 203/208/219/441, 203/208/326/353, 203/413/503/506, 205/208/307/353/376/413, 205/208/414, 205/219/307/353, 205/307, 205/307/376/414/441/495, 205/307/441/460/488, 205/326/488/503/506, 208/488/506, 300/395/4193/160/205/208/219/307/353/371/376/413/414/441/488, 326/353/371/376/414, and 441, and/or any combinations thereof.

16. The engineered terminal deoxynucleotidyl transferase of claim 1, wherein the polypeptide sequence comprises at least one substitution or substitution set comprising substitutions as compared to the reference sequence of SEQ ID NO: 1830 at amino acid positions selected from 163/179/277/338/340, 163/414/441, 171/200/334/406/490, 177, 194, 196, 266, 267, 268, 269, 273, 273/501, 274, 277, 278, 292/406, 297, 298, 299, 301, 302, 309, 312, 347, 359, 390, 392, 394, 407, 408, 413, 416, 454, 468, 473, 477, 479, and 493, and/or any combinations thereof.

17. The engineered terminal deoxynucleotidyl transferase of claim 1, wherein the polypeptide sequence comprises at least one substitution or substitution set comprising substitutions as compared to the reference sequence of SEQ ID NO: 1950 at amino acid positions selected from 163/169, 185, 186, 192, 193, 194, 197, 198, 245, 251, 258, 259, 261, 263, 271, 274, 278, 280, 284, 286, 290, 291, 297, 304, 306, 308, 316, 347, 352, 359, 362, 378, 393, 396, 398, 399, 405, 407, 409/414, 410/414, 411/414, 413/414, 414, 414/415, 414/417, 415, 455, 465, 466, 468, 494, and 509, and/or any combinations thereof.

18. The engineered terminal deoxynucleotidyl transferase of claim 1, wherein the polypeptide sequence comprises at least one substitution or substitution set comprising substitutions as compared to the reference sequence of SEQ ID NO: 2008 at amino acid positions selected from 3, 8, 9, 11, 12, 15, 16, 26, 26/27, 36, 38, 39, 50, 58, 62, 70, 76, 79, 81, 90, 116, 147, 151, 157, 158, 158/274/411/413/414, 185/274/413/414, 185/411/413/414, 189, 196, 205, 206, 246, 248, 249, 253, 255, 262, 263/411/413/414/468, 263/413/414, 274/286/411/413/417/468, 274/411/417/468, 274/411/468, 274/413/414/417/468, 274/468, 278/411/413/468, 307, 314, 318, 324, 353, 397, 408, 410, 411/413/417, 411/413/417/468, 411/413/468, 411/414, 413, 413/414, 413/414/468, 413/417/468, 413/468, 414, and 468, 469, 473, 480, 481, 491, and 493, and/or any combinations thereof.

19. The engineered terminal deoxynucleotidyl transferase of claim 1, wherein the polypeptide sequence comprises at least one substitution or substitution set comprising substitutions as compared to the reference sequence of SEQ ID NO: 2254 at amino acid positions selected from 197/407/455, 197/455, 284/398/466, 362/407/455, 396/398/410/466, 398/466, 399/411/416, and 466, and/or any combinations thereof.

20. The engineered terminal deoxynucleotidyl transferase of claim 1, wherein the polypeptide sequence comprises at least one substitution or substitution set comprising substitutions as compared to the reference sequence of SEQ ID NO: 2514 at amino acid positions selected from 26/90/94/248/261/266/362/455, 26/90/246, 26/90/246/248, 26/90/246/248/261, 26/90/246/248/362/455, 26/90/246/248/455, 26/90/246/266/362, 26/90/246/362, 26/90/246/455, 26/90/248, 26/90/248/266/455, 26/90/248/455, 26/90/248/455/459, 26/90/266, 26/90/362/455, 26/173/248, 26/246, 26/246/248/362, 26/246/248/362/455, 26/246/248/455, 26/248, 26/248/261/266, 26/248/261/266/362/455, 26/248/266/362, 26/248/362/455, 26/248/455, 26/362, 26/362/455, 58/197/249/407/410, 62/249, 90/246/248, 90/246/248/261/266/362/455, 90/246/248/266/362/455, 246/248, 246/248/362, 246/266/455, 248, 248/266/362, 248/362/455, 248/455, and 362, and/or any combinations thereof.

21. The engineered terminal deoxynucleotidyl transferase of claim 1, wherein the polypeptide sequence comprises at least one substitution or substitution set comprising substitutions as compared to the reference sequence of SEQ ID NO: 2524 at amino acid positions selected from 9/16/62/157/246/249/362, 11/58/227/246, 12/16/158/246/248/249, 30/189/261/266/353/465/468, 47/326, 30/189/266, 30/261/266/353/468, 30/266, 30/266/303, 30/266/353, 38, 38/81/318, 38/197, 39, 39/79, 58/157/158/362, 70/353, 79/81, 81, 189, 189/261, 189/353, 246/249, 261/353, 266/307/353/468, 266/353/468, 266/468, 169, 175, 179, 191/413, 200, 203, 292, 293, 304/329, 325, 327/406, 329, 340, 353/459, 373, 379, 382, 402, 403, 404, 406, 427, 429, 459, 461, 484, 490, 495, 504, 506, and 508, and/or any combinations thereof.

22. The engineered terminal deoxynucleotidyl transferase of claim 1, wherein the polypeptide sequence comprises at least one substitution or substitution set comprising substitutions as compared to the reference sequence of SEQ ID NO: 2638 at amino acid positions selected from 11/30/79/189/480, 30/58/79/189/307/480, 79/189/307/410, and 79/307, and/or any combinations thereof.

23. The engineered terminal deoxynucleotidyl transferase of claim 1, wherein the polypeptide sequence comprises at least one substitution or substitution set comprising substitutions as compared to the reference sequence of SEQ ID NO: 2804 at amino acid positions selected from 169/304/340/402/427, 169/304/340/427/429/504, 169/304/340/429/506, 169/304/340/504, 169/304/402/403/427/506, 169/304/403/427/504, 169/304/427/504/506, 169/304/504, 169/340/402/403/427/429/504, 169/340/402/403/427/504/506, 169/340/402/504, 169/340/427, 169/340/506, 169/402/403/504/506, 169/402/427/429/504, 169/402/504, 169/402/504/506, 169/403/427/506, 266/327/329/404/410, 292/327/329/468, 304, 304/340/402, 304/340/402/403/427/429/504/506, 304/340/402/403/504/506, 304/340/402/403/506, 304/340/402/427/504/506, 304/340/402/506, 304/340/403/427/429/504/506, 304/340/427, 304/402/403, 304/402/403/427/504, 304/402/403/429, 304/402/403/504/506, 304/402/403/506, 304/403/504, 304/504, 304/504/506, 327, 327/329, 327/329/379/404/406/410, 327/329/404/410, 327/329/465/484, 327/382/406/410/484, 327/410/484, 340, 340/402/403, 340/402/427/429/506, 340/402/429/504/506, 340/402/504, 340/403/504, 340/504, 340/506, 379/382/468, 379/404/410, 379/410, 379/465/468/484, 379/468, 402/403/427/504/506, 402/403/429/504, 402/427/504, 402/504, 403/427, 427, 484, 504, 504/506, and 506, and/or any combinations thereof.

24. The engineered terminal deoxynucleotidyl transferase of claim 1, wherein the polypeptide sequence comprises at least one substitution or substitution set comprising substitutions as compared to the reference sequence of SEQ ID NO: 2812 at amino acid positions selected from 30, 186, 188, 198, 231, 233, 235, 243, 248, 253, 264, 266, 278, 287, 297/440, 326, 366, 367, 368, 370, 396, 414, 433, 435, 437, 439, 442, 444, 446, 485, 499, 503, 515, 520, 525, 30/179/200/373/403, 30/179/373/379, 30/184/246/325/379/429/495, 30/184/246/327/329/459, 30/184/246/379/404, 30/184/246/459/461/495/500/504, 30/200/373, 30/200/373/379, 30/246/325/327/329/404/461, 30/246/325/379/404/427/429/461, 30/246/427/459/461, 30/325/327, 30/325/327/379/404/429/495, 30/327/404/504, 30/329/379, 30/373, 30/373/403, 30/379/429/459/461, 30/379/459/461/504, 30/403/441/460, 200/373/379, 246/325/329/379/461, 246/327/404/461/495, 327/329/379/504, 327/459/461/495, 353/403, 373/379, 373/379/403, 379/403/406/468, and 403/441, and/or any combinations thereof.

25. The engineered terminal deoxynucleotidyl transferase of claim 1, wherein the polypeptide sequence comprises at least one substitution or substitution set comprising substitutions as compared to the reference sequence of SEQ ID NO: 2956 at amino acid positions selected from 140/142/153/177/427/434/441/444/461/502, 140/142/153/427/484, 140/142/177, 140/142/177/441, 140/142/365/373/404/427/484, 140/142/373/427/484, 140/148/161/177/404, 140/150/153/365/373/427/484, 140/150/177/404/436/441/484/502, 140/161/177/404/427/484, 140/177/404, 140/177/404/484, 142/150/158/177/427/445, 142/150/177/404, 142/153/177/441/444, 142/177/373, 142/177/373/441, 142/461/484/502, 150/177, 153, 153/161/325/404/427/441/484, 153/484, 177, 177/365/427/434, 325/427, 427, and 484, and/or any combinations thereof.

26. The engineered terminal deoxynucleotidyl transferase of claim 1, wherein the polypeptide sequence comprises at least one substitution or substitution set comprising substitutions as compared to the reference sequence of SEQ ID NO: 3174 at amino acid positions selected from 122/473, 189, 190, 193, 194, 196, 263, 264, 266, 267, 268, 269, 273, 273/501, 274, 278, 279, 281, 297, 298, 299, 300, 301, 302, 304, 309, 312, 315, 347, 350, 352, 353, 359, 390, 392, 394, 407, 408, 410, 411, 413, 414, 416, 436, 454, 468, 472, 473, 477, 479, 480, 493, 186/188/248/253/365/366/444/445, 186/231/248/253/484, 186/231/248/365/366/368/484, 186/231/248/366/368/444, 186/231/248/484, 186/231/368/416/441/442/444/484/485, 186/231/441/444/445, 186/484/485, 188/231/248/253/365/441/442/444/445/484, 198/243/264/431/441, 198/243/396/414/431/433/441/499, 198/243/396/414/433/437/441/515, 198/264/266/414, 198/264/396/414/433/441/515, 198/266/396/414/433/441/499/501, 198/266/414/433/441/515, 198/266/437/441/499, 198/414/431/441/499/520, 198/414/433/441/515/520, 231/248/441/484, 243/264/515/520, 243/396/414/433/441/515/520, 243/414/433/437/441, 243/414/437/441/515, 248/442/444/445/484, 264/266/396/414/433/441/515, 264/266/414/441/499/501/515/520, 264/414, 264/414/441, 264/433/441, 266/437/441/499/515/520, 365/366/441/484/485, 396/414/441, 396/414/441/515, and 414/441/520, and/or any combinations thereof.

27. The engineered terminal deoxynucleotidyl transferase of claim 1, wherein the polypeptide sequence comprises at least one substitution or substitution set comprising substitutions as compared to the reference sequence of SEQ ID NO: 3222 at amino acid positions selected from 186/194/248/396, 186/198/243/248/366/368/394/501, 186/231/243/368/394/485/499, 186/231/248/485, 186/231/366/368/394/485, 186/243, 186/243/248/366/368/394, 186/243/248/394/484/485, 186/243/484/520, 186/248, 186/365, 186/365/366/368/394/499, 186/365/366/394/485, 186/366/368/394/396/484, 186/366/368/394/396/484/485, 186/394/396/485, 194/198/243/366/368/499, 194/515/520, 198/231/243/248/485, 198/243/248/365/394/501, 198/248/394/396/484/485/499, 198/394/396/484/485/499, 198/394/396/499/515/520, 198/394/499/501, 231/365/368/394/499/520, 231/368/394, 231/484/485/499/501, 243/248/394/396/484/485, 243/484, 243/484/485/499, 248/365/366/368/394/484/520, 248/394/484, 365/366/368/394, 365/368/394/396/520, 394/396, and 394/499, and/or any combinations thereof.

28. The engineered terminal deoxynucleotidyl transferase of claim 1, wherein the polypeptide sequence comprises at least one substitution or substitution set comprising substitutions as compared to the reference sequence of SEQ ID NO: 3670 at amino acid positions selected from 168, 198, 267, 273, 273/493, 273/493/499, 273/309/493/499, 273/309/413/499, 273/499, 274/299/408/416, 274/408/416, 274/416, 288/299/416, 298/299/416, 301, 303/396, 307, 308, 308/361, 309, 309/413, 309/499, 313, 372, 392, 397, 413, 413/493/499, 413/499, 415, 416, 419, 451, 452, 456, 472, 473, 475, 493/499, and 528, and/or any combinations thereof.

29. The engineered terminal deoxynucleotidyl transferase of claim 1, wherein the polypeptide sequence comprises at least one substitution or substitution set comprising substitutions as compared to the reference sequence of SEQ ID NO: 3674 at amino acid positions selected from 194/196/390, 194/196/390/394/460/480, 194/196/390/394/480, 194/196/390/454/480, 194/196/390/480, 194/196/394/454/480, 194/196/454, 194/390, 194/394, 194/394/454, 194/394/454/480, 196, 196/390, 196/390/394, 196/390/394/454, 196/390/394/454/480, 196/390/394/480, 196/390/454, 196/394, 196/394/454, 196/394/454/480, 196/394/480, 196/454, 297/470/473, 297/473/493, 390, 390/394, 390/394/454, 390/394/454/480, 390/394/480, 390/454, 390/480, 394, 394/480, and 454, and/or any combinations thereof.

30. The engineered terminal deoxynucleotidyl transferase of claim 1, wherein the polypeptide sequence comprises at least one substitution or substitution set comprising substitutions as compared to the reference sequence of SEQ ID NO: 3796 at amino acid positions selected from 198, 198/267/313/451/475/494/499, 198/267/314/451, 198/267/314/475, 198/267/409/451, 198/267/475, 198/451/493, 208/308, 208/308/461, 267, 267/314/328/451/494/499, 267/451, 267/451/494/499, 308, 314/328/451/499, 314/451, 328/409/451, 328/451, 409, 409/475/494, 451, 451/493/494, 451/493/499, 451/494, and 475, and/or any combinations thereof.

31. The engineered terminal deoxynucleotidyl transferase of claim 1, wherein the polypeptide sequence comprises at least one substitution or substitution set comprising substitutions as compared to the reference sequence of SEQ ID NO: 2 or 660 at amino acid positions selected from 26, 30, 38, 79, 81, 90, 92, 94, 101, 108, 137, 140 141, 142, 153, 155, 160, 163, 165, 177, 184, 189, 194, 196, 201, 203, 205, 213, 219, 231, 246, 248, 258, 263, 264, 266, 267, 301, 304, 307, 314, 318, 325, 333, 340, 344, 353, 362, 379, 390, 392, 394, 395, 396, 397, 398, 402, 403, 406, 408, 410, 411, 413, 414, 416, 425, 427, 429, 433, 434, 441, 442, 444, 446, 451, 455, 460, 461, 466, 468, 476, 481, 484, 485, 488, 495, 499, 501, 502, 506, 515, 525, 26/30/38/79/81/90/92/94/101/108/137/140/141/142/153/155/160/163/165/177/184/189/194/196/201/203/205/213/219/231/248/258/263/264/266/267/301/304/307/314/318/325/333/340/344/353/362/379/390/392/394/395/397/398/402/403/406/408/410/4 11/413/414/416/425/427/429/433/434/441/442/444/446/451/455/460/461/466/468/476/481/484/485/488/495/499/501/502/506/515/525, 26/30/38/79/81/90/92/94/101/108/137/140/141/142/153/155/160/163/165/177/184/189/201/203/205/213/219/231/248/258/263/264/266/304/307/314/318/325/333/340/344/353/362/379/392/394/395/397/398/402/403/406/408/410/411/413/414/416/425/4 27/429/433/434/441/442/444/446/455/460/461/466/468/476/481/484/485/488/495/499/501/502/506/515/525, 26/38/79/81/90/92/94/101/108/137/141/155/160/163/165/189/201/203/205/213/219/246/248/258/263/264/304/307/314/318/333/340/344/353/362/392/394/395/396/397/398/402/403/406/408/410/411/413/414/425/441/442/446/455/460/461/466/468/476/ 481/485/488/506/525, 92/94/101/108/137/141/155/160/163/165/201/203/205/213/219/258/263/264/314/333/344/353/392/394/39 5/397/406/408/411/413/414/425/441/442/446/460/461/468/476/481/485/488/525, and 92/94/101/108/137/141/155/201/213/264/314/333/344/392/394/395/397/406/408/425/442/446/461/476/48 1/485/525, and/or any combinations thereof.

32. The engineered terminal deoxynucleotidyl transferase of claim 1, wherein the polypeptide sequence comprises at least one substitution or substitution set comprising substitutions as compared to the reference sequence of SEQ ID NO: 3870 at amino acid positions selected from 165, 169, 171, 173, 175, 176, 179, 183, 187, 191, 192, 195, 197, 199, 200, 203, 204, 210, 257, 259, 267, 291, 293, 295, 301, 319, 325, 340, 341, 342, 374, 387, 398, 399, 403, 404, 406, 429, 480, 481, 483, 484, 490, 491,493, 494, 495, 521, 507, 508, 509, and 522, and/or any combinations thereof.

33. The engineered terminal deoxynucleotidyl transferase of claim 1, wherein the polypeptide sequence comprises at least one substitution or substitution set comprising substitutions as compared to the reference sequence of SEQ ID NO: 3918 at amino acid positions selected from 3, 7/400/459/504, 164, 173/367/459/500, 184, 203, 203/367/459, 203/367/459/500/501, 203/400/459/501, 203/459, 203/459/499/504, 203/459/500, 215, 218, 294, 335, 335/402/481/484, 335/402/512, 335/481/484, 335/481/493, 335/481/512, 336, 338, 339, 367, 367/459/500, 370, 373, 376, 380, 384, 390, 395, 395/402, 395/402/481/484, 395/481, 395/481/512, 395/484, 400, 400/459, 400/459/499/500/504, 400/459/500/501, 400/459/501/504, 400/501, 400/504, 402, 402/481, 402/481/484, 402/481/484/512, 402/481/493, 402/484/493, 402/512, 458, 459, 459/501, 460, 481, 481/512, 484, 485, 493, 499, 500, 501, 504, 512, 515, and 516, and/or any combinations thereof.

33. The engineered terminal deoxynucleotidyl transferase of claim 1, wherein the polypeptide sequence comprises at least one substitution or substitution set comprising substitutions as compared to the reference sequence of SEQ ID NO: 4266 at amino acid positions selected from 95/428/480, 163/190, 163/203/366, 163/328/363/480, 163/363/480/485, 172/174/178/340, 178, 188, 190/202/203/363/366/480/483/485, 190/202/203/480, 190/480/485, 192, 192/498/499/503, 202, 202/203/328/362/363/366/428/480/485/498/499/503, 202/203/485, 203/328/363/428/483, 203/328/428, 203/328/480/485, 203/362/366, 203/498/499/503, 272, 280, 280/498/499/503, 296, 297, 299, 299/498/499/503, 301, 301/503, 308, 308/503, 311, 328/428, 328/480/483, 328/485, 343, 346, 349, 358, 359/498/499/503, 362/363, 362/363/366/428/480/483/498/499/503, 392/498/499/503, 406, 407, 410, 411, 418/498/499/503, 418/503, 419/503, 465, 471, 472/498/499/503, 473, 480/483, 491, and 498/499/503, and/or any combinations thereof.

34. The engineered terminal deoxynucleotidyl transferase of claim 1, wherein the polypeptide sequence comprises at least one substitution or substitution set comprising substitutions as compared to the reference sequence of SEQ ID NO: 4558 at amino acid positions selected from 94/365/367, 172/174/178/401/403, 172/174/178/401/403/507, 172/174/178/402/508, 172/174/401/403/507, 172/178, 172/178/401, 174/178, 178/401/403, 178/402/403, 318/375/380, 324/379/405/483, 340, 340/394, 365, 365/367/428, 365/389/394, 367, 375/376, 375/379/483, 375/380, 375/380/400/483, 375/380/483, 376/483, 379/483, 389/394, 394, 401, 401/402/403/507, 402/403, 405/483, and 483, and/or any combinations thereof.

35. The engineered terminal deoxynucleotidyl transferase of claim 1, wherein the polypeptide sequence comprises at least one substitution or substitution set comprising substitutions as compared to the reference sequence of SEQ ID NO: 4442 at amino acid positions selected from 174/296/299, 182, 185/190, 189/190, 190, 190/193, 192/280, 192/402/507, 257, 259, 260, 281, 289, 296/299, 305, 306, 307, 308, 312, 313, 316, 318, 327, 374, 381, 394, 395, 402, 402/507, 404, 405, 414, 432, 451, 455, 460, 461, 476/480, 480, 480/481, 493, 494, and 522, and/or any combinations thereof.

36. The engineered terminal deoxynucleotidyl transferase of claim 1, wherein the polypeptide sequence comprises at least one substitution or substitution set comprising substitutions as compared to the reference sequence of SEQ ID NO: 4654 at amino acid positions selected from 190, 190/197/308, 190/308/380/405, 190/375, 190/375/380, 190/380/405, 190/405/406, 272/301/393/394/480, 272/318/480/483, 301/394/480, 318, 375, 375/380, 375/405, 375/405/406, 380, 394, 394/480, and 480/483, and/or any combinations thereof.

37. The engineered terminal deoxynucleotidyl transferase of claim 1, wherein the polypeptide sequence comprises at least one substitution or substitution set comprising substitutions as compared to the reference sequence of SEQ ID NO: 4850 at amino acid positions selected from 189, 189/193/207/307/353, 190/322, 193, 193/307, 261/322/421, 297/298/300/392, 297/300, 297/300/328, 298/300/328, 298/300/328/395, 298/300/360, 298/300/392/395, 298/300/392/395/492, 298/300/395, 298/300/481, 300, 300/392/395, 319, 322, 392, 421, and 492, and/or any combinations thereof.

38. The engineered terminal deoxynucleotidyl transferase of claim 1, wherein the polypeptide sequence comprises at least one substitution or substitution set comprising substitutions as compared to the reference sequence of SEQ ID NO: 4856 at amino acid positions selected from 10/413, 260, 268, 302/307, 317, 353, 354, 362, 364, 392, 393, 394, 395, 397, 402, 404, 412, 413, 419, 436/512, 460, 477, 486, 490, 495, and 518, and/or any combinations thereof.

39. The engineered terminal deoxynucleotidyl transferase of claim 1, wherein the polypeptide sequence comprises at least one substitution or substitution set comprising substitutions as compared to the reference sequence of SEQ ID NO: 4904 at amino acid positions selected from 190, 190/287/300/302, 190/300/477/490, 194/300/302/413, 194/300/302/481, 297/298/308/392/395, 298/392/525, 300, 300/317, 300/490, and 395, and/or any combinations thereof.

40. The engineered terminal deoxynucleotidyl transferase of claim 1, wherein the polypeptide sequence comprises at least one substitution or substitution set comprising substitutions as compared to the reference sequence of SEQ ID NO: 5002 at amino acid positions selected from 11/523, 190/194, 194, 194/198, 202, 208, 264, 273, 273/347/354, 274, 281, 290, 298/300/302, 298/302/392/393/394/433, 298/302/392/394, 298/392/393/394, 298/392/393/394/477, 298/392/394/490, 298/393/394/395/433/477, 298/393/394/477/495, 298/394/433, 300/302/303, 308/402/460, 309, 313, 314, 324, 352, 359, 360, 361, 392/393/394/433, 392/393/394/477, 392/393/394/477/495, 392/393/394/490, 392/394, 392/394/395, 392/394/433/477, 392/394/433/495, 392/394/477/495, 392/394/495, 393/394, 393/394/433/477/490, 394/477, 394/490, 405, 408/413, 411/413, 413, 460/525, 463, 466, 467, 472, 473, 477, 492, 523, and 526, and/or any combinations thereof.

41. The engineered terminal deoxynucleotidyl transferase of claim 1, wherein the polypeptide sequence comprises at least one substitution or substitution set comprising substitutions as compared to the reference sequence of SEQ ID NO: 5028 at amino acid positions selected from 82/194/198/313, 194, 194/198, 194/198/208/313, 194/198/309, 194/198/313, 194/198/411, 194/208/411, 194/309, 194/313, 194/411, 198, 198/208, 198/208/309/411, 198/208/313/411, 273/274, 274, 274/281/526, 274/359/526, 274/523, 309, 309/313/411, 324/526, 411, 466, 466/526, 523, and 526, and/or any combinations thereof.

42. The engineered terminal deoxynucleotidyl transferase of claim 1, wherein the polypeptide sequence comprises at least one substitution or substitution set comprising substitutions as compared to the reference sequence of SEQ ID NO: 5192 at amino acid positions selected from 96/295, 169, 176, 177, 179, 184, 187, 193, 195, 197, 197/307, 198, 199, 200, 203, 292, 295, 300/394, 304, 325, 326, 326/380, 329, 373, 376, 377, 383, 394, 403, 409, 430, 485, 508, and 520/526, and/or any combinations thereof.

43. The engineered terminal deoxynucleotidyl transferase of claim 1, wherein the polypeptide sequence comprises at least one substitution or substitution set comprising substitutions as compared to the reference sequence of SEQ ID NO: 5246 at amino acid positions selected from 177/198/200, 177/200/203, 177/200/203/295, 177/200/295/326, 180, 184/198/200/203/295, 184/200/295/326, 190/198/200/203, 190/200/203/295/380, 190/200/295, 197, 198, 198/200, 198/200/203, 198/200/203/295, 200/326, 200/380, 203, 203/380, 233, 252, 295, 336, 364, 365, 367, 381, 384, 441, 459, and 485, and/or any combinations thereof.

44. The engineered terminal deoxynucleotidyl transferase of any of claims 1-43, wherein the polypeptide sequence further comprises an N-terminal truncation of 1-156 amino acids.

45. The engineered terminal deoxynucleotidyl transferase of any of claims 1-44, wherein the engineered terminal deoxynucleotidyl transferase polypeptide is fused with a second polypeptide; optionally, wherein the second polypeptide has inorganic pyrophosphatase (IPP) activity

46. The engineered terminal deoxynucleotidyl transferase claim 45, wherein the second polypeptide with IPP activity comprises an amino acid sequence selected from SEQ ID NO: 3942 and 3944.

47. The engineered terminal deoxynucleotidyl transferase claim 45, wherein the engineered terminal deoxynucleotidyl transferase polypeptide fused with the second polypeptide comprises a sequence selected from SEQ ID NO: 5468, 5470, 5472, and 5474.

48. The engineered terminal deoxynucleotidyl transferase of any of claims 1-47, wherein said engineered terminal deoxynucleotidyl transferase is capable of template-independent synthesis.

49. The engineered terminal deoxynucleotidyl transferase of any of claims 1-47, having at least one improved property, as compared to a wild-type or reference terminal deoxynucleotidyl transferase or template-independent polymerase.

50. The engineered terminal deoxynucleotidyl transferase of claim 49, wherein said improved property is selected from increased thermostability, increased activity at elevated temperatures, increased soluble expression or isolated protein yield, decreased by-product formation, increased specific activity on one or more NTP-3′-O-RBG or natural or modified NTP substrates, increased incorporation efficiency in extension of oligo acceptor substrates, and increased activity on one or more oligo acceptor substrates.

51. The engineered terminal deoxynucleotidyl transferase of any of claims 1-50, wherein said engineered terminal deoxynucleotidyl transferase comprises increased soluble expression or isolated protein yield as compared to a wild-type or reference terminal deoxynucleotidyl transferase or template-independent polymerase.

52. The engineered terminal deoxynucleotidyl transferase of any of claims 1-51, wherein said engineered terminal deoxynucleotidyl transferase comprises increased thermal stability as compared to a wild-type or reference terminal deoxynucleotidyl transferase or template-independent polymerase.

53. The engineered terminal deoxynucleotidyl transferase of any of claims 1-52, wherein said engineered terminal deoxynucleotidyl transferase comprises increased activity on one or more NTP-3′-O-RBG or natural or modified NTP substrates as compared to a wild-type or reference terminal deoxynucleotidyl transferase or template-independent polymerase.

54. The engineered terminal deoxynucleotidyl transferase of any of claims 1-53, wherein said terminal deoxynucleotidyl transferase is purified.

55. A polynucleotide sequence encoding at least one engineered terminal deoxynucleotidyl transferase of any of claims 1-54.

56. A polynucleotide sequence comprising at least 85%, 90%, 91%, 92%, 93%, 94%, 95%, 96%, 97%, 98%, 99%, or more sequence identity to the reference sequence of SEQ ID NOs: 1, 7, 11, 15, 23, 35, 53, 267, 647, 659, 881, 1099, 1335, 1347, 1595, 1653, 1829, 1949, 2007, 2253, 2513, 2523, 2637, 2803, 2811, 2955, 3173, 3221, 3669, 3673, 3795, 3869, 3917, 4265, 4441, 4653, 4849, 4855, 4903, 5001, 5027, 5191, and 5245, and/or or a functional fragment thereof, wherein said polynucleotide sequence encodes an engineered polypeptide comprising at least one substitution at one or more amino acid positions.

57. The polynucleotide sequence of claim 56, wherein said polynucleotide sequence encodes at least one engineered terminal deoxynucleotidyl transferase comprising a sequence having at least 85%, 90%, 91%, 92%, 93%, 94%, 95%, 96%, 97%, 98%, 99%, or more sequence identity to the reference sequence of SEQ ID NOs: 2, 8, 12, 16, 24, 36, 54, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192, and 5246.

58. The polynucleotide sequence of claim 56, wherein said sequence comprises SEQ ID NOs: 7, 11, 15, 23, 35, 53, 267, 647, 659, 881, 1099, 1335, 1347, 1595, 1653, 1829, 1949, 2007, 2253, 2513, 2523, 2637, 2803, 2811, 2955, 3173, 3221, 3669, 3673, 3795, 3869, 3917, 4265, 4441, 4653, 4849, 4855, 4903, 5001, 5027, 5191, and 5245.

59. The polynucleotide sequence of claim 56, wherein said polynucleotide sequence is operably linked to a control sequence.

60. The polynucleotide sequence of claim 56, wherein said polynucleotide sequence is codon-optimized.

61. An expression vector comprising at least one polynucleotide sequence of any of claims 55-60.

62. A host cell comprising at least one expression vector of claim 61.

63. A method of producing an engineered terminal deoxynucleotidyl transferase polypeptide in a host cell comprising culturing a host cell of claim 62, under suitable culture conditions, such that at least one engineered terminal deoxynucleotidyl transferase is produced.

64. The method of claim 63, further comprising recovering at least one engineered terminal deoxynucleotidyl transferase from the culture and/or host cells.

65. The method of claim 63 or 64, further comprising the step of purifying said at least one engineered terminal deoxynucleotidyl transferase.

66. A composition comprising at least one engineered terminal deoxynucleotidyl transferase of any of claims 1-54.

67. A method for template-independent synthesis of an oligonucleotide, the method comprising:
a) providing at least one terminal deoxynucleotidyl transferase or template-independent polymerase;
b) providing at least one oligo acceptor substrate, wherein the oligo acceptor substrate comprises a 3′-OH, or an equivalent thereof;
c) contacting the oligo acceptor substrate, the terminal deoxynucleotidyl transferase or template-independent polymerase, and one or more nucleotide triphosphates, modified nucleotide triphosphates, or NTP-3′-O-RBG under conditions sufficient for the addition of the nucleotide, modified nucleotide, or nucleotide-3′-O-RBG to the 3′ end of the oligo acceptor substrate.

68. The method of claim 67, wherein the method further comprises (d) deblocking the oligonucleotide formed in step (c) at the protected 3-O-position of the oligonucleotide product.

69. The method of claim 67 or 68, wherein the method further comprises e) deactivating unreacted nucleotide triphosphates, modified nucleotide triphosphates, or NTP-3′-O-RBGs.

70. The method of any of claims 67-69, wherein the method further comprises an optional step (f) of removing excess nucleoside and/or excess inorganic phosphate or pyrophosphate from the reaction.

71. The method of any of claims 67-70, wherein the method further comprises repeating steps (a)-(c) or (a)-(d) or (a)-(e) or (a)-(f) until a desired oligonucleotide sequence is obtained.

72. The method of any of claims 67-71, wherein the method further comprises (g) cleaving or releasing the growing or completed oligonucleotide chain.

73. The method of any of claims 68-72, wherein step (d) deblocking the oligonucleotide formed in step (c) at the protected 3-O-position and step (e) deactivating the unreacted nucleotide triphosphates, modified nucleotide triphosphates, or NTP-3′-O-RBGs occur simultaneously.

74. The method of any of claims 67-73, wherein a phosphatase is used to deblock the NTP-3′-O-RBG and to deactivate unreacted NTP-3′-O-RBGs by removing at least one of the 5′ phosphates.

75. The method of claim 74, wherein the phosphatase comprises an alkaline phosphatase set forth in SEQ ID NO: 3922, 3924, 3926, 3928, 3930, 3932, or 3934.

76. The method of any of claims 67-75, wherein step (c) optionally includes contacting the oligo acceptor substrate, the terminal deoxynucleotidyl transferase or template-independent polymerase, and either a nucleotide triphosphate, or a modified nucleotide triphosphate, or an NTP-3′-O-RBG with a phosphatase, such as an inorganic pyrophosphatase, to convert pyrophosphate to inorganic phosphate.

77. The method of claim 76, wherein the phosphatase comprises an inorganic pyrophosphatase set forth in SEQ ID NO: 3936, 3938, 3940, 3942, 3944, 3946, or 3948.

78. The method of any of claims 67-77, further comprising an NTP-3′-O-RBG comprising a carbonitrile, phosphate, carbonate, carbamate, ester, ether, borate, nitrate, sugar, phosphoramidate, phenylsulfenate, or sulfate.

79. The method of any of claims 67-78, wherein the nucleotide triphosphate, the modified nucleotide triphosphate, or the NTP-3′-O-RBG comprise a 2′ modification.

80. The method of claim 79, wherein the 2′ modification comprises a 2′-F, 2′-O-alkyl, 2′-O-methoxyethyl, or a locked or constrained ethyl.

81. The method of claim 80, wherein the modification is 2′-F and the 2′ modified nucleotide triphosphate, the modified nucleotide triphosphate, or the NTP-3′-O-RBG is selected from: 2′-fluoro-2′-deoxyadenosine-5′-triphosphate, 2′-fluoro-2′-deoxycytidine-5′-triphosphate, 2′-fluoro-2′-deoxyguanosine-5′-triphosphate, and 2′-fluoro-2′-deoxyuridine-5′-triphosphate.

82. The method of claim 80, wherein the modification is 2′-O-alkyl and the 2′ modified nucleotide triphosphate, the modified nucleotide triphosphate, or the NTP-3′-O-RBG is selected from: 2′-O-methyladenosine-5′-triphosphate, 2′-O-methylcytidine-5′-triphosphate, 2′-O-methylguanosine-5′-triphosphate, 2′-O-methyluridine-5′-triphosphate, and 2′-O-methylinosine-5′-triphosphate.

83. The method of any of claims 67-82, wherein the oligonucleotide chain comprises one or more phosphorothioate linkages.

84. The method of any of claims 67-83, wherein the oligonucleotide, oligonucleotide sequence, or oligonucleotide chain comprises RNA.

85. The method of any of claims 67-84, wherein the oligonucleotide, oligonucleotide sequence, or oligonucleotide chain comprises DNA.

86. The method of any of claims 67-85, wherein the terminal deoxynucleotidyl transferase or template-independent polymerase comprises one or more engineered terminal deoxynucleotidyl transferases.

87. The method of claim 86, wherein the engineered terminal deoxynucleotidyl transferase of any one of claims 1-54.

88. The method of claim 86, wherein the engineered terminal deoxynucleotidyl transferase comprises a polypeptide sequence having at least 85%, 90%, 91%, 92%, 93%, 94%, 95%, 96%, 97%, 98%, 99%, or more sequence identity to a reference sequence of SEQ ID NO: 2, 8, 12, 16, 24, 36, 54, 268, 648, 660, 882, 1100, 1336, 1348, 1596, 1654, 1830, 1950, 2008, 2254, 2514, 2524, 2638, 2804, 2812, 2956, 3174, 3222, 3670, 3674, 3796, 3870, 3918, 4266, 4442, 4654, 4850, 4856, 4904, 5002, 5028, 5192, and/or 5246, or a functional fragment thereof, and comprises an amino acid residue difference or set of amino acid residue differences in its polypeptide sequence relative to the reference sequence, wherein the amino acid residue difference or set of differences is selected from the amino acid differences listed in: Tables 5.1, 6.2, 7.2, 8.2, 9.2, 10.2, 11.2, 12.2, 13.2, 14.2, 15.2, 16.2, 17.2, 18.2, 19.2, 20.2, 21.2, 22.2, 23.2, 24.2, 25.2, 26.2, 26.3, 26.4, 27.2, 27.3, 27.4, 27.5, 28.1, 28.2, 28.3, 29.2, 30.2, 31.2, 32.2, 33.2, 34.2, 35.2, 36.2, 37.2, 38.2, 39.2, 40.2, 41.2, 42.2, 43.2, 44.2, 45.2 46.2, 47.2, 48.2, 49.2, 50.2, 51.2, 52.2, 53.2, 54.2, 55.2, 56.2, 56.3, 56.4, 61.2, 63.2, 64.2, 65.2, 66.2, 67.2, 68.2, 69.2, 70.2, 71.2, 72.2, 73.2, 74.2, 75.2, 76.2, 77.2, 78.2, 79.2, and 80.1.

89. The method of claim 86, wherein the engineered terminal deoxynucleotidyl transferase comprises a polypeptide sequence having at least 85%, 90%, 91%, 92%, 93%, 94%, 95%, 96%, 97%, 98%, 99%, or more, sequence identity to an amino acid sequence selected from the even-numbered sequences SEQ ID NO: 4-1960, 2004-3920, 4048-5466, and 5476.

90. The method of any of claims 67-89, wherein the terminal deoxynucleotidyl transferase or template-independent polymerase is immobilized.

91. The method of claim 90, wherein the method further comprises a column solid support for immobilization of the terminal deoxynucleotidyl transferase or template-independent polymerase.

92. The method of claim 90, wherein the method further comprises a batch method with a solid support for immobilization of the terminal deoxynucleotidyl transferase or template-independent polymerase.

93. The method of any of claims 67-89, wherein the oligoacceptor substrate and subsequent oligonucleotide products are immobilized.

94. The method of any of claims 67-89, wherein neither the oligoacceptor substrate nor the terminal deoxynucleotidyl transferase of template-independent polymerase are immobilized.

95. The method of any claims 67-94, further comprising an aqueous liquid phase.

96. The engineered terminal deoxynucleotidyl transferase of any of claims 1-54, wherein the engineered terminal deoxynucleotidyl transferase is immobilized.
